SEQUENCE LISTING (193 sequences)

SEQ ID NO: 1 - protein, 214 aa, synthetic construct
  REGION 1..214: note = Synthetic: 9G4 kappa LC (aka. ILT4.1-kappa)
EIVLTQSPATLSLSPGERATLSCRASQSVSSYLAWYQQKPGQAPRLLIYDASNRATGIPARFSGSGSGTDFTLTISSLEPEDFAGYYCQQRSYWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, synthetic construct
  REGION 1..446: note = Synthetic: 9G4-IgG1.1f HC (aka. ILT4.1-IgG1.1f)
QVQLQESGPGLVKPSETLSLTCTVSGGSISSYYWNWIRQPPGKGLEWLGYIYYSGSTKYNPSLKSRVTISVDTSKNQFSLKLSSVTAADTAVYYCASSGWYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
  REGION 1..214: note = Synthetic: 9C8 kappa LC ILT4.2-kappa
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 455 aa, synthetic construct
  REGION 1..455: note = Synthetic: 9C8-IgG1.1f HC ILT4.2-IgG1.1f
QVQLVQSGAEVKKPGASVKVSCKASGYTFTSSDINWVRQATGQGLEWMGWMNPNSGHTGYAQKFQDRVTLTRDTSISTAYMELSSLRSEDSAVYYCARGGNSIDWGFSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, synthetic construct
  REGION 1..214: note = Synthetic: 2H2 kappa LC ILT4.3-kappa
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 458 aa, synthetic construct
  REGION 1..458: note = Synthetic: 2H2-IgG1.1fHC ILT4.3-IgG1.1f
QVQLVQSGAEVKKPGSSVEVSCKASGGTFSNYAISWVRQAPGQGLEWMGGIIPILATANYAPKFQGRVTITADEFTSSAYMELSSLRSEDTAVYYCAKSSITMIRGAYLYYYDGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 215 aa, synthetic construct
  REGION 1..215: note = Synthetic: 2E5 kappa LC ILT4.4-kappa
EIVLTQSPATLSLSPGERATLSCRASQSVSSYLAWYQQKPGQAPRLLIYDASNRATGIPARFSGSGSGTDFTLTISSLEPEDFAVYYCQQRSNWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, synthetic construct
  REGION 1..451: note = Synthetic: 2E5-IgG1.1f HC ILT4.4-IgG1.1f
QVQLVESGGGVVQPGRSLRLSCTASGFTFSNYGMHWVRQAPGKGLEWVAVIWYDGSNEYYAESVKGRLTISRDNSKNTLYLQVNSLRAEDTAVYYCARDPFYGSGNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, synthetic construct
  REGION 1..214: note = Synthetic: 24E5 kappa LC ILT4.5-kappa
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
  REGION 1..451: note = Synthetic: 24E5-IgG1.1 HC ILT4.5-IgG1.1f
EVQLLESGGGLVQPGGSLRLSCAASGFTFSSYVMSWVRQAPGKGLEWVSGISGSGGSTYYADSVKGRFTISRDNSKNTLYLQMNSLRAEDTAVYYCAKDQDIIAAYYFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, synthetic construct
  REGION 1..215: note = Synthetic: 21D9 kappa LC ILT4.6-kappa
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 455 aa, synthetic construct
  REGION 1..455: note = Synthetic: 21D9-IgG1.1f HC ILT4.6-IgG1.1f
EGQLLESGGDLVQPGGSLRLSCATSGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 454 aa, synthetic construct
  REGION 1..454: note = Synthetic: 21D9.e.IgG1.3 HC ILT4.8.IgG1.3
EVQLLESGGGLVQPGGSLRLSCAASGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 212 aa, synthetic construct
  REGION 1..212: note = Synthetic: 21A5 kappa LC
EIALTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 453 aa, synthetic construct
  REGION 1..453: note = Synthetic: 21A5.IgG1.3 HC
QVQLVQSGAEVKKPGASVKVSCKASGYTFTDYYLHWVRQAPGQGLEWMGIFHPSGDITSSAQNFQGRVTMIRDTSTSTVYMELSSLRSEDTAVYYCARGGVLRYLDWSHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 212 aa, synthetic construct
  REGION 1..212: note = Synthetic: 21A5.1 kappa LC (ILT4.9.1K)
EIVLTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 453 aa, synthetic construct
  REGION 1..453: note = Synthetic: 21A5.a.IgG1.3 HC (ILT4.9.IgG1.3)
QVQLVQSGAEVKKPGASVKVSCKASGYTFTDYYLHWVRQAPGQGLEWMGIFHPSGDITSSAQNFQGRVTMTRDTSTSTVYMELSSLRSEDTAVYYCARGGVLRYLDWSHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 214 aa, synthetic construct
  REGION 1..214: note = Synthetic: 10F10 kappa LC
AIQLTQSPSSLSASVGDRVTITCRASQGISSALAWFQQKPGKAPKLLIYDASSLESGVPSRFTGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 445 aa, synthetic construct
  REGION 1..445: note = Synthetic: 10F10.IgG1.3 HC ILT4.10.IgG1.3
QVQLVESGGGVVQPGRSLRLSCAASGFTFSSYAMHWVRQAPGKGLEWVAIISYDEYNKYYADSVKGRFTISRDNSKNTLYLQMNSLRAEDTAVYYCAREWVGIRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 214 aa, synthetic construct
  REGION 1..214: note = Synthetic: 10F10.1 kappa LC
AIQLTQSPSSLSASVGDRVTITCRASQGISSALAWYQQKPGKAPKLLIYDASSLESGVPSRFTGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 214 aa, synthetic construct
  REGION 1..214: note = Synthetic: 10F10.3 kappa LC
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 645 bp, synthetic construct
  misc_feature 1..645: note = Synthetic: 9G4 LC DNA ILT4.1-kappa DNA
  source 1..645: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcgtccaacagggccactggcatcccagccaggttcagtggcagtgggtctgggacagacttcactctcaccatcagcagcctagagcctgaagattttgcaggttattactgtcagcagcgtagctactggccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 23 - DNA, 1341 bp, synthetic construct
  source 1..1341: mol_type = other DNA
  misc_feature 1..1341: note = Synthetic: 9G4 IgG1.1f HC DNA ILT4.1-IgG1.1f DNA
caggtgcagctgcaggagtcgggcccaggactggtgaagccttcggagaccctgtccctcacctgcactgtctctggtggctccatcagtagttactactggaactggattcggcagcccccagggaagggactggagtggcttgggtacatctattacagtgggagtaccaagtacaacccctccctcaagagtcgagtcaccatatcagtagacacgtccaagaaccagttctccctgaagctgagctctgtgaccgctgcggacacggccgtgtattattgtgccagcagtggctggtactactttgactattggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 24 - DNA, 645 bp, synthetic construct
  misc_feature 1..645: note = Synthetic: 9C8 kappa LC DNA ILT4.2-kappa DNA
  source 1..645: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 25 - DNA, 1368 bp, synthetic construct
  source 1..1368: mol_type = other DNA
  misc_feature 1..1368: note = Synthetic: 9C8 IgG1.1f DNA ILT4.2-IgG1.1f DNA
caggtgcaactggtgcagtctggggctgaggtaaagaagcctggggcctcagtgaaggtctcctgcaaggcttctggatacaccttcaccagctctgatatcaactgggtgcgacaggccactggacaagggcttgagtggatgggatggatgaaccctaacagtggtcacacaggctatgcacagaagttccaggacagagtcaccttgacccgggacacctccataagcacagcctacatggagctgagcagcctgagatctgaggactcggccgtgtattactgtgcgagaggtgggaatagcattgactgggggttctcctactacggtctggacgtctggggccaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 26 - DNA, 645 bp, synthetic construct
  source 1..645: mol_type = other DNA
  misc_feature 1..645: note = Synthetic: 2H2 kappa LC DNA ILT4.3-kappa DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcgtacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - DNA, 1377 bp, synthetic construct
  misc_feature 1..1377: note = Synthetic: 2H2 IgG1.1f HC DNA ILT4.3-IgG1.1f DNA
  source 1..1377: mol_type = other DNA
caggtccagttggtgcagtctggggctgaggtgaagaagcctgggtcctcggtggaggtctcctgcaaggcttctgggggcaccttcagcaactatgctatcagctgggtgcgacaggcccctggacaagggcttgagtggatgggagggatcatccccatcttggctacagcaaactacgcaccgaagttccagggcagagtcacgattaccgcggacgaattcacgagctcagcttacatggagctgagcagcctgagatctgaggacacggccgtgtattactgtgcgaagtctagtattactatgattcggggagcctatctttactactacgacggtatggacgtctggggccaagggaccacggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 28 - DNA, 648 bp, synthetic construct
  misc_feature 1..648: note = Synthetic: 2E5 kappa LC DNA ILT4.4-kappa DNA
  source 1..648: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtggacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 29 - DNA, 1356 bp, synthetic construct
  misc_feature 1..1356: note = Synthetic: 2E5 IgG1.1f HC DNA ILT4.4-IgG1.1f DNA
  source 1..1356: mol_type = other DNA
caggtgcagctggtggagtctgggggaggcgtggtccagcctgggaggtccctgagactctcctgtacagcgtctggattcaccttcagtaactatggcatgcactgggtccgccaggctccaggcaagggactggagtgggtggcagttatctggtatgatggaagtaatgaatactatgcagaatccgtgaagggccgactcaccatctccagagacaattccaagaacacgctgtatctgcaagtgaacagcctgagagccgaggacacggctgtgtattattgtgcgagagatcctttctatggttcggggaattactttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 30 - DNA, 645 bp, synthetic construct
  source 1..645: mol_type = other DNA
  misc_feature 1..645: note = Synthetic: 24E5 kappa LC DNA ILT4.5-kappa DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 31 - DNA, 1356 bp, synthetic construct
  source 1..1356: mol_type = other DNA
  misc_feature 1..1356: note = Synthetic: 24E5 IgG1.1f HC DNA ILT4.5-IgG1.1f DNA
gaggtgcagctgttggagtctgggggaggcttggtacagcctggggggtccctgagactctcctgtgcagcctctggattcacctttagcagctatgtcatgagctgggtccgccaggctccagggaaggggctggagtgggtctcaggtattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatcaggatattatagcagcatactactttgtctactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 32 - DNA, 648 bp, synthetic construct
  source 1..648: mol_type = other DNA
  misc_feature 1..648: note = Synthetic: 21D9 kappa LC DNA ILT4.6-kappa DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 33 - DNA, 1368 bp, synthetic construct
  source 1..1368: mol_type = other DNA
  misc_feature 1..1368: note = Synthetic: 21D9 IgG1.1f HC DNA ILT4.6-IgG1.1f DNA
gagggacagctgttggagtctgggggagacttggtacagcctggggggtccctgagactctcctgtgcaacctctggattcacctttagcaactatgccatgaactgggtccgccaggctccagggaaggggctggagtggatctcagttattagtgttagtggtggtagcacatactacgcagactccgtgaagggccggttcaccatctccagagacaattccaagaacacgctgtatctgcaaatgaatagcctgagagccgaggacacggccgtatattactgtgcgaaagattattactatgattcggggagttattatgactctttctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaagcagcatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 34 - DNA, 1365 bp, synthetic construct
  misc_feature 1..1365: note = Synthetic: 21D9.IgG1.3 HC DNA ILT4.6-IgG1.3 HC DNA
  source 1..1365: mol_type = other DNA
gagggacagctgttggagtctgggggagacttggtacagcctggggggtccctgagactctcctgtgcaacctctggattcacctttagcaactatgccatgaactgggtccgccaggctccagggaaggggctggagtggatctcagttattagtgttagtggtggtagcacatactacgcagactccgtgaagggccggttcaccatctccagagacaattccaagaacacgctgtatctgcaaatgaatagcctgagagccgaggacacggccgtatattactgtgcgaaagattattactatgattcggggagttattatgactctttctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 35 - DNA, 1365 bp, synthetic construct
  misc_feature 1..1365: note = Synthetic: 21D9.b.IgG1.3 HC DNA
  source 1..1365: mol_type = other DNA
gaggtgcagctgctggagtctggaggaggactggtgcagccaggaggcagcctgaggctgtcctgcgccacctctggcttcacattttccaactatgccatgaattgggtgcgccaggcacctggcaagggactggagtggatctctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 36 - protein, 454 aa, synthetic construct
  REGION 1..454: note = Synthetic: 21D9.b.IgG1.3 HC
EVQLLESGGGLVQPGGSLRLSCATSGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - DNA, 1365 bp, synthetic construct
  source 1..1365: mol_type = other DNA
  misc_feature 1..1365: note = Synthetic: 21D9.c.IgG1.3 HC DNA
gaggtgcagctgctggagtctggaggagacctggtgcagccaggaggcagcctgaggctgtcctgcgccgcctctggcttcacattttccaactatgccatgaattgggtgcgccaggcacctggcaagggactggagtggatctctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 38 - protein, 454 aa, synthetic construct
  REGION 1..454: note = Synthetic: 21D9.c.IgG1.3 HC
EVQLLESGGDLVQPGGSLRLSCAASGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - DNA, 1365 bp, synthetic construct
  misc_feature 1..1365: note = Synthetic: 21D9.d.IgG1.3 HC DNA
  source 1..1365: mol_type = other DNA
gaggtgcagctgctggagtctggaggagacctggtgcagccaggaggcagcctgaggctgtcctgcgccacctctggcttcacattttccaactatgccatgaattgggtgcgccaggcacctggcaagggactggagtgggtgtctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 40 - protein, 454 aa, synthetic construct
  REGION 1..454: note = Synthetic: 21D9.d.IgG1.3 HC
EVQLLESGGDLVQPGGSLRLSCATSGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - DNA, 1413 bp, synthetic construct
  misc_feature 1..1413: note = Synthetic: 21D9.e.IgG1.3 HC DNA ILT4.8.IgG1.3 HC DNA With signal peptide
  source 1..1413: mol_type = other DNA
atgagggcttggatcttctttctgctctgcctggccgggagagcgctcgcagaggtgcagctgttggagtctgggggaggcttggtacagcctggggggtccctgagactctcctgtgcagcctctggattcacctttagcaactatgccatgaactgggtccgccaggctccagggaaggggctggagtgggtctcagttattagtgttagtggtggtagcacatactacgcagactccgtgaagggccggttcaccatctccagagacaattccaagaacacgctgtatctgcaaatgaatagcctgagagccgaggacacggccgtatattactgtgcgaaagattattactatgattcggggagttattatgactctttctttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 42 - DNA, 1020 bp, synthetic construct
  source 1..1020: mol_type = other DNA
  misc_feature 1..1020: note = Synthetic: 21A5 LC DNA
gagatcgtgctgacccagagcccaggcacactgtctctgagcccaggagagagggccaccctgtcctgcagagcctcccagtctgtgagctcctcttacctggcctggtatcagcagaagccaggacaggcacctaggctgctgatctacggagccagctccagggcaaccggcatccctgaccgcttcagcggctccggctctggcacagacttcaccctgacaatctctaggctggagcccgaggacttcgccgtgtactattgtcagcagtatggctccacctttggcggcggcacaaaggtggagatcaaggaattcgccaccatgagggcttggatcttctttctgctctgcctggccgggcgcgccctcgcagagatcgtgctgacccagagcccaggcacactgtctctgagcccaggagagagggccaccctgtcctgcagagcctcccagtctgtgagctcctcttacctggcctggtatcagcagaagccaggacaggcacctaggctgctgatctacggagccagctccagggcaaccggcatccctgaccgcttcagcggctccggctctggcacagacttcaccctgacaatctctaggctggagcccgaggacttcgccgtgtactattgtcagcagtatggctccacctttggcggcggcacaaaggtggagatcaagcgtacggtggccgccccgtccgtgtttatcttccctccatcggacgagcagctcaagtccggtaccgcgagcgtggtctgcctgctgaacaatttctacccgcgcgaagctaaagtgcaatggaaggtcgataacgcacttcagtccgggaacagccaggaatctgtgaccgagcaggactccaaggattcgacctattccctgtcctcgactctcaccctgtcaaaggccgactacgagaagcacaaggtctacgcctgcgaagtgacccatcagggcttgtcctcacccgtgactaagagcttcaaccggggagagtgttagtga

SEQ ID NO: 43 - DNA, 1362 bp, synthetic construct
  misc_feature 1..1362: note = Synthetic: 21A5.IgG1.3 HC DNA
  source 1..1362: mol_type = other DNA
caggtgcagctggtgcagtccggagcagaggtgaagaagccaggagcctctgtgaaggtgagctgcaaggcctccggctacaccttcacagattactatctgcactgggtgcggcaggcaccaggacagggactggagtggatgggcatcttccacccttctggcgacatcacaagctccgcccagaactttcagggccgggtgaccatgacaagagataccagcacatccaccgtgtacatggagctgtctagcctgaggtctgaggacaccgccgtgtactattgtgcaaggggaggcgtgctgaggtatctggactggagccacgcctttgatatctggggccagggcacaatggtgaccgtgtcctctgctagcaccaaaggaccttcagtgttcccgctcgcgccgtcatccaagtccacctccggcggaaccgctgccctgggatgccttgtgaaggactacttccctgagcctgtgactgtgtcctggaactccggagccctgacctccggcgtgcataccttccctgctgtgctgcaatcaagcggcctctactcattgagctccgtcgtgaccgtgccgagctccagcctcggtactcaaacctacatctgcaatgtcaaccacaagcccagcaacaccaaggtcgataagagagtggagcccaagtcgtgcgacaagactcacacttgtcccccatgcccggcccccgaggccgagggggccccgagcgtctttctgttcccgcctaagcccaaggataccctgatgatttcgcggactcccgaagtgacctgtgtcgtggtggacgtgtcccacgaagatcccgaagtcaagttcaactggtacgtggacggagtcgaggtgcacaacgcaaagaccaagcctcgcgaggaacagtacaactcgacctatcgggtggtgtccgtgctgacagtgctgcatcaggactggctcaacggaaaggagtacaagtgcaaagtgtccaacaaggccctgccagcgcccattgaaaagactatcagcaaggccaaggggcagccaagggaaccccaagtgtacaccctgcctccgtcccgcgaagaaatgaccaagaaccaggtgtccctgacgtgcttggtcaagggcttttacccttccgacatcgccgtggagtgggagtcgaacgggcagccggaaaacaactacaagaccactccaccggtgctcgactcggatggctcgttcttcctgtattcgaagctgactgtcgacaaaagccggtggcagcagggcaatgtgttctcctgctccgtgatgcatgaggccctccacaaccactacacccagaagtctctgagcctttccccgggatga

SEQ ID NO: 44 - DNA, 687 bp, synthetic construct
  source 1..687: mol_type = other DNA
  misc_feature 1..687: note = Synthetic: 21A5.1 kappa LC DNA (ILT4.9.1K) DNA With signal peptide
atgagggcttggatcttctttctgctctgcctggccgggcgcgccttggccgaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 45 - DNA, 1410 bp, synthetic construct
  misc_feature 1..1410: note = Synthetic: 21A5.a.IgG1.3 HC DNA (ILT4.9.IgG1.3) DNA With signal peptide
  source 1..1410: mol_type = other DNA
atgagggcttggatcttctttctgctctgcctggccgggagagcgctcgcacaggtgcagctggtgcagtctggggctgaggtgaagaagcctggggcctcagtgaaggtttcctgcaaggcatctggatacaccttcaccgactactatttgcactgggtgcgacaggcccctggacaagggcttgagtggatgggaatattccaccctagtggtgatatcacaagctccgcacagaacttccagggcagagtcaccatgaccagggacacgtccacgagcacagtctacatggagctgagcagcctgagatctgaggacacggccgtgtattactgtgcgagaggaggcgtgctgaggtatctggactggagccatgcttttgatatctggggccaagggacaatggtcaccgtctcttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggt

SEQ ID NO: 46 - DNA, 693 bp, synthetic construct
  source 1..693: mol_type = other DNA
  misc_feature 1..693: note = Synthetic: 10F10 kappa LC DNA ILT4.10K DNA With signal peptide
atgagggcttggatcttctttctgctctgcctggccgggcgcgccctcgcagccatccagctgacccagtctcctagctccctgtctgccagcgtgggcgacagggtgaccatcacatgcagagcctcccagggaatctctagcgccctggcctggttccagcagaagccaggcaaggcccctaagctgctgatctacgatgcctcctctctggagagcggagtgccatccaggttcaccggctccggctctggcacagactttaccctgacaatcagctccctgcagccagaggattttgccacatactattgtcagcagttcaacagctatcccatcacctttggccagggcacacggctggagatcaagcgtacggtggccgccccgtccgtgtttatcttccctccatcggacgagcagctcaagtccggtaccgcgagcgtggtctgcctgctgaacaatttctacccgcgcgaagctaaagtgcaatggaaggtcgataacgcacttcagtccgggaacagccaggaatctgtgaccgagcaggactccaaggattcgacctattccctgtcctcgactctcaccctgtcaaaggccgactacgagaagcacaaggtctacgcctgcgaagtgacccatcagggcttgtcctcacccgtgactaagagcttcaaccggggagagtgt

SEQ ID NO: 47 - DNA, 1386 bp, synthetic construct
  source 1..1386: mol_type = other DNA
  misc_feature 1..1386: note = Synthetic: 10F10.IgG1.3 HC DNA ILT4.10.IgG1.3 DNA With signal peptide
atgagggcttggatcttctttctgctctgcctggccgggcgcgccctcgcacaggtgcagctggtggagtctggaggaggagtggtgcagccaggccggtccctgagactgtcttgcgccgccagcggcttcacctttagctcctacgcaatgcactgggtgaggcaggcacctggcaagggactggagtgggtggccatcatcagctacgacgagtataacaagtactatgccgattccgtgaagggcaggttcaccatctcccgcgacaactctaagaatacactgtacctgcagatgaatagcctgagagccgaggatacagccgtgtactattgtgcccgggagtgggtgggcatcagatattggggccagggcaccctggtgacagtgtctagcgctagcaccaaaggaccttcagtgttcccgctcgcgccgtcatccaagtccacctccggcggaaccgctgccctgggatgccttgtgaaggactacttccctgagcctgtgactgtgtcctggaactccggagccctgacctccggcgtgcataccttccctgctgtgctgcaatcaagcggcctctactcattgagctccgtcgtgaccgtgccgagctccagcctcggtactcaaacctacatctgcaatgtcaaccacaagcccagcaacaccaaggtcgataagagagtggagcccaagtcgtgcgacaagactcacacttgtcccccatgcccggcccccgaggccgagggggccccgagcgtctttctgttcccgcctaagcccaaggataccctgatgatttcgcggactcccgaagtgacctgtgtcgtggtggacgtgtcccacgaagatcccgaagtcaagttcaactggtacgtggacggagtcgaggtgcacaacgcaaagaccaagcctcgcgaggaacagtacaactcgacctatcgggtggtgtccgtgctgacagtgctgcatcaggactggctcaacggaaaggagtacaagtgcaaagtgtccaacaaggccctgccagcgcccattgaaaagactatcagcaaggccaaggggcagccaagggaaccccaagtgtacaccctgcctccgtcccgcgaagaaatgaccaagaaccaggtgtccctgacgtgcttggtcaagggcttttacccttccgacatcgccgtggagtgggagtcgaacgggcagccggaaaacaactacaagaccactccaccggtgctcgactcggatggctcgttcttcctgtattcgaagctgactgtcgacaaaagccggtggcagcagggcaatgtgttctcctgctccgtgatgcatgaggccctccacaaccactacacccagaagtctctgagcctttccccggga

SEQ ID NO: 48 - DNA, 642 bp, synthetic construct
  misc_feature 1..642: note = Synthetic: 10F10.1 kappa LC DNA
  source 1..642: mol_type = other DNA
gccatccagctgacccagtctcctagctccctgtctgccagcgtgggcgacagggtgaccatcacatgcagagcctcccagggaatctctagcgccctggcctggtaccagcagaagccaggcaaggcccctaagctgctgatctatgatgcctcctctctggagagcggagtgccatccaggttcaccggctccggctctggcacagactttaccctgacaatcagctccctgcagccagaggatttcgccacatactattgtcagcagttcaacagctaccccatcacctttggccagggcacacggctggagatcaagcgtacggtggccgccccgtccgtgtttatcttccctccatcggacgagcagctcaagtccggtaccgcgagcgtggtctgcctgctgaacaatttctacccgcgcgaagctaaagtgcaatggaaggtcgataacgcacttcagtccgggaacagccaggaatctgtgaccgagcaggactccaaggattcgacctattccctgtcctcgactctcaccctgtcaaaggccgactacgagaagcacaaggtctacgcctgcgaagtgacccatcagggcttgtcctcacccgtgactaagagcttcaaccggggagagtgt

SEQ ID NO: 49 - DNA, 693 bp, synthetic construct
  source 1..693: mol_type = other DNA
  misc_feature 1..693: note = Synthetic: 10F10.3 kappa LC DNA ILT4.11K DNA With signal peptide
atgagggcttggatcttctttctgctctgcctggccgggcgcgccctcgcagccatccagctgacccagtctcctagctccctgtctgccagcgtgggcgacagggtgaccatcacatgcagagcctcccagggaatctctagcgccctggcctggtaccagcagaagccaggcaaggcccctaagctgctgatctatgatgcctcctctctggagagcggagtgccatccaggttcagcggctccggctctggcacagactttaccctgacaatcagctccctgcagccagaggatttcgccacatactattgtcagcagttcaacagctaccccatcacctttggccagggcacacggctggagatcaagcgtacggtggccgccccgtccgtgtttatcttccctccatcggacgagcagctcaagtccggtaccgcgagcgtggtctgcctgctgaacaatttctacccgcgcgaagctaaagtgcaatggaaggtcgataacgcacttcagtccgggaacagccaggaatctgtgaccgagcaggactccaaggattcgacctattccctgtcctcgactctcaccctgtcaaaggccgactacgagaagcacaaggtctacgcctgcgaagtgacccatcagggcttgtcctcacccgtgactaagagcttcaaccggggagagtgt

SEQ ID NO: 50 - protein, 125 aa, synthetic construct
  REGION 1..125: note = Synthetic: 9G4 VL ILT4.1K VL (including signal sequence, residues 1-18)
APAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAGYYCQQRSYWPWTFGQGTKVEIK

SEQ ID NO: 51 - protein, 135 aa, synthetic construct
  REGION 1..135: note = Synthetic: 9G4.IgG1.1 VH ILT4.1.IgG1.1 VH (including signal sequence, residues 1-19)
MKHLWFFLLLVAAPRWVLSQVQLQESGPGLVKPSETLSLTCTVSGGSISSYYWNWIRQPPGKGLEWLGYIYYSGSTKYNPSLKSRVTISVDTSKNQFSLKLSSVTAADTAVYYCASSGWYYFDYWGQGTLVTVSS

SEQ ID NO: 52 - DNA, 375 bp, synthetic construct
  source 1..375: mol_type = other DNA
  misc_feature 1..375: note = Synthetic: 9G4 VL DNA ILT4.1K VL DNA
gccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcgtccaacagggccactggcatcccagccaggttcagtggcagtgggtctgggacagacttcactctcaccatcagcagcctagagcctgaagattttgcaggttattactgtcagcagcgtagctactggccgtggacgttcggccaagggaccaaggtggaaatcaaa

SEQ ID NO: 53 - DNA, 405 bp, synthetic construct
  misc_feature 1..405: note = Synthetic: 9G4.IgG1.1 VH DNA ILT4.1.IgG1.1 VH DNA
  source 1..405: mol_type = other DNA
atgaaacatctgtggttcttccttctcctggtggcagctcccagatgggtcctgtcccaggtgcagctgcaggagtcgggcccaggactggtgaagccttcggagaccctgtccctcacctgcactgtctctggtggctccatcagtagttactactggaactggatccggcagcccccagggaagggactggagtggcttgggtacatctattacagtgggagtaccaagtacaacccctccctcaagagtcgagtcaccatatcagtagacacgtccaagaaccagttctccctgaagctgagctctgtgaccgctgcggacacggccgtgtattattgtgccagcagtggctggtactactttgactattggggccagggaaccctggtcaccgtctcctca

SEQ ID NO: 54 - protein, 107 aa, synthetic construct
  REGION 1..107: note = Synthetic: 9C8 kappa VL ILT4.2K VL
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 55 - protein, 125 aa, synthetic construct
  REGION 1..125: note = Synthetic: 9C8.IgG1.1 VH ILT4.2.IgG1.1 VH
QVQLVQSGAEVKKPGASVKVSCKASGYTFTSSDINWVRQATGQGLEWMGWMNPNSGHTGYAQKFQDRVTLTRDTSISTAYMELSSLRSEDSAVYYCARGGNSIDWGFSYYGLDVWGQGTTVTVSS

SEQ ID NO: 56 - DNA, 321 bp, synthetic construct
  source 1..321: mol_type = other DNA
  misc_feature 1..321: note = Synthetic: 9C8 kappa VL DNA ILT4.2K VL DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 57 - DNA, 375 bp, synthetic construct
  misc_feature 1..375: note = Synthetic: 9C8.IgG1.1 VH DNA ILT4.2.IgG1.1 VH DNA
  source 1..375: mol_type = other DNA
caggtgcaactggtgcagtctggggctgaggtaaagaagcctggggcctcagtgaaggtctcctgcaaggcttctggatacaccttcaccagctctgatatcaactgggtgcgacaggccactggacaagggcttgagtggatgggatggatgaaccctaacagtggtcacacaggctatgcacagaagttccaggacagagtcaccttgacccgggacacctccataagcacagcctacatggagctgagcagcctgagatctgaggactcggccgtgtattactgtgcgagaggtgggaatagcattgactgggggttctcctactacggtctggacgtctggggccaagggaccacggtcaccgtctcctca

SEQ ID NO: 58 - protein, 128 aa, synthetic construct
  REGION 1..128: note = Synthetic: 2H2.IgG1.1 VH ILT4.3.IgG1.1 VH
QVQLVQSGAEVKKPGSSVEVSCKASGGTFSNYAISWVRQAPGQGLEWMGGIIPILATANYAPKFQGRVTITADEFTSSAYMELSSLRSEDTAVYYCAKSSITMIRGAYLYYYDGMDVWGQGTTVTVSS

SEQ ID NO: 59 - protein, 107 aa, synthetic construct
  REGION 1..107: note = Synthetic: 2H2 kappa VL ILT4.3K VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSYTFGQGTKLEIK

SEQ ID NO: 60 - DNA, 384 bp, synthetic construct
  source 1..384: mol_type = other DNA
  misc_feature 1..384: note = Synthetic: 2H2.IgG1.1 VH DNA ILT4.3.IgG1.1 VH DNA
caggtccagttggtgcagtctggggctgaggtgaagaagcctgggtcctcggtggaggtctcctgcaaggcttctgggggcaccttcagcaactatgctatcagctgggtgcgacaggcccctggacaagggcttgagtggatgggagggatcatccccatcttggctacagcaaactacgcaccgaagttccagggcagagtcacgattaccgcggacgaattcacgagctcagcttacatggagctgagcagcctgagatctgaggacacggccgtgtattactgtgcgaagtctagtattactatgattcggggagcctatctttactactacgacggtatggacgtctggggccaagggaccacggtcaccgtctcctca

SEQ ID NO: 61 - DNA, 321 bp, synthetic construct
  misc_feature 1..321: note = Synthetic: 2H2 kappa VL DNA ILT4.3K VL DNA
  source 1..321: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcgtacacttttggccaggggaccaagctggagatcaaa

SEQ ID NO: 62 - protein, 108 aa, synthetic construct
  REGION 1..108: note = Synthetic: 2E5 kappa VL ILT4.4K VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPWTFGQGTKVEIK

SEQ ID NO: 63 - protein, 121 aa, synthetic construct
  REGION 1..121: note = Synthetic: 2E5.IgG1.1 VH ILT4.4.IgG1.1 VH
QVQLVESGGGVVQPGRSLRLSCTASGFTFSNYGMHWVRQAPGKGLEWVAVIWYDGSNEYYAESVKGRLTISRDNSKNTLYLQVNSLRAEDTAVYYCARDPFYGSGNYFDYWGQGTLVTVSS

SEQ ID NO: 64 - DNA, 324 bp, synthetic construct
  source 1..324: mol_type = other DNA
  misc_feature 1..324: note = Synthetic: 2E5 kappa VL DNA ILT4.4K VL
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtggacgttcggccaagggaccaaggtggaaatcaaa

SEQ ID NO: 65 - DNA, 363 bp, synthetic construct
  source 1..363: mol_type = other DNA
  misc_feature 1..363: note = Synthetic: 2E5.IgG1.1 VH DNA ILT4.4.IgG1.1 VH
caggtgcagctggtggagtctgggggaggcgtggtccagcctgggaggtccctgagactctcctgtacagcgtctggattcaccttcagtaactatggcatgcactgggtccgccaggctccaggcaagggactggagtgggtggcagttatctggtatgatggaagtaatgaatactatgcagaatccgtgaagggccgactcaccatctccagagacaattccaagaacacgctgtatctgcaagtgaacagcctgagagccgaggacacggctgtgtattattgtgcgagagatcctttctatggttcggggaattactttgactactggggccagggaaccctggtcaccgtctcctca

SEQ ID NO: 66 - protein, 107 aa, synthetic construct
  REGION 1..107: note = Synthetic: 24E5 kappa VL ILT4.5K VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 67 - protein, 121 aa, synthetic construct
  REGION 1..121: note = Synthetic: 24E5.IgG1.1 VH ILT4.5.IgG1.1 VH
EVQLLESGGGLVQPGGSLRLSCAASGFTFSSYVMSWVRQAPGKGLEWVSGISGSGGSTYYADSVKGRFTISRDNSKNTLYLQMNSLRAEDTAVYYCAKDQDIIAAYYFVYWGQGTLVTVSS

SEQ ID NO: 68 - DNA, 321 bp, synthetic construct
  misc_feature 1..321: note = Synthetic: 24E5 kappa VL DNA ILT4.5K VL
  source 1..321: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctctcactttcggcggagggaccaaggtggagatcaaa

SEQ ID NO: 69 - DNA, 363 bp, synthetic construct
  source 1..363: mol_type = other DNA
  misc_feature 1..363: note = Synthetic: 24E5.IgG1.1 VH DNA ILT4.5.IgG1.1 VH
gaggtgcagctgttggagtctgggggaggcttggtacagcctggggggtccctgagactctcctgtgcagcctctggattcacctttagcagctatgtcatgagctgggtccgccaggctccagggaaggggctggagtgggtctcaggtattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatcaggatattatagcagcatactactttgtctactggggccagggaaccctggtcaccgtctcctca

SEQ ID NO: 70 - protein, 108 aa, synthetic construct
  REGION 1..108: note = Synthetic: 21D9K kappa VL ILT4.6K VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 71 - protein, 125 aa, synthetic construct
  REGION 1..125: note = Synthetic: 21D9.IgG1.1 VH ILT4.6.IgG1.1 VH
EGQLLESGGDLVQPGGSLRLSCATSGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSS

SEQ ID NO: 72 - DNA, 324 bp, synthetic construct
  misc_feature 1..324: note = Synthetic: 21D9K kappa VL DNA ILT4.6K VL DNA
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggcggagggaccaaggtggagatcaaa

SEQ ID NO: 73 - DNA, 375 bp, synthetic construct
  source 1..375: mol_type = other DNA
  misc_feature 1..375: note = Synthetic: 21D9.IgG1.1 VH DNA ILT4.6.IgG1.1 VH DNA
gagggacagctgttggagtctgggggagacttggtacagcctggggggtccctgagactctcctgtgcaacctctggattcacctttagcaactatgccatgaactgggtccgccaggctccagggaaggggctggagtggatctcagttattagtgttagtggtggtagcacatactacgcagactccgtgaagggccggttcaccatctccagagacaattccaagaacacgctgtatctgcaaatgaatagcctgagagccgaggacacggccgtatattactgtgcgaaagattattactatgattcggggagttattatgactctttctttgactactggggccagggaaccctggtcaccgtctcctca

SEQ ID NO: 74 - protein, 125 aa, synthetic construct
  REGION 1..125: note = Synthetic: 21D9.b.IgG1.3 VH
EVQLLESGGGLVQPGGSLRLSCATSGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSS

SEQ ID NO: 75 - protein, 125 aa, synthetic construct
  REGION 1..125: note = Synthetic: 21D9.c.IgG1.3 VH
EVQLLESGGDLVQPGGSLRLSCAASGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSS

SEQ ID NO: 76 - DNA, 375 bp, synthetic construct
  misc_feature 1..375: note = Synthetic: 21D9.b.IgG1.3 VH DNA
  source 1..375: mol_type = other DNA
gaggtgcagctgctggagtctggaggaggactggtgcagccaggaggcagcctgaggctgtcctgcgccacctctggcttcacattttccaactatgccatgaattgggtgcgccaggcacctggcaagggactggagtggatctctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctcc

SEQ ID NO: 77 - DNA, 375 bp, synthetic construct
  misc_feature 1..375: note = Synthetic: 21D9.c.IgG1.3 VH DNA
  source 1..375: mol_type = other DNA
gaggtgcagctgctggagtctggaggagacctggtgcagccaggaggcagcctgaggctgtcctgcgccgcctctggcttcacattttccaactatgccatgaattgggtgcgccaggcacctggcaagggactggagtggatctctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctcc

SEQ ID NO: 78 - protein, 125 aa, synthetic construct
  REGION 1..125: note = Synthetic: 21D9.d.IgG1.3 VH
EVQLLESGGDLVQPGGSLRLSCATSGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSS

SEQ ID NO: 79 - DNA, 375 bp, synthetic construct
  misc_feature 1..375: note = Synthetic: 21D9.d.IgG1.3 VH DNA
  source 1..375: mol_type = other DNA
gaggtgcagctgctggagtctggaggagacctggtgcagccaggaggcagcctgaggctgtcctgcgccacctctggcttcacattttccaactatgccatgaattgggtgcgccaggcacctggcaagggactggagtgggtgtctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctcc

SEQ ID NO: 80 - protein, 125 aa, synthetic construct
  REGION 1..125: note = Synthetic: 21D9.e.IgG1.3 VH ILT4.8.IgG1.3 VH
EVQLLESGGGLVQPGGSLRLSCAASGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSS

SEQ ID NO: 81 - DNA, 375 bp, synthetic construct
  misc_feature 1..375: note = Synthetic: 21D9.e.IgG1.3 VH DNA ILT4.8.IgG1.3 VH DNA
  source 1..375: mol_type = other DNA
gaggtgcagctgctggagtctggaggaggactggtgcagccaggaggcagcctgaggctgtcctgcgccgcctctggcttcacattttccaactatgccatgaattgggtgcgccaggcacctggcaagggactggagtgggtgtctgtgatctctgtgagcggcggctctacctactatgccgacagcgtgaagggccggttcaccatcagcagagataactccaagaatacactgtatctgcagatgaacagcctgcgggccgaggacacagccgtgtactattgtgccaaggactactattacgattccggctcttattacgactccttctttgattactggggccagggcaccctggtgacagtgagctcc

SEQ ID NO: 82 - protein, 105 aa, synthetic construct
  REGION 1..105: note = Synthetic: 21A5 kappa VL
EIALTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIK

SEQ ID NO: 83 - protein, 124 aa, synthetic construct
  REGION 1..124: note = Synthetic: 21A5.IgG1.3 VH
QVQLVQSGAEVKKPGASVKVSCKASGYTFTDYYLHWVRQAPGQGLEWMGIFHPSGDITSSAQNFQGRVTMIRDTSTSTVYMELSSLRSEDTAVYYCARGGVLRYLDWSHAFDIWGQGTMVTVSS

SEQ ID NO: 84 - DNA, 315 bp, synthetic construct
  source 1..315: mol_type = other DNA
  misc_feature 1..315: note = Synthetic: 21A5 kappa VL DNA
gaaattgcgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagcactttcggcggagggaccaaggtggagatcaaa

SEQ ID NO: 85 - DNA, 372 bp, synthetic construct
  misc_feature 1..372: note = Synthetic: 21A5.IgG1.3 VH DNA
  source 1..372: mol_type = other DNA
caggtgcagctggtgcagtctggggctgaggtgaagaagcctggggcctcagtgaaggtttcctgcaaggcatctggatacaccttcaccgactactatttacactgggtgcgacaggcccctggacaagggcttgagtggatgggaatattccaccctagtggtgatatcacaagcagcgcacagaacttccagggcagagtcaccatgatcagggacacgtccacgagcaccgtctacatggaactgagcagcctgagatctgaagacacggccgtgtattactgtgcgagagggggtgtattacgatatcttgactggtcccatgcttttgatatctggggccaagggacaatggtcaccgtctcttca

SEQ ID NO: 86 - protein, 105 aa, synthetic construct
  REGION 1..105: note = Synthetic: 21A5.a kappa VL 21A5.1K ILT4.9K VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIK

SEQ ID NO: 87 - protein, 124 aa, synthetic construct
  REGION 1..124: note = Synthetic: 21A5.a.IgG1.3 VH ILT4.9.IgG1.3 VH
QVQLVQSGAEVKKPGASVKVSCKASGYTFTDYYLHWVRQAPGQGLEWMGIFHPSGDITSSAQNFQGRVTMTRDTSTSTVYMELSSLRSEDTAVYYCARGGVLRYLDWSHAFDIWGQGTMVTVSS

SEQ ID NO: 88 - DNA, 315 bp, synthetic construct
  misc_feature 1..315: note = Synthetic: 21A5.a kappa VL DNA 21A5.1K ILT4.9K VL
  source 1..315: mol_type = other DNA
gagatcgtgctgacccagagcccaggcacactgtctctgagcccaggagagagggccaccctgtcctgcagagcctcccagtctgtgagctcctcttacctggcctggtatcagcagaagccaggacaggcacctaggctgctgatctacggagccagctccagggcaaccggcatccctgaccgcttcagcggctccggctctggcacagacttcaccctgacaatctctaggctggagcccgaggacttcgccgtgtactattgtcagcagtatggctccacctttggcggcggcacaaaggtggagatcaag

SEQ ID NO: 89 - DNA, 372 bp, synthetic construct
  misc_feature 1..372: note = Synthetic: 21A5.a.IgG1.3 VH DNA ILT4.9.IgG1.3 VH
  source 1..372: mol_type = other DNA
caggtgcagctggtgcagtccggagcagaggtgaagaagccaggagcctctgtgaaggtgagctgcaaggcctccggctacaccttcacagattactatctgcactgggtgcggcaggcaccaggacagggactggagtggatgggcatcttccacccttctggcgacatcacaagctccgcccagaactttcagggccgggtgaccatgacaagagataccagcacatccaccgtgtacatggagctgtctagcctgaggtctgaggacaccgccgtgtactattgtgcaaggggaggcgtgctgaggtatctggactggagccacgcctttgatatctggggccagggcacaatggtgaccgtgtcctct

SEQ ID NO: 90 - protein, 107 aa, synthetic construct
  REGION 1..107: note = Synthetic: 10F10 kappa VL ILT4.10K VL
AIQLTQSPSSLSASVGDRVTITCRASQGISSALAWFQQKPGKAPKLLIYDASSLESGVPSRFTGSGSGTDFTLTISSLQPEDFATYYCQQFNSYPITFGQGTRLEIK

SEQ ID NO: 91 - protein, 116 aa, synthetic construct
  REGION 1..116: note = Synthetic: 10F10.IgG1.3 VH ILT4.10.IgG1.3 VH
QVQLVESGGGVVQPGRSLRLSCAASGFTFSSYAMHWVRQAPGKGLEWVAIISYDEYNKYYADSVKGRFTISRDNSKNTLYLQMNSLRAEDTAVYYCAREWVGIRYWGQGTLVTVSS

SEQ ID NO: 92 - DNA, 321 bp, synthetic construct
  source 1..321: mol_type = other DNA
  misc_feature 1..321: note = Synthetic: 10F10 kappa VL DNA ILT4.10K VL
gccatccagttgacccagtctccatcctccctgtctgcatctgtaggagacagagtcaccatcacttgccgggcaagtcagggcattagcagtgctttagcctggtttcagcagaaaccagggaaagctcctaagctcctgatctatgatgcctccagtttggaaagtggggtcccatcaaggttcaccggcagtggatctgggacagatttcactctcaccatcagcagcctgcagcctgaagattttgcaacttattactgtcaacagtttaatagttacccgatcaccttcggccaagggacacgactggagattaaa

SEQ ID NO: 93 - DNA, 348 bp, synthetic construct
  misc_feature 1..348: note = Synthetic: 10F10.IgG1.3 VH DNA ILT4.10.IgG1.3 VH
  source 1..348: mol_type = other DNA
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcaattatatcatatgatgaatacaataaatactacgcagactccgtgaagggccgattcaccatctccagagacaattccaagaacacgctgtatctgcaaatgaacagcctgagagctgaggacacggctgtgtattactgtgcgagagagtgggtggggatacgttactggggccagggaaccctggtcaccgtctcctca

SEQ ID NO: 94 - protein, 107 aa, synthetic construct
  REGION 1..107: note = Synthetic: 10F10.1 kappa VL
AIQLTQSPSSLSASVGDRVTITCRASQGISSALAWYQQKPGKAPKLLIYDASSLESGVPSRFTGSGSGTDFTLTISSLQPEDFATYYCQQFNSYPITFGQGTRLEIK

SEQ ID NO: 95 - DNA, 321 bp, synthetic construct
  source 1..321: mol_type = other DNA
  misc_feature 1..321: note = Synthetic: 10F10.1 kappa VL DNA
gccatccagctgacccagtctcctagctccctgtctgccagcgtgggcgacagggtgaccatcacatgcagagcctcccagggaatctctagcgccctggcctggtaccagcagaagccaggcaaggcccctaagctgctgatctatgatgcctcctctctggagagcggagtgccatccaggttcaccggctccggctctggcacagactttaccctgacaatcagctccctgcagccagaggatttcgccacatactattgtcagcagttcaacagctaccccatcacctttggccagggcacacggctggagatcaag

SEQ ID NO: 96 - protein, 107 aa, synthetic construct
  REGION 1..107: note = Synthetic: 10F10.3 kappa VL ILT4.11K VL
AIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIK

SEQ ID NO: 97 - DNA, 321 bp, synthetic construct
  source 1..321: mol_type = other DNA
  misc_feature 1..321: note = Synthetic: 10F10.3 kappa VL DNA ILT4.11K VL DNA
gccatccagctgacccagtctcctagctccctgtctgccagcgtgggcgacagggtgaccatcacatgcagagcctcccagggaatctctagcgccctggcctggtaccagcagaagccaggcaaggcccctaagctgctgatctatgatgcctcctctctggagagcggagtgccatccaggttcagcggctccggctctggcacagactttaccctgacaatcagctccctgcagccagaggatttcgccacatactattgtcagcagttcaacagctaccccatcacctttggccagggcacacggctggagatcaag

SEQ ID NO: 98 - protein, 330 aa, Homo sapiens
  REGION 1..330: note = misc_feature - Human IgG1f Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 993 bp, synthetic construct
  source 1..993: mol_type = other DNA
  misc_feature 1..993: note = Synthetic: IgG1f Constant Region DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 100 - protein, 329 aa, synthetic construct
  REGION 1..329: note = Synthetic: IgG1.3 (or IgG1.3f) heavy chain constant region (L234A, L235E, G237A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 107 aa, synthetic construct [sequence omitted]

SEQ ID NO: 102 - protein, 329 aa, Homo sapiens
  REGION 1..329: note = misc_feature - IgG1f (human wild-type allotype f) heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 329 aa, synthetic construct
  REGION 1..329: note = Synthetic: IgG1.1f Heavy chain constant region (L234A, L235E, G237A, A330S, P331S)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 329 aa, synthetic construct
  REGION 1..329: note = Synthetic: IgG1fa.P238K (or IgG1.P238K) heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGKSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - DNA, 990 bp, synthetic construct
  misc_feature 1..990: note = Synthetic: IgG1.3 heavy chain constant region DNA
  source 1..990: mol_type = other DNA
gcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgaaggggccccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 106 - DNA, 324 bp, synthetic construct
  misc_feature 1..324: note = Synthetic: Exemplary light chain constant region DNA
  source 1..324: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 107 - protein, 598 aa, Homo sapiens
  REGION 1..598: note = misc_feature - Human ILT4 precursor, with signal peptide
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 108 - protein, 577 aa, Homo sapiens
  REGION 1..577: note = misc_feature - Human mature ILT4, without signal peptide
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 109 - protein, 438 aa, Homo sapiens
  REGION 1..438: note = misc_feature - Human ILT4 ECD region
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGV

SEQ ID NO: 110 - protein, 597 aa, Homo sapiens
  REGION 1..597: note = misc_feature - Human ILT4 precursor, with signal peptide, isoform 2
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 111 - protein, 576 aa, Homo sapiens
  REGION 1..576: note = misc_feature - Human mature ILT4, isoform 2, without signal peptide
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 112 - protein, 437 aa, Homo sapiens
  REGION 1..437: note = misc_feature - Human ILT4 isoform 2, ECD
GTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGV

SEQ ID NO: 113 - protein, 454 aa, synthetic construct
  REGION 1..454: note = Synthetic: 21D9.IgG1.3 HC
EGQLLESGGDLVQPGGSLRLSCATSGFTFSNYAMNWVRQAPGKGLEWISVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 107 aa, synthetic construct
  REGION 1..107: note = Synthetic: 10F10.4 VL
AIQLTQSPSSLSASVGDRVTITCRASQGISSALAWFQQKPGKAPKLLIYDASSLESGVPSRFSGSGSGTDFTLTISSLQPEDFATYYCQQFNSYPITFGQGTRLEIK

SEQ ID NO: 115 - DNA, 321 bp, synthetic construct
  source 1..321: mol_type = other DNA
  misc_feature 1..321: note = Synthetic: 10F10.4 VL DNA
gccatccagctgacccagagccctagctccctgtctgccagcgtgggcgacagggtgaccatcacatgcagagcctcccagggaatctctagcgccctggcctggttccagcagaagccaggcaaggcccctaagctgctgatctacgatgcctcctctctggagtccggcgtgccctctaggttctccggctctggcagcggcaccgactttaccctgacaatcagctccctgcagccagaggattttgccacatactattgtcagcagttcaactcttatcccatcacctttggccagggcacacggctggagatcaag

SEQ ID NO: 116 - protein, 214 aa, synthetic construct
  REGION 1..214: note = Synthetic: 10F10.4 LC
AIQLTQSPSSLSASVGDRVTITCRASQGISSALAWFQQKPGKAPKLLIYDASSLESGVPSRFSGSGSGTDFTLTISSLQPEDFATYYCQQFNSYPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 642 bp, synthetic construct
  misc_feature 1..642: note = Synthetic: 10F10.4 LC DNA
  source 1..642: mol_type = other DNA
gccatccagctgacccagagccctagctccctgtctgccagcgtgggcgacagggtgaccatcacatgcagagcctcccagggaatctctagcgccctggcctggttccagcagaagccaggcaaggcccctaagctgctgatctacgatgcctcctctctggagtccggcgtgccctctaggttctccggctctggcagcggcaccgactttaccctgacaatcagctccctgcagccagaggattttgccacatactattgtcagcagttcaactcttatcccatcacctttggccagggcacacggctggagatcaagcgtacggtggccgccccgtccgtgtttatcttccctccatcggacgagcagctcaagtccggtaccgcgagcgtggtctgcctgctgaacaatttctacccgcgcgaagctaaagtgcaatggaaggtcgataacgcacttcagtccgggaacagccaggaatctgtgaccgagcaggactccaaggattcgacctattccctgtcctcgactctcaccctgtcaaaggccgactacgagaagcacaaggtctacgcctgcgaagtgacccatcagggcttgtcctcacccgtgactaagagcttcaaccggggagagtgt

SEQ ID NO: 118 - protein, 371 aa, synthetic construct
  REGION 1..371: note = Synthetic: Cyno ILT4 9152 extracellular domain, including His-Avi Tag
QAGILPKPMLWAEPDRVITQGSPVTLRCQGNLEARGYHLYRERKSASWITLIRPELVKKGQFPIPSITWEDAGRYRCQYYSHSWWSEHSDPLELVVTGAYRKPTLSALPSPVVASGGNVTLQCDSRVALDGFILCKEGEDEHSQRLNSQPRTRGSSRAVFSVGPVSPSRRWSYRCYGYELHSRYVWSLPSDLLELLVPGVSKKPSLSVQPGPVVAGGDKLTLQCGSDAGYDRFALYKEGERDFLQRPGQQLQAGLAQANFTLDPVRGSHGGQYRCYGAHNLSSEWSAPSDPLDILISAGPHSGLRRECDPAVSVTGMDGHFLSDQGGSSSPGGGSGGGSEQKLISEEDLGHHHHHHGLNDIFEAQKIEWHE

SEQ ID NO: 119 - protein, 481 aa, synthetic construct
  REGION 1..481: note = Synthetic: hILT4H extracellular domain plus HIS-Avi tag (used in Example 2)
QTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSESSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGSPGGGSGGGSEQKLISEEDLGHHHHHHGLNDIFEAQKIEWHE

SEQ ID NO: 120 - protein, 8 aa, synthetic construct
  REGION 1..8: note = Synthetic: Residues 70-77 of hILT4
ITRIRPEL

SEQ ID NO: 121 - protein, 23 aa, synthetic construct
  REGION 1..23: note = Synthetic: Residues 78-100 of hILT4
VKNGQFHIPSITWEHTGRYGCQY

SEQ ID NO: 122 - protein, 31 aa, synthetic construct
  REGION 1..31: note = Synthetic: Residues 70-100 of hILT4
ITRIRPELVKNGQFHIPSITWEHTGRYGCQY

SEQ ID NO: 123 - protein, 28 aa, synthetic construct
  REGION 1..28: note = Synthetic: Residues 154 to 181 of hILT4
ILCKEGEEEHPQCLNSQPHARGSSRAIF

SEQ ID NO: 124 - protein, 10 aa, synthetic construct
  REGION 1..10: note = Synthetic: Residues 425 to 434 of hILT4
SSPPPTGPIS

SEQ ID NO: 125 - protein, 5 aa, synthetic construct
  REGION 1..5: note = Synthetic: 9G4 (ILT4.1) VH CDR1
SYYWN

SEQ ID NO: 126 - protein, 16 aa, synthetic construct
  REGION 1..16: note = Synthetic: 9G4 VH CDR2
YIYYSGSTKYNPSLKS

SEQ ID NO: 127 - protein, 8 aa, synthetic construct
  REGION 1..8: note = Synthetic: 9G4 VH CDR3
SGWYYFDY

SEQ ID NO: 128 - protein, 11 aa, synthetic construct
  REGION 1..11: note = Synthetic: 9G4 VL CDR1
RASQSVSSYLA

SEQ ID NO: 129 - protein, 7 aa, synthetic construct
  REGION 1..7: note = Synthetic: 9G4 VL CDR2
DASNRAT

SEQ ID NO: 130 - protein, 9 aa, synthetic construct
  REGION 1..9: note = Synthetic: 9G4 VL CDR3
QQRSYWPWT

SEQ ID NO: 131 - protein, 5 aa, synthetic construct
  REGION 1..5: note = Synthetic: 9C8 VH CDR1
SSDIN

SEQ ID NO: 132 - protein, 17 aa, synthetic construct
  REGION 1..17: note = Synthetic: 9C8 VH CDR2
WMNPNSGHTGYAQKFQD

SEQ ID NO: 133 - protein, 16 aa, synthetic construct
  REGION 1..16: note = Synthetic: 9C8 VH CDR3
GGNSIDWGFSYYGLDV

SEQ ID NO: 134 - protein, 11 aa, synthetic construct
  REGION 1..11: note = Synthetic: 9C8 VL CDR1
RASQGISSALA

SEQ ID NO: 135 - protein, 7 aa, synthetic construct
  REGION 1..7: note = Synthetic: 9C8 VL CDR2
DASSLES

SEQ ID NO: 136 - protein, 9 aa, synthetic construct
  REGION 1..9: note = Synthetic: 9C8 VL CDR3
QQFNSYPFT

SEQ ID NO: 137 - protein, 5 aa, synthetic construct
  REGION 1..5: note = Synthetic: 2H2 VH CDR1
NYAIS

SEQ ID NO: 138 - protein, 17 aa, synthetic construct
  REGION 1..17: note = Synthetic: 2H2 VH CDR2
GIIPILATANYAPKFQG

SEQ ID NO: 139 - protein, 19 aa, synthetic construct
  REGION 1..19: note = Synthetic: 2H2 VH CDR3
SSITMIRGAYLYYYDGMDV

SEQ ID NO: 140 - protein, 12 aa, synthetic construct
  REGION 1..12: note = Synthetic: 2H2 VL CDR1
RASQSVSSSYLA

SEQ ID NO: 141 - protein, 7 aa, synthetic construct
  REGION 1..7: note = Synthetic: 2H2 VL CDR2
GASSRAT

SEQ ID NO: 142 - protein, 8 aa, synthetic construct
  REGION 1..8: note = Synthetic: 2H2 VL CDR3
QQYGSSYT

SEQ ID NO: 143 - protein, 5 aa, synthetic construct
  REGION 1..5: note = Synthetic: 2E5 VH CDR1
NYGMH

SEQ ID NO: 144 - protein, 17 aa, synthetic construct
  REGION 1..17: note = Synthetic: 2E5 VH CDR2
VIWYDGSNEYYAESVKG

SEQ ID NO: 145 - protein, 12 aa, synthetic construct
  REGION 1..12: note = Synthetic: 2E5 VH CDR3
DPFYGSGNYFDY

SEQ ID NO: 146 - protein, 11 aa, synthetic construct
  REGION 1..11: note = Synthetic: 2E5 VL CDR1
RASQSVSSYLA

SEQ ID NO: 147 - protein, 7 aa, synthetic construct
  REGION 1..7: note = Synthetic: 2E5 VL CDR2
DASNRAT

SEQ ID NO: 148 - protein, 10 aa, synthetic construct
  REGION 1..10: note = Synthetic: 2E5 VL CDR3
QQRSNWPPWT

SEQ ID NO: 149 - protein, 5 aa, synthetic construct
  REGION 1..5: note = Synthetic: 24E5 VH CDR1
SYVMS

SEQ ID NO: 150 - protein, 17 aa, synthetic construct
  REGION 1..17: note = Synthetic: 24E5 VH CDR2
GISGSGGSTYYADSVKG

SEQ ID NO: 151 - protein, 12 aa, synthetic construct
  REGION 1..12: note = Synthetic: 24E5 VH CDR3
DQDIIAAYYFVY

SEQ ID NO: 152 - protein, 11 aa, synthetic construct
  REGION 1..11: note = Synthetic: 24E5 VL CDR1
RASQGISSWLA

SEQ ID NO: 153 - protein, 7 aa, synthetic construct
  REGION 1..7: note = Synthetic: 24E5 VL CDR2
AASSLQS

SEQ ID NO: 154 - protein, 9 aa, synthetic construct
  REGION 1..9: note = Synthetic: 24E5 VL CDR3
QQYNSYPLT

SEQ ID NO: 155 - protein, 5 aa, synthetic construct
  REGION 1..5: note = Synthetic: 21D9 VH CDR1
NYAMN

SEQ ID NO: 156 - protein, 17 aa, synthetic construct
  REGION 1..17: note = Synthetic: 21D9 VH CDR2
VISVSGGSTYYADSVKG

SEQ ID NO: 157 - protein, 16 aa, synthetic construct
  REGION 1..16: note = Synthetic: 21D9 VH CDR3
DYYYDSGSYYDSFFDY

SEQ ID NO: 158 - protein, 12 aa, synthetic construct
  REGION 1..12: note = Synthetic: 21D9 VL CDR1
RASQSVSSSYLA

SEQ ID NO: 159 - protein, 7 aa, synthetic construct
  REGION 1..7: note = Synthetic: 21D9 VL CDR2
GASSRAT

SEQ ID NO: 160 - protein, 9 aa, synthetic construct
  REGION 1..9: note = Synthetic: 21D9 VL CDR3
QQYGSSPLT

SEQ ID NO: 161 - protein, 5 aa, synthetic construct
  REGION 1..5: note = Synthetic: 21A5 VH CDR1
DYYLH

SEQ ID NO: 162 - protein, 17 aa, synthetic construct
  REGION 1..17: note = Synthetic: 21A5 VH CDR2
IFHPSGDITSSAQNFQG

SEQ ID NO: 163 - protein, 15 aa, synthetic construct
  REGION 1..15: note = Synthetic: 21A5 VH CDR3
GGVLRYLDWSHAFDI

SEQ ID NO: 164 - protein, 12 aa, synthetic construct
  REGION 1..12: note = Synthetic: 21A5 VL CDR1
RASQSVSSSYLA

SEQ ID NO: 165 - protein, 7 aa, synthetic construct
  REGION 1..7: note = Synthetic: 21A5 VL CDR2
GASSRAT

SEQ ID NO: 166 - protein, 6 aa, synthetic construct
  REGION 1..6: note = Synthetic: 21A5 VL CDR3
QQYGST

SEQ ID NO: 167 - protein, 5 aa, synthetic construct
  REGION 1..5: note = Synthetic: 10F10 VH CDR1
SYAMH

SEQ ID NO: 168 - protein, 17 aa, synthetic construct
  REGION 1..17: note = Synthetic: 10F10 VH CDR2
IISYDEYNKYYADSVKG

SEQ ID NO: 169 - protein, 7 aa, synthetic construct
  REGION 1..7: note = Synthetic: 10F10 VH CDR3
EWVGIRY

SEQ ID NO: 170 - protein, 11 aa, synthetic construct
  REGION 1..11: note = Synthetic: 10F10 VL CDR1
RASQGISSALA

SEQ ID NO: 171 - protein, 7 aa, synthetic construct
  REGION 1..7: note = Synthetic: 10F10 VL CDR2
DASSLES

SEQ ID NO: 172 - protein, 9 aa, synthetic construct
  REGION 1..9: note = Synthetic: 10F10 VL CDR3
QQFNSYPIT

SEQ ID NO: 173 - protein, 16 aa, synthetic construct
  REGION 1..16: note = Synthetic: Residues 127-142 of hILT4
SAQPSPVVTSGGRVTL

SEQ ID NO: 174 - protein, 32 aa, synthetic construct
  REGION 1..32: note = Synthetic: Residues 182-213 of hILT4
SVGPVSPNRRWSHRCYGYDLNSPYVWSSPSDL

SEQ ID NO: 175 - protein, 15 aa, synthetic construct
  REGION 1..15: note = Synthetic: Residues 378-392 of hILT4
QAEFPMSPVTSAHAG

SEQ ID NO: 176 - protein, 454 aa, synthetic construct
  REGION 1..454: note = Synthetic: 21D9.e,IgG1f (Same Fc as SEQ ID NO: 98 w/o C-term. K)
EVQLLESGGGLVQPGGSLRLSCAASGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 177 - protein, 454 aa, synthetic construct
  REGION 1..454: note = Synthetic: 21D9.e.IgG1.1f (Fc is the same as SEQ ID NO: 103)
EVQLLESGGGLVQPGGSLRLSCAASGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 178 - protein, 449 aa, synthetic construct
  REGION 1..449: note = Synthetic: 21D9.e.IgG4_S228P
EVQLLESGGGLVQPGGSLRLSCAASGFTFSNYAMNWVRQAPGKGLEWVSVISVSGGSTYYADSVKGRFTISRDNSKNTLYLQMNSLRAEDTAVYYCAKDYYYDSGSYYDSFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 179 - protein, 324 aa, synthetic construct
  REGION 1..324: note = Synthetic: hIgG4 S228P (Fc of SEQ ID NO: 178)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQQGNVFSCSVMHEALHNHYTQKSLSLS

SEQ ID NO: 180 - protein, 116 aa, synthetic construct
  REGION 1..116: note = Synthetic: 9G4 VH amino acid
QVQLQESGPGLVKPSETLSLTCTVSGGSISSYYWNWIRQPPGKGLEWLGYIYYSGSTKYNPSLKSRVTISVDTSKNQFSLKLSSVTAADTAVYYCASSGWYYFDYWGQGTLVTVSS

SEQ ID NO: 181 - DNA, 348 bp, synthetic construct
  misc_feature 1..348: note = Synthetic: 9G4 VH DNA
  source 1..348: mol_type = other DNA
caggtgcagctgcaggagtcgggcccaggactggtgaagccttcggagaccctgtccctcacctgcactgtctctggtggctccatcagtagttactactggaactggatccggcagcccccagggaagggactggagtggcttgggtacatctattacagtgggagtaccaagtacaacccctccctcaagagtcgagtcaccatatcagtagacacgtccaagaaccagttctccctgaagctgagctctgtgaccgctgcggacacggccgtgtattattgtgccagcagtggctggtactactttgactattggggccagggaaccctggtcaccgtctcctca

SEQ ID NO: 182 - protein, 107 aa, synthetic construct
  REGION 1..107: note = Synthetic: 9G4 VL amino acid
EIVLTQSPATLSLSPGERATLSCRASQSVSSYLAWYQQKPGQAPRLLIYDASNRATGIPARFSGSGSGTDFTLTISSLEPEDFAGYYCQQRSYWPWTFGQGTKVEIK

SEQ ID NO: 183 - DNA, 321 bp, synthetic construct
  misc_feature 1..321: note = Synthetic: 9G4 VL DNA
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcgtccaacagggccactggcatcccagccaggttcagtggcagtgggtctgggacagacttcactctcaccatcagcagcctagagcctgaagattttgcaggttattactgtcagcagcgtagctactggccgtggacgttcggccaagggaccaaggtggaaatcaaa

SEQ ID NO: 184 - protein, 98 aa, synthetic construct
  REGION 1..98: note = Synthetic: Germline hIGHV3-23*01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 185 - protein, 11 aa, synthetic construct
  REGION 1..11: note = Synthetic: Germline J Gene IGHJ4
WGQGTLVTVSS

SEQ ID NO: 186 - protein, 96 aa, synthetic construct
  REGION 1..96: note = Synthetic: Germline hIGKV3-20*01
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 187 - protein, 10 aa, synthetic construct
  REGION 1..10: note = Synthetic: Germline J Gene IGKJ4
FGGGTKVEIK

SEQ ID NO: 188 - protein, 98 aa, synthetic construct
  REGION 1..98: note = Synthetic: Germline hIGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 189 - protein, 11 aa, synthetic construct
  REGION 1..11: note = Synthetic: Germline J Gene IGHJ3
WGQGTMVTVSS

SEQ ID NO: 190 - protein, 98 aa, synthetic construct
  REGION 1..98: note = Synthetic: Germline hIGHV3-30*01
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 191 - protein, 11 aa, synthetic construct
  REGION 1..11: note = Synthetic: Germline J Gene IGHJ4
WGQGTLVTVSS

SEQ ID NO: 192 - protein, 95 aa, synthetic construct
  REGION 1..95: note = Synthetic: Germline hIGKV1-13*02
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 193 - protein, 10 aa, synthetic construct
  REGION 1..10: note = Synthetic: Germline J Gene IGKJ5
FGQGTRLEIK

(19) United States
(12) Patent Application Publication
(10) Pub. No.: US 2026/0125466 A1
(43) Pub. Date: May 7, 2026

(54) Antibodies Binding to ILT4

(71) Applicants: Five Prime Therapeutics, Inc., Thousand Oaks, CA (US); Bristol-Myers Squibb Company, Princeton, NJ (US)

(72) Inventors: Xiao Min Schebye, San Carlos, CA (US); Diana Yuhui Chen, San Francisco, CA (US); Andrew Rankin, Redwood City, CA (US); Xiaodi Deng, San Mateo, CA (US); Joseph Toth, Belmont, MA (US); Linda Liang, Mountain View, CA (US); Michelle Minhua Han, Piedmont, CA (US); Christine Bee, San Francisco, CA (US); Hong-an Truong, San Francisco, CA (US); Mark J. Selby, San Francisco, CA (US); Nils Lonberg, Woodside, CA (US); Guodong Chen, East Brunswick, NJ (US); Richard Y. Huang, Bridgewater, NJ (US); Ekaterina G. Deyanova, Lawrenceville, NJ (US); Alan J. Korman, Piedmont, CA (US)

(73) Assignees: Five Prime Therapeutics, Inc., Thousand Oaks, CA (US); Bristol-Myers Squibb Company, Princeton, NJ (US)

(21) Appl. No.: 19/322,468

(22) Filed: Sep. 8, 2025

Related U.S. Application Data
(62) Division of application No. 17/845,742, filed on Jun. 21, 2022, now Pat. No. 12,435,136.
Application No. 17/845,742 is a division of application No. 16/506,754, filed on Jul. 9, 2019, now Pat. No. 11,401,328.
Application No. 16/506,754 is a continuation of application No. PCT/US2019/040820, filed on Jul. 8, 2019.
(60) Provisional application No. 62/695,600, filed on Jul. 9, 2018.
(60) Provisional application No. 62/744,611, filed on Oct. 11, 2018.

(51) Int. Cl. C07K 16/28; A61K 39/00; A61K 39/395; A61K 45/06; A61P 35/00; G01N 33/563; G01N 33/577

(52) U.S. Cl. CPC C07K 16/2803; A61K 39/3955; A61K 45/06; A61P 35/00; C07K 16/2827; G01N 33/563; G01N 33/577; A61K 2039/505; C07K 2317/21; C07K 2317/33; C07K 2317/34; C07K 2317/40; C07K 2317/55; C07K 2317/565; C07K 2317/567; C07K 2317/71; C07K 2317/76; C07K 2317/92

(57) ABSTRACT

The present application relates to antibodies specifically binding to immunoglobulin-like transcript 4 (ILT4), which is also known as LILRB2, LIR2, MIR10, and CD85d, and corresponding nucleic acids, host cells, compositions, and uses. In some embodiments, the antibodies bind specifically to human ILT4, but do not significantly bind to ILT2, ILT3, or ILT5, or to other members of the LILRA or LILRB families.

### PRIORITY INFORMATION

The present application is a divisional application of U.S. patent application Ser. No. 17/845,742, filed Jun. 21, 2022, which is a divisional of U.S. patent application Ser. No. 16/506,754, filed Jul. 9, 2019, now U.S. Pat. No. 11,401,328, which is a continuation of International Patent Application PCT/US2019/40820, filed Jul. 8, 2019, which claims priority to US Provisional Patent Application Nos. 62/695,600, filed Jul. 9, 2018, and 62/744,611, filed Oct. 11, 2018. All of these applications are incorporated in their entirety by reference herein.

### SEQUENCE LISTING

The present application contains a Sequence Listing which has been submitted electronically in XML format. Said XML copy, created on Sep. 4, 2025, is named “01134-0060-02US-ST26.xml” and is 303,540 bytes in size. The information in the electronic format of the sequence listing is incorporated herein by reference in its entirety.

### FIELD

The present application relates to antibodies specifically binding to and inhibiting ILT4, and their use in cancer treatment.

### BACKGROUND

ILT4 (immunoglobulin-like transcript 4; also known as LILRB2, LIR2, MIR10, and CD85d) is expressed on myeloid cells, such as monocytes, macrophages, and dendritic cells. ILT4 is part of a family of structurally-related receptors that also includes the proteins LILRA1, LILRA2, LILRA3, LILRA4, LTLRA5, LTLRA6, ILT2, ILT3, ILT5, and LIR8.

The extracellular domains of proteins in this family contain several immunoglobulin-like repeats, while their cytoplasmic tails contain several tyrosine-based inhibitory motifs (ITIMs), which recruit tyrosine phosphatases.

### BRIEF DESCRIPTION OF THE FIGURES

The present application claims priority to US provisional patent applications that contain at least one drawing from the list below executed in color. Once this international application publishes and its priority provisional applications become available to the public, it is assumed that the color drawings will be provided by the US Patent and Trademark Office upon request and payment of the necessary fee.

FIG. 1 shows the nucleotide and amino acid sequences of the heavy chain variable region (“VH”) of anti-hILT4 antibody 9G4, indicating the location of the VH Cmdrs.

FIG. 2 shows the nucleotide and amino acid sequences of the light chain variable region (“VL”) of anti-hILT4 antibody 9G4, indicating the location of the VL CDRs.

FIG. 3 shows the nucleotide and amino acid sequences of the VH of anti-hILT4 antibody 9C8, indicating the location of the VH CDRs.

FIG. 4 shows the nucleotide and amino acid sequences of the VL of anti-hILT4 antibody 9C8, indicating the location of the VL CDRs.

FIG. 5 shows the nucleotide and amino acid sequences of the VH of anti-hILT4 antibody 2H2, indicating the location of the VH CDRs.

FIG. 6 shows the nucleotide and amino acid sequences of the VL of anti-hILT4 antibody 2H2, indicating the location of the VL CDRs.

FIG. 7 shows the nucleotide and amino acid sequences of the VH of anti-hILT4 antibody 2E5, indicating the location of the VH CDRs.

FIG. 8 shows the nucleotide and amino acid sequences of the VL of anti-hILT4 antibody 2E5, indicating the location of the VL CDRs.

FIG. 9 shows the nucleotide and amino acid sequences of the VH of anti-hILT4 antibody 24E5, indicating the location of the VH CDRs.

FIG. 10 shows the nucleotide and amino acid sequences of the VL of anti-hILT4 antibody 24E5, indicating the location of the VL CDRs.

FIG. 11 shows the nucleotide and amino acid sequences of the VH of anti-hILT4 antibody 21D9, indicating the location of the VH CDRs.

FIG. 12 shows the nucleotide and amino acid sequences of the VL of anti-hILT4 antibody 21D9, indicating the location of the VL CDRs.

FIGS. 13A and 13C show alignments of the amino acid sequences of the variable domains of the heavy (VH) (FIG. 13A) and light (VH) (FIG. 13C) chains of ILT4 antibody 21D9 with germline immunoglobulin sequences hIGHV3-23*01 and hIGKV3-20*01, respectively. Arrows point to germline mutations. AbM VH CDR1 and Kabat VH CDR2, VH CDR3, and VL CDR1-3 sequences are shown in light text. FIG. 13B shows the VH amino acid sequences of the four germline reversion mutants 21D9.b, 21D9.c, 21D9.d and 21D9.e, with an IgG1.3 heavy chain constant region (i.e. 21D9.b-.e hIgG1.3). Amino acid residues represented in upper case in FIG. 13B are germline reversion substitutions.

FIGS. 14A and 14B show alignments of the amino acid sequences of the variable domains of the heavy and light chains of ILT4 antibody 21A5 with germline immunoglobulin sequences hIGHV1-46*01 (FIG. 14A) and hIGKV3-20*01 (FIG. 14B), respectively. AbM VH CDR1 and Kabat VH CDR2, VH CDR3, and VL CDR1-3 sequences are shown in light text. Arrows point to germline reversion mutations.

FIG. 15 shows alignments of the amino acid sequences of variable domains of the heavy and light chains of ILT4 antibody 10F10 with germline immunoglobulin sequences hIGHV3-30*01 (top 3 lines) and hIGKV1-13*02 (bottom 3 lines), respectively. AbM VH CDR1 and Kabat VH CDR2, VH CDR3, and VL CDR1-3 sequences are shown in light text. Arrows point to germline reversion mutations.

FIG. 16 shows sensorgrams of binding of the indicated anti-hILT4 antibodies to hILT4. A schematic of the method used is shown above the sensorgram panels.

FIG. 17 shows binding of anti-hILT4 antibodies 2H2, 10F10 wildtype (WT, i.e., parent antibody), 21A5.a, 21A5 WT, 21D9 WT, 21D9e and isotype control hIgG1.3 to hILT4 transfected CHO cells, as a function of antibody concentration, and as determined by flow cytometry. EC₅₀ values are provided below a graph of the binding data.

FIG. 18A shows binding of the specified anti-hILT4 antibodies and isotype controls to hILT4 transfected CHO cells, as a function of antibody concentration, and as determined by flow cytometry. EC₅₀ values are provided to the right of the binding data graph. FIG. 18B shows binding of anti-hILT4 antibodies to human monocytes isolated from a normal healthy donor's peripheral blood. The antibody species are listed next to the graph and the EC50 values are provided in the tables just below the graph.

FIG. 19A shows flow cytometry diagrams of binding of anti-hILT4 antibodies 21D9, 21D9.e, 21A5, 21A5.a, 10F10 and 2H2 (left to right) to human LILRA1, human LILRA2, human LILRA3, human LILRA4, human LILRA5, human LILRA6, human ILT2, human ILT3, human ILT4, human ILT5 and human LIR8 (top to bottom). FIG. 19B shows flow cytometry diagrams of binding of anti-hILT4 antibodies 21A5 and germline reversion mutant 21A5.a to human LIRA1, LIRA3 and ILT4.

FIGS. 20A and B show the percentage of maximum binding of the specified antibodies 21D9 (“21D9 IgG1.1”) and 21D9.e (FIG. 20A) and 21A5 (“21A5WT”) and 21A5.a (FIG. 20B) to the specified human LILRA/LILRB family members, LILRA5, ILT4 in FIG. 20A and LILRA1, LILRA3, and ILT4 in FIG. 20B, as a function of antibody concentration, indicating that the anti-ILT4 antibodies 21D9, 21D9.e, 21A5 and 21A5.a show weak cross-reactivity to other LILRA family members.

FIGS. 21A-D show T cell activity of anti-hILT4 antibodies. FIG. 21A is a histogram of ³H thymidine incorporation of T co-cells cultured with CHO cells transfected with hILT4 and OKT3 incubated with (from left to right on x-axis) no antibody, isotype control hIgG1.3 antibody, and antibodies 21D9 (“21D9 WT”), 21D9.e, 21A5 (“21A5 WT”), 21A5.a and 10F10 (“10F10 WT”) as a function of antibody concentration (20, 4, 0.8, 0.16, 0.032, and 0.0064 μg/ml). FIG. 21B provides the associated EC50 values. FIG. 21C shows T cell activity of anti-hILT4 antibodies. FIG. 21C is a histogram of IFNγ production of T cells co-cultured with CHO cells transfected with hILT4 and OKT3 incubated with (from left to right on x-axis) no antibody, isotype control hIgG1.3 antibody, and antibodies 21D9 (“21D9 WT”), 21D9.e, 21A5 (“21A5 WT”), 21A5.a and 10F10 (“10F10 WT”) as a function of antibody concentration (20, 4, 0.8, 0.16, 0.032, and 0.0064 μg/ml). FIG. 21D provides the associated EC50 values.

FIGS. 22A-D show proliferation (FIGS. 22A and 22C) and IFNγ production (FIGS. 22B and 22D) of T cells in an allo-MLR (mixed lymphocyte reaction) of monocyte-derived dendritic cells (MoDC) co-cultured with T cells. Anti-hILT4 antibody 21A5.a or 21D9.e (FIGS. 22A and 22B) or 2H2 or 21D9 (FIGS. 22C and 22D), or isotype control or no antibody (“untreated”) were added during monocyte differentiation to MoDC and each antibody was not added during the allo-MLR assay.

FIGS. 23A-D show expression levels of CD83 and CD86 on monocyte-derived dendritic cells (MoDC). Anti-hILT4 antibodies 21D9.e, 21A5.a (FIG. 23A), 21A5, 10F10, 21D9 (FIG. 23B) or 2H2 (FIGS. 23C and 23D) or isotype control were incubated during the monocyte differentiation to MoDC or left untreated (no antibody).

FIGS. 24A and B show the level of TNF-alpha secreted from in vitro differentiated macrophages activated by lipopolysaccharide (LPS) (FIG. 24A) or STING agonist 2′3′-cGAMP (FIG. 24B). Anti-ILT4 antibodies 21D9, 21D9.e, 21A5, 21A5.a, 2H2 and 10F10 or isotype control were included during activation.

FIGS. 25A and B show the percentage of CD4 T cell proliferation (FIG. 25A) and amount of IFN-gamma (FIG. 25B) produced from the allo-MLR of CD4 T cells co-cultured with in vitro differentiated macrophages in the presence of anti-hILT4 antibodies 21D9, 21D9.e, 21A5 or 21A5.a or anti-PD-L1 antibody or isotype control antibody. FIG. 25A shows the percentage of CD4 T cell proliferation by FACS analysis based on CFSE dilution, and FIG. 25B shows IFNγ production measured by Alphalisa, both as a function of the ILT4 antibodies or controls.

FIGS. 26A and B show that the combination of anti-ILT4 antibody 21D9 with the anti-PD-L1 antibody clone 29E.2A3 enhances the production of IFNγ to a higher level than relative to each antibody alone in a macrophage:CD4 T cell allo-MLR. FIG. 26A shows one experiment from one macrophage:CD4 T allogenic pair. FIG. 26B shows responses from multiple allogeneic pairs, wherein each line denotes one experiment from one allogeneic pair. Statistical analyses were performed using the non-parametric one-way Anova multiple comparison (Friedman test). *p<0.05, **p<0.01, ***p<0.001, ****p<0.0001.

FIGS. 27A, B and C show HDX epitope mapping of anti-hILT4 Abs 21D9 and 2H2 Fabs. FIG. 27A shows the hILT4 sequence coverage by Pepsin. FIG. 27B shows differential HDX of the 21D9 and 2H2 Fabs. Of the sequence portions delineated in the figure, those that are underlined have less HDX protection than those that are not underlined, as also noted below the figure drawing. FIG. 27C shows the HDX epitopes of the two Fabs mapped to the linear sequence of hILT4. The epitope of antibody 21D9 is shown by a single underline. The epitope of antibody 2H2 is shown in the double underline and shaded/hatched underline portions of the sequence.

FIGS. 28A, B, C, and D show inhibition of binding of hILT4 to HLA-A and HLA-B by antibodies 21D9 (FIG. 28A), 2H2 (FIG. 28B), 10F10 (FIG. 28C) and 21A5 (FIG. 28D).

FIGS. 29A-D show that 21D9e.IgG1.3 enhances both IFN-γ and TNF-α secretion by CD4+ and CD8+ T cells, as determined by intracellular IFN-γ and TNF-α staining. FIG. 29A shows IFN-γ secretion in CD4+ T cells; FIG. 29B shows TNF-α secretion in CD4+ T cells; FIG. 29C shows IFN-γ secretion in CD8+ T cells; and FIG. 29D shows TNF-α secretion in CD8+ T cells.

FIG. 30 shows that anti-ILT4 antibodies 21D9, 21A5, 21D9 Fab enhance IFN-g secretion upon antigen stimulation in a CMV Lysate Assay. Nivolumab was used as a positive control and the absence of antibody (“no Ab”) and isotype control were used as negative controls. The concentrations of anti-ILT4 antibodies are, from left to right: 0.00128; 0.0064; 0.032; 0.16; 0.8; 4; and 20 μg/ml (a bar for each concentration is shown in the histogram). The antibodies and controls are shown from left to right in the same order as shown on the right of the histogram (e.g., “No Ab” corresponds to the bar on the left and “Nivo” corresponds to the bar on the right).

FIG. 31 shows that anti-ILT4 antibodies 2H2 and 21D9, either as IgG1.1f or as Fab, enhance T cell proliferation (measured by ³H incorporation) in a monocyte:T allo-MLR.

FIGS. 32A-C show that anti-ILT4 antibodies 21D9e.IgG1.3 (“ILT4.8” in the figure) and 9G4.IgG1.3 (“ILT4.1”) (see Table 1 for nomenclature of antibodies) promote expression of the costimulatory molecules CD86 (FIG. 32A) and CD80 (FIG. 32B) and the dendritic cell activation marker CD83 (FIG. 32C) on cynomolgus monocyte-derived dendritic cells in vitro. Each antibody was added during cynomolgus monocyte differentiation to monocyte-derived dendritic cells (DC).

FIGS. 33A and B show the level of TNF-alpha secreted from in vitro differentiated macrophages activated by lipopolysaccharide (LPS) (FIG. 33A) or STING agonist 2′3′cGAMP (FIG. 33B). Anti-ILT4 antibodies 21D9.e and 10F10.4 or isotype control were included during activation.

FIG. 34 shows that the level of TNF-alpha secreted by in vitro differentiated macrophages incubated with 21D9.e in the context of IgG1, IgG4 or IgG1.3 is similar.

FIG. 35 shows the percentage of monomer, high molecular weight (HMW) species and low molecular weight (LMW) species of 21D9.e.IgG1.3 after 0, 1, 2 and 3 months at 25° C. or 40° C. The monomer, HMW and LMW species correspond to the bottom, middle and top portions of each of the bars, respectively, in the histogram.

FIG. 36 shows the percentage of activated basophils of a total of 8 donors after incubation with 21D9e.IgG1.3 (“ILT4.8” in the figure), 21A5.a.IgG1.3 (“ILT4.9”), an isotype control antibody “anti-DT”, anti-FcεRI or N-formylmethionine-leucyl-phenyalanine (fMLP), showing a lack of basophil activation by 21D9e.IgG1.3 (ILT4.8) and 21A5.a.IgG1.3 (“ILT4.9”).

FIGS. 37A-D show results from experiments to determine the epitopes of certain antibodies. FIGS. 37A and B show competition (or cross-blocking) assay results of 2H2 (FIG. 37A) and 21D9 (FIG. 37B) by 10F10 and 21A5 antibodies of hILT4 expressed on the surface of CHO cells. FIG. 37C is a diagram showing a three-dimensional representation of hILT4 with the location of Ig-like domains 1 and 2, and indicating the areas of hILT4 to which antibodies 21D9, 29A5, 2H2 and 10F10 bind. FIG. 37D shows a further three-dimensional representation of hILT4 with the location of Ig-like domains 1 and 2, and indicating the areas of hILT4 to which antibodies 21D9, 21A5, and 10F10 bind.

### SUMMARY OF THE DISCLOSURE

Certain embodiments of this disclosure are summarized in the claims are the end of the disclosure. For example, the present disclosure includes the following embodiments, as well as other embodiments described in further sections of the text and in the figures and sequences herein.

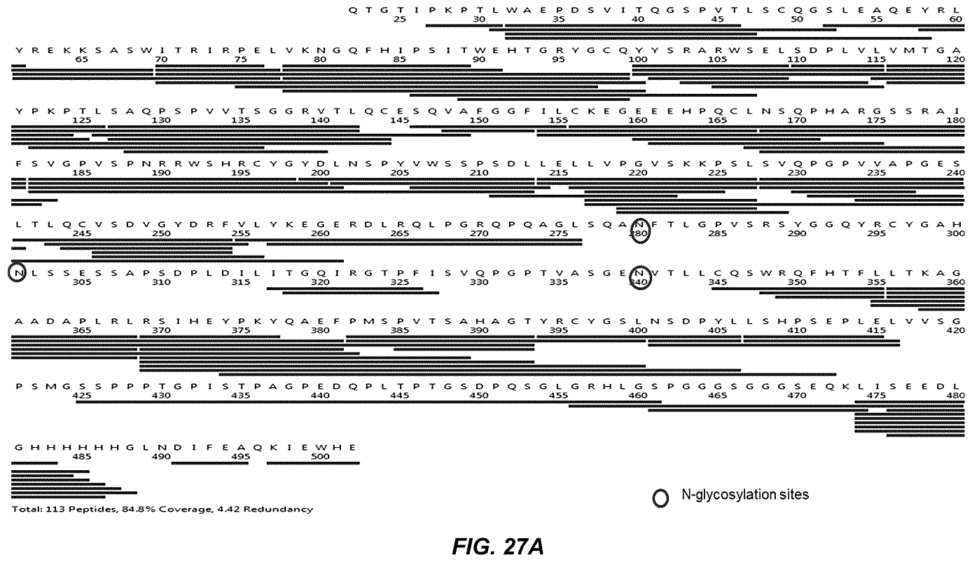

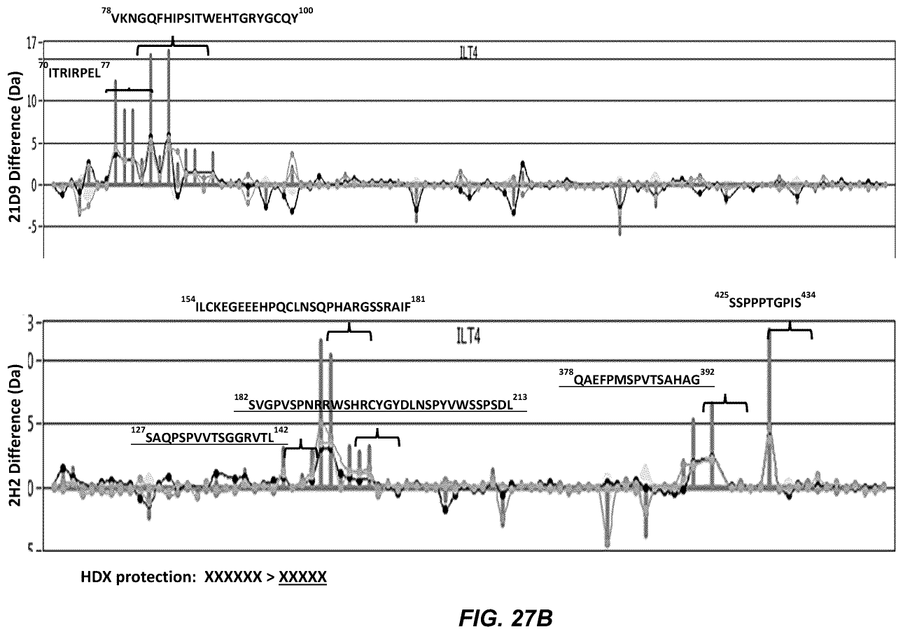

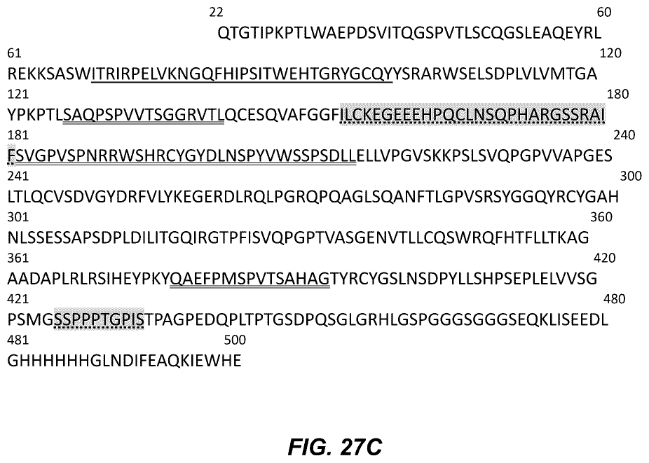

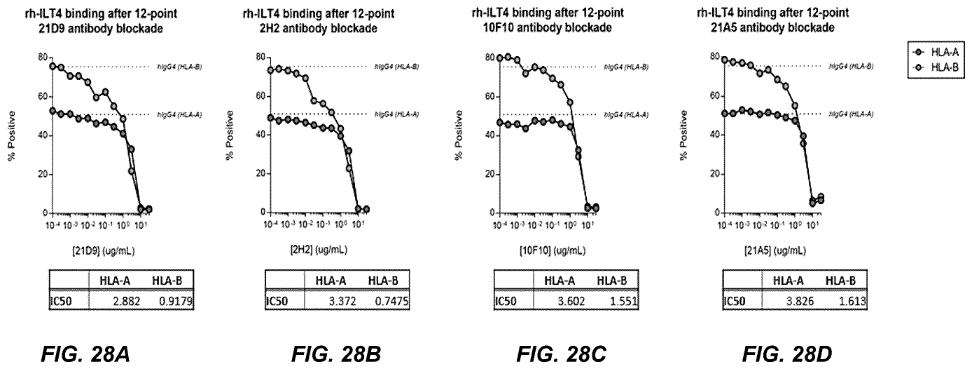

Embodiment 1: An isolated antibody that binds specifically to human ILT4 (hILT4 or ILT4), the antibody comprising a heavy chain and a light chain, and further comprising one or more of the following characteristics:
specific binding to hILT4 (e.g. comprising the amino acid sequence of SEQ ID NO: 108, 109, 111, 112, or 119), e.g., with a K_{D} of 10⁻⁸ M or less, or of 10⁻⁹ M or less;
lack of specific binding to hILT2, hILT3, and/or hILT5;
lack of specific binding to one or more members of the LILRA and/or LILRB families;
stimulates T cell activation, e.g., in a mixed lymphocyte reaction (MLR) assay, as measured by increased T cell proliferation or IFN-gamma secretion, e.g., as shown in an assay described in the Examples;
stimulates differentiation or activation of monocytes into macrophages, e.g., stimulates differentiation of monocytes into pro-inflammatory macrophages, e.g., as shown in an assay described in the Examples;
promotes expression of CD83 and CD86 on human monocyte derived immature dendritic cells (Mo-iDC), e.g., as shown in an assay described in the Examples;
enhances IFN-γ secretion upon antigen stimulation in a cytomegalovirus (CMV) lysate assay, e.g., as shown in an assay described in the Examples;
enhances IFN-γ and TNF-α secretion by CD4+ and CD8+ T cells in an allo-mixed lymphocyte reaction (MLR) assay upon CD3 stimulation, e.g., as shown in an assay described in the Examples;
inhibits binding of HLA-A and/or HLA-B to ILT4;
binds to ⁷⁰ITRIRPELVKNGQFHIPSITWEHTGRYGCQY¹⁰⁰ (SEQ ID NO: 122) as determined by hydrogen deuterium exchange (HDX), e.g., as shown in an HDX assay described in the Examples;
competes for binding to hILT4 with an antibody described herein;
binds specifically to a cyno ILT4 comprising SEQ ID NO: 118, e.g., as shown in a binding assay described in the Examples;
inhibits binding of human ILT4 (hILT4) to an ILT4 binding partner, such as an MHC class I molecule, such as HLA-A and HLA-B (for example, inhibits binding of hILT4 to both HLA-A and HLA-B);
has an HLA-A and HLA-B binding profile shown in FIG. 28;
promotes pro-inflammatory polarization of macrophages towards M1 macrophages;
has a binding profile as shown in FIG. 27;
does not induce (or trigger) basophil activation;
binds to the following regions of hILT4: (i) ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122); or (iii) ¹⁵⁴ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), and, if binding to (i) or (ii), then optionally, not binding significantly to other regions of the extracellular domain of ILT4, such as regions or residues located N-terminal to amino acid 170, wherein the amino acid numbering of hILT4 is that of immature hILT4 (i.e., ILT4 comprising its native signal sequence); and
interacts with one or more (or all of) amino acid residues Lys43, Ile49, Thr50 and Arg51 of mature hILT4 or interacts with one or more (or all of) amino acid residues Gly117, Val119, Try120, Leu134, Lys136, Gln149, Pro150, Ile159, Ser161, Val162, Gly163, Pro164, Pro167, His173, Try178, Pro183 and Tyr184 of mature hILT4 or interacts with one or more (or all of) amino acid residues Glu42, Lys43, Gly76, Cys77, Leu88, Pro91, Pro183 and Tyr184 of mature hILT4, as determined by carbene foot-printing, e.g., as described in the Examples.

Embodiment 2: The isolated antibody of embodiment 1, comprising a heavy chain and a light chain, wherein the heavy chain comprises VH CDR1, CDR2 and CDR3 of anti-ILT4 antibody 9G4 (SEQ ID Nos: 125-127), 9C8 (SEQ ID Nos: 131-133), 2H2 (SEQ ID Nos: 137-139), 2E5 (SEQ ID Nos: 143-145), 24E5 (SEQ ID Nos: 149-151), 21D9 (SEQ ID Nos: 155-157), 21A5 (SEQ ID Nos: 161-163), or 10F10 (SEQ ID Nos: 167-169).

Embodiment 3: The isolated antibody of embodiment 1 or 2, comprising a heavy chain and a light chain, wherein the light chain comprises VL CDR1, CDR2 and CDR3 of anti-ILT4 antibody 9G4 (SEQ ID Nos: 128-130), 9C8 (SEQ ID Nos: 134-136), 2H2 (SEQ ID Nos: 140-142), 2E5 (SEQ ID Nos: 146-148), 24E5 (SEQ ID Nos: 152-154), 21D9 (SEQ ID Nos: 158-160), 21A5 (SEQ ID Nos: 164-166), or 10F10 (SEQ ID Nos: 170-172).

Embodiment 4: The isolated antibody of embodiment 2, wherein the heavy chain comprises VH CDR1, CDR2 and CDR3 and VL CDR1, CDR2 and CDR3 of anti-ILT4 antibody 9G4 (SEQ ID Nos: 143-148), 24E5 (SEQ ID Nos: 149-154), 21D9 (SEQ ID Nos: 155-160), 21A5 (SEQ ID Nos: 161-166), or 10F10 (SEQ ID Nos: 167-172).

Embodiment 5: The isolated antibody of any one of embodiments 1-4, which comprises:
a VH comprising the amino acid sequence of the VH CDR1, CDR2 and CDR3 of 9G4 (SEQ ID Nos: 125-127) and a VL comprising the amino acid sequence of the VL CDR1, CDR2 and CDR3 of 9G4 (SEQ ID Nos: 128-130);
a VH comprising the amino acid sequence of the VH CDR1, CDR2 and CDR3 of 9C8 (SEQ ID Nos: 131-133) and a VL comprising the amino acid sequence of the VL CDR1, CDR2 and CDR3 of 9C8 (SEQ ID Nos: 134-136);
a VH comprising the amino acid sequence of the VH CDR1, CDR2 and CDR3 of 2H2 (SEQ ID Nos: 137-139) and a VL comprising the VL CDR1, CDR2 and CDR3 of 2H2 (SEQ ID Nos: 140-142);
a VH comprising the amino acid sequence of the VH CDR1, CDR2 and CDR3 of 2E5 (SEQ ID Nos: 143-145) and a VL comprising the amino acid sequence of the VL CDR1, CDR2 and CDR3 of 2E5 (SEQ ID Nos: 146-148);
a VH comprising the amino acid sequence of the VH CDR1, CDR2 and CDR3 of 24E5 (SEQ ID Nos: 149-151) and a VL comprising the amino acid sequence of the VL CDR1, CDR2 and CDR3 of 24E5 (SEQ ID Nos: 152-154);
a VH comprising the amino acid sequence of the VH CDR1, CDR2 and CDR3 of 21D9 (SEQ ID Nos: 155-157) and a VL comprising the amino acid sequence of the VL CDR1, CDR2 and CDR3 of 21D9 (SEQ ID Nos: 158-160);
a VH comprising the amino acid sequence of the VH CDR1, CDR2 and CDR3 of 21D9.b (SEQ ID Nos: 155-157) and a VL comprising the amino acid sequence of the VL CDR1, CDR2 and CDR3 of 21D9.b (SEQ ID Nos: 158-160);
a VH comprising the amino acid sequence of the VH CDR1, CDR2 and CDR3 of 21D9.c (SEQ ID Nos: 155-157) and a VL comprising the amino acid sequence of the VL CDR1, CDR2 and CDR3 of 21D9.c (SEQ ID Nos: 158-160);
a VH comprising the amino acid sequence of the VH CDR1, CDR2 and CDR3 of 21D9.d (SEQ ID Nos: 155-157) and a VL comprising the amino acid sequence of the VL CDR1, CDR2 and CDR3 of 21D9.d (SEQ ID Nos: 158-160);
a VH comprising the amino acid sequence of the VH CDR1, CDR2 and CDR3 of 21D9.e (SEQ ID Nos: 155-157) and a VL comprising the amino acid sequence of the VL CDR1, CDR2 and CDR3 of 21D9.e (SEQ ID Nos: 158-160);
a VH comprising the amino acid sequence of the VH CDR1, CDR2 and CDR3 of 21A5 (SEQ ID Nos: 161-163) and a VL comprising the amino acid sequence of the VL CDR1, CDR2 and CDR3 of 21A5 (SEQ ID Nos: 164-166);
a VH comprising the amino acid sequence of the VH CDR1, CDR2 and CDR3 of 21A5.a (SEQ ID Nos: 161-163) and a VL comprising the amino acid sequence of the VL CDR1, CDR2 and CDR3 of 21A5.a (SEQ ID Nos: 164-166);
a VH comprising the amino acid sequence of the VH CDR1, CDR2 and CDR3 of 10F10 (SEQ ID Nos: 167-169) and a VL comprising the amino acid sequence of the VL CDR1, CDR2 and CDR3 of 10F10 (SEQ ID Nos: 170-172);
a VH comprising the amino acid sequence of the VH CDR1, CDR2 and CDR3 of 10F10.1 (SEQ ID Nos: 167-169) and a VL comprising the amino acid sequence of VL CDR1, CDR2 and CDR3 of 10F10.1 (SEQ ID Nos: 170-172);
a VH comprising the amino acid sequence of the VH CDR1, CDR2 and CDR3 of 10F10.3 (SEQ ID Nos: 167-169) and a VL comprising the amino acid sequence of the VL CDR1, CDR2 and CDR3 of 10F10.3 (SEQ ID Nos: 170-172); or
a VH comprising the amino acid sequence of the VH CDR1, CDR2 and CDR3 of 10F10.4 (SEQ ID Nos: 167-169) and a VL comprising the amino acid sequence of the VL CDR1, CDR2 and CDR3 of 10F10.4 (SEQ ID Nos: 170-172).

Embodiment 6: The isolated antibody of any one of embodiments 1-5, wherein the antibody heavy chain comprises a VH with an amino acid sequence at least 95%, at least 96%, at least 97%, at least 98%, or at least 99% identical to the amino acid sequence of the VH of 9G4 (SEQ ID NO: 51, amino acids 20-135), 9C8 (SEQ ID NO: 55), 2H2 (SEQ ID NO: 58), 2E5 (SEQ ID NO: 63), 24E5 (SEQ ID NO: 67), 21D9 (SEQ ID NO: 71), 21D9.b (SEQ ID NO: 74), 21D9.c (SEQ ID NO: 75), 21D9.d (SEQ ID NO: 78), 21D9.e (SEQ ID NO: 80), 21A5 (SEQ ID NO: 83), 21A5.a (SEQ ID NO: 87), 10F10 (SEQ ID NO: 91), 10F10.1 (SEQ ID NO: 91), 10F10.3 (SEQ ID NO: 91), or 10F10.4 (SEQ ID NO: 91).

Embodiment 7: The isolated antibody of any one of embodiments 1-6, wherein the antibody light chain comprises a VL with an amino acid sequence at least 95%, at least 96%, at least 97%, at least 98%, or at least 99% identical to the amino acid sequence of the VL of 9G4 (SEQ ID NO: 50, amino acids 19-125), 9C8 (SEQ ID NO: 54), 2H2 (SEQ ID NO: 59), 2E5 (SEQ ID NO: 62), 24E5 (SEQ ID NO: 66), 21D9 (SEQ ID NO: 70), 21D9.b (SEQ ID NO: 70), 21D9.c (SEQ ID NO: 70), 21D9.d (SEQ ID NO: 70), 21D9.e (SEQ ID NO: 70), 21A5 (SEQ ID NO: 82), 21A5.a (SEQ ID NO: 86), 10F10 (SEQ ID NO: 90), 10F10.1 (SEQ ID NO: 94), 10F10.3 (SEQ ID NO: 96), or 10F10.4 (SEQ ID NO: 114).

Embodiment 8: The isolated antibody of any one of embodiments 1-7, wherein the antibody heavy chain comprises a VH with an amino acid sequence comprising 1, 2, 3, 4, or 5 amino acid substitutions, conservative substitutions, or reversion substitutions compared to the amino acid sequence of the VH of 9G4 (SEQ ID NO: 51, amino acids 20-135), 9C8 (SEQ ID NO: 55), 2H2 (SEQ ID NO: 58), 2E5 (SEQ ID NO: 63), 24E5 (SEQ ID NO: 67), 21D9 (SEQ ID NO: 71), 21D9.b (SEQ ID NO: 74), 21D9.c (SEQ ID NO: 75), 21D9.d (SEQ ID NO: 78), 21D9.e (SEQ ID NO: 80), 21A5 (SEQ ID NO: 83), 21A5.a (SEQ ID NO: 87), 10F10 (SEQ ID NO: 91), 10F10.1 (SEQ ID NO: 91), 10F10.3 (SEQ ID NO: 91), or 10F10.4 (SEQ ID NO: 91).

Embodiment 9: The isolated antibody of any one of embodiments 1-8, wherein the antibody light chain comprises a VL with an amino acid sequence comprising 1, 2, 3, 4, or 5 amino acid substitutions, conservative substitutions, or reversion substitutions compared to the amino acid sequence of the VL of 9G4 (SEQ ID NO: 50, amino acids 19-125), 9C8 (SEQ ID NO: 54), 2H2 (SEQ ID NO: 59), 2E5 (SEQ ID NO: 62), 24E5 (SEQ ID NO: 66), 21D9 (SEQ ID NO: 70), 21D9.b (SEQ ID NO: 70), 21D9.c (SEQ ID NO: 70), 21D9.d (SEQ ID NO: 70), 21D9.e (SEQ ID NO: 70), 21A5 (SEQ ID NO: 82), 21A5.a (SEQ ID NO: 86), 10F10 (SEQ ID NO: 90), 10F10.1 (SEQ ID NO: 94), 10F10.3 (SEQ ID NO: 96), or 10F10.4 (SEQ ID NO: 114).

Embodiment 10: The isolated antibody of any one of embodiments 1-9, wherein the heavy chain comprises the VH of anti-ILT4 antibody 9G4 (SEQ ID NO: 51, amino acids 20-135), 9C8 (SEQ ID NO: 55), 2H2 (SEQ ID NO: 58), 2E5 (SEQ ID NO: 63), 24E5 (SEQ ID NO: 67), 21D9 (SEQ ID NO: 71), 21D9.b (SEQ ID NO: 74), 21D9.c (SEQ ID NO: 75), 21D9.d (SEQ ID NO: 78), 21D9.e (SEQ ID NO: 80), 21A5 (SEQ ID NO: 83), 21A5.a (SEQ ID NO: 87), 10F10 (SEQ ID NO: 91), 10F10.1 (SEQ ID NO: 91), 10F10.3 (SEQ ID NO: 91), or 10F10.4 (SEQ ID NO: 91).

Embodiment 11: The isolated antibody of any one of embodiments 1-10, wherein the light chain comprises the VL of anti-ILT4 antibody 9G4 (SEQ ID NO: 50, amino acids 19-125), 9C8 (SEQ ID NO: 54), 2H2 (SEQ ID NO: 59), 2E5 (SEQ ID NO: 62), 24E5 (SEQ ID NO: 66), 21D9 (SEQ ID NO: 70), 21D9.b (SEQ ID NO: 70), 21D9.c (SEQ ID NO: 70), 21D9.d (SEQ ID NO: 70), 21D9.e (SEQ ID NO: 70), 21A5 (SEQ ID NO: 82), 21A5.a (SEQ ID NO: 86), 10F10 (SEQ ID NO: 90), 10F10.1 (SEQ ID NO: 94), 10F10.3 (SEQ ID NO: 96), or 10F10.4 (SEQ ID NO: 114).

Embodiment 12: The isolated antibody of any one of embodiments 1-11, which comprises VH and VL of anti-ILT4 antibody 9G4 (SEQ ID NO: 51, amino acids 20-135 and SEQ ID NO: 50, amino acids 19-125), 9C8 (SEQ ID NO: 55 and SEQ ID NO: 54), 2H2 (SEQ ID NO: 58 and SEQ ID NO: 59), 2E5 (SEQ ID NO: 63 and SEQ ID NO: 62), 24E5 (SEQ ID NO: 67 and SEQ ID NO: 66), 21D9 (SEQ ID NO: 71 and SEQ ID NO: 70), 21D9.b (SEQ ID NO: 74 and SEQ ID NO: 70), 21D9.c (SEQ ID NO: 75 and SEQ ID NO: 70), 21D9.d (SEQ ID NO: 78 and SEQ ID NO: 70), 21D9.e (SEQ ID NO: 80 and SEQ ID NO: 70), 21A5 (SEQ ID NO: 83 and SEQ ID NO: 82), 21A5.a (SEQ ID NO: 87 and SEQ ID NO: 86), 10F10 (SEQ ID NO: 91 and SEQ ID NO: 90), 10F10.1 (SEQ ID NO: 91 and SEQ ID NO: 94), 10F10.3 (SEQ ID NO: 91 and SEQ ID NO: 96), or 10F10.4 (SEQ ID NO: 91 and SEQ ID NO: 114).

Embodiment 13: The isolated antibody of any one of embodiments 1-11, which comprises:
a VH comprising the VH CDRs of the VH of 9G4 (SEQ ID Nos: 125-127), and a VL comprising the VL CDRs of 9G4 (SEQ ID Nos: 128-130), and VH and VL amino acid sequences that are at least 90%, at least 91%, at least 92%, at least 93%, at least 94%, at least 95%, at least 96%, at least 97%, at least 98%, or at least 99% identical to the VH and VL of 9G4 (SEQ ID NO: 51, amino acids 20-135 and SEQ ID NO: 50, amino acids 19-125);
a VH comprising the VH CDRs of the VH of 9C8 (SEQ ID Nos: 131-133), and a VL comprising the VL CDRs of 9C8 (SEQ ID Nos: 134-136), and VH and VL amino acid sequences that are at least 90%, at least 91%, at least 92%, at least 93%, at least 94%, at least 95%, at least 96%, at least 97%, at least 98%, or at least 99% identical to the VH and VL of 9C8 (SEQ ID NO: 55 and SEQ ID NO: 54);
a VH comprising the VH CDRs of the VH of 2H2 (SEQ ID Nos: 137-139), and a VL comprising the VL CDRs of 2H2 (SEQ ID Nos: 140-142), and VH and VL amino acid sequences that are at least 90%, at least 91%, at least 92%, at least 93%, at least 94%, at least 95%, at least 96%, at least 97%, at least 98%, or at least 99% identical to the VH and VL of 2H2 (SEQ ID NO: 58 and SEQ ID NO: 59);
a VH comprising the VH CDRs of the VH of 2E5 (SEQ ID Nos: 143-145), and a VL comprising the VL CDRs of 2E5 (SEQ ID Nos: 146-148), and VH and VL amino acid sequences that are at least 90%, at least 91%, at least 92%, at least 93%, at least 94%, at least 95%, at least 96%, at least 97%, at least 98%, or at least 99% identical to the VH and VL of 2E5 (SEQ ID NO: 63 and SEQ ID NO: 62);
a VH comprising the VH CDRs of the VH of 24E5 (SEQ ID Nos: 149-151), and a VL comprising the VL CDRs of 24E5 (SEQ ID Nos: 152-154), and VH and VL amino acid sequences that are at least 90%, at least 91%, at least 92%, at least 93%, at least 94%, at least 95%, at least 96%, at least 97%, at least 98%, or at least 99% identical to the VH and VL of 24E5 (SEQ ID NO: 67 and SEQ ID NO: 66);
a VH comprising the VH CDRs of the VH of 21D9 (SEQ ID Nos: 155-157), and a VL comprising the VL CDRs of 21D9 (SEQ ID Nos: 158-160), and VH and VL amino acid sequences that are at least 90%, at least 91%, at least 92%, at least 93%, at least 94%, at least 95%, at least 96%, at least 97%, at least 98%, or at least 99% identical to the VH and VL of 21D9 (SEQ ID NO: 71 and SEQ ID NO: 70);
a VH comprising the VH CDRs of the VH of 21D9.b (SEQ ID Nos: 155-157), and a VL comprising the VL CDRs of 21D9.b (SEQ ID Nos: 158-160), and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.b (SEQ ID NO: 74 and SEQ ID NO: 70);
a VH comprising the VH CDRs of the VH of 21D9.c (SEQ ID Nos: 155-157), and a VL comprising the VL CDRs of 21D9.c (SEQ ID Nos: 158-160), and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.c (SEQ ID NO: 75 and SEQ ID NO: 70);
a VH comprising the VH CDRs of the VH of 21D9.d (SEQ ID Nos: 155-157), and a VL comprising the VL CDRs of 21D9.d (SEQ ID Nos: 158-160), and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.d (SEQ ID NO: 78 and SEQ ID NO: 70);
a VH comprising the VH CDRs of the VH of 21D9.e (SEQ ID Nos: 155-157), and a VL comprising the VL CDRs of 21D9.e (SEQ ID Nos: 158-160), and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.e (SEQ ID NO: 80 and SEQ ID NO: 70);
a VH comprising the VH CDRs of the VH of 21A5 (SEQ ID Nos: 161-163), and a VL comprising the VL CDRs of 21A5 (SEQ ID Nos: 164-166), and VH and VL amino acid sequences that are at least 90%, at least 91%, at least 92%, at least 93%, at least 94%, at least 95%, at least 96%, at least 97%, at least 98%, or at least 99% identical to the VH and VL of 21A5 (SEQ ID NO: 83 and SEQ ID NO: 82);
a VH comprising the VH CDRs of the VH of 21A5.a (SEQ ID Nos: 161-163), and a VL comprising the VL CDRs of 21A5.a (SEQ ID Nos: 164-166), and VH and VL amino acid sequences that are at least 90%, at least 91%, at least 92%, at least 93%, at least 94%, at least 95%, at least 96%, at least 97%, at least 98%, or at least 99% identical to the VH and VL of 21A5.a (SEQ ID NO: 87 and SEQ ID NO: 86);
a VH comprising the VH CDRs of the VH of 10F10 (SEQ ID Nos: 167-169), and a VL comprising the VL CDRs of 10F10 (SEQ ID Nos: 170-172), and VH and VL amino acid sequences that are at least 90%, at least 91%, at least 92%, at least 93%, at least 94%, at least 95%, at least 96%, at least 97%, at least 98%, or at least 99% identical to the VH and VL of 10F10 (SEQ ID NO: 91 and SEQ ID NO: 90);
a VH comprising the VH CDRs of the VH of 10F10.1 (SEQ ID Nos: 167-169), and a VL comprising the VL CDRs of 10F10.1 (SEQ ID Nos: 170-172), and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.1 (SEQ ID NO: 91 and SEQ ID NO: 94);
a VH comprising the VH CDRs of the VH of 10F10.3 (SEQ ID Nos: 167-169), and a VL comprising the VL CDRs of 10F10.3 (SEQ ID Nos: 170-172), and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.3 (SEQ ID NO: 91 and SEQ ID NO: 96); or
a VH comprising the VH CDRs of the VH of 10F10.4 (SEQ ID Nos: 167-169), and a VL comprising the VL CDRs of 10F10.4 (SEQ ID Nos: 170-172), and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.4 (SEQ ID NO: 91 and SEQ ID NO: 114).

Embodiment 14: The isolated antibody of any one of embodiments 1-13, which comprises:
a VH comprising the amino acid sequence of the VH of 9G4 (SEQ ID NO: 51 residues 20-135) and a VL comprising the amino acid sequence of the VL of 9G4 (SEQ ID NO: 50 residues 19-125);
a VH comprising the amino acid sequence of the VH of 9C8 and a VL comprising the amino acid sequence of the VL of 9C8 (SEQ ID NO: 55 and SEQ ID NO: 54);
a VH comprising the amino acid sequence of the VH of 2H2 and a VL comprising the amino acid sequence of the VL of 2H2 (SEQ ID NO: 58 and SEQ ID NO: 59);
a VH comprising the amino acid sequence of the VH of 2E5 and a VL comprising the amino acid sequence of the VL of 2E5 (SEQ ID NO: 63 and SEQ ID NO: 62);
a VH comprising the amino acid sequence of the VH of 24E5 and a VL comprising the amino acid sequence of the VL of 24E5 (SEQ ID NO: 67 and SEQ ID NO: 66);
a VH comprising the amino acid sequence of the VH of 21D9 and a VL comprising the amino acid sequence of the VL of 21D9 (SEQ ID NO: 71 and SEQ ID NO: 70);
a VH comprising the amino acid sequence of the VH of 21D9.b and a VL comprising the amino acid sequence of the VL of 21D9.b (SEQ ID NO: 74 and SEQ ID NO: 70);
a VH comprising the amino acid sequence of the VH of 21D9.c and a VL comprising the amino acid sequence of the VL of 21D9.c (SEQ ID NO: 75 and SEQ ID NO: 70);
a VH comprising the amino acid sequence of the VH of 21D9.d and a VL comprising the amino acid sequence of the VL of 21D9.d (SEQ ID NO: 78 and SEQ ID NO: 70);
a VH comprising the amino acid sequence of the VH of 21D9.e and a VL comprising the amino acid sequence of the VL of 21D9.e (SEQ ID NO: 80 and SEQ ID NO: 70);
a VH comprising the amino acid sequence of the VH of 21A5 and a VL comprising the amino acid sequence of the VL of 21A5 (SEQ ID NO: 83 and SEQ ID NO: 82);
a VH comprising the amino acid sequence of the VH of 21A5.a and a VL comprising the amino acid sequence of the VL of 21A5.a (SEQ ID NO: 87 and SEQ ID NO: 86);
a VH comprising the amino acid sequence of the VH of 10F10 and a VL comprising the amino acid sequence of the VL of 10F10 (SEQ ID NO: 91 and SEQ ID NO: 90);
a VH comprising the amino acid sequence of the VH of 10F10.1 and a VL comprising the amino acid sequence of the VL of 10F10.1 (SEQ ID NO: 91 and SEQ ID NO: 94);
a VH comprising the amino acid sequence of the VH of 10F10.3 and a VL comprising the amino acid sequence of the VL of 10F10.3 (SEQ ID NO: 91 and SEQ ID NO: 96); or
a VH comprising the amino acid sequence of the VH of 10F10.4 and a VL comprising the amino acid sequence of the VL of 10F10.4 (SEQ ID NO: 91 and SEQ ID NO: 114).

Embodiment 15: The isolated antibody of any one of embodiments 1-14, which is an IgG antibody.

Embodiment 16: The isolated antibody of embodiment 15, which is an IgG1, IgG2 or IgG4 antibody, wherein the IgG4 optionally comprises an S228P substitution (EU numbering).

Embodiment 17: The isolated antibody of any one of embodiments 1-16, wherein the antibody is an effectorless antibody.

Embodiment 18: The isolated antibody of embodiment 17, wherein the heavy chain constant region comprises 1, 2, 3, 4, or 5 mutations in an otherwise wild type human heavy chain constant region that reduce the effector function of the antibody compared to an antibody without the 1, 2, 3, 4, or 5 mutations but otherwise with the same amino acid sequence.

Embodiment 19: The isolated antibody of any one of embodiments 1-18, wherein the heavy chain constant region of the antibody comprises an IgG1.3 heavy chain constant region, an IgG1.1 heavy chain constant region, or an IgG1 heavy chain constant region with a P238K (EU numbering) substitution, or an IgG1 heavy chain constant region comprising the amino acid sequence of any one of SEQ ID Nos: 98, 100, 102, 103, or 104.

Embodiment 20: The isolated antibody of any one of embodiments 1-16 or 19, wherein the antibody has effector function.

Embodiment 21: The isolated antibody of embodiment 20, wherein the antibody is afucosylated (e.g., an afucosylated IgG1 antibody).

Embodiment 22: The isolated antibody of embodiment 20 or 21, wherein the heavy chain constant region comprises 1, 2, 3, 4, or 5 mutations in an otherwise wild type human heavy chain constant region that enhance the effector function of the antibody compared to an antibody without the 1, 2, 3, 4, or 5 mutations but otherwise with the same amino acid sequence.

Embodiment 23: The isolated antibody of any one of embodiments 1-22, which comprises the HC of anti-ILT4 antibody 9G4, 9C8, 2H2, 2E5, 24E5, 21D9, 21D9.b, 21D9.c, 21D9.d, 21D9.e, 21A5, 21A5.a, 10F10, 10F10.1 or 10F10.3, wherein the constant region of the HC is IgG1 (e.g. 9G4.IgG1, etc.), IgG1.3 (e.g. 9G4.IgG1.3, etc.), IgG1.1f (e.g. 9G4.IgG1.1f, etc.), IgG4 (e.g., 9G4.IgG4, etc.), or IgG4 S228P (EU numbering) (e.g., 9G4.IgG4_S228P).

Embodiment 24: The isolated antibody of embodiment 23, wherein the antibody comprises a HC amino acid sequence as follows:
IgG1, e.g. 9G4.IgG1, 9G4 (SEQ ID NO: 2), 9C8 (SEQ ID NO: 4), 2H2 (SEQ ID NO: 6), 2E5 (SEQ ID NO: 8), 24E5 (SEQ ID NO: 10), 21D9 (SEQ ID NO: 12), 21D9.b (SEQ ID NO: 74 and SEQ ID NO: 98), 21D9.c (SEQ ID NO: 75 and SEQ ID NO: 98), 21D9.d (SEQ ID NO: 78 and SEQ ID NO: 98), 21D9.e (SEQ ID NO: 80 and SEQ ID NO: 98), 21A5 (SEQ ID NO: 83 and SEQ ID NO: 98), 21A5.a (SEQ ID NO: 87 and SEQ ID NO: 98), 10F10 (SEQ ID NO: 91 and SEQ ID NO: 98), 10F10.1 (SEQ ID NO: 91 and SEQ ID NO: 98), 10F10.3 (SEQ ID NO: 91 and SEQ ID NO: 98), or 10F10.4 (SEQ ID NO: 91 and SEQ ID NO: 98),
IgG1, e.g. 9G4.IgG1, 9G4 (SEQ ID NO: 51, amino acids 20-135, and SEQ ID NO: 102), 9C8 (SEQ ID NO: 55 and SEQ ID NO: 102), 2H2 (SEQ ID NO: 58 and SEQ ID NO: 102), 2E5 (SEQ ID NO: 63 and SEQ ID NO: 102), 24E5 (SEQ ID NO: 67 and SEQ ID NO: 102), 21D9 (SEQ ID NO: 71 and SEQ ID NO: 102), 21D9.b (SEQ ID NO: 74 and SEQ ID NO: 102), 21D9.c (SEQ ID NO: 75 and SEQ ID NO: 102), 21D9.d (SEQ ID NO: 78 and SEQ ID NO: 102), 21D9.e (SEQ ID NO: 80 and SEQ ID NO: 102), 21A5 (SEQ ID NO: 83 and SEQ ID NO: 102), 21A5.a (SEQ ID NO: 87 and SEQ ID NO: 102), 10F10 (SEQ ID NO: 91 and SEQ ID NO: 102), 10F10.1 (SEQ ID NO: 91 and SEQ ID NO: 102), 10F10.3 (SEQ ID NO: 91 and SEQ ID NO: 102), or 10F10.4 (SEQ ID NO: 91 and SEQ ID NO: 102),
IgG1.3 (e.g. 9G4.IgG1.3, etc.), 9G4 (SEQ ID NO: 51, amino acids 20-135, and SEQ ID NO: 100), 9C8 (SEQ ID NO: 55 and SEQ ID NO: 100), 2H2 (SEQ ID NO: 58 and SEQ ID NO: 100), 2E5 (SEQ ID NO: 63 and SEQ ID NO: 100), 24E5 (SEQ ID NO: 67 and SEQ ID NO: 100), 21D9 ((i) SEQ ID NO: 113, or (ii) SEQ ID NO: 71 and SEQ ID NO: 100), 21D9.b (SEQ ID NO: 36), 21D9.c (SEQ ID NO: 38), 21D9.d (SEQ ID NO: 40), 21D9.e (SEQ ID NO: 13), 21A5 (SEQ ID NO: 15), 21A5.a (SEQ ID NO: 17), 10F10 (SEQ ID NO: 19), 10F10.1 (SEQ ID NO: 91 and SEQ ID NO: 100), 10F10.3 (SEQ ID NO: 91 and SEQ ID NO: 100), or 10F10.4 (SEQ ID NO: 91 and SEQ ID NO: 100);
IgG1.1f (e.g. 9G4.IgG1.1f, etc.), 9G4 (SEQ ID NO: 51, amino acids 20-135, and SEQ ID NO: 103), 9C8 (SEQ ID NO: 55 and SEQ ID NO: 103), 2H2 (SEQ ID NO: 58 and SEQ ID NO: 103), 2E5 (SEQ ID NO: 63 and SEQ ID NO: 103), 24E5 (SEQ ID NO: 67 and SEQ ID NO: 103), 21D9 (SEQ ID NO: 71 and SEQ ID NO: 103), 21D9.b (SEQ ID NO: 74 and SEQ ID NO: 103), 21D9.c (SEQ ID NO: 75 and SEQ ID NO: 103), 21D9.d (SEQ ID NO: 78 and SEQ ID NO: 103), 21D9.e (SEQ ID NO: 80 and SEQ ID NO: 103), 21A5 (SEQ ID NO: 83 and SEQ ID NO: 103), 21A5.a (SEQ ID NO: 87 and SEQ ID NO: 103), 10F10 (SEQ ID NO: 91 and SEQ ID NO: 103), 10F10.1 (SEQ ID NO: 91 and SEQ ID NO: 103), 10F10.3 (SEQ ID NO: 91 and SEQ ID NO: 103), or 10F10.4 (SEQ ID NO: 91 and SEQ ID NO: 103),
IgG1fa.P238K (e.g., 9G4.IgG1fa.P238K, etc.) 9G4 (SEQ ID NO: 51, amino acids 20-135, and SEQ ID NO: 104), 9C8 (SEQ ID NO: 55 and SEQ ID NO: 104), 2H2 (SEQ ID NO: 58 and SEQ ID NO: 104), 2E5 (SEQ ID NO: 63 and SEQ ID NO: 104), 24E5 (SEQ ID NO: 67 and SEQ ID NO: 104), 21D9 (SEQ ID NO: 71 and SEQ ID NO: 104), 21D9.b (SEQ ID NO: 74 and SEQ ID NO: 104), 21D9.c (SEQ ID NO: 75 and SEQ ID NO: 104), 21D9.d (SEQ ID NO: 78 and SEQ ID NO: 104), 21D9.e (SEQ ID NO: 80 and SEQ ID NO: 104), 21A5 (SEQ ID NO: 83 and SEQ ID NO: 104), 21A5.a (SEQ ID NO: 87 and SEQ ID NO: 104), 10F10 (SEQ ID NO: 91 and SEQ ID NO: 104), 10F10.1 (SEQ ID NO: 91 and SEQ ID NO: 104), 10F10.3 (SEQ ID NO: 91 and SEQ ID NO: 104), or 10F10.4 (SEQ ID NO: 91 and SEQ ID NO: 104), or
IgG4 S228P (e.g. 9G4.IgG4 S228P, etc.), 9G4 (SEQ ID NO: 51, amino acids 20-135, and SEQ ID NO: 179), 9C8 (SEQ ID NO: 55 and SEQ ID NO: 179), 2H2 (SEQ ID NO: 58 and SEQ ID NO: 179), 2E5 (SEQ ID NO: 63 and SEQ ID NO: 179), 24E5 (SEQ ID NO: 67 and SEQ ID NO: 179), 21D9 (SEQ ID NO: 71 and SEQ ID NO: 179), 21D9.b (SEQ ID NO: 74 and SEQ ID NO: 179), 21D9.c (SEQ ID NO: 75 and SEQ ID NO: 179), 21D9.d (SEQ ID NO: 78 and SEQ ID NO: 179), 21D9.e (SEQ ID NO: 80 and SEQ ID NO: 179), 21A5 (SEQ ID NO: 83 and SEQ ID NO: 179), 21A5.a (SEQ ID NO: 87 and SEQ ID NO: 179), 10F10 (SEQ ID NO: 91 and SEQ ID NO: 179), 1° F. 10.1 (SEQ ID NO: 91 and SEQ ID NO: 179), 10F10.3 (SEQ ID NO: 91 and SEQ ID NO: 179), or 10F10.4 (SEQ ID NO: 91 and SEQ ID NO: 179).

Embodiment 25: The isolated antibody of embodiment 23, wherein the HC of the antibody lacks a C-terminal lysine residue.

Embodiment 26: The isolated antibody of embodiment 23, wherein the HC of the antibody comprises the heavy chain constant region amino acid sequence of any one of SEQ ID Nos: 98, 100, 102, 103, 104, or 179.

Embodiment 27: The isolated antibody of any one of embodiments 1-26, which comprises the LC of anti-ILT4 antibody 9G4, 9C8, 2H2, 2E5, 24E5, 21D9, 21D9.b, 21D9.c, 21D9.d, 21D9.e, 21A5, 21A5.a, 10F10, 10F10.1, 10F10.3, or 10F10.4.

Embodiment 28: The isolated antibody of embodiment 27, wherein the light chain constant region is a human Kappa light chain constant region.

Embodiment 29: The isolated antibody of embodiment 27, wherein the LC comprises the sequence of: 9G4 (SEQ ID NO: 1), 9C8 (SEQ ID NO: 3), 2H2 (SEQ ID NO: 5), 2E5 (SEQ ID NO: 7), 24E5 (SEQ ID NO: 9), 21D9 (SEQ ID NO: 11), 21D9.b, 21D9.c, 21D9.d, 21D9.e, 21A5 (SEQ ID NO: 14), 21A5.a (SEQ ID NO: 16), 10F10 (SEQ ID NO: 18), 10F10.1 (SEQ ID NO: 20), 10F10.3 (SEQ ID NO: 21), or 10F10.4 (SEQ ID NO: 116).

Embodiment 30: The isolated antibody of any one of embodiments 1-29, which comprises HC and LC of anti-ILT4 antibody 9G4, 9C8, 2H2, 2E5, 24E5, 21D9, 21D9.b, 21D9.c, 21D9.d, 21D9.e, 21A5, 21A5.a, 10F10, 10F10.1, 10F10.3, or 10F10.4, wherein the constant region of the HC is IgG1 (e.g. 9G4.IgG1, etc.), IgG1.3 (e.g. 9G4.IgG1.3, etc.), IgG1.1f (e.g. 9G4.IgG1.1f, etc.), IgG4 (e.g., 9G4.IgG4, etc.), or IgG4 S228P (EU numbering) (e.g., 9G4.IgG4_S228P).

Embodiment 31: The isolated antibody of embodiment 30, which comprises:
a heavy chain (HC) comprising the amino acid sequence of the heavy chain of 9G4 ((i) SEQ ID NO: 2, or (ii) SEQ ID NO: 51, amino acids 20-135, and one of SEQ ID NO: 98, 100, 102, 103, 104, or 179) and a light chain (LC) comprising the light chain amino acid sequence of 9G4 (SEQ ID NO: 1);
a heavy chain comprising the amino acid sequence of the heavy chain of 9C8 ((i) SEQ ID NO: 4 or (ii) SEQ ID NOs: 55 and one of SEQ ID NO: 98, 100, 102, 103, 104, or 179) and a light chain comprising the light chain amino acid sequence of 9C8 (SEQ ID NO: 3);
a heavy chain comprising the amino acid sequence of the heavy chain of 2H2 ((i) SEQ ID NO: 6 or (ii) SEQ ID NOs: 58 and one of SEQ ID NO: 98, 100, 102, 103, 104, or 179) and a light chain comprising the light chain amino acid sequence of 2H2 (SEQ ID NO: 5);
a heavy chain comprising the amino acid sequence of the heavy chain of 2E5 ((i) SEQ ID NO: 8 or (ii) SEQ ID NOs: 63 and one of SEQ ID NO: 98, 100, 102, 103, 104, or 179) and a light chain comprising the light chain amino acid sequence of 2E5 (SEQ ID NO: 7);
a heavy chain comprising the amino acid sequence of the heavy chain of 24E5 ((i) SEQ ID NO: 10 or (ii) SEQ ID NOs: 67 and one of SEQ ID NO: 98, 100, 102, 103, 104, or 179) and a light chain comprising the light chain amino acid sequence of 24E5 (SEQ ID NO: 9);
a heavy chain comprising the amino acid sequence of the heavy chain of 21D9 ((i) SEQ ID NO: 12 or 113, or (ii) SEQ ID NOs: 71 and one of SEQ ID NO: 98, 100, 102, 103, 104, or 179) and a light chain comprising the light chain amino acid sequence of 21D9 (SEQ ID NO: 11);
a heavy chain comprising the amino acid sequence of the heavy chain of 21D9.b (SEQ ID NOs: 74 and one of SEQ ID NO: 98, 100, 102, 103, 104, or 179) and a light chain comprising the light chain amino acid sequence of 21D9.b (SEQ ID NO: 11);
a heavy chain comprising the amino acid sequence of the heavy chain of 21D9.c (SEQ ID NOs: 75 and one of SEQ ID NO: 98, 100, 102, 103, 104, or 179) and a light chain comprising the light chain amino acid sequence of 21D9.c (SEQ ID NO: 11);
a heavy chain comprising the amino acid sequence of the heavy chain of 21D9.d (SEQ ID NOs: 78 and one of SEQ ID NO: 98, 100, 102, 103, 104, or 179) and a light chain comprising the light chain amino acid sequence of 21D9.d (SEQ ID NO: 11);
a heavy chain comprising the amino acid sequence of the heavy chain of 21D9.e ((i) SEQ ID NO: 13, 176, 177, or 178; or (ii) SEQ ID NOs: 80 and one of SEQ ID NO: 98, 100, 102, 103, 104, or 179) and a light chain comprising the light chain amino acid sequence of 21D9.e (SEQ ID NO: 11);
a heavy chain comprising the amino acid sequence of the heavy chain of 21A5 ((i) SEQ ID NO: 15 or (ii) SEQ ID NOs: 83 and one of SEQ ID NO: 98, 100, 102, 103, 104, or 179) and a light chain comprising the light chain amino acid sequence of 21A5 (SEQ ID NO: 14);
a heavy chain comprising the amino acid sequence of the heavy chain of 21A5.a (i) SEQ ID NO: 17 or (ii) SEQ ID NOs: 87 and one of SEQ ID NO: 98, 100, 102, 103, 104, or 179) and a light chain comprising the light chain amino acid sequence of 21A5.a (SEQ ID NO: 16);
a heavy chain comprising the amino acid sequence of the heavy chain of 10F10 ((i) SEQ ID NO: 19 or (ii) SEQ ID NOs: 91 and one of SEQ ID NO: 98, 100, 102, 103, 104, or 179) and a light chain comprising the light chain amino acid sequence of 10F10 (SEQ ID NO: 18);
a heavy chain comprising the amino acid sequence of the heavy chain of 10F10.1 (i) SEQ ID NO: 19 or (ii) SEQ ID NOs: 91 and one of SEQ ID NO: 98, 100, 102, 103, 104, or 179) and a light chain comprising the light chain amino acid sequence of 10F10.1 (SEQ ID NO: 20);
a heavy chain comprising the amino acid sequence of the heavy chain of 10F10.3 ((i) SEQ ID NO: 19 or (ii) SEQ ID NOs: 91 and one of SEQ ID NO: 98, 100, 102, 103, 104, or 179) and a light chain comprising the light chain amino acid sequence of 10F10.3 (SEQ ID NO: 21); or
a heavy chain comprising the amino acid sequence of the heavy chain of 10F10.4 ((i) SEQ ID NO: 19 or (ii) SEQ ID NOs: 91 and one of SEQ ID NO: 98, 100, 102, 103, 104, or 179) and a light chain comprising the light chain amino acid sequence of 10F10.4 (SEQ ID NO: 116).

Embodiment 32: An isolated antibody that binds specifically to human ILT4, comprising a heavy chain comprising the amino acid sequence of SEQ ID NO: 13 and a light chain comprising the light chain amino acid sequence of SEQ ID NO: 11.

Embodiment 33: An isolated antibody that binds specifically to human ILT4, comprising a heavy chain comprising the amino acid sequence of SEQ ID NO: 176 and a light chain comprising the light chain amino acid sequence of SEQ ID NO: 11.

Embodiment 34: An isolated antibody that binds specifically to human ILT4, comprising a heavy chain comprising the amino acid sequence of SEQ ID NO: 177 and a light chain comprising the light chain amino acid sequence of SEQ ID NO: 11.

Embodiment 35: An isolated antibody that binds specifically to human ILT4, comprising a heavy chain comprising the amino acid sequence of SEQ ID NO: 178 and a light chain comprising the light chain amino acid sequence of SEQ ID NO: 11.

Embodiment 36: The isolated antibody of any one of embodiments 32-35, wherein the antibody has one or more of the properties of the antibodies of embodiment 1.

Embodiment 37: The isolated antibody of any one of embodiments 1-36, which is a full length antibody.

Embodiment 38: The isolated antibody of any one of embodiments 1-14, which is an antibody fragment.

Embodiment 39: The isolated antibody of any one of embodiments 1-38, which is a multimeric (e.g., dimeric or trimeric) antibody.

Embodiment 40: The isolated antibody of any one of embodiments 1-39, which is linked (e.g., covalently) to another molecule.

Embodiment 41: The isolated antibody of embodiment 40, wherein the other molecule is a label.

Embodiment 42: The isolated antibody of embodiment 40, wherein the other molecule is a peptide.

Embodiment 43: The isolated antibody of embodiment 40, which is an antibody drug conjugate (ADC).

Embodiment 44: An isolated nucleic acid encoding an antibody of any one of embodiments 1-43.

Embodiment 45: An isolated nucleic acid encoding the heavy chain and/or the light chain of an antibody of any one of embodiments 1-43.

Embodiment 46: A set of at least two isolated nucleic acids encoding the heavy chain and the light chain of an antibody of any one of embodiments 1-43.

Embodiment 47: A composition comprising a nucleic acid encoding the heavy chain of an antibody of any one of embodiments 1-43 and a nucleic acid encoding the light chain of the antibody of any one of embodiments 1-43.

Embodiment 48: A cell comprising the isolated nucleic acid of any one of embodiments 44-46 or the composition of embodiment 47.

Embodiment 49: A method of preparing an antibody, comprising culturing the cell of embodiment 48 in conditions under which the antibody is expressed.

Embodiment 50: A composition comprising an isolated antibody, nucleic acid, composition or cell of any one of embodiments 1-49 and a pharmaceutically acceptable carrier.

Embodiment 51: The composition of embodiment 50, comprising a second therapeutic agent.

Embodiment 52: The composition of embodiment 51, wherein the second therapeutic agent is an immunostimulatory agent.

Embodiment 53: The composition of embodiment 52, wherein the immunostimulating agent is an antagonist of an immunosuppressive molecule, e.g., the PD-1/PD-L1, a CTLA-4 and LAG-3, or an agonist of an immunostimulating molecule, e.g., GITR and OX40.

Embodiment 54: A method of treating a subject having cancer, comprising administering to the subject a therapeutically effective amount of a composition of any one of embodiments 50-53 or isolated antibody of any one of embodiments 1-43 that stimulates an immune response and/or is an ILT-4 antagonist.

Embodiment 55: The method of embodiment 54, wherein the method further comprises administering a second therapy.

Embodiment 56: The method of embodiment 55, wherein the second therapy is radiotherapy, surgery or administration of a second agent.

Embodiment 57: The method of embodiment 55, wherein the second therapy is a second agent and the second agent is an immunostimulatory agent.

Embodiment 58: The method of embodiment 57, wherein the immunostimulatory agent is an antagonist of an immunosuppressive molecule, e.g., the PD-1/PD-L1, an CTLA-4 and LAG-3, or an agonist of an immunostimulating molecule, e.g., GITR and OX40.

Embodiment 60: A method of treating an infectious disease (e.g., viral disease) in a subject, comprising administering to the subject a therapeutically effective amount of a composition of any one of embodiments 50-53 or isolated antibody of any one of embodiments 1-43 that stimulates an immune response and/or is an ILT4 antagonist.

Embodiment 61: A method of detecting ILT4 in a sample, comprising contacting the sample with an ILT4 antibody of any one of embodiments 1-43.

Embodiment 62: The isolated antibody of any one of embodiments 1-43, having the following characteristics:
a. specific binding to hILT4 (e.g. comprising the amino acid sequence of SEQ ID NO: 108, 109, 111, 112, or 119), e.g., with a K_{D} of 10⁻⁸ M or less, or of 10⁻⁹ M or less;
b. stimulates differentiation or activation of monocytes into macrophages, e.g., stimulates differentiation of monocytes into pro-inflammatory macrophages, e.g., as shown in an assay described in the Examples;
c. has a binding profile as shown in FIG. 27; and
d. binds to the Ig-like domains 1, 2 or 1 and 2 of hILT4, such as comprising the following regions of hILT4: (i) ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122); or (iii) ¹⁵4ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), and does not bind significantly to other regions of the extracellular domain of ILT4, such as regions or residues that located N-terminal to amino acid 170, wherein the amino acid numbering of hILT4 is that of immature hILT4 (i.e., ILT4 comprising its native signal sequence).

Embodiment 63: The isolated antibody of any one of embodiments 1-43, having the following characteristics:
a. specific binding to hILT4 (e.g. comprising the amino acid sequence of SEQ ID NO: 108, 109, 111, 112, or 119), e.g., with a K_{D} of 10⁻⁸ M or less, or of 10⁻⁹ M or less;
b. lack of specific binding to hILT2, hILT3, and/or hILT5;
c. lack of specific binding to one or more members of the LILRA and/or LILRB families;
d. stimulates T cell activation, e.g., in a mixed lymphocyte reaction (MLR) assay, as measured by increased T cell proliferation or IFN-gamma secretion, e.g., as shown in an assay described in the Examples;
e. stimulates differentiation or activation of monocytes into macrophages, e.g., stimulates differentiation of monocytes into pro-inflammatory macrophages, e.g., as shown in an assay described in the Examples;
f. inhibits binding of hILT4 to HLA-A and HLA-B;
g. has a binding profile as shown in FIG. 27;
h. binds to the Ig-like domains 1, 2 or 1 and 2 of hILT4, such as comprising the following regions of hILT4: (i) ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122), and does not bind significantly to other regions of the extracellular domain of ILT4, such as regions or residues that located N-terminal to amino acid 170; wherein the amino acid numbering of hILT4 is that of immature hILT4 (i.e., ILT4 with its signal sequence).
i. promotes pro-inflammatory polarization of macrophages towards M1 macrophages;
j. does not induce (or trigger) basophil activation; and
k. comprises less than 5% of high and low molecular weight species after 3 months incubation at 25° C. and/or less than 10% of high and low molecular weight species after 3 months incubation at 40° C.

Embodiment 64: The isolated antibody of any one of embodiments 1-43, having the following characteristics:
a. promotes expression of CD83 and CD86 on human monocyte derived immature dendritic cells (Mo-iDC), e.g., as shown in an assay described in the Examples;
b. enhances IFN-γ secretion upon antigen stimulation in a cytomegalovirus (CMV) lysate assay, e.g., as shown in an assay described in the Examples;
c. enhances IFN-γ and TNF-α secretion by CD4+ and CD8+ T cells in an allo-mixed lymphocyte reaction (MLR) assay upon CD3 stimulation, e.g., as shown in an assay described in the Examples;
d. inhibits binding of HLA-A and/or HLA-B to ILT4;
e. binds to Ig-like domains 1, 2 or 1 and 2 of hILT4, such as to a region comprising (i) ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122); or (iii) ¹⁵4ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), as determined by hydrogen deuterium exchange (HDX), e.g., as shown in an HDX assay described in the Examples;
f. interacts with one or more (or all of) amino acid residues Lys43, Ile49, Thr50 and Arg51 of mature hILT4 or interacts with one or more (or all of) amino acid residues Gly117, Val119, Try120, Leu134, Lys136, Gln149, Pro150, Ile159, Ser161, Val162,Gly163, Pro164, Pro167, His173, Try178, Pro183 and Tyr184 of mature hILT4 or interacts with one or more (or all of) amino acid residues Glu42, Lys43, Gly76, Cys77, Leu88, Pro91, Pro183 and Tyr184 of mature hILT4, as determined by carbene foot-printing, e.g., as described in the Examples;
g. competes for binding to hILT4 with an antibody described herein; and
h. binds specifically to a cyno ILT4 comprising SEQ ID NO: 118, e.g., as shown in a binding assay described in the Examples.

### DETAILED DESCRIPTION OF CERTAIN EMBODIMENTS

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. The terms “comprising,” “including,” and “having” can be used interchangeably herein. According to the present invention, an “isolated” molecule is a molecule that has been removed from its natural milieu. As such, the term “isolated” does not necessarily reflect the extent to which the molecule has been purified.

The term “polypeptide” refers to a polymer of amino acid residues, and is not limited to a minimum length. A “protein” may comprise one or more polypeptides. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” or “protein” refers to a polypeptide or protein, respectively, which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts that produce the proteins or errors due to PCR amplification. A protein may comprise two or more polypeptides. The letter “h” in front of a protein name denotes a native, human protein herein, e.g. “hILT4.”

The terms “ILT4,” “human ILT4,” “hILT4,” “immunoglobulin-like transcript 4,” “Ig-like transcript 4,” “leukocyte immunoglobulin-like receptor B2,” “LIR2,” “LILRB2,” “MIR10,” and “CD85d” are all used interchangeably and refer to a native, human ILT4, unless otherwise specifically indicated (e.g. mouse ILT4, cynomolgus ILT4, etc.). The term includes full-length, unprocessed ILT4 as well as any form of ILT4 that results from processing in the cell. The term encompasses naturally occurring variants of human ILT4, e.g., splice variants or allelic variants. In some embodiments, ILT4 comprises or consists of an amino acid sequence of SEQ ID NO: 107 (precursor, with signal peptide) or SEQ ID NO: 108 (mature, without signal peptide). In some embodiments, ILT4 comprises an amino acid sequence of SEQ ID NO: 110 (precursor, with signal peptide) or SEQ ID NO: 111 (mature, without signal peptide).

As used herein, the term “ILT4 fragment” refers to ILT4 having one or more residues deleted from the N- and/or C-terminus of the full-length ILT4. The ILT4 fragment may or may not include an N-terminal signal peptide but that retains the ability to bind to T cells. As used herein, the term “ILT4 variant” refers to ILT4 that contains naturally occurring amino acid additions, deletions, and substitutions but that retains the ability to bind to T cells.

The term “antagonist” is used in the broadest sense, and includes any molecule that partially or fully inhibits or neutralizes a biological activity of a polypeptide, such as ILT4. Exemplary antagonist molecules include antagonist antibodies. The term “ILT4 antagonist” refers to a molecule that inhibits or blocks the biological activity of ILT4, by, e.g., blocking or inhibiting the interaction between ILT4 and a target cell, e.g., a T cell, and/or a target molecule. Exemplary ILT4 antagonists include antibodies that block binding of ILT4 to a target cell, e.g., a T cell, and/or a target molecule. An ILT4 antagonist is considered to “block binding of ILT4 to target cells or target molecules” when it reduces the amount of detectable binding to at least one of ILT4 to a target cell, e.g., a T cell, and/or a target molecule by at least 50% in a cell binding assay. In some embodiments, an ILT4 antagonist reduces the amount of detectable binding by at least 60%, at least 70%, at least 80%, or at least 90%. In some such embodiments, the antagonist is said to block ligand binding by at least 50%, at least 60%, at least 70%, etc. Blocking of binding of ILT4 to target cells, e.g., T cells, may be demonstrated, e.g., by binding of cells transfected with ILT4, e.g., ILT4 ECD and transmembrane domain, or recombinant ILT4 Fc fusion protein e.g., recombinant ILT4 ECD Fc fusion proteins on cells, such as T cells, in the presence or absence of the antagonist.

The terms “inhibition” or “inhibit” refer to a decrease, reduction, or cessation of any phenotypic characteristic or to the decrease, reduction, or cessation in the incidence, degree, or likelihood of that characteristic. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

The terms “enhancing T cell activity” or “enhancement of T cell activity” refer to an enhancement or increase of at least one of T cell activation, cytokine secretion, such as interferon-gamma (IFN-γ) secretion, or T cell proliferation in a subject. “Enhancing T cell activity” can be by using an agent that is an agonist of T cell activity, and/or an agent that is an antagonist of (i.e., inhibits or blocks) a mechanism that inhibits T cell activity. Changes in T cell activity may be measured by, e.g., T cell proliferation assay or an IFN-γ ELISA, e.g., as described in the Examples.

The terms “ILT4 antibody” or “hILT4 antibody” or “anti-ILT4 antibody” or “anti-hILT4 antibody” or “antibody that binds ILT4,” as used herein, refer to an antibody that binds to ILT4 and that optionally inhibits the biological activity of ILT4, such as by blocking or inhibiting the binding of ILT4 to target cells, such as T cells, or target molecule. In some embodiments, the extent of binding of an ILT4 antibody to an unrelated, non-ILT4 protein is less than 10% of the binding of the antibody to ILT4 as measured, e.g., by SPR (Biacore®) or in a radioimmunoassay (RIA). In some embodiments, the extent of binding of an ILT4 antibody to other ILT family proteins, such as LILRA is less than 20%, less than 10%, less than 5% of the binding of the antibody to ILT4 as measured, e.g., by SPR or in a radioimmunoassay (RIA). In some embodiments, an ILT4 antibody binds ILT4 but does not bind at least one protein selected from ILT2, ILT3, and ILT5.

The term “leader peptide” or “leader sequence” or “signal peptide” or “signal sequence” refers to a peptide or sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

The term “antibody” or “Ab” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody also includes, but is not limited to, chimeric antibodies, humanized antibodies, human antibodies, and antibodies of various species such as mouse, cynomolgus monkey, etc. The term “antibody fragment” includes, but is not limited to, fragments that are capable of binding antigen, such as Fv, single-chain Fv (scFv), Fab, Fab′, and (Fab′)₂.

The term “heavy chain” or “HUC” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence, and with or without a C-terminal lysine (K). The term “mature full-length heavy chain” refers to a polypeptide comprising a heavy chain variable region and a heavy chain constant region, without a leader sequence, and with or without a C-terminal lysine (K).

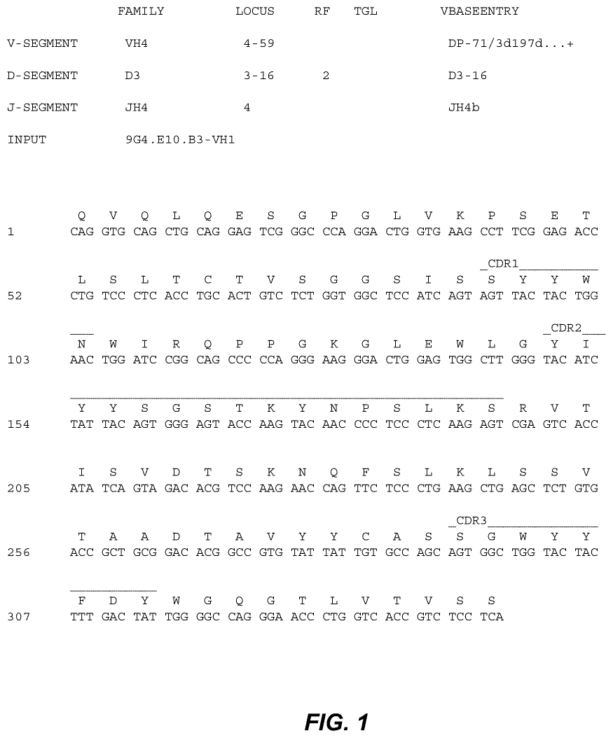

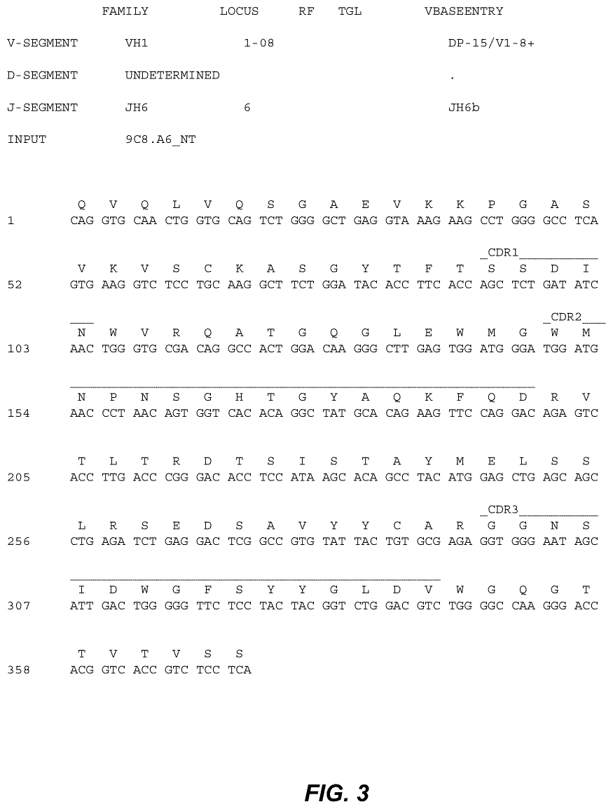

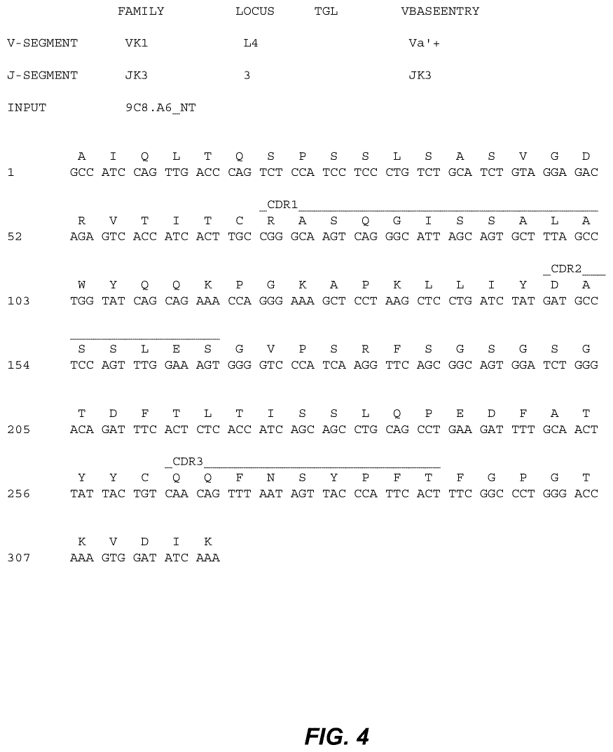

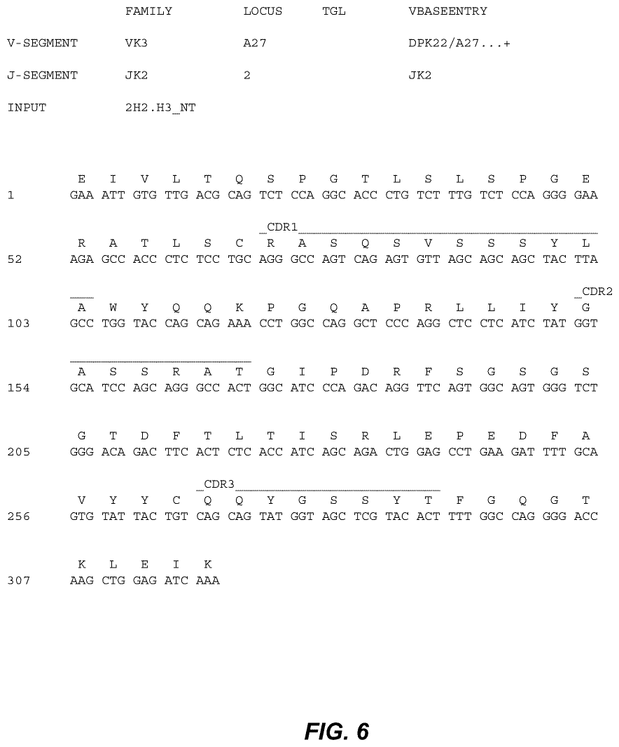

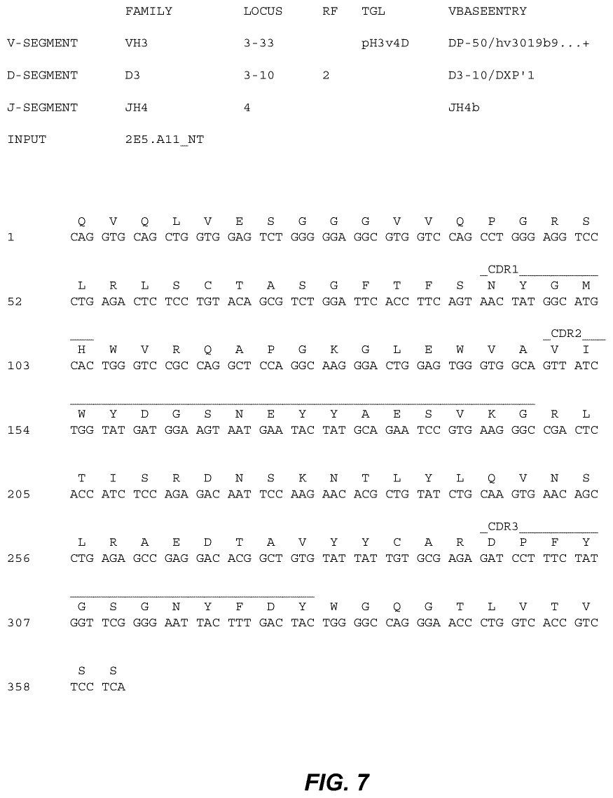

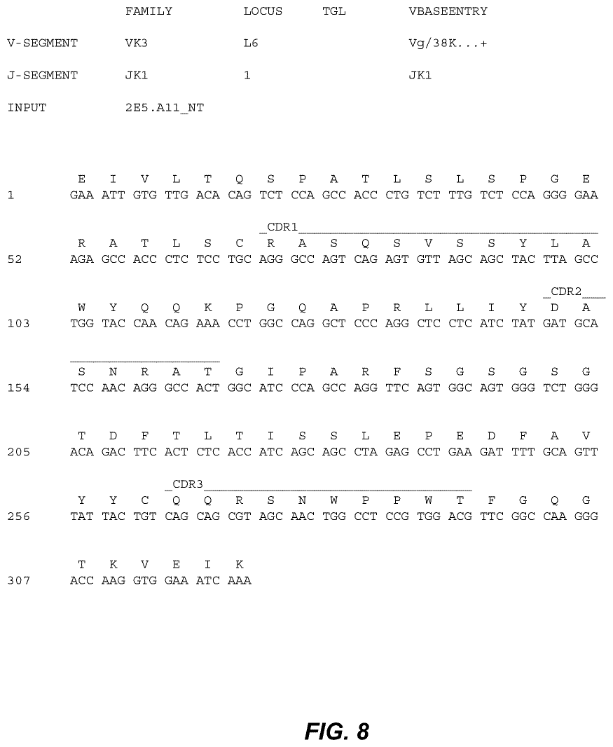

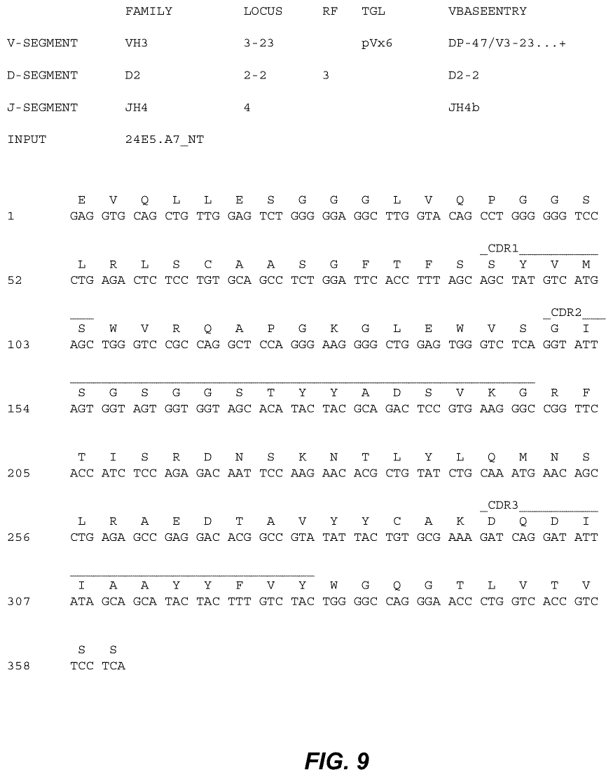

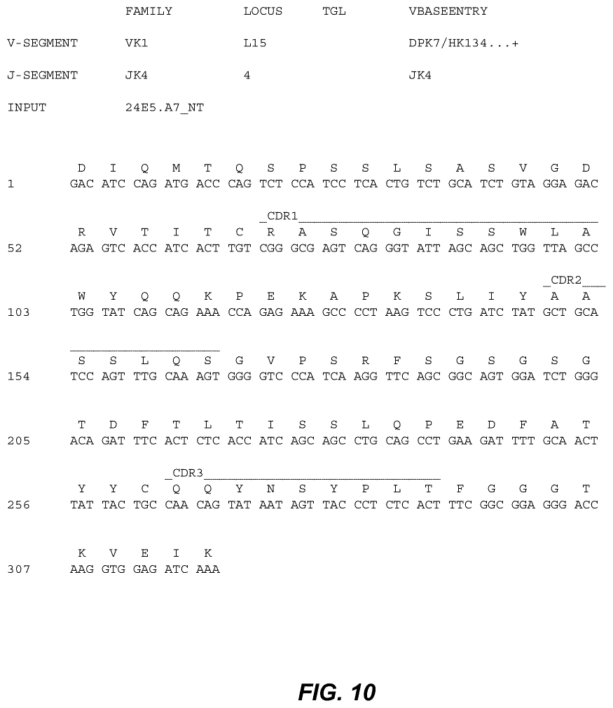

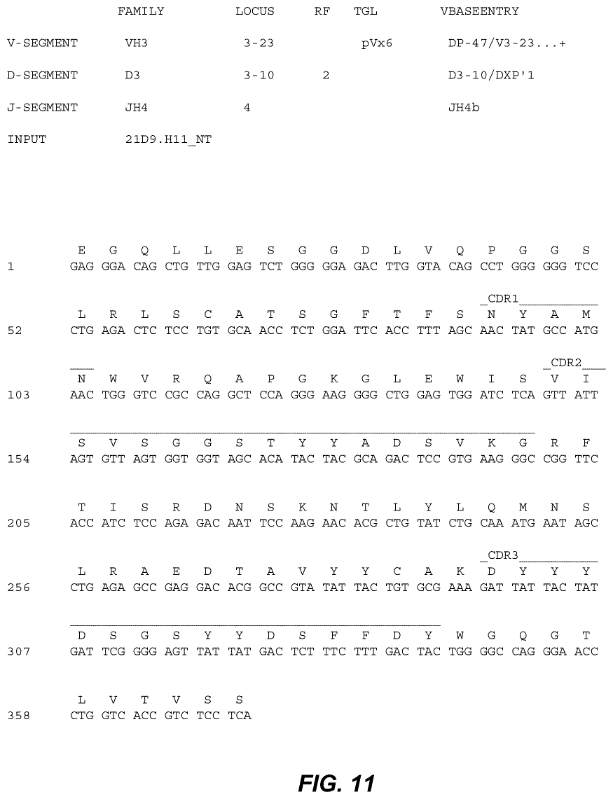

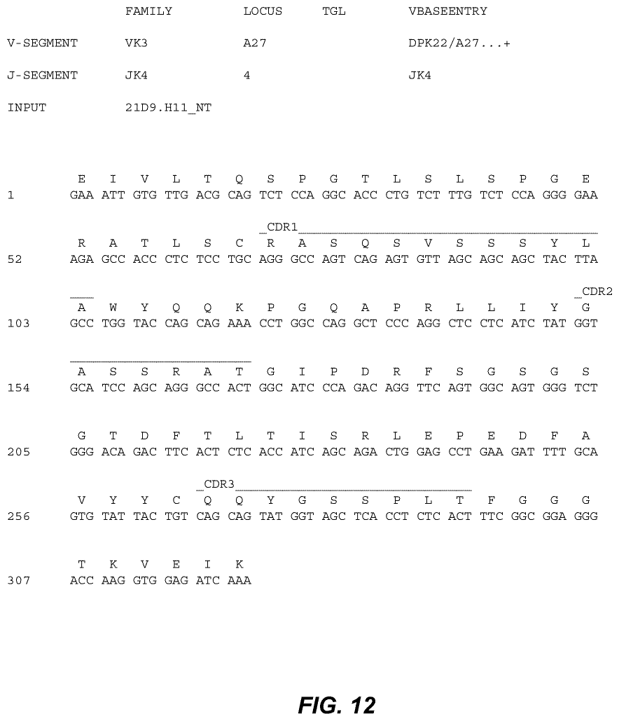

The term “heavy chain variable region” or “VH” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments the heavy chain CDRs are as specified herein, such as in the sequence table below or in FIGS. 1-12. As used herein, the VH CDR1, CDR2, and CDR3 are Kabat CDRs as provided in FIGS. 1-12, for example.

The term “light chain” or “LC” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “mature full-length light chain” refers to a polypeptide comprising a light chain variable region and a light chain constant region, without a leader sequence.

The term “light chain variable region” or “VL” refers to a region comprising a light chain CDR1, FR2, HVR2, FR3, and HVR3. In some embodiments, a light chain variable region also comprises an FR1 and/or an FR4. In some embodiments, the light chain CDRs are as specified herein such as in the sequence table or in FIGS. 1-12. As used herein, the VL CDR1, CDR2, and CDR3 are Kabat CDRs as provided in FIGS. 1-12, for example.

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

In some embodiments, an antibody herein may contain one or more “conservative substitutions” compared to a particular, specified sequence. “Conservative amino acid substitutions” herein refer to substitutions of an amino acid residue with an amino acid residue having a similar side chain. Families of amino acid residues having similar side chains include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, a predicted nonessential amino acid residue in an antibody herein is replaced with another amino acid residue from the same side chain family (e.g., basic, acidic, beta-branched, aromatic, uncharged polar). Methods of identifying nucleotide and amino acid conservative substitutions which do not eliminate antigen binding have been described, for example, in Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

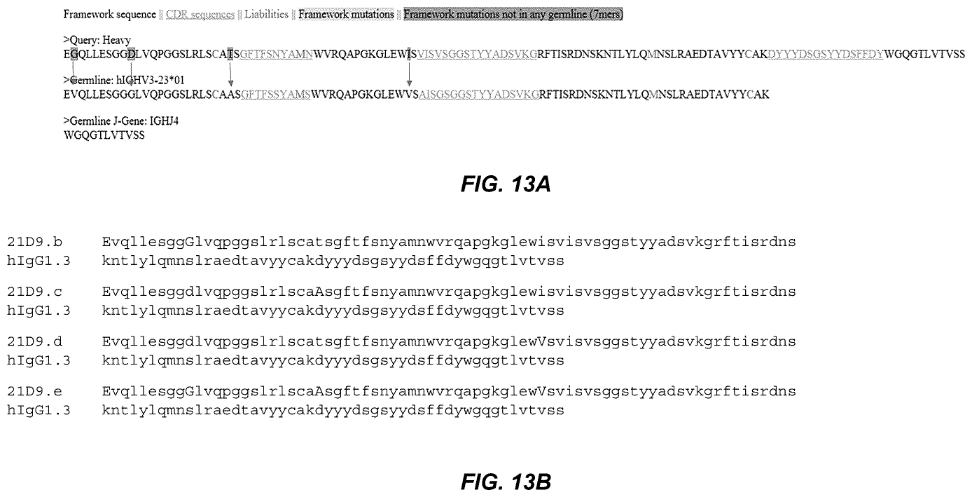

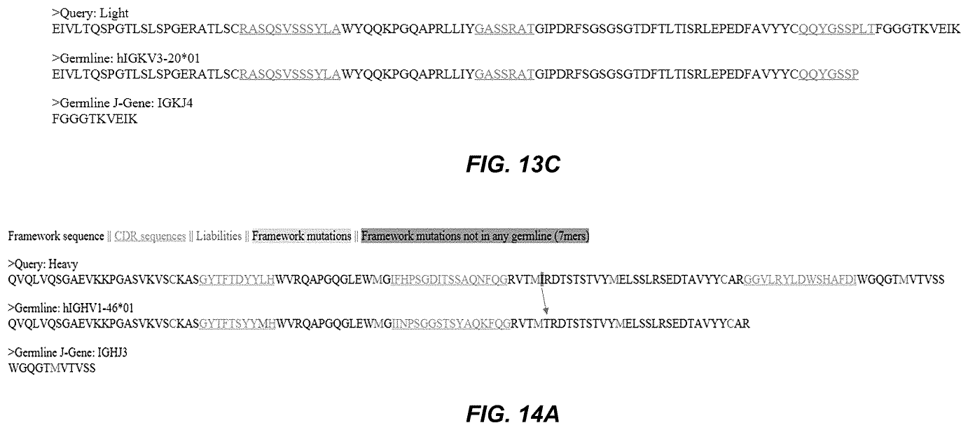

In some embodiments, an antibody herein may contain one or more “reversion substitutions.” Examples are depicted in FIGS. 13-15 herein. Reversion substitutions are mutations back to a germ line amino acid sequence from which an antibody heavy chain or light chain was derived.

A “K_{D}” or “dissociation constant” for binding of an antibody to a protein, e.g., ILT4, is a measure of the affinity or specific binding of the antibody to the protein, e.g., ILT4. A lower K_{D} indicates improved binding or affinity over a higher K_{D}. A K_{D} is composed of a ratio between an “off-rate” or k_{off} or k_{d} and an “on-rate” or kₒₙ or kₐ for the antibody and polypeptide.

The terms “specific binding” or “specifically binds” or like terms signify that the K_{D} for the binding of two polypeptides, such as an antibody and its polypeptide target, is less than would be the case between two random polypeptides existing under the same conditions. In other words, the K_{D} is less than that due to nonspecific aggregation of polypeptides in the system.

A “tumor model,” as used herein, refers to an in vivo preclinical assay, which may be used for studying the biological activity of an ILT4 antibody, and includes xenograft or native mouse tumor assay systems. In some cases, a tumor model may allow for tracking of tumor size or growth upon treatment with the antibody, and/or tracking of the presence of immune cells in the tumor, such as specific types of T-cells or NK cells, in order to determine whether an antibody has triggered or enhanced an immune response.

The term “immune stimulating agent” as used herein refers to a molecule that stimulates the immune system by either acting as an agonist of an immune-stimulatory molecule, including a co-stimulatory molecule, or acting as an antagonist of an immune inhibitory molecule, including a co-inhibitory molecule. The immune-stimulatory molecule or immune inhibitory molecule may be an immune checkpoint regulator, such as a checkpoint inhibitor or checkpoint stimulator. An immune stimulating agent may be a biologic, such as an antibody or antibody fragment, other protein, or vaccine, or may be a small molecule drug. An “immune stimulatory molecule” includes a receptor or ligand that acts to enhance, stimulate, induce, or otherwise “turn-on” an immune response. Immune stimulatory molecules as defined herein include co-stimulatory molecules. An “immune inhibitory molecule” includes a receptor or ligand that acts to reduce, inhibit, suppress, or otherwise “turn-off” an immune response. Immune inhibitory molecules as defined herein include co-inhibitory molecules. Such immune stimulatory and immune inhibitory molecules may be, for example, receptors or ligands found on immune cells such as a T cells, or found on cells involved in innate immunity such as NK cells.

“Percent (%) amino acid sequence identity,” % identity,” “percent amino acid sequence homology,” and “% homology” with respect to a peptide, polypeptide or antibody sequence mean the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

“Treatment” as used herein, covers any administration or application of a therapeutic for disease in a human, and includes inhibiting disease progression of the disease or one or more disease symptoms, slowing the disease or its progression or one or more of its symptoms, arresting its development, partially or fully relieving the disease or one or more of its symptoms, or preventing a recurrence of one or more symptoms of the disease.

The terms “subject” and “patient” are used interchangeably herein to refer to a human unless specifically stated otherwise.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “tumor growth” is used herein to refer to proliferation or growth by a cell or cells that comprise a cancer that leads to a corresponding increase in the size or extent of the cancer.

Examples of cancers applicable to methods of treatment herein include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

Administration “in combination with” one or more further therapeutic agents includes simultaneous (concurrent) and consecutive (sequential) administration in any order.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

A “chemotherapeutic agent” is a chemical compound useful in the treatment of cancer. Examples of chemotherapeutic agents that can be administered in methods herein include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Illinois), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapeutic agents that can be administered in methods herein include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent that can be administered in methods herein can include an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12):1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent that can be administered in methods herein may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA4 inhibitors (e.g., anti-CTLA antibody ipilimumab (YERVOY®)), PD-1 ore PD-L1 inhibitors (e.g., OPDIVO®, KEYTRUDA®, TECENTRIQ®, BAVENCIO®, IMFINZI®), TIM3 inhibitors (e.g., anti-TIM3 antibodies), LAG-3 inhibitors, cytokines, antagonists (e.g., neutralizing antibodies) that bind to one or more of the following targets ErbB2, ErbB3, ErbB4, PDGFR-beta, BlyS, APRIL, BCMA, CTLA4, TIM3, or VEGF receptor(s), TRAIL/Apo2, and other bioactive and organic chemical agents, etc. Combinations thereof are also included in this disclosure.

### Antibodies Specifically Binding to ILT4

The Sequence Table below provides the amino acid sequence of human ILT4 with or without signal peptide, respectively (see SEQ ID Nos: 107-108 and 110-111). The extracellular domain (ECD) comprises the amino acid residues shown in SEQ ID Nos: 109 and 112. An exemplary ECD coupled to a His-Avi tag comprises the amino acid sequence of SEQ ID NO: 119.

Anti-ITL4 antibodies (Abs) may specifically bind to the ILT4-ECD or fragments thereof (see SEQ ID Nos: 109 and 112, as well as 119).

Provided herein are Abs that bind to ILT4 with a K_{D} of 10⁻⁶ M or less, 10⁻⁷ M or less, 10⁻⁸ M or less, 10⁻⁹ M or less or 10⁻¹⁰ M or less, as measured, e.g., at 25° C. or at 37° C.

Determining how well an Ab binds to an ILT4 protein can be conducted using several different methods. For example, by surface plasmon resonance (SPR), such as by BIACORE® assays. An exemplary SPR assay comprises capturing one or several antibodies on a CM4 sensor chip with immobilized capture reagent (e.g., using Biacore® anti-human Fc capture kit, GE Healthcare catalog #BR-1008-39, or Biacore® anti-mouse capture kit, GE Healthcare catalog #BR-1008-39), and flowing ILT4 antigen (e.g. ILT4 ECD) as analyte in a concentration series to determine binding kinetics and affinities in a running buffer. In one embodiment, ILT4 is injected at two to five concentrations in the range of 0.1 nM to 500 nM (e.g., 0.1 nM, 1 nM, 10 nM, 100 nM, 500 nM) with a flow rate of 30 uL/min, up to four minutes association time and up to ten minutes dissociation time. Between binding cycles, the capture surface is regenerated following the manufacturer's instructions for the respective capture kit. All data are double-referenced using a reference flow cell and a blank injection. Data with simple 1:1 kinetics are fitted to a Langmuir binding model with mass transfer using the Biacore® T200 evaluation software. The SPR methods described in the Examples may also be used.

The affinity of an Ab for an ILT4 ECD polypeptide may be determined using cells expressing an ILT4 polypeptide on their surface, which method comprises flow cytometry. An exemplary flow cytometry assay comprises the following: T cells or other cells ectopically expressing ILT4 are re-suspended in a buffer in which the Ab has been serially diluted from approximately 20 μg/mL and incubated with the re-suspended cells for 30 minutes at 4° C. Cells are then washed twice with the same buffers, maintaining the desired buffer conditions, and incubated with a fluorophore-conjugated secondary antibody that recognizes the primary antibody (e.g., human IgG). Cells are then washed as before and acquired immediately, without fixation, on a BD Fortessa® or other flow cytometer. The affinity of an Ab for an ILT4 polypeptide may be determined as described in the Examples.

In certain embodiments, Abs that bind to ILT4 block binding of ILT4 to target cells, such as T cells. Inhibition or blocking may be 100% or at least 99%, 95%, 90%, 85%, 80%, 75%, or 50%.

Inhibition of binding between ILT4 and ILT4-binding cells such as T cells can be determined by measuring the inhibition of binding of cells to which ILT4 binds in the presence and absence of the antibody. An exemplary experiment that can be used to determine if an antibody inhibits the binding of ILT4 to ILT4-binding cells is a flow cytometry assay, e.g., an assay that comprises the following: human peripheral blood mononuclear cells from donor blood, buffy coat, or leukopak are re-suspended in a buffer consisting of HBSS+1% BSA. The cells are then incubated for 30 minutes at 4° C. with 20 μg/mL of, for example, ILT4 ECD or an ILT4 ECD fusion protein fused to human IgG1 Fc and with varying concentrations of candidate ILT4 blocking antibodies or control antibodies. Cells are then washed twice in the same buffers and incubated for another 30 minutes at 4° C. with a fluorophore-conjugated secondary antibody that recognizes ILT4, but not the candidate blocking antibodies or control antibodies. Cells are then washed as before and acquired immediately, without fixation, on a BD Fortessa® or other flow cytometer. Inhibition of binding can also be determined, e.g., as described in the Examples.

### Exemplary ILT4 Binding Abs

Provided herein are Abs that bind specifically to ILT4, e.g., to ILT4 ECD.

In certain embodiments, an ILT4 Ab comprises a heavy chain variable region (“VH”) comprising VH CDR1, CDR2 and/or CDR3 of any of the ILT4 Abs provided herein. VH CDRs herein are Kabat CDRs as shown in FIGS. 1, 3, 5, 7, 9, and 11 herein, unless specified otherwise (e.g. an AbM CDR1, etc.) In certain embodiments, an ILT4 Ab comprises a VH comprising the VH CDR1, CDR2 and CDR3 of any of the ILT4 Abs provided herein. In certain embodiments, an ILT4 Ab comprises a VH comprising VH CDR1, CDR2 and/or CDR3 of any one of 9G4, 9C8, 2H2, 2E5, 24E5, 21D9, 21D9.b, 21D9.c, 21D9.d, 21D9.e, 21A5, 21A5.a, 10F10, 10F10.1, 10F10.3, or 10F10.4, (i.e., any one of 9G4 (SEQ ID Nos: 125-127), 9C8 (SEQ ID Nos: 131-133), 2H2 (SEQ ID Nos: 137-139), 2E5 (SEQ ID Nos: 143-145), 24E5 (SEQ ID Nos: 149-151), 21D9 (SEQ ID Nos: 155-157), 21D9.b (SEQ ID Nos: 155-157), 21D9.c (SEQ ID Nos: 155-157), 21D9.d (SEQ ID Nos: 155-157), 21D9.e (SEQ ID Nos: 155-157), 21A5 (SEQ ID Nos: 161-163), 10F10 (SEQ ID Nos: 167-169), 10F10.1 (SEQ ID Nos: 167-169), 10F10.3 (SEQ ID Nos: 167-169), or 10F10.4 (SEQ ID Nos: 167-169)).

In certain embodiments, an ILT4 Ab comprises a VH comprising VH CDR1, CDR2 and CDR3 of any of the ILT4 Abs provided herein. In certain embodiments, an ILT4 Ab comprises a VH comprising VH CDR1, CDR2 and CDR3 of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

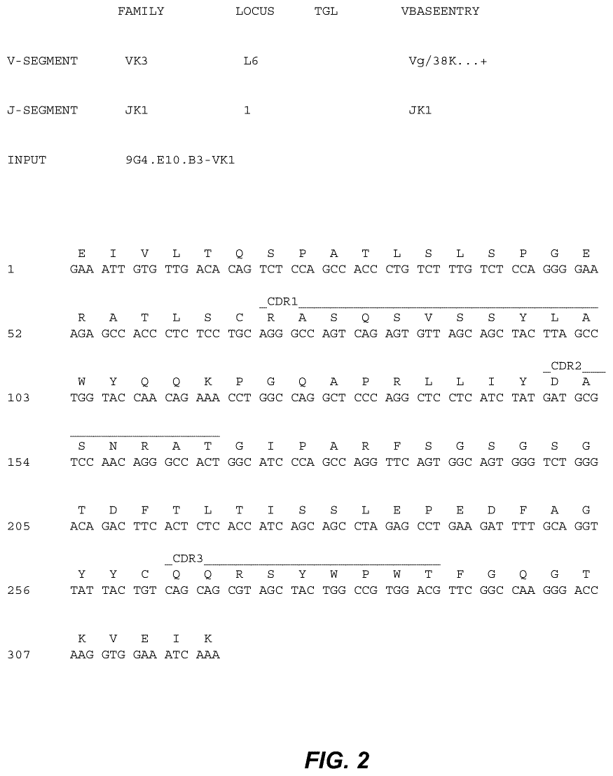

In certain embodiments, an ILT4 Ab comprises a VH comprising VH CDR1, CDR2 and/or CDR3 of any of the ILT4 Abs provided herein and a VL comprising CDR1, CDR2 and/or CDR3 of any of the ILT4 Abs provided herein. VL CDRs herein are Kabat CDRs as shown in FIGS. 2, 4, 6, 8, 10, and 12 herein, unless specified otherwise (e.g. an AbM CDR1, etc.) In certain embodiments, an ILT4 Ab comprises a VH comprising VH CDR1, CDR2 and CDR3 of any of the ILT4 Abs provided herein and a VL comprising CDR1, CDR2 and CDR3 of any of the ILT4 Abs provided herein. In certain embodiments, an ILT4 Ab comprises a VH comprising VH CDR1, CDR2 and/or CDR3 of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4 and a VL comprising VL CDR1, CDR2, and/or CDR3 of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4 (i.e., any one of 9G4 (SEQ ID Nos: 128-130), 9C8 (SEQ ID Nos: 134-136), 2H2 (SEQ ID Nos: 140-142), 2E5 (SEQ ID Nos: 146-148), 24E5 (SEQ ID Nos: 152-154), 21D9 (SEQ ID Nos: 158-160), 21D9.b (SEQ ID Nos: 158-160), 21D9.c (SEQ ID Nos: 158-160), 21D9.d (SEQ ID Nos: 158-160), 21D9.e (SEQ ID Nos: 158-160), 21A5 (SEQ ID Nos: 164-166), 10F10 (SEQ ID Nos: 170-172), 10F10.1 (SEQ ID Nos: 170-172), 10F10.3 (SEQ ID Nos: 170-172), or 10F10.4 (SEQ ID Nos: 170-172)).

In some embodiments, an ILT4 Ab may comprise:
(a) a VH comprising the amino acid sequence of the VH CDR1, CDR2 and CDR3 of 9G4 and a VL comprising the VL CDR1, CDR2 and CDR3 of 9G4;
(b) a VH comprising the amino acid sequence of the VH CDR1, CDR2 and CDR3 of 9C8 and a VL comprising the VL CDR1, CDR2 and CDR3 of 9C8;
(c) a VH comprising the amino acid sequence of the VH CDR1, CDR2 and CDR3 of 2H2 and a VL comprising the VL CDR1, CDR2 and CDR3 of 2H2;
(d) a VH comprising the amino acid sequence of the VH CDR1, CDR2 and CDR3 of 25E5 and a VL comprising the VL CDR1, CDR2 and CDR3 of 25E5;
(e) a VH comprising the amino acid sequence of the VH CDR1, CDR2 and CDR3 of 24E5 and a VL comprising the VL CDR1, CDR2 and CDR3 of 24E5;
(f) a VH comprising the amino acid sequence of the VH CDR1, CDR2 and CDR3 of 21D9 and a VL comprising the VL CDR1, CDR2 and CDR3 of 21D9;
(g) a VH comprising the amino acid sequence of the VH CDR1, CDR2 and CDR3 of 21D9.b and a VL comprising the VL CDR1, CDR2 and CDR3 of 21D9.b;
(h) a VH comprising the amino acid sequence of the VH CDR1, CDR2 and CDR3 of 21D9.c and a VL comprising the VL CDR1, CDR2 and CDR3 of 21D9.c;
(i) a VH comprising the amino acid sequence of the VH CDR1, CDR2 and CDR3 of 21D9.d and a VL comprising the VL CDR1, CDR2 and CDR3 of 21D9.d;
(j) a VH comprising the amino acid sequence of the VH CDR1, CDR2 and CDR3 of 21D9.e and a VL comprising the VL CDR1, CDR2 and CDR3 of 21D9.e;
(k) a VH comprising the amino acid sequence of the VH CDR1, CDR2 and CDR3 of 21A5 and a VL comprising the VL CDR1, CDR2 and CDR3 of 21A5;
(l) a VH comprising the amino acid sequence of the VH CDR1, CDR2 and CDR3 of 21A5.a and a VL comprising the VL CDR1, CDR2 and CDR3 of 21A5.a;
(m) a VH comprising the amino acid sequence of the VH CDR1, CDR2 and CDR3 of 10F10 and a VL comprising the VL CDR1, CDR2 and CDR3 of 10F10;
(n) a VH comprising the amino acid sequence of the VH CDR1, CDR2 and CDR3 of 10F10.1 and a VL comprising the VL CDR1, CDR2 and CDR3 of 10F10.1;
(o) a VH comprising the amino acid sequence of the VH CDR1, CDR2 and CDR3 of 10F10.3 and a VL comprising the VL CDR1, CDR2 and CDR3 of 10F10.3; or
(p) a VH comprising the amino acid sequence of the VH CDR1, CDR2 and CDR3 of 10F10.4 and a VL comprising the VL CDR1, CDR2 and CDR3 of 10F10.4.

Again, the Sequence Table below provides the heavy and light chain variable region sequences and full length heavy and light chain sequences of the antibodies listed above.

In certain embodiments, an ILT4 Ab comprises a VH comprising the amino acid sequence of the VH of any of the ILT4 Abs provided herein. The individual VH sequences for particular antibody species provided herein are listed in the Sequence Table. In certain embodiments, an ILT4 Ab comprises a VH comprising the amino acid sequence of the VH of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, (i.e., any one of 9G4 (SEQ ID NO: 51, amino acids 20-135), 9C8 (SEQ ID NO: 55), 2H2 (SEQ ID NO: 58), 2E5 (SEQ ID NO: 63), 24E5 (SEQ ID NO: 67), 21D9 (SEQ ID NO: 71), 21D9.b (SEQ ID NO: 74), 21D9.c (SEQ ID NO: 75), 21D9.d (SEQ ID NO: 78), 21D9.e (SEQ ID NO: 80), 21A5 (SEQ ID NO: 83), 21A5.a (SEQ ID NO: 87), 10F10 (SEQ ID NO: 91), 10F10.1 (SEQ ID NO: 91), 10F10.3 (SEQ ID NO: 91), or 10F10.4 (SEQ ID NO: 91)).

In some embodiments, an ILT4 Ab comprises the VH of any of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, but with 1, 2, 3, 4, or 5 amino acid substitutions in the framework regions of the VH sequence, such as 1, 2, 3, 4, or 5 conservative substitutions. In some embodiments, an ILT4 Ab comprises the VH of any of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, but with 1, 2, 3, 4, or 5 reversion substitutions in the framework regions of the VH sequence.

In some embodiments, an ILT4 Ab comprises the VH and VL CDRs of any of the ILT4 Abs described herein, wherein the CDRs comprise 1, 2 or 3 amino acid additions, substitutions (e.g., conservative substitutions), or deletion among all of the CDRs.

In certain embodiments, an ILT4 Ab comprises a VH CDR1, CDR2, and CDR3 comprising the amino acid sequences of the VH CDRs of any of the ILT4 Abs provided herein and comprises a VH that is at least 90%, at least 91%, at least 92%, at least 93%, at least 94%, at least 95%, at least 96%, at least 97%, at least 98%, or at least 99% identical to the VH of any of the ILT4 Abs provided herein. In certain embodiments, an ILT4 Ab comprises a VH comprising an amino acid sequence that is at least 90%, at least 91%, at least 92%, at least 93%, at least 94%, at least 95%, at least 96%, at least 97%, at least 98%, or at least 99% identical to the amino acid sequence of the VH of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4. In certain embodiments, the VH of the antibody differs from that of the VH sequences shown in the Sequence Table due to 1, 2, 3, 4, or 5 amino acid substitutions in the framework regions of the VH sequence, such as 1, 2, 3, 4, or 5 conservative substitutions. In certain embodiments, the VH of the antibody differs from that of the VH sequences shown in the Sequence Table due to 1, 2, 3, 4, or 5 reversion substitutions in the framework regions of the VH sequence.

In certain embodiments, an ILT4 Ab comprises a VH consisting of the amino acid sequence of the VH of any of the ILT4 Abs provided herein. In certain embodiments, an ILT4 Ab comprises a VH that consists of the amino acid sequence of the VH of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

In certain embodiments, an ILT4 Ab comprises a VL comprising the amino acid sequence of the VL of any of the ILT4 Abs provided herein. In certain embodiments, an ILT4 Ab comprises a VL comprising the amino acid sequence of the VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, (i.e., 9G4 (SEQ ID NO: 50, amino acids 19-125), 9C8 (SEQ ID NO: 54), 2H2 (SEQ ID NO: 59), 2E5 (SEQ ID NO: 62), 24E5 (SEQ ID NO: 66), 21D9 (SEQ ID NO: 70), 21D9.b (SEQ ID NO: 70), 21D9.c (SEQ ID NO: 70), 21D9.d (SEQ ID NO: 70), 21D9.e (SEQ ID NO: 70), 21A5 (SEQ ID NO: 82), 21A5.a (SEQ ID NO: 86), 10F10 (SEQ ID NO: 90), 10F10.1 (SEQ ID NO: 94), 10F10.3 (SEQ ID NO: 96), or 10F10.4 (SEQ ID NO: 114)). In certain embodiments, an ILT4 Ab comprises a VL CDR1, CDR2, and CDR3 comprising the amino acid sequences of the VL CDRs of any of the ILT4 Abs provided herein and comprises a VL that is at least 90%, at least 91%, at least 92%, at least 93%, at least 94%, at least 95%, at least 96%, at least 97%, at least 98%, or at least 99% identical to the VL of any of the ILT4 Abs provided herein. In certain embodiments, an ILT4 Ab comprises a VL comprising an amino acid sequence that is at least 90%, at least 91%, at least 92%, at least 93%, at least 94%, at least 95%, at least 96%, at least 97%, at least 98%, or at least 99% identical to the amino acid sequence of the VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4. In certain embodiments, the VL of the antibody differs from that of the VL sequences shown in the Sequence Table due to 1, 2, 3, 4, or 5 amino acid substitutions in the framework regions of the VL sequence, such as 1, 2, 3, 4, or 5 conservative substitutions. In certain embodiments, the VL of the antibody differs from that of the VL sequences shown in the Sequence Table due to 1, 2, 3, 4, or 5 reversion substitutions.

In certain embodiments, an ILT4 Ab comprises a VL consisting of the amino acid sequence of the VL of any of the ILT4 Abs provided herein. In certain embodiments, an ILT4 Ab comprises a VL that consists of the amino acid sequence of the VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

In certain embodiments, an ILT4 Ab comprises a VH comprising the amino acid sequence of the VH of any of the ILT4 Abs provided herein and comprises a VL comprising the amino acid sequence of the VL of any of the ILT4 Abs provided herein. In certain of these embodiments, an ILT4 Ab comprises a VH comprising the amino acid sequence of the VH of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4 and a VL comprising the amino acid sequence of the VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

In certain embodiments, however, the VH of the antibody is that of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, but with 1, 2, 3, 4, or 5 amino acid substitutions in the framework regions of the VH sequence, such as 1, 2, 3, 4, or 5 conservative substitutions, and the VL is that of any one of the same species from the list above. In certain embodiments, however, the VH of the antibody is that of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, but with 1, 2, 3, 4, or 5 reversion substitutions in the framework regions of the VH sequence.

In certain embodiments, an ILT4 Ab comprises a VH and a VL comprising the amino acid sequences of the VH and VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

In certain embodiments, an ILT4 Ab comprises a VH CDR1, CDR2, and CDR3 comprising the amino acid sequences of the VH CDRs of any of the ILT4 Abs provided herein as well as a VL CDR1, CDR2, and CDR3 comprising the amino acid sequences of the VL CDRs of any of the ILT4 Abs provided herein, and also comprises a VH and a VL that are each at least 90%, at least 91%, at least 92%, at least 93%, at least 94%, at least 95%, at least 96%, at least 97%, at least 98%, or at least 99% identical to the corresponding VH and VL of any of the ILT4 Abs provided herein. In certain embodiments, the VH and the VL of the antibody differ from the VH and VL sequences shown in the Sequence Table due to 1, 2, 3, 4, or 5 amino acid substitutions in the framework regions of the sequences, such as 1, 2, 3, 4, or 5 conservative substitutions, or such as 1, 2, 3, 4 or 5 reversion substitutions.

In certain embodiments, an ILT4 Ab comprises a VH and a VL consisting of the amino acid sequence of the VH and VL of any of the ILT4 Abs provided herein. In certain embodiments, an ILT4 Ab comprises a VH and a VL that each consist of the amino acid sequences of the VH and VL of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4.

An ILT4 Ab may comprise:
(a) a VH comprising the amino acid sequence of the VH of 9G4 and a VL comprising the amino acid sequence of the VL of 9G4;
(b) a VH comprising the amino acid sequence of the VH of 9C8 and a VL comprising the amino acid sequence of the VL of 9C8;
(c) a VH comprising the amino acid sequence of the VH of 2H2 and a VL comprising the amino acid sequence of the VL of 2H2;
(d) a VH comprising the amino acid sequence of the VH of 25E5 and a VL comprising the amino acid sequence of the VL of 25E5;
(e) a VH comprising the amino acid sequence of the VH of 24E5 and a VL comprising the amino acid sequence of the VL of 24E5;
(f) a VH comprising the amino acid sequence of the VH of 21D9 and a VL comprising the amino acid sequence of the VL of 21D9;
(g) a VH comprising the amino acid sequence of the VH of 21D9.b and a VL comprising the amino acid sequence of the VL of 21D9.b;
(h) a VH comprising the amino acid sequence of the VH of 21D9.c and a VL comprising the amino acid sequence of the VL of 21D9.c;
(i) a VH comprising the amino acid sequence of the VH of 21D9.d and a VL comprising the amino acid sequence of the VL of 21D9.d;
(j) a VH comprising the amino acid sequence of the VH of 21D9.e and a VL comprising the amino acid sequence of the VL of 21D9.e;
(k) a VH comprising the amino acid sequence of the VH of 21A5 and a VL comprising the amino acid sequence of the VL of 21A5;
(l) a VH comprising the amino acid sequence of the VH of 21A5.a and a VL comprising the amino acid sequence of the VL of 21A5.a;
(m) a VH comprising the amino acid sequence of the VH of 10F10 and a VL comprising the amino acid sequence of the VL of 10F10;
(n) a VH comprising the amino acid sequence of the VH of 10F10.1 and a VL comprising the amino acid sequence of the VL of 10F10.1;
(o) a VH comprising the amino acid sequence of the VH of 10F10.3 and a VL comprising the amino acid sequence of the VL of 10F10.3; or
(p) a VH comprising the amino acid sequence of the VH of 10F10.4 and a VL comprising the amino acid sequence of the VL of 10F10.4.

An ILT4 Ab may comprise:
(a) a VH comprising the VH CDRs of the VH of 9G4, and a VL comprising the VL CDRs of 9G4, and VH and VL amino acid sequences that are at least 90%, at least 91%, at least 92%, at least 93%, at least 94%, at least 95%, at least 96%, at least 97%, at least 98%, or at least 99% identical to the VH and VL of 9G4;
(b) a VH comprising the VH CDRs of the VH of 9C8, and a VL comprising the VL CDRs of 9C8, and VH and VL amino acid sequences that are at least 90%, at least 91%, at least 92%, at least 93%, at least 94%, at least 95%, at least 96%, at least 97%, at least 98%, or at least 99% identical to the VH and VL of 9C8;
(c) a VH comprising the VH CDRs of the VH of 2H2, and a VL comprising the VL CDRs of 2H2, and VH and VL amino acid sequences that are at least 90%, at least 91%, at least 92%, at least 93%, at least 94%, at least 95%, at least 96%, at least 97%, at least 98%, or at least 99% identical to the VH and VL of 2H2;
(d) a VH comprising the VH CDRs of the VH of 25E5, and a VL comprising the VL CDRs of 25E5, and VH and VL amino acid sequences that are at least 90%, at least 91%, at least 92%, at least 93%, at least 94%, at least 95%, at least 96%, at least 97%, at least 98%, or at least 99% identical to the VH and VL of 25E5;
(e) a VH comprising the VH CDRs of the VH of 24E5, and a VL comprising the VL CDRs of 24E5, and VH and VL amino acid sequences that are at least 90%, at least 91%, at least 92%, at least 93%, at least 94%, at least 95%, at least 96%, at least 97%, at least 98%, or at least 99% identical to the VH and VL of 24E5;
(f) a VH comprising the VH CDRs of the VH of 21D9, and a VL comprising the VL CDRs of 21D9, and VH and VL amino acid sequences that are at least 90%, at least 91%, at least 92%, at least 93%, at least 94%, at least 95%, at least 96%, at least 97%, at least 98%, or at least 99% identical to the VH and VL of 21D9;
(g) a VH comprising the VH CDRs of the VH of 21D9.b, and a VL comprising the VL CDRs of 21D9.b, and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.b;
(h) a VH comprising the VH CDRs of the VH of 21D9.c, and a VL comprising the VL CDRs of 21D9.c, and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.c;
(i) a VH comprising the VH CDRs of the VH of 21D9.d, and a VL comprising the VL CDRs of 21D9.d, and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.d;
(j) a VH comprising the VH CDRs of the VH of 21D9.e, and a VL comprising the VL CDRs of 21D9.e, and VH and VL amino acid sequences that are at least 90%, at least 91%, at least 92%, at least 93%, at least 94%, at least 95%, at least 96%, at least 97%, at least 98%, or at least 99% identical to the VH and VL of 21D9.e;
(k) a VH comprising the VH CDRs of the VH of 21A5, and a VL comprising the VL CDRs of 21A5, and VH and VL amino acid sequences that are at least 90%, at least 91%, at least 92%, at least 93%, at least 94%, at least 95%, at least 96%, at least 97%, at least 98%, or at least 99% identical to the VH and VL of 21A5;
(l) a VH comprising the VH CDRs of the VH of 21A5.a, and a VL comprising the VL CDRs of 21A5.a, and VH and VL amino acid sequences that are at least 90%, at least 91%, at least 92%, at least 93%, at least 94%, at least 95%, at least 96%, at least 97%, at least 98%, or at least 99% identical to the VH and VL of 21A5.a;
(m) a VH comprising the VH CDRs of the VH of 10F10, and a VL comprising the VL CDRs of 10F10, and VH and VL amino acid sequences that are at least 90%, at least 91%, at least 92%, at least 93%, at least 94%, at least 95%, at least 96%, at least 97%, at least 98%, or at least 99% identical to the VH and VL of 10F10;
(n) a VH comprising the VH CDRs of the VH of 10F10.1, and a VL comprising the VL CDRs of 10F10.1, and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.1;
(o) a VH comprising the VH CDRs of the VH of 10F10.3, and a VL comprising the VL CDRs of 10F10.3, and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.3; or
(p) a VH comprising the VH CDRs of the VH of 10F10.3, and a VL comprising the VL CDRs of 10F10.4, and VH and VL amino acid sequences that are at least 90%, at least 91%, at least 92%, at least 93%, at least 94%, at least 95%, at least 96%, at least 97%, at least 98%, or at least 99% identical to the VH and VL of 10F10.4.

In some of the above embodiments, the VH and/or VL may differ from the sequence of each of the species (a) to (p) by the presence of 1, 2, 3, 4, or 5 amino acid substitutions, such as 1, 2, 3, 4, or 5 conservative substitutions. In some embodiments, the VH may comprise 1, 2, 3, 4, or 5 reversion substitutions.

An ILT4 Ab may comprise:
(a) a VH consisting of the amino acid sequence of the VH of 9G4 and a VL consisting of the VL of 9G4;
(b) a VH consisting of the amino acid sequence of the VH of 9C8 and a VL consisting of the VL of 9C8;
(c) a VH consisting of the amino acid sequence of the VH of 2H2 and a VL consisting of the VL of 2H2;
(d) a VH consisting of the amino acid sequence of the VH of 25E5 and a VL consisting of the VL of 25E5;
(e) a VH consisting of the amino acid sequence of the VH of 24E5 and a VL consisting of the VL of 24E5;
(f) a VH consisting of the amino acid sequence of the VH of 21D9 and a VL consisting of the VL of 21D9;
(g) a VH consisting of the amino acid sequence of the VH of 21D9.b and a VL consisting of the VL of 21D9.b;
(h) a VH consisting of the amino acid sequence of the VH of 21D9.c and a VL consisting of the VL of 21D9.c;
(i) a VH consisting of the amino acid sequence of the VH of 21D9.d and a VL consisting of the VL of 21D9.d;
(j) a VH consisting of the amino acid sequence of the VH of 21D9.e and a VL consisting of the VL of 21D9.e;
(k) a VH consisting of the amino acid sequence of the VH of 21A5 and a VL consisting of the VL of 21A5;
(l) a VH consisting of the amino acid sequence of the VH of 21A5.a and a VL consisting of the VL of 21A5.a;
(m) a VH consisting of the amino acid sequence of the VH of 10F10 and a VL consisting of the VL of 10F10;
(n) a VH consisting of the amino acid sequence of the VH of 10F10.1 and a VL consisting of the VL of 10F10.1;
(o) a VH consisting of the amino acid sequence of the VH of 10F10.3 and a VL consisting of the VL of 10F10.3; or
(p) a VH consisting of the amino acid sequence of the VH of 10F10.4 and a VL consisting of the VL of 10F10.4.

In certain embodiments, an ILT4 Ab comprises any of the variable regions and/or variable region CDRs 1-3 of the antibodies described above and elsewhere herein, such as:
(1) one or more of VH CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(2) the VH CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(3) the VH of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(4) one or more of VH CDR1, CDR2 and CDR3 and one or more of VL CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(5) the VH CDR1, CDR2 and CDR3 and VL CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(6) the VH and the VLs of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
or
(7) the VL and the VH, with the exception of 1, 2, 3, 4, or 5 reversion or conservative substitutions in the VH and/or VL of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4; and the ILT4 Ab is also an IgG antibody, such as IgG1, IgG2, IgG3 or IgG4 antibody or a modified form thereof as described in the section below. In some embodiments, the constant region has effector function, and in some embodiments, the constant region is effectorless. In certain embodiments, the constant region is that of IgG1.3 or IgG1.1f or another constant region described herein, e.g., IgG1 and IgG1.238K.

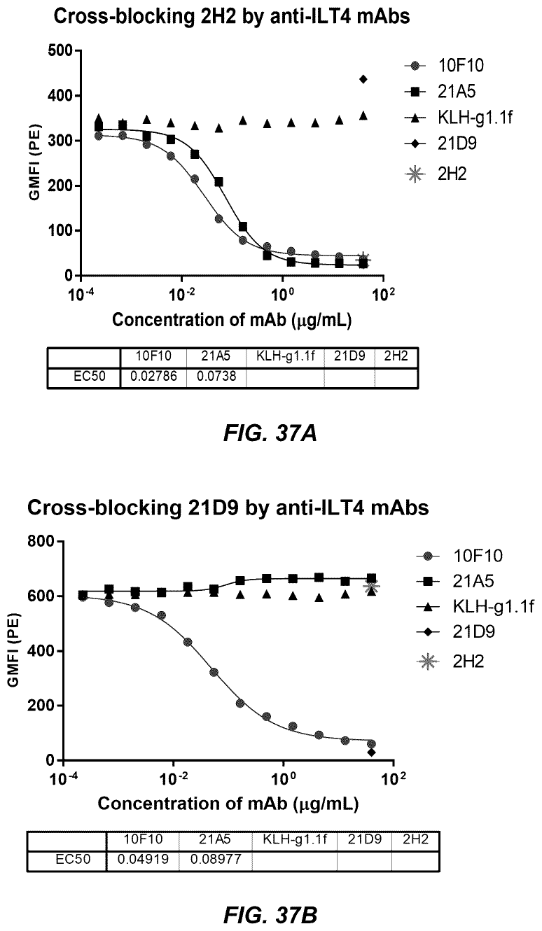

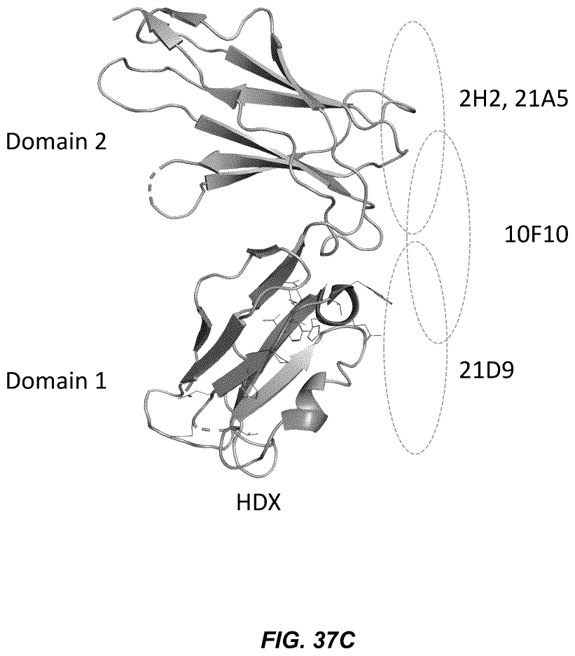

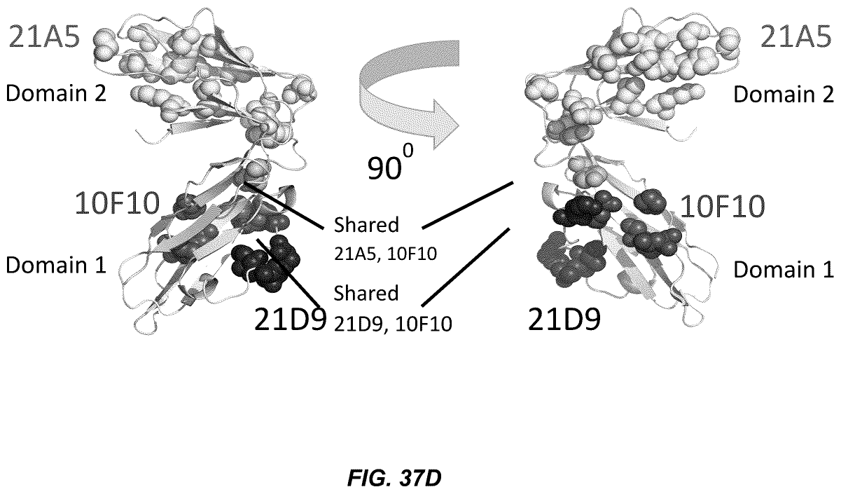

In certain embodiments, an ILT4 Ab comprises any of the variable regions and/or variable region CDRs 1-3 of the antibodies described above and elsewhere herein, such as:
(1) one or more of VH CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(2) the VH CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(3) the VH of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(4) one or more of VH CDR1, CDR2 and CDR3 and one or more of VL CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(5) the VH CDR1, CDR2 and CDR3 and VL CDR1, CDR2 and CDR3 of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
(6) the VH and the VLs of: 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4;
or
(7) the VL and the VH of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, with the exception of 1, 2, 3, 4, or 5 reversion or conservative substitutions in the VH and/or VL;
and
the antibody further comprises one or more of the following characteristics:
   specific binding to hILT4, e.g., with a K_{D} of 10⁻⁸ M, 10⁻⁹ M or less;
   lack of specific binding to hILT2, hILT3, hILT5;
   lack of specific binding to one or more members of the LILRA and/or LILRB families;
   stimulates T cell activation, e.g., in an MLR, as measured by increased T cell proliferation or IFN-g secretion, e.g., as shown in an assay described in the Examples;
   stimulates differentiation or activation of monocytes into macrophages, e.g., stimulates differentiation of monocytes into pro-inflammatory macrophages, e.g., as shown in an assay described in the Examples;
   promotes expression of CD83 and CD86 on human monocyte derived immature dendritic cells (Mo-iDC), e.g., as shown in an assay described in the Examples;
   enhances IFN-γ secretion upon antigen stimulation in a CMV lysate assay, e.g., as shown in an assay described in the Examples;
   enhances IFN-γ and TNF-α secretion by CD4+ and CD8+ T cells in an allo-mixed lymphocyte reaction (allo-MLR) assay upon CD3 stimulation, e.g., as shown in an assay described in the Examples;
   does not induce (or trigger) basophil activation;
   inhibits binding of HLA-A and/or HLA-B to ILT4;
   has a binding profile to hILT4 as shown in FIG. 27 or FIG. 37C;
   binds to Ig-like domains 1, 2 or 1 and 2 of hILT4 (which correspond to amino acids 27-110 and 111-229 of SEQ ID NO: 107, respectively), such as to a region comprising (i) ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122); or (iii) ¹⁵⁴ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), as determined by hydrogen deuterium exchange (HDX), e.g., as shown in an HDX assay described in the Examples;
   does not bind significantly to other regions of the extracellular domain of ILT4, such as regions or residues that located N-terminal to amino acid 170; wherein the amino acid numbering of hILT4 is that of immature hILT4 (i.e., ILT4 with its signal sequence);
   competes for binding to hILT4 with an antibody described herein;
   binds specifically to cyno ILT4 comprising SEQ ID NO: 118, e.g., as shown in a binding assay described in the Examples;
   promotes pro-inflammatory polarization of macrophages towards M1 macrophages;
   does not induce (or trigger) basophil activation; and
   comprises less than 5% of high and low molecular weight species after 3 months incubation at 25° C. and/or less than 10% of high and low molecular weight species after 3 months incubation at 40° C.

In certain embodiments, an ILT4 Ab comprises a heavy chain (HC) comprising the amino acid sequence of the heavy chain of any of the ILT4 Abs provided herein. In certain embodiments, an ILT4 Ab comprises a heavy chain comprising the amino acid sequence of the heavy chain of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, as shown below in the Sequence Table (i.e., a complete HC sequence as provided in the Table, or a composite of a VH sequence for the antibody with an HC constant region sequence such as SEQ ID NO: 98, 100, 102, 103, 104, or 179, as provided in the Table).

Thus, for example, in some embodiments, the antibody may comprise a HC amino acid sequence of one of the types below, comprising an amino acid sequence as follows:
a. IgG1, e.g. 9G4.IgG1, 9G4 (SEQ ID NO: 2), 9C8 (SEQ ID NO: 4), 2H2 (SEQ ID NO: 6), 2E5 (SEQ ID NO: 8), 24E5 (SEQ ID NO: 10), 21D9 (SEQ ID NO: 12), 21D9.b (SEQ ID NO: 74 and SEQ ID NO: 98), 21D9.c (SEQ ID NO: 75 and SEQ ID NO: 98), 21D9.d (SEQ ID NO: 78 and SEQ ID NO: 98), 21D9.e (SEQ ID NO: 80 and SEQ ID NO: 98), 21A5 (SEQ ID NO: 83 and SEQ ID NO: 98), 21A5.a (SEQ ID NO: 87 and SEQ ID NO: 98), 10F10 (SEQ ID NO: 91 and SEQ ID NO: 98), 10F10.1 (SEQ ID NO: 91 and SEQ ID NO: 98), 10F10.3 (SEQ ID NO: 91 and SEQ ID NO: 98), or 10F10.4 (SEQ ID NO: 91 and SEQ ID NO: 98),
b. IgG1, e.g. 9G4.IgG1, 9G4 (SEQ ID NO: 51, amino acids 20-135, and SEQ ID NO: 102), 9C8 (SEQ ID NO: 55 and SEQ ID NO: 102), 2H2 (SEQ ID NO: 58 and SEQ ID NO: 102), 2E5 (SEQ ID NO: 63 and SEQ ID NO: 102), 24E5 (SEQ ID NO: 67 and SEQ ID NO: 102), 21D9 (SEQ ID NO: 71 and SEQ ID NO: 102), 21D9.b (SEQ ID NO: 74 and SEQ ID NO: 102), 21D9.c (SEQ ID NO: 75 and SEQ ID NO: 102), 21D9.d (SEQ ID NO: 78 and SEQ ID NO: 102), 21D9.e (SEQ ID NO: 80 and SEQ ID NO: 102), 21A5 (SEQ ID NO: 83 and SEQ ID NO: 102), 21A5.a (SEQ ID NO: 87 and SEQ ID NO: 102), 10F10 (SEQ ID NO: 91 and SEQ ID NO: 102), 10F10.1 (SEQ ID NO: 91 and SEQ ID NO: 102), 10F10.3 (SEQ ID NO: 91 and SEQ ID NO: 102), or 10F10.4 (SEQ ID NO: 91 and SEQ ID NO: 102),
c. IgG1.3 (e.g. 9G4.IgG1.3, etc.), 9G4 (SEQ ID NO: 51, amino acids 20-135, and SEQ ID NO: 100), 9C8 (SEQ ID NO: 55 and SEQ ID NO: 100), 2H2 (SEQ ID NO: 58 and SEQ ID NO: 100), 2E5 (SEQ ID NO: 63 and SEQ ID NO: 100), 24E5 (SEQ ID NO: 67 and SEQ ID NO: 100), 21D9 ((i) SEQ ID NO: 113, or (ii) SEQ ID NO: 71 and SEQ ID NO: 100), 21D9.b (SEQ ID NO: 36), 21D9.c (SEQ ID NO: 38), 21D9.d (SEQ ID NO: 40), 21D9.e (SEQ ID NO: 13), 21A5 (SEQ ID NO: 15), 21A5.a (SEQ ID NO: 17), 10F10 (SEQ ID NO: 19), 10F10.1 (SEQ ID NO: 91 and SEQ ID NO: 100), 10F10.3 (SEQ ID NO: 91 and SEQ ID NO: 100), or 10F10.4 (SEQ ID NO: 91 and SEQ ID NO: 100);
d. IgG1.1f (e.g. 9G4.IgG1.1f, etc.), 9G4 (SEQ ID NO: 51, amino acids 20-135, and SEQ ID NO: 103), 9C8 (SEQ ID NO: 55 and SEQ ID NO: 103), 2H2 (SEQ ID NO: 58 and SEQ ID NO: 103), 2E5 (SEQ ID NO: 63 and SEQ ID NO: 103), 24E5 (SEQ ID NO: 67 and SEQ ID NO: 103), 21D9 (SEQ ID NO: 71 and SEQ ID NO: 103), 21D9.b (SEQ ID NO: 74 and SEQ ID NO: 103), 21D9.c (SEQ ID NO: 75 and SEQ ID NO: 103), 21D9.d (SEQ ID NO: 78 and SEQ ID NO: 103), 21D9.e (SEQ ID NO: 80 and SEQ ID NO: 103), 21A5 (SEQ ID NO: 83 and SEQ ID NO: 103), 21A5.a (SEQ ID NO: 87 and SEQ ID NO: 103), 10F10 (SEQ ID NO: 91 and SEQ ID NO: 103), 10F10.1 (SEQ ID NO: 91 and SEQ ID NO: 103), 10F10.3 (SEQ ID NO: 91 and SEQ ID NO: 103), or 10F10.4 (SEQ ID NO: 91 and SEQ ID NO: 103),
e. IgG1fa.P238K (e.g., 9G4.IgG1fa.P238K, etc.) 9G4 (SEQ ID NO: 51, amino acids 20-135, and SEQ ID NO: 104), 9C8 (SEQ ID NO: 55 and SEQ ID NO: 104), 2H2 (SEQ ID NO: 58 and SEQ ID NO: 104), 2E5 (SEQ ID NO: 63 and SEQ ID NO: 104), 24E5 (SEQ ID NO: 67 and SEQ ID NO: 104), 21D9 (SEQ ID NO: 71 and SEQ ID NO: 104), 21D9.b (SEQ ID NO: 74 and SEQ ID NO: 104), 21D9.c (SEQ ID NO: 75 and SEQ ID NO: 104), 21D9.d (SEQ ID NO: 78 and SEQ ID NO: 104), 21D9.e (SEQ ID NO: 80 and SEQ ID NO: 104), 21A5 (SEQ ID NO: 83 and SEQ ID NO: 104), 21A5.a (SEQ ID NO: 87 and SEQ ID NO: 104), 10F10 (SEQ ID NO: 91 and SEQ ID NO: 104), 10F10.1 (SEQ ID NO: 91 and SEQ ID NO: 104), 10F10.3 (SEQ ID NO: 91 and SEQ ID NO: 104), or 10F10.4 (SEQ ID NO: 91 and SEQ ID NO: 104), or
f. IgG4 S228P (e.g. 9G4.IgG4 S228P, etc.), 9G4 (SEQ ID NO: 51, amino acids 20-135, and SEQ ID NO: 179), 9C8 (SEQ ID NO: 55 and SEQ ID NO: 179), 2H2 (SEQ ID NO: 58 and SEQ ID NO: 179), 2E5 (SEQ ID NO: 63 and SEQ ID NO: 179), 24E5 (SEQ ID NO: 67 and SEQ ID NO: 179), 21D9 (SEQ ID NO: 71 and SEQ ID NO: 179), 21D9.b (SEQ ID NO: 74 and SEQ ID NO: 179), 21D9.c (SEQ ID NO: 75 and SEQ ID NO: 179), 21D9.d (SEQ ID NO: 78 and SEQ ID NO: 179), 21D9.e (SEQ ID NO: 80 and SEQ ID NO: 179), 21A5 (SEQ ID NO: 83 and SEQ ID NO: 179), 21A5.a (SEQ ID NO: 87 and SEQ ID NO: 179), 10F10 (SEQ ID NO: 91 and SEQ ID NO: 179), 10F10.1 (SEQ ID NO: 91 and SEQ ID NO: 179), 10F10.3 (SEQ ID NO: 91 and SEQ ID NO: 179), or 10F10.4 (SEQ ID NO: 91 and SEQ ID NO: 179).

In certain embodiments, an ILT4 Ab comprises a heavy chain comprising the amino acid sequence of the heavy chain of any of the ILT4 Abs provided herein, which comprise an IgG1.3 heavy chain constant region, and the amino acid sequence of the light chain of any of the ILT4 Abs provided herein. In certain embodiments, an ILT4 Ab comprises a heavy chain comprising the amino acid sequence of the VH any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4, which comprise an IgG1.3 HC constant region (which is denoted by the nomenclature 9G4.IgG1.3, etc.); and a light chain comprising the amino acid sequence of the light chain of any one of 9G4, 9C8, 2H2, 25E5, 24E5, 21D9, 21D9.b, 21D9.c, 21D9.d, 21D9.e, 21A5, 21A5.a, 10F10, 10F10.1, 10F10.3, or 10F10.4. In some embodiments, the light chain is a Kappa light chain comprising an amino acid sequence as follows: 9G4 (SEQ ID NO: 1), 9C8 (SEQ ID NO: 3), 2H2 (SEQ ID NO: 5), 2E5 (SEQ ID NO: 7), 24E5 (SEQ ID NO: 9), 21D9 (SEQ ID NO: 11), 21D9.b, 21D9.c, 21D9.d, 21D9.e, 21A5 (SEQ ID NO: 14), 21A5.a (SEQ ID NO: 16), 10F10 (SEQ ID NO: 18), 10F10.1 (SEQ ID NO: 20), 10F10.3 (SEQ ID NO: 21), or 10F10.4 (SEQ ID NO: 116).

An ILT4 Ab may comprise:
(a) a heavy chain comprising the amino acid sequence of the heavy chain of 9G4 and a light chain comprising the light chain amino acid sequence of 9G4;
(b) a heavy chain comprising the amino acid sequence of the heavy chain of 9C8 and a light chain comprising the light chain amino acid sequence of 9C8;
(c) a heavy chain comprising the amino acid sequence of the heavy chain of 2H2 and a light chain comprising the light chain amino acid sequence of 2H2;
(d) a heavy chain comprising the amino acid sequence of the heavy chain of 25E5 and a light chain comprising the light chain amino acid sequence of 25E5;
(e) a heavy chain comprising the amino acid sequence of the heavy chain of 24E5 and a light chain comprising the light chain amino acid sequence of 24E5;
(f) a heavy chain comprising the amino acid sequence of the heavy chain of 9G4 and a light chain comprising the light chain amino acid sequence of 21D9;
(g) a heavy chain comprising the amino acid sequence of the heavy chain of 21D9.b and a light chain comprising the light chain amino acid sequence of 21D9.b;
(h) a heavy chain comprising the amino acid sequence of the heavy chain of 21D9.c and a light chain comprising the light chain amino acid sequence of 21D9.c;
(i) a heavy chain comprising the amino acid sequence of the heavy chain of 21D9.d and a light chain comprising the light chain amino acid sequence of 21D9.d;
(j) a heavy chain comprising the amino acid sequence of the heavy chain of 21D9.e and a light chain comprising the light chain amino acid sequence of 21D9.e;
(k) a heavy chain comprising the amino acid sequence of the heavy chain of 21A5 and a light chain comprising the light chain amino acid sequence of 21A5;
(l) a heavy chain comprising the amino acid sequence of the heavy chain of 21A5.a and a light chain comprising the light chain amino acid sequence of 21A5.a;
(m) a heavy chain comprising the amino acid sequence of the heavy chain of 10F10 and a light chain comprising the light chain amino acid sequence of 10F10;
(n) a heavy chain comprising the amino acid sequence of the heavy chain of 10F10.1 and a light chain comprising the light chain amino acid sequence of 10F10.1;
(o) a heavy chain comprising the amino acid sequence of the heavy chain of 10F10.3 and a light chain comprising the light chain amino acid sequence of 10F10.3; or
(p) a heavy chain comprising the amino acid sequence of the heavy chain of 10F10.4 and a light chain comprising the light chain amino acid sequence of 10F10.4.

An ILT4 Ab may comprise:
(a) a heavy chain (HC) comprising the HC CDRs of the HC of 9G4 and a light chain (LC) comprising the LC CDRs of 9G4 and HC and LC amino acid sequences that are at least 90%, at least 91%, at least 92%, at least 93%, at least 94%, at least 95%, at least 96%, at least 97%, at least 98%, or at least 99% identical to the HC and LC of 9G4, respectively;
(b) a HC comprising the HC CDRs of the HC of 9C8, and a light chain (LC) comprising the LC CDRs of 9C8 and HC and LC amino acid sequences that are at least 90%, at least 91%, at least 92%, at least 93%, at least 94%, at least 95%, at least 96%, at least 97%, at least 98%, or at least 99% identical to the HC and LC of 9C8, respectively;
(c) a HC comprising the HC CDRs of the HC of 2H2, and a light chain (LC) comprising the LC CDRs of 2H2 and HC and LC amino acid sequences that are at least 90%, at least 91%, at least 92%, at least 93%, at least 94%, at least 95%, at least 96%, at least 97%, at least 98%, or at least 99% identical to the HC and LC of 2H2, respectively;
(d) a HC comprising the HC CDRs of the HC of 25E5, and a light chain (LC) comprising the LC CDRs of 25E5 and HC and LC amino acid sequences that are at least 90%, at least 91%, at least 92%, at least 93%, at least 94%, at least 95%, at least 96%, at least 97%, at least 98%, or at least 99% identical to the HC and LC of 25E5, respectively;
(e) a HC comprising the HC CDRs of the HC of 24E5, and a light chain (LC) comprising the LC CDRs of 24E5 and HC and LC amino acid sequences that are at least 90%, at least 91%, at least 92%, at least 93%, at least 94%, at least 95%, at least 96%, at least 97%, at least 98%, or at least 99% identical to the HC and LC of 24E5, respectively;
(f) a HC comprising the HC CDRs of the HC of 21D9, and a light chain (LC) comprising the LC CDRs of 21D9 and HC and LC amino acid sequences that are at least 90%, at least 91%, at least 92%, at least 93%, at least 94%, at least 95%, at least 96%, at least 97%, at least 98%, or at least 99% identical to the HC and LC of 21D9, respectively;
(g) a HC comprising the HC CDRs of the HC of 21D9.b, and a light chain (LC) comprising the LC CDRs of 21D9.b and HC and LC amino acid sequences that are at least 90%, at least 91%, at least 92%, at least 93%, at least 94%, at least 95%, at least 96%, at least 97%, at least 98%, or at least 99% identical to the HC and LC of 21D9.b, respectively;
(h) a HC comprising the HC CDRs of the HC of 21D9.c, and a light chain (LC) comprising the LC CDRs of 21D9.c and HC and LC amino acid sequences that are at least 90%, at least 91%, at least 92%, at least 93%, at least 94%, at least 95%, at least 96%, at least 97%, at least 98%, or at least 99% identical to the HC and LC of 21D9.c, respectively;
(i) a HC comprising the HC CDRs of the HC of 21D9.d, and a light chain (LC) comprising the LC CDRs of 21D9.d and HC and LC amino acid sequences that are at least 90%, at least 91%, at least 92%, at least 93%, at least 94%, at least 95%, at least 96%, at least 97%, at least 98%, or at least 99% identical to the HC and LC of 21D9.d, respectively;
(j) a HC comprising the HC CDRs of the HC of 21D9.e, and a light chain (LC) comprising the LC CDRs of 21D9.e and HC and LC amino acid sequences that are at least 90%, at least 91%, at least 92%, at least 93%, at least 94%, at least 95%, at least 96%, at least 97%, at least 98%, or at least 99% identical to the HC and LC of 21D9.e, respectively;
(k) a HC comprising the HC CDRs of the HC of 21A5, and a light chain (LC) comprising the LC CDRs of 21A5 and HC and LC amino acid sequences that are at least 90%, at least 91%, at least 92%, at least 93%, at least 94%, at least 95%, at least 96%, at least 97%, at least 98%, or at least 99% identical to the HC and LC of 21A5, respectively;
(l) a HC comprising the HC CDRs of the HC of 21A5.a, and a light chain (LC) comprising the LC CDRs of 21A5.a and HC and LC amino acid sequences that are at least 90%, at least 91%, at least 92%, at least 93%, at least 94%, at least 95%, at least 96%, at least 97%, at least 98%, or at least 99% identical to the HC and LC of 21A5.a, respectively;
(m) a HC comprising the HC CDRs of the HC of 10F10, and a light chain (LC) comprising the LC CDRs of 10F10 and HC and LC amino acid sequences that are at least 90%, at least 91%, at least 92%, at least 93%, at least 94%, at least 95%, at least 96%, at least 97%, at least 98%, or at least 99% identical to the HC and LC of 10F10, respectively;
(n) a HC comprising the HC CDRs of the HC of 10F10.1, and a light chain (LC) comprising the LC CDRs of 10F10.1 and HC and LC amino acid sequences that are at least 90%, at least 91%, at least 92%, at least 93%, at least 94%, at least 95%, at least 96%, at least 97%, at least 98%, or at least 99% identical to the HC and LC of 10F10.1, respectively;
(o) a HC comprising the HC CDRs of the HC of 10F10.3, and a light chain (LC) comprising the LC CDRs of 10F10.3 and HC and LC amino acid sequences that are at least 90%, at least 91%, at least 92%, at least 93%, at least 94%, at least 95%, at least 96%, at least 97%, at least 98%, or at least 99% identical to the HC and LC of 10F10.3, respectively; or
(p) a HC comprising the HC CDRs of the HC of 10F10.3, and a light chain (LC) comprising the LC CDRs of 10F10.4 and HC and LC amino acid sequences that are at least 90%, at least 91%, at least 92%, at least 93%, at least 94%, at least 95%, at least 96%, at least 97%, at least 98%, or at least 99% identical to the HC and LC of 10F10.4, respectively.

In some of the above embodiments, the HC and/or LC may differ from the sequence of each of the species (a) to (p) by the presence of 1, 2, 3, 4, or 5 amino acid substitutions, such as 1, 2, 3, 4, or 5 conservative substitutions. In some of the above embodiments, the HC and/or LC may differ from the sequence of each of the species (a) to (p) by the presence of 1, 2, 3, 4, or 5 amino acid substitutions, such as 1, 2, 3, 4, or 5 reversion substitutions.

In some embodiments, an ILT4 Ab may comprise:
(a) a heavy chain consisting of the amino acid sequence of the heavy chain of 9G4 and a light chain consisting of the amino acid sequence of the light chain of 9G4;
(b) a heavy chain consisting of the amino acid sequence of the heavy chain of 9C8 and a light chain consisting of the amino acid sequence of the light chain of 9C8;
(c) a heavy chain consisting of the amino acid sequence of the heavy chain of 2H2 and a light chain consisting of the amino acid sequence of the light chain of 2H2;
(d) a heavy chain consisting of the amino acid sequence of the heavy chain of 25E5 and a light chain consisting of the amino acid sequence of the light chain of 25E5;
(e) a heavy chain consisting of the amino acid sequence of the heavy chain of 24E5 and a light chain consisting of the amino acid sequence of the light chain of 24E5;
(f) a heavy chain consisting of the amino acid sequence of the heavy chain of 21D9 and a light chain consisting of the amino acid sequence of the light chain of 21D9;
(g) a heavy chain consisting of the amino acid sequence of the heavy chain of 21D9.b and a light chain consisting of the amino acid sequence of the light chain of 21D9.b;
(h) a heavy chain consisting of the amino acid sequence of the heavy chain of 21D9.c and a light chain consisting of the amino acid sequence of the light chain of 21D9.c;
(i) a heavy chain consisting of the amino acid sequence of the heavy chain of 21D9.d and a light chain consisting of the amino acid sequence of the light chain of 21D9.d;
(j) a heavy chain consisting of the amino acid sequence of the heavy chain of 21D9.e and a light chain consisting of the amino acid sequence of the light chain of 21D9.e;
(k) a heavy chain consisting of the amino acid sequence of the heavy chain of 21A5 and a light chain consisting of the amino acid sequence of the light chain of 21A5;
(l) a heavy chain consisting of the amino acid sequence of the heavy chain of 21A5.a and a light chain consisting of the amino acid sequence of the light chain of 21A5.a;
(m) a heavy chain consisting of the amino acid sequence of the heavy chain of 10F10 and a light chain consisting of the amino acid sequence of the light chain of 10F10;
(n) a heavy chain consisting of the amino acid sequence of the heavy chain of 10F10.1 and a light chain consisting of the amino acid sequence of the light chain of 10F10.1;
(o) a heavy chain consisting of the amino acid sequence of the heavy chain of 10F10.3 and a light chain consisting of the amino acid sequence of the light chain of 10F10.3; or
(p) a heavy chain consisting of the amino acid sequence of the heavy chain of 10F10.4 and a light chain consisting of the amino acid sequence of the light chain of 10F10.4.

In some embodiments, an ILT4 Ab may comprise a heavy chain amino acid sequence comprising the VH amino acid sequence of the antibody species herein, but rather than an IgG1.3 heavy chain constant region, as provided in the HC sequences in the Sequence Table herein (and see SEQ ID NO: 100), the antibody may comprise a different heavy chain constant region sequence, such as a human wild-type IgG1 constant region such as those provided in SEQ ID Nos: 98 and 102-104, or an IgG4 heavy chain constant region, or an IgG4 constant region with an S228P substitution (EU numbering), or any of the other heavy chain constant regions described in the section on constant regions that follows below.

In some embodiments, such modified ILT4 Abs possess one or more of the following characteristics:
specific binding to hILT4, e.g., with a K_{D} of 10⁻⁸ M or less, or of 10⁻⁹ M or less;
lack of specific binding to one, two, or all three of hILT2, hILT3, hILT5;
lack of specific binding to one or more members of the LILRA and/or LILRB families;
stimulates T cell activation, e.g., in an MLR assay, as measured by increased T cell proliferation or IFN-gamma secretion; and
stimulates differentiation or activation of monocytes into macrophages.

In some embodiments, the antibody binds to Ig-like domains 1, 2 or 1 and 2 of hILT4, which correspond to amino acids 27-110 on the sequence of SEQ ID NO: 107 (domain 1) and 111-229 on that sequence (domain 2), at the locations comprising ⁷⁰ITRIRPEL⁷⁷ (SEQ ID NO: 120) and/or ⁷⁸VKNGQFHIPSITWEHTGRYGCQY¹⁰⁰ (SEQ ID NO: 121); (ii) ⁷⁰ITRIRPELVKNGQFHIPSITWEHTGRYGCQY¹⁰⁰ (SEQ ID NO: 122); or (iii) ¹⁵⁴ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), as determined by hydrogen deuterium exchange (HDX), e.g., as shown in an HDX assay described in the Examples. (See FIG. 27)

### Exemplary Antibody Constant Regions

In some embodiments, an antibody described herein comprises one or more human constant regions. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region, such as an IgG1, IgG2, IgG3, or IgG4. In some embodiments, an antibody described herein comprises a human IgG4 heavy chain constant region. In some such embodiments, an antibody described herein comprises an S241P mutation in the human IgG4 constant region. In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. In some methods of treatment, including methods of treating some cancers, cell killing may be desirable, for example, when the antibody binds to a cell that supports the maintenance or growth of the tumor. Exemplary cells that may support the maintenance or growth of a tumor include, but are not limited to, tumor cells themselves, cells that aid in the recruitment of vasculature to the tumor, and cells that provide ligands, growth factors, or counter-receptors that support or promote tumor growth or tumor survival. In some embodiments, when effector function is desirable, an antibody comprising a human IgG1 heavy chain or a human IgG3 heavy chain is selected.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibodies with certain improved properties. For example, in some embodiments an antibody may be afucosylated, for example, by mutating residues such as Asn297 that are normally glycosylated with fucose-containing glycosylations, or through other means. In some embodiments, antibodies herein may comprise an afucosylated human IgG1 constant region.

Antibodies are further provided with bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibodies may have improved CDC function. Such antibodies are described, e.g., in WO 1997/30087 (Patel et al.); WO 1998/58964 (Raju, S.); and WO 1999/22764 (Raju, S.).

Antibodies are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody.

The in vivo or serum half-life of human FcRn high affinity binding polypeptides can be assayed, e.g., in transgenic mice, in humans, or in non-human primates to which the polypeptides with a variant Fc region are administered. See also, e.g., Petkova et al. International Immunology 18(12):1759-1769 (2006).

In some embodiments of the invention, an afucosylated antibody mediates ADCC in the presence of human effector cells more effectively than a parent antibody that comprises fucose, Generally, ADCC activity may be determined using the in vitro ADCC assay as herein disclosed, but other assays or methods for determining ADCC activity, e.g. in an animal model etc., are contemplated.

In certain embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320, 322, 330, and/or 331 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In some examples, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In some examples, one or more amino acid residues within amino acid positions 231 and 239 are altered to thereby alter the ability of the antibody to fix complement. This approach is described further in PCT Publication WO 94/29351 by Bodmer et al. In some examples, the Fc region can be modified to decrease antibody dependent cellular cytotoxicity (ADCC) and/or to decrease the affinity for an Fcγ receptor by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F/324T. Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, 328, 330, and/or 331 (e.g., 330 and 331), wherein numbering is according to the EU index. Exemplary substitutions include but are not limited to 234A, 235E, 236R, 237A, 267R, 269R, 325L, 328R, 330S, and 331S (e.g., 330S, and 331S), wherein numbering is according to the EU index. An Fc variant can comprise 236R/328R. Other modifications for reducing FcγR and complement interactions include substitutions 297A, 234A, 235A, 237A, 318A, 228P, 236E, 268Q, 309L, 330S, 331 S, 2205, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691. For example, the human IgG1.3 Fc constant region contains L234A, L235E, and G237A substitutions. The IgG1fa.P238K (or IgG1.P238K) contains a P238K substitution. The IgG1.1f comprises L234A, L235E, G237A, A330S, and P331S substitutions.

Fc variants that enhance affinity for an inhibitory receptor FcγRIIb can also be used. Such variants can provide an Fc fusion protein with immunomodulatory activities related to FcγRIIb cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, 330, 331, and 332, according to the EU index. Exemplary substitutions for enhancing FcγRIIb affinity include but are not limited to 234A, 234D, 234E, 234F, 234W, 235D, 235E, 235F, 235R, 235Y, 236D, 236N, 237A, 237D, 237N, 239D, 239E, 266M, 267D, 267E, 268D, 268E, 327D, 327E, 328F, 328W, 328Y, 330S, 33IS, and 332E. Exemplary substitutions include 235Y, 236D, 239D, 266M, 267E, 268D, 268E, 328F, 328W, and 328Y. Other Fc variants for enhancing binding to FcγRIIb include 235Y/267E, 236D/267E, 239D/268D, 239D/267E, 267E/268D, 267E/268E, and 267E/328F.

Other modifications for enhancing FcγR and complement interactions include but are not limited to substitutions 298 A, 333A, 334A, 326A, 2471, 339D, 339Q, 280H, 290S, 298D, 298V, 243L, 292P, 300L, 396L, 3051, and 396L. These and other modifications are reviewed in Strohl, 2009, Current Opinion in Biotechnology 20:685-691. Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in Patent Publication No. WO 00/42072.

Optionally, the Fc region can comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; PCX Patent Publications WO 00/42072; WO 01/58957; WO 02/06919; WO 04/016750; WO 04/029207; WO 04/035752; WO 04/074455; WO 04/099249; WO 04/063351; WO 05/070963; WO 05/040217, WO 05/092925 and WO 06/020114).

The affinities and binding properties of an Fc region for its ligand can be determined by a variety of in vitro assay methods (biochemical or immunological based assays) known in the art including but not limited to, equilibrium methods (e.g., enzyme-linked immunoabsorbent assay (ELISA), or radioimmunoassay (RIA)), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods can utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this can be done by increasing the binding affinity of the Fc region for FcRn, For example, one or more of more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 2591, 308F, 428L, 428M, 434S, 4341 1. 434F, 434Y, and 434X1. Other variants that increase Fc binding to FcRn include: 250E, 250Q, 428 L, 428F, 250Q/428L (Hinton et al. 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al., Journal of Biological Chemistry, 2001, 276(9):6591-6604), 252F, 252T, 252Y, 252W, 254T, 256S, 256R, 256Q, 256E, 256D, 256T, 309P, 311 S, 433R, 433S, 4331, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671.

In certain embodiments, hybrid IgG isotypes with particular biological characteristics can be used. For example, an IgG1/IgG3 hybrid variant can be constructed by substituting IgG1 positions in the CH2 and/or CH3 region with the amino acids from IgG3 at positions where the two isotypes differ. Thus a hybrid variant IgG antibody can be constructed that comprises one or more substitutions, e.g., 274Q, 276K, 300F, 339T, 356E, 358M, 384S, 392N, 397M, 4221, 435R, and 436F. In some embodiments described herein, an IgG1/IgG2 hybrid variant can be constructed by substituting IgG2 positions in the CH2 and/or CH3 region with amino acids from IgG1 at positions where the two isotypes differ. Thus a hybrid variant IgG antibody can be constructed that comprises one or more substitutions, e.g., one or more of the following amino acid substitutions: 233E, 234L, 235L, -236G (referring to an insertion of a glycine at position 236), and 327A.

Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al., 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgG1 mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that can be used include: S298A/E333A/L334A, S239D/1332E, S239D/1332E/A330L, L235V/F243L/R292P/Y300L/P396L, and M428L/N434S.

In certain embodiments, an Fc is chosen that has reduced binding to FcγRs. An exemplary Fc, e.g., IgG1 Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, e.g., IgG1 Fc, that is effectorless comprises the following five mutations: L234A, L235E, G237A, A330S and P331S.

When using an IgG4 constant domain, it can include the substitution S228P, which mimics the hinge sequence in IgG1 and thereby stabilizes IgG4 molecules.

Fc modifications described in WO 2017/087678 or WO2016081746 may also be used.

In certain embodiments, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in further detail in U.S. Pat. Nos. 5,714,350 and 6,350,861 by Co et al.

Glycosylation of the constant region on N297 can be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases {e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

Another modification of the antibodies described herein is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some embodiments, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP 0 401 384 by Ishikawa et al.

Specific exemplary antibody embodiments herein are further listed in the following sections.

### Embodiments Relating to Antibody 21D9.e

Embodiment 1: An isolated antibody (“Ab”) that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70.

Embodiment 2: An isolated antibody that binds to human ILT4, wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the antibody stimulates T cell activation (or potentiates a T cell response), e.g., (i) in a mixed lymphocyte reaction (MLR) assay, as evidenced, e.g., by enhanced T cell proliferation or IFN-gamma secretion or TNF-alpha production, or (ii) in a T cell: CHO-OKT3-ILT4 assay (e.g., as described in Example 5).

Embodiment 3: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab enhances T cell activation in a monocyte:T cell allo-MLR.

Embodiment 4: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab enhances IFN-gamma secretion from PBMCs upon antigen stimulation in a cytomegalovirus (CMV) lysate assay.

Embodiment 5: An isolated antibody that binds to human ILT4, wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the antibody potentiates TNF-alpha secretion from macrophages, e.g., differentiated macrophages.

Embodiment 6: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the antibody increases expression of CD83 and/or CD86 on monocyte-derived dendritic cells (MoDCs).

Embodiment 7: An isolated antibody that binds to human ILT4, wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the antibody inhibits binding of hILT4 to a T cell and/or to a binding partner, e.g., HLA-A and/or HLA-B.

Embodiment 8: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab (a) binds to the Ig-like domain 1 of hILT4 and/or the region of hILT4 comprising ⁷⁰ITRIRPEL⁷⁷ (SEQ ID NO: 120) and/or ⁷⁸VKNGQFHIPSITWEHTGRYGCQY¹⁰⁰ (SEQ ID NO: 121) and/or ⁷⁰ITRIRPELVKNGQFHIPSITWEHTGRYGCQY¹⁰⁰ (SEQ ID NO: 122), as determined by HDX; and/or (b) interacts with one or more (or all of) amino acid residues Lys43, Ile49, Thr50 and Arg51 of mature hILT4, as determined by carbene foot-printing.

Embodiment 9: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab inhibits binding of (or competes with) an antibody comprising a HC comprising SEQ ID NO: 12 or 13 and a light chain comprising SEQ ID NO: 11 and/or inhibits binding of an antibody comprising VH and VL of 21D9.IgG1.1f and/or 21D9.e.IgG1.3, to hILT4.

Embodiment 10: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab binds to hILT4 with a KD of 10⁻⁸ M or 10⁻⁹ M or less.

Embodiment 11: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab binds to cyno ILT4 with a KD of 10⁻⁷ M or 10⁻⁸ M or less and/or promote expression of CD80, CD83 and/or CD86 on cyno monocyte derived dendritic cells.

Embodiment 12: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab does not significantly bind to the human proteins LILRA1, LTLRA2, LTLRA3, LILRA4, LILRA6, ILT2, ILT3, ILT5 or LIR8.

Embodiment 13: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab promotes pro-inflammatory polarization of macrophages towards M1 macrophages.

Embodiment 14: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab does not induce (or trigger) basophil activation.

Embodiment 15: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein a composition comprising the Ab comprises less than 5% of high and low molecular weight species after 3 months incubation at 25° C. and/or less than 10% of high and low molecular weight species after 3 months incubation at 40° C.

Embodiment 16: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively, and/or (ii) the heavy chain comprises VH comprising SEQ ID NO: 80 and the light chain comprises VL comprising SEQ ID NO: 70, wherein the Ab comprises an IgG heavy chain constant region, e.g., an IgG1 (e.g., SEQ ID NO: 98 or 102), IgG1.If (SEQ ID NO: 103), IgG1.3 (SEQ ID NO: 100), IgGP238K (SEQ ID NO: 104), IgG4 or IgG4.S228P (SEQ ID NO: 179).

Embodiment 17: An isolated antibody that binds to hILT4, wherein the Ab comprises a heavy chain comprising SEQ ID NO: 13 and a light chain comprising SEQ ID NO: 11, wherein, optionally, the heavy chain comprises a C-terminal lysine.

Embodiment 18: An isolated antibody that binds to hILT4, wherein the Ab comprises two heavy chains, each heavy chain comprising SEQ ID NO: 13, and two light chains, each light chain comprising SEQ ID NO: 11, wherein, optionally one or both of the heavy chains comprise a C-terminal lysine.

Embodiment 19: isolated antibody that binds to hILT4, wherein the Ab comprises a heavy chain comprising SEQ ID NO: 13 and a light chain comprising SEQ ID NO: 11, wherein, optionally the heavy chain comprises a C-terminal lysine, and wherein the Ab comprises at least one disulfide bond linking the heavy chains.

Embodiment 20: An isolated antibody that binds to hILT4, wherein the Ab comprises a heavy chain comprising SEQ ID NO: 176 and a light chain comprising SEQ ID NO: 11, wherein, optionally the heavy chain comprises a C-terminal lysine.

Embodiment 21: An isolated antibody that binds to hILT4, wherein the Ab comprises a heavy chain comprising SEQ ID NO: 177 and a light chain comprising SEQ ID NO: 11, wherein, optionally the heavy chain comprises a C-terminal lysine.

Embodiment 22: An isolated antibody that binds to hILT4, wherein the Ab comprises a heavy chain comprising SEQ ID NO: 178 and a light chain comprising SEQ ID NO: 11, wherein, optionally the heavy chain comprises a C-terminal lysine.

Embodiment 23: An isolated nucleic acid or set of nucleic acids encoding the antibody of any one of Embodiments 1-22.

Embodiment 24: A host cell for producing any one of the antibodies of Embodiments 1-22, for example comprising the isolated nucleic acid or set of nucleic acids of Embodiment 23.

Embodiment 25: A method of treating cancer in a subject, comprising administering to the subject a therapeutically effective dose of the antibody of any one of Embodiments 1-22, or of an antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively; (ii) the heavy chain comprises VH comprising SEQ ID NO: 80 and the light chain comprises VL comprising SEQ ID NO: 70 and/or (iii) the heavy chain comprises SEQ ID NO: 13 and the light chain comprises SEQ ID NO: 11.

Embodiment 26: A method of treating cancer in a subject, comprising administering to the subject a therapeutically effective dose of:
(A) (1) the antibody of any one of Embodiments 1-19, or (2) an antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively; (ii) the heavy chain comprises VH comprising SEQ ID NO: 80 and the light chain comprises VL comprising SEQ ID NO: 70 and/or (iii) the heavy chain comprises SEQ ID NO: 13 and the light chain comprises SEQ ID NO: 11, and
(B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

Embodiment 27: A composition comprising:
(A) (1) the antibody of any one of Embodiments 1-19, or (2) an antibody that binds to human ILT4 (hILT4), wherein the Ab comprises a heavy chain and a light chain, wherein (i) the heavy chain comprises VH CDR1, CDR2 and CDR3 comprising SEQ ID Nos: 155, 156, and 157, respectively, and the light chain comprises VL CDR1, CDR2 and CDR3 comprising SEQ ID Nos: 158, 159 and 160, respectively; (ii) the heavy chain comprises VH comprising SEQ ID NO: 80 and the light chain comprises VL comprising SEQ ID NO: 70 and/or (iii) the heavy chain comprises SEQ ID NO: 13 and the light chain comprises SEQ ID NO: 11 and
(B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

### Embodiments Relating to Antibody 21D5.a

Embodiment 1: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86.

Embodiment 2: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the antibody stimulates T cell activation (or potentiates a T cell response), e.g., (i) in a mixed lymphocyte reaction (MLR) assay, as evidenced, e.g., by enhanced T cell proliferation or IFN-gamma secretion or TNF-alpha production, or (ii) in a T cell: CHO-OKT3-ILT4 assay (e.g., as described in Example 5).

Embodiment 3: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab enhances T cell activation in a monocyte:T cell allo-MLR.

Embodiment 4: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab enhances IFN-gamma secretion from PBMCs upon antigen stimulation in a CMV lysate assay.

Embodiment 5: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the antibody potentiates TNF-alpha secretion from macrophages, e.g., differentiated macrophages.

Embodiment 6: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the antibody increases expression of CD83 and/or CD86 on monocyte-derived dendritic cells (Mo-DCs).

Embodiment 7: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the antibody inhibits binding of hILT4 to a T cell and/or to a binding partner, e.g., HLA-A and/or HLA-B.

Embodiment 8: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab (a) binds to the Ig-like 2 domain of hILT4 and/or the region of hILT4 comprising: ¹⁵⁴ILCKEGEEEHPQCLNSQPHARGSSRAIF¹⁸¹ (SEQ ID NO: 123) and/or ⁴²⁵SSPPPTGPIS⁴³⁴ (SEQ ID NO: 124), as determined by HDX; and/or (b) interacts with one or more (or all of) amino acid residues Gly117, Val119, Try120, Leu134, Lys136, Gln149, Pro150, Ile159, Ser161, Val162,Gly163, Pro164, Pro167, His173, Try178, Pro183 and Tyr184 of mature hILT4, determined by carbene foot-printing.

Embodiment 9: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab inhibits binding of (or competes with) an antibody comprising a HC comprising SEQ ID NO: 15 or 17 and a light chain comprising SEQ ID NO: 14 or 16, respectively, and/or inhibits binding of an antibody comprising VH and VL of 21D5 or 21D5.a, to hILT4.

Embodiment 10: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab binds to hILT4 with a KD of 10⁻⁸ M or 10⁻⁹ M or less.

Embodiment 11: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab binds to cyno ILT4 with a KD of 10⁻⁷ M or 108 M or less and/or promote expression of CD80, CD83 and/or CD86 on cyno monocyte derived dendritic cells.

Embodiment 12: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab does not significantly bind to the human proteins LILRA1, LTLRA2, LTLRA4, LILRA5, LILRA6, ILT2, ILT3, ILT5 or LIR8.

Embodiment 13: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab promotes pro-inflammatory polarization of macrophages towards M1 macrophages.

Embodiment 14: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab does not induce (or trigger) basophil activation.

Embodiment 15: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein a composition comprising the Ab comprises less than 5% of high and low molecular weight species after 3 months incubation at 25° C. and/or less than 10% of high and low molecular weight species after 3 months incubation at 40° C.

Embodiment 16: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86, wherein the Ab comprises an IgG heavy chain constant region, e.g., an IgG1 (e.g., SEQ ID NO: 98 or 102), IgG1.If (SEQ ID NO: 103), IgG1.3 (SEQ ID NO: 100), IgGP238K (SEQ ID NO: 104), IgG4 or IgG4.S228P (SEQ ID NO: 179).

Embodiment 17: An isolated antibody that binds to hILT4, wherein the Ab comprises a heavy chain comprising SEQ ID NO: 17 and a light chain comprising SEQ ID NO: 16, wherein, optionally the heavy chain comprises a C-terminal lysine.

Embodiment 18: An isolated antibody that binds to hILT4, wherein the Ab comprises two heavy chains, each heavy chain comprising SEQ ID NO: 17, and two light chains, each light chain comprising SEQ ID NO: 16, wherein, optionally one or both of the heavy chains comprise a C-terminal lysine.

Embodiment 19: An isolated antibody that binds to hILT4, wherein the Ab comprises a heavy chain comprising SEQ ID NO: 17 and a light chain comprising SEQ ID NO: 16, wherein, optionally the heavy chain comprises a C-terminal lysine, and wherein the Ab comprises at least one disulfide bond linking the heavy chains.

Embodiment 20: An isolated antibody that binds to hILT4, wherein the Ab comprises a heavy chain comprising SEQ ID NO: 15 and a light chain comprising SEQ ID NO: 14, wherein, optionally the heavy chain comprises a C-terminal lysine.

Embodiment 21: An isolated antibody that binds to hILT4, wherein the Ab comprises a heavy chain comprising SEQ ID NO: 17 and a light chain comprising SEQ ID NO: 16, wherein, optionally the heavy chain comprises a C-terminal lysine.

Embodiment 22: An isolated antibody that binds to hILT4, wherein the Ab comprises a heavy chain comprising SEQ ID NO: 15 or 17 and a light chain comprising SEQ ID NO: 14 or 16, wherein, optionally the heavy chain comprises a C-terminal lysine.

Embodiment 23: An isolated nucleic acid or set of nucleic acids encoding the antibody of any one of Embodiments 1-22.

Embodiment 24: A host cell for producing any one of the antibodies of Embodiments 1-22, for example comprising the isolated nucleic acid or set of nucleic acids of Embodiment 23.

Embodiment 25: A method of treating cancer in a subject, comprising administering to the subject a therapeutically effective dose of an antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86 and/or (iii) the heavy chain comprises SEQ ID NO: 17 and the light chain comprises SEQ ID NO: 16.

Embodiment 26: A method of treating cancer in a subject, comprising administering to the subject a therapeutically effective dose of (A) an antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86 and/or (iii) the heavy chain comprises SEQ ID NO: 17 and the light chain comprises SEQ ID NO: 16, and (B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

Embodiment 27: A composition comprising (A) an antibody that binds to human ILT4 (hILT4), wherein the Ab comprises a heavy chain and a light chain, wherein (i) the heavy chain comprises VH CDR1, CDR2 and CDR3 comprising SEQ ID Nos: 161, 162 and 163, respectively, and the light chain comprises VL CDR1, CDR2 and CDR3 comprising SEQ ID Nos: 164, 165 and 166, respectively, and/or (ii) the heavy chain comprises VH comprising SEQ ID NO: 87 and the light chain comprises VL comprising SEQ ID NO: 86 and/or (iii) the heavy chain comprises SEQ ID NO: 17 and the light chain comprises SEQ ID NO: 16, and (B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

### Embodiments Related to Antibodies 10F10, 10F10.1, 10F10.3 and 10F10.4

Embodiment 1: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114.

Embodiment 2: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the antibody stimulates T cell activation (or potentiates a T cell response), e.g., (i) in a mixed lymphocyte reaction (MLR) assay, as evidenced, e.g., by enhanced T cell proliferation or IFN-gamma secretion or TNF-alpha production, or (ii) in a T cell: CHO-OKT3-ILT4 assay (e.g., as described in Example 5).

Embodiment 3: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab enhances T cell activation in a monocyte:T cell allo-MLR.

Embodiment 4: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab enhances IFN-gamma secretion from PBMCs upon antigen stimulation in a CMV lysate assay.

Embodiment 5: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab inhibits binding of hILT4 to a T cell and/or to a binding partner, e.g., HLA-A and/or HLA-B.

Embodiment 6: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the antibody potentiates TNF-alpha secretion from macrophages, e.g., differentiated macrophages.

Embodiment 7: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the antibody increases expression of CD83 and/or CD86 on monocyte-derived dendritic cells (MoDCs).

Embodiment 8: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab (i) binds to the Ig-like domain 2 of hILT4, close to the Ig-like domain 1, and/or (ii) interacts with one or more (or all of) amino acid residues Glu42, Lys43, Gly76, Cys77, Leu88, Pro91, Pro183 and Tyr184 of mature hILT4, as determined by carbene foot-printing.

Embodiment 9: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab inhibits binding of (or competes with) an antibody comprising a HC comprising SEQ ID NO: 19 and a light chain comprising SEQ ID NO: 18, 20, 21, or 116, respectively, and/or inhibits binding of an antibody comprising VH and VL of 10F10, 10F10.1, 10F10.3 and/or 10F10.4, to hILT4.

Embodiment 10: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab binds to hILT4 with a KD of 10⁻⁸ M or 10⁻⁹ M or less.

Embodiment 11: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab binds to cyno ILT4 with a KD of 10⁻⁷ M or 10⁻⁸ M or less and/or promote expression of CD80, CD83 and/or CD86 on cyno monocyte derived dendritic cells.

Embodiment 12: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab promotes pro-inflammatory polarization of macrophages towards M1 macrophages.

Embodiment 13: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab does not induce (or trigger) basophil activation.

Embodiment 14: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein a composition comprising the Ab comprises less than 5% of high and low molecular weight species after 3 months incubation at 25° C. and/or less than 10% of high and low molecular weight species after 3 months incubation at 40° C.

Embodiment 15: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab does not significantly bind to the human proteins LILRA1, LILRA2, LILRA3, LILRA4, LILRA5, LTLRA6, ILT2, ILT3, ILT5 or LIR8.

Embodiment 16: An isolated antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, wherein the Ab comprises an IgG heavy chain constant region, e.g., an IgG1 (e.g., SEQ ID NO: 98 or 102), IgG1.If (SEQ ID NO: 103), IgG1.3 (SEQ ID NO: 100), IgGP238K (SEQ ID NO: 104), IgG4 or IgG4.S228P (SEQ ID NO: 179).

Embodiment 17: An isolated antibody that binds to hILT4, wherein the Ab comprises a heavy chain comprising SEQ ID NO: 19 and a light chain comprising SEQ ID NO: 18, 20, 21 or 116, wherein, optionally the heavy chain comprises a C-terminal lysine.

Embodiment 18: An isolated antibody that binds to hILT4, wherein the Ab comprises a heavy chain comprising SEQ ID NO: 19 and a light chain comprising SEQ ID NO: 18, 20, 21 or 116, wherein, optionally one or both of the heavy chains comprise a C-terminal lysine.

Embodiment 19: An isolated antibody that binds to hILT4, wherein the Ab comprises a heavy chain comprising SEQ ID NO: 19 and a light chain comprising SEQ ID NO: 18, 20, 21 or 116, wherein, optionally the heavy chain comprises a C-terminal lysine, and wherein the Ab comprises at least one disulfide bond linking the heavy chains.

Embodiment 20: An isolated antibody that binds to hILT4, wherein the Ab comprises a heavy chain comprising SEQ ID NO: 19 and a light chain comprising SEQ ID NO: 18, wherein, optionally the heavy chain comprises a C-terminal lysine.

Embodiment 21: An isolated antibody that binds to hILT4, wherein the Ab comprises a heavy chain comprising SEQ ID NO: 19 and a light chain comprising SEQ ID NO: 20, wherein, optionally the heavy chain comprises a C-terminal lysine.

Embodiment 22: An isolated antibody that binds to hILT4, wherein the Ab comprises a heavy chain comprising SEQ ID NO: 19 and a light chain comprising SEQ ID NO: 21, wherein, optionally the heavy chain comprises a C-terminal lysine.

Embodiment 23: An isolated antibody that binds to hILT4, wherein the Ab comprises a heavy chain comprising SEQ ID NO: 19 and a light chain comprising SEQ ID NO: 116, wherein, optionally the heavy chain comprises a C-terminal lysine.

Embodiment 24: An isolated nucleic acid or set of nucleic acids encoding the antibody of any one of Embodiments 1-23.

Embodiment 25: A host cell for producing any one of the antibodies of Embodiments 1-23, for example comprising the isolated nucleic acid or set of nucleic acids of Embodiment 24.

Embodiment 26: A method of treating cancer in a subject, comprising administering to the subject a therapeutically effective dose of an antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, and/or (iii) the heavy chain comprises SEQ ID NO: 19 and the light chain comprises SEQ ID NO: 18, 20, 21 or 116.

Embodiment 27: A method of treating cancer in a subject, comprising administering to the subject a therapeutically effective dose of an antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, and/or (iii) the heavy chain comprises SEQ ID NO: 19 and the light chain comprises SEQ ID NO: 18, 20, 21 or 116, and (B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

Embodiment 28: A composition comprising (A) an antibody that binds to human ILT4 (hILT4), wherein the Ab comprises a heavy chain and a light chain, wherein (i) the heavy chain comprises VH CDR1, CDR2 and CDR3 comprising SEQ ID Nos: 167, 168 and 169, respectively, and the light chain comprises VL CDR1, CDR2 and CDR3 comprising SEQ ID Nos: 170, 171 and 172, respectively, and/or (ii) the heavy chain comprises VH comprising SEQ ID NO: 91 and the light chain comprises VL comprising SEQ ID NO: 90, 94, 96 or 114, and/or (iii) the heavy chain comprises SEQ ID NO: 19 and the light chain comprises SEQ ID NO: 18, 20, 21 or 116, and (B) an antagonist of PD-1 or PD-L1, such as an antagonist antibody binding to human PD-1 or human PD-L1, e.g., nivolumab, pembrolizumab, cemiplimab, toripalimab, sintilimab, atezolizumab, durvalumab or avelumab.

### Nucleic Acids and Host Cells

Also provided are nucleic acids encoding an antibody or a heavy or light chain thereof or a portion thereof. Exemplary nucleic acids are provided in the Sequence Table. Any nucleic acid that is at least 80%, 85%, 90%, 95%, 97%, 98% or 99% to a nucleic acid in the Sequence Table is encompassed herein. Compositions comprising nucleic acids encoding an antibody provided herein are also encompassed, as are cells comprising these and methods for preparing antibodies, comprising culturing a cell transformed with a nucleic acid encoding an ILT4 antibody, and isolating the antibody from the medium or the cell.

### Methods of Treatment Using ILT4 Binding Abs and Related Pharmaceutical Compositions

The antibodies described herein may be used, for example, for treating cancer. In some embodiments, methods for treating cancer are provided, comprising administering an effective amount of an antibody described herein to a patient. In some embodiments, the Abs may trigger or enhance an immune response in the patient, such as an antigen-specific immune response. In some embodiments, the Abs may stimulate T cell activity. In some embodiments, the Abs may inhibit the growth of at least one tumor in the patient.

Provided herein are methods for treating a subject having cancer, comprising administering to the subject a therapeutically effective amount of an ILT4 antibody described herein, such that the subject is treated. An ILT4 antibody can be used alone. Alternatively, an ILT4 antibody can be used in conjunction with another agent, as described further below.

Cancers can be cancers with solid tumors or blood malignancies (liquid tumors).

Non-limiting examples of cancers for treatment include squamous cell carcinoma, small-cell lung cancer, non-small cell lung cancer, squamous non-small cell lung cancer (NSCLC), nonsquamous NSCLC, glioma, gastrointestinal cancer, renal cancer (e.g., clear cell carcinoma), ovarian cancer, liver cancer, colorectal cancer, endometrial cancer, kidney cancer (e.g., renal cell carcinoma (RCC)), prostate cancer (e.g., hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain cancer, brain stem glioma, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CMIL), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers. The methods described herein can also be used for treatment of metastatic cancers, unresectable, refractory cancers (e.g., cancers refractory to previous immunotherapy, e.g., with a blocking CTLA-4 or PD-1 antibody), and/or recurrent cancers.

In certain embodiments, an antibody described herein is administered to patients having a cancer that has exhibited an inadequate response to, or progressed on, a prior treatment, e.g., a prior treatment with an immuno-oncology or immunotherapy drug. In some embodiments, the cancer is refractory or resistant to a prior treatment, either intrinsically refractory or resistant (e.g., refractory to a PD-1 pathway antagonist), or a resistance or refractory state is acquired. For example, an antibody described herein may be administered to subjects who are not responsive or not sufficiently responsive to a first therapy or who have disease progression following treatment, e.g., anti-PD-1 pathway antagonist treatment, either alone or in combination with another therapy (e.g., with an anti-PD-1 pathway antagonist therapy). In other embodiments, an antibody described herein is administered to patients who have not previously received (i.e., been treated with) an immuno-oncology agent, e.g., a PD-1 pathway antagonist.

Combinations with Immune Stimulating Agents

In some embodiments, an antibody as described herein, e.g., an ILT4 antibody described herein, is administered in combination with and at least one immune stimulating agent. For example, the therapeutics may be infused together or injected at roughly the same time. In some embodiments, the antibody and the at least one immune stimulating agent are administered sequentially. For example, in some embodiments the antibody is administered sequentially before or after at least one immune stimulating agent such that the two therapeutics are administered 30 minutes, 60 minutes, 90 minutes, 120 minutes, 3 hours, 6 hours, 12 hours, 24 hours, 36 hours, 48 hours, 3 days, 5 days, 7 days, or two weeks apart.

In some embodiments, at least one, at least two, at least three doses, at least five doses, or at least ten doses of the antibody are administered prior to administration of at least one immune stimulating agent. In some embodiments, at least one, at least two, at least three doses, at least five doses, or at least ten doses of at least one immune stimulating agent is administered prior to administration of the antibody. In some embodiments, the last dose of immune stimulating agent is administered at least one, two, three, five, days or ten, or one, two, three, five, twelve, or twenty four weeks prior to the first dose of the antibody. In some embodiments, the last dose of the antibody is administered at least one, two, three, five, days or ten, or one, two, three, five, twelve, or twenty four weeks prior to the first dose of at least one immune stimulating agent. In some embodiments, a subject has received, or is receiving, therapy with at least one immune stimulating agent and an ILT4 antibody is added to the therapeutic regimen.

In some embodiments, the at least one immune stimulating agent comprises an antagonist of an inhibitor of the activation of immune cells, such as T cells, while in some embodiments, the at least one immune stimulating agent comprises an agonist of a stimulator of the activation of an immune cell, such as a T cell. In some embodiments, the at least one immune stimulating agent comprises an antagonist of CTLA4, LAG-3, PD-1, PD-L1, Galectin 1, Galectin 9, CEACAM-1, BTLA, CD25, CD69, TIGIT, CD 113, GPR56, VISTA, B7-H3, B7-H4, 2B4, CD48, GARP, PD1H, LAIR1, TIM1, TIM3, TIM4, IL-6, IL-10, TGFβ, VEGF, KIR, adenosine A2A receptor, PI3Kdelta, or IDO. In some embodiments, the at least one immune stimulating agent comprises an agonist of B7-1, B7-2, CD28, 4-1BB (CD137), 4-1BBL, ICOS, ICOS-L, OX40, OX40L, GITR, GITRL, CD27, CD40, CD40L, DR3, CD28H, IL-2, IL-7, IL-12, IL-15, IL-21, IFNα, STING, or a Toll-like receptor agonist such as a TLR2/4 agonist. In some embodiments, the at least one immune stimulating agent comprises an agent that binds to another member of the B7 family of membrane-bound proteins such as B7-1, B7-2, B7-H2 (ICOS-L), B7-H3, B7-H4, and B7-H6. In some embodiments, the at least one immune stimulating agent comprises an agent that binds to a member of the TNF receptor family or a co-stimulatory or co-inhibitory molecule binding to a member of the TNF receptor family such as CD40, CD40L, OX40, OX40L, GITR, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, EDA1, EDA2, TACI, APRIL, BCMA, LTOR, LIGHT, DeR3, HVEM, VEGL/TL1A, TRAMP/DR3, TNFR1, TNFβ, TNFR2, TNFα, 1β2, FAS, FASL, RELT, DR6, TROY, or NGFβ. In some embodiments, the at least one immune stimulating agent comprises an agent that antagonizes or inhibits a cytokine that inhibits T cell activation such as IL-6, IL-10, TGFβ, VEGF. In some embodiments, the at least one immune stimulating agent comprises an agonist of a cytokine that stimulates T cell activation such as IL-2, IL-7, IL-12, IL-15, IL-21, and IFNα. In some embodiments, the at least one immune stimulating agent comprises an antagonist of a chemokine, such as CXCR2, CXCR4, CCR2, or CCR4. In some embodiments, the at least one immune stimulating agent comprises an antibody. In some embodiments, the at least one immune stimulating agent may comprise a vaccine, such as a mesothelin-targeting vaccine or attenuated listeria cancer vaccine such as CRS-207.

For example, an ILT4 antibody described herein could be administered with one or more of the following agents:

(1) An antagonist (inhibitor or blocking agent) of a protein that inhibits T cell activation (e.g., immune checkpoint inhibitors), such as CTLA-4, PD-1, PD-L1, PD-L2, and LAG-3, Galectin 9, CEACAM-1, BTLA, CD69, Galectin-1, TIGIT, CD 113, GPR56, B7-H3, B7-H4, 2B4, CD48, GARP, PD1H, LAIR1, TIM-1,TIM-3 and TIM-4; and/or (2) An agonist of a protein that stimulates T cell activation, such as B7-1, B7-2, CD28, 4-1BB (CD137), 4-1BBL, GITR, ICOS, ICOS-L, OX40, OX40L, CD70, CD27, CD40, DR3 and CD28H.

Exemplary agents that can be combined with ILT4 antibodies described herein for treating cancer include: YERVOY® (ipilimumab) or Tremelimumab (to CTLA-4), galiximab (to B7.1), BMS-936558 (to PD-1), MK-3475 (to PD-1), atezolizumab (TECENTRIQ®), Avelumab, Durvalumab, AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3); anti-GITR antibodies MK4166, TRX518, Medi1873, INBRX-110, LK2-145, GWN-323, GITRL-Fc, or any combination thereof.

Other molecules that can be combined with ILT4 antibodies for the treatment of cancer include antagonists of inhibitory receptors on NK cells or agonists of activating receptors on NK cells, for example, antagonists of KIR (e.g., lirilumab).

T cell activation may also be regulated by soluble cytokines. In some embodiments, ILT4 antibodies can be administered in combination with antagonists of cytokines that are intended to inhibit T cell activation or agonists of cytokines that stimulate T cell activation. For example, ILT4 antibodies can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13/69264; WO14/036357).

ILT4 antibodies can also be administered with agents that inhibit TGF-β signaling.

Additional agents that can be combined with an ILT4 antibody include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that can be combined with an ILT4 antibody include therapies that deplete or block Treg cells, e.g., an agent that specifically binds to CD25.

Another therapy that can be combined with an ILT4 antibody is a therapy that inhibits a metabolic enzyme such as indoleamine dioxigenase (IDO), dioxigenase, arginase, or nitric oxide synthetase.

Another class of agents that can be used with an ILT4 antibody includes agents that inhibit the formation of adenosine, e.g., CD73 inhibitors, or inhibit the adenosine A2A receptor.

Other therapies that can be combined with an ILT4 antibody for treating cancer include therapies that reverse/prevent T cell anergy or exhaustion and therapies that trigger an innate immune activation and/or inflammation at a tumor site.

Other therapies that can be combined with an ILT4 antibody for treating cancer include therapies that block IL-8, e.g., with HuMax®-IL8.

An ILT4 antibody can be combined with more than one immuno-oncology agent, and can be, e.g., combined with a combinatorial approach that is intended to target multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells; a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

ILT4 antibodies described herein can be used together with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In certain embodiments, an ILT4 antibody is administered to a subject together with a BRAF inhibitor if the subject is BRAF V600 mutation positive.

Suitable PD-1 antagonists for use in the combination therapy described herein, include, without limitation, ligands, antibodies (e.g., monoclonal antibodies and bispecific antibodies), and multivalent agents. In one embodiment, the PD-1 antagonist is a fusion protein, e.g., an Fc fusion protein, such as AMP-244. In one embodiment, the PD-1 antagonist is an anti-PD-1 or anti-PD-L1 antibody.

An exemplary anti-PD-1 antibody is nivolumab (BMS-936558) or an antibody that comprises the CDRs or variable regions of one of antibodies 17D8, 2D3, 4H1, 5C4, 7D3, 5F4 and 4A11 described in WO 2006/121168. In certain embodiments, an anti-PD-1 antibody is MK-3475 (Lambrolizumab) described in WO2012/145493; AMP-514 described in WO 2012/145493; or PDR001. Further known PD-1 antibodies and other PD-1 inhibitors include those described in WO 2009/014708, WO 03/099196, WO 2009/114335, WO 2011/066389, WO 2011/161699, WO 2012/145493, U.S. Pat. Nos. 7,635,757 and 8,217,149, and U.S. Patent Publication No. 2009/0317368. Any of the anti-PD-1 antibodies disclosed in WO2013/173223 can also be used. An anti-PD-1 antibody that competes for binding with, and/or binds to the same epitope on PD-1 as, as one of these antibodies can also be used in combination treatments.

In some embodiments, the anti-PD-L1 antibody useful for the combination therapy is BMS-936559 (referred to as 12A4 in WO 2007/005874 and U.S. Pat. No. 7,943,743), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment an anti-PD-L1 antibody is MEDI4736 (also known as durvalumab and Anti-B7-H1), MPDL3280A (also known as atezolizumab and RG7446), MSB0010718C (also known as avelumab; WO2013/79174), or rHigM12B7. Any of the anti-PD-L1 antibodies disclosed in WO2013/173223, WO2011/066389, WO2012/145493, U.S. Pat. Nos. 7,635,757 and 8,217,149 and U.S. Publication No. 2009/145493 can also be used. Anti-PD-L1 antibodies that compete with and/or bind to the same epitope as that of any of these antibodies can also be used in combination treatments.

In certain embodiments, the ILT4 antibody of the disclosure can be used with a CTLA-4 antagonist, e.g., an anti-CTLA-4 antibody. In one embodiment, an anti-CTLA-4 antibody is an antibody selected from the group of: YERVOY® (ipilimumab or antibody 10D1, described in PCT Publication WO 01/14424), tremelimumab (formerly ticilimumab, CP-675,206), monoclonal or an anti-CTLA-4 antibody described in any of the following publications: WO 98/42752; WO 00/37504; U.S. Pat. No. 6,207,156; Hurwitz et al. (1998) Pro. Natl. Acad. Sci. USA 95(17): 10067-10071; Camacho et al. (2004) J. Clin. Oncology 22(145): Abstract No. 2505 (antibody CP-675206); and Mokyr et al. (1998) Cancer Res. 58:5301-5304. Any of the anti-CTLA-4 antibodies disclosed in WO2013/173223 can also be used.

In some embodiments, an ILT4 antibody of the disclosure is used in combination with a LAG3 antagonist. Examples of anti-LAG3 antibodies include antibodies comprising the CDRs or variable regions of antibodies 25F7, 26H10, 25E3, 8B7, 11F2 or 17E5, which are described in U.S. Patent Publication No. US2011/0150892, WO10/19570 and WO2014/008218. In one embodiment, an anti-LAG-3 antibody is BMS-986016. Other art recognized anti-LAG-3 antibodies that can be used include IMP731 and IMP-321, described in US 2011/007023, WO08/132601, and WO09/44273. Anti-LAG-3 antibodies that compete with and/or bind to the same epitope as that of any of these antibodies can also be used in combination treatments.

In some embodiments, an ILT4 antibody of the disclosure can be administered in combination with a CD137 (4-1iB) agonist, such as an agonistic CD137 antibody. Suitable CD137 antibodies include, for example, urelumab or PF-05082566 (WO12/32433).

In some embodiments, an ILT4 antibody can be administered in combination with an OX40 agonist, such as an agonistic OX40 antibody. Suitable OX40 antibodies include, for example, MEDI-6383, MEDI-6469 or MOXR0916 (RG7888; WO06/029879).

In one embodiment, an ILT4 antibody is administered in combination with a CD40 agonist, such as an agonistic CD40 antibody. In certain embodiments, the immuno-oncology agent is a CD40 antagonist, such as an antagonistic CD40 antibody. Suitable CD40 antibodies include, for example, lucatumumab (HCD122), dacetuzumab (SGN-40), CP-870,893 or Chi Lob 7/4.

In one embodiment, an ILT4 antibody is administered in combination with a CD27 agonist, such as an agonistic CD27 antibody. Suitable CD27 antibodies include, for example, varlilumab (CDX-1127).

In certain embodiments, the ILT4 antibody is administered together with an anti-GITR antibody, e.g., an antibody having the CDR sequences of 6C8, e.g., a humanized antibody having the CDRs of 6C8, as described, e.g., in WO2006/105021; an antibody comprising the CDRs of an anti-GITR antibody described in WO2011/028683; an antibody comprising the CDRs of an anti-GITR antibody described in JP2008278814, an antibody comprising the CDRs of an anti-GITR antibody described in WO2015/031667, WO2015/187835, WO2015/184099, WO2016/054638, WO2016/057841 or WO2016/057846 or other anti-GITR antibody described or referred to herein.

In some embodiments, an ILT4 antibody is administered in combination with MGA271 (to B7H3) (WO11/109400).

In some embodiments, an ILT4 antibody is administered in combination with a KIR antagonist, such as lirilumab.

In some embodiments, an ILT4 antibody is administered in combination with an IDO antagonist. Suitable IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) or F001287.

In some embodiments, an ILT4 antibody is administered in combination with a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In one embodiment, an ILT4 is administered in combination with a TGF-β inhibitor, e.g., GC1008, LY2157299, TEW7197, or IMC-TR1.

### Additional Combination Therapy

The Abs herein may also be provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof. For example, an ILT4 antibody as described herein could be administered as adjunctive therapy when there is a risk that micrometastases can be present and/or in order to reduce the risk of a relapse.

For treatment of cancer, the combinations may be administered in conjunction with one or more additional anti-cancer agents, such as a chemotherapeutic agent, growth inhibitory agent, anti-cancer vaccine such as a gene therapy vaccine, anti-angiogenesis agent and/or anti-neoplastic composition. Nonlimiting examples of chemotherapeutic agent, growth inhibitory agent, anti-cancer vaccine, anti-angiogenesis agent and anti-neoplastic composition that can be used in combination with the antibodies of the present invention are provided herein under “Definitions.”

In some embodiments, an anti-inflammatory drug may be administered with the combination, such as a steroid or a non-steroidal anti-inflammatory drug (NSAID). In cases where it is desirable to render aberrantly proliferative cells quiescent in conjunction with or prior to treatment with ILT4 antibodies described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX®, can also be administered to the patient. When employing the methods or compositions described herein, other agents used in the modulation of tumor growth or metastasis in a clinical setting, such as antimimetics, can also be administered as desired.

Antibodies described herein can also be combined with an immunogenic agent, such as cancerous cells, purified tumor antigens (including recombinant proteins, peptides, and carbohydrate molecules), cells, and cells transfected with genes encoding immune stimulating cytokines (He et al., (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below).

An ILT4 antibody described herein, can also be combined with a vaccination protocol. Many experimental strategies for vaccination against tumors have been devised (see Rosenberg, S., 2000, Development of Cancer Vaccines, ASCO Educational Book Spring: 60-62; Logothetis, C, 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. ILT4 inhibition can be used in conjunction with a collection of recombinant proteins and/or peptides expressed in a tumor in order to generate an immune response to these proteins. These proteins are normally viewed by the immune system as self antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which can be used in conjunction with ILT4 Abs is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269: 1585-1588; Tamura et al. (1997) Science 278: 117-120).

Dendritic cells (DC) are potent antigen presenting cells that can be used to prime antigen-specific responses. DCs can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be combined with ILT4 inhibition to activate more potent anti-tumor responses.

### Infectious Disease Treatments

Methods described herein can also be used to treat patients that have been exposed to particular toxins or pathogens. Accordingly, this disclosure also contemplates methods of treating an infectious disease in a subject comprising administering to the subject an antibody as described herein, e.g., an ILT4 antibody, such that the subject is treated for the infectious disease. Similar to its application to tumors as discussed above, antibody-mediated ILT4 inhibition can be used alone, or as an adjuvant, in combination with vaccines, to stimulate the immune response to pathogens, toxins, and self-antigens. Examples of pathogens for which this therapeutic approach might be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. ILT4 inhibition can be useful against established infections by agents such as HIV that present altered antigens over the course of the infections.

Some examples of pathogenic viruses causing infections that may be treatable by methods described herein include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections that may be treatable by methods described herein include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections that may be treatable by methods described herein include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections that may be treatable by methods described herein include Entamoeba histolytica, Balantidium coli, Naegleria fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, and Nippostrongylus brasiliensis.

In all of the above methods, ILT4 inhibition can be combined with other forms of immunotherapy, e.g., those described herein, such as cytokine treatment (e.g., interferons, GM-CSF, G-CSF, IL-2), or bispecific antibody therapy, which may provide for enhanced presentation of tumor antigens (see, e.g., Holliger (1993) Proc. Natl. Acad. Sci. USA 90:6444-6448; Poljak (1994) Structure 2: 1121-1123).

### Routes of Administration and Carriers

In various embodiments, antibodies may be administered in vivo by various routes, including, but not limited to, intra-venous (i.v.), subcutaneous, oral, intra-arterial, parenteral, intranasal, intramuscular, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. A nucleic acid molecule encoding an antibody may be coated onto gold microparticles and delivered intradermally by a particle bombardment device, or “gene gun,” as described in the literature (see, e.g., Tang et al., Nature 356:152-154 (1992)). The appropriate formulation and route of administration may be selected according to the intended application.

In various embodiments, compositions comprising antibodies are provided in formulations with a wide variety of pharmaceutically acceptable carriers (see, e.g., Gennaro, Remington: The Science and Practice of Pharmacy with Facts and Comparisons: Drugfacts Plus, 20^{th} ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising antibodies may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

Pharmaceutical packs and kits comprising one or more containers, each containing one or more doses of an antibody or combination of antibodies are also provided. In some embodiments, a unit dosage is provided wherein the unit dosage contains a predetermined amount of a composition comprising an antibody or combination of antibodies, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective Ph range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In general, antibodies may be administered in an amount in the range of about 10 g/kg body weight to about 100 mg/kg body weight per dose. In some embodiments, antibodies may be administered in an amount in the range of about 50 g/kg body weight to about 5 mg/kg body weight per dose. In some embodiments, antibodies may be administered in an amount in the range of about 100 g/kg body weight to about 10 mg/kg body weight per dose. In some embodiments, antibodies may be administered in an amount in the range of about 100 g/kg body weight to about 20 mg/kg body weight per dose. In some embodiments, antibodies may be administered in an amount in the range of about 0.5 mg/kg body weight to about 20 mg/kg body weight per dose.

The antibody compositions may be administered as needed to subjects. Determination of the frequency of administration may be made by persons skilled in the art, such as an attending physician based on considerations of the condition being treated, age of the subject being treated, severity of the condition being treated, general state of health of the subject being treated and the like. In some embodiments, an effective dose of an antibody is administered to a subject one or more times. In various embodiments, an effective dose of an antibody is administered to the subject once a month, less than once a month, such as, for example, every two months or every three months. In other embodiments, an effective dose of an antibody is administered more than once a month, such as, for example, every three weeks, every two weeks or every week. In some embodiments, an effective dose of an antibody is administered once per 1, 2, 3, 4, or 5 weeks. In some embodiments, an effective dose of an antibody is administered twice or three times per week. An effective dose of an antibody is administered to the subject at least once. In some embodiments, the effective dose of an antibody may be administered multiple times, including for periods of at least a month, at least six months, or at least a year.

In certain embodiments, the combination of the ILT4 antibody and a second agent discussed herein can be administered concurrently as a single composition in a pharmaceutically acceptable carrier, or concurrently as separate compositions with the ILT4 antibody and the second agent in a pharmaceutically acceptable carrier. In one embodiment, the combination of the ILT4 antibody and the second agent can be administered sequentially. The administration of the two agents can start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent can start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Antibodies Binding to Human ILT4

Anti-human ILT4 monoclonal antibodies (ILT4 antibodies or hILT4 antibodies) were generated using transgenic mice that express human antibody genes. Fully human monoclonal antibodies to human ILT4 were generated by immunizing 2 types of human immunoglobulin transgenic animals. KM [M/K] mice were immunized with hILT4-mFc recombinant protein, which was composed of the extracellular portion of hILT4 with a C-terminal mouse Fc tag. The antigen was mixed 1:1 with Ribi adjuvant and mice were immunized at weekly intervals intraperitoneally and subcutaneously. The HCo42:01 [J/K] strain was immunized with hILT4-his tagged recombinant protein, which was composed of the extracellular portion of hILT4 with a his-tag at the C-terminus. The antigen was mixed 1:1 with Ribi adjuvant and mice were immunized at weekly intervals in the footpad. The serum titers in both types of mice were monitored following four and six injections. Mice received final boosts prior to the final harvest. Depending on the immunization route, draining lymph nodes and the spleen were harvested for subsequent fusions.

Mouse lymphocytes were isolated from immunized mice. Hybridomas were generated by fusions with a mouse myeloma fusion partner by electric field based electrofusion using a Cyto Pulse Hybrimmune large chamber cell fusion electroporator (BTX/Harvard Apparatus). Single cell suspensions of lymphocytes from immunized mice were fused to an equal number of P3X63 Ag8.6.53 (ATCC) non secreting mouse myeloma cells (fusion numbers 4865 and 6951). Resulting cells were plated in flat bottom microtiter plates in Medium E (StemCell Technologies) supplemented with aminopterin for selection of hybridomas (Sigma).

Individual wells were screened for the presence of human IgG/human kappa light chain (hIgG/hK) antibodies using a homogenous HTRF assay after 10-12 days of culture. Hybridoma supernatants from wells positive for hIgG/hK were tested by FACS or by FMAT for binding to cells transfected with hILT4 or control CHO cells. The following antibodies that interacted specifically with hILT4 were further characterized: 9C8.A6, 24E5.A7, 2H2.H3, 21D9.H11, 2E5.A11 and 9G4. Antibodies 21A5 and 10F10 were isolated from a later immunization.

Antibodies from the hybridomas were sequenced using an NGS high throughput sequencing method. Briefly, for each hybridoma clone in a well of a 96 well plate, PCR amplification of the VH and VL regions was done using unique DNA barcodes to identify each well. The 5′ PCR primer hybridizes to the leader region so that the entire variable region sequence is obtained. A particular hybridoma antibody sequence is identified by matching the barcode and the location of the clone on the plate.

Anti-ILT4 antibodies 9G4, 9C8, 2H2, 2E5, 24E5, 21D9, 21A5 and 10F10 were recombinantly expressed in the context of an IgG1, IgG1.1 or IgG1.3 heavy chain constant region, and are also referred to as provided in Table 1. The amino acid sequence of, and nucleotide sequences encoding, the HCs, LCs, VHs, and VLs are provided in FIGS. 1-12 and the Sequence Table below. The location of each of the CDRs is provided in the Figures as well as indicated by underlining in the variable region sequences of the Sequence Table, and the CDRs are included also as separate SEQ ID Nos toward the end of the Sequence Table.

An analysis of the amino acid sequences of the variable domains of antibodies 21D9, 21A5, 2H2 and 10F10 indicated that certain framework amino acid residues were not present in any human germline. To reduce potential immunogenicity issues when these antibodies are administered to humans, germline reversion mutants of these ILT4 antibodies were generated.

For the antibody 21D9, four framework mutations in the heavy chain variable region relative to germline sequences were identified (V2G, G10D, A24T and V481), and are shown in FIG. 13. Among the various combinations of these 4 reverse mutations, four reversion mutants (21D9.b, 21D9.c, 21D9.d and 21D9.e) were generated (see FIG. 13) and tested for binding to hILT4. All 4 reversion mutants comprise the N-terminal EGQ to EVQ (or G2V) mutation, which is unlikely to affect binding to ILT4. In addition to this substitution, mutants 21D9.b, 21D9.c, and 21D9.d each comprise one of the additional substitutions shown in FIG. 13 (D10G; I24A; and/or I48V), and 21D9.e comprises substitutions of all three (see FIG. 13). The four mutants were generated in the context of an IgG1.3 heavy chain constant region, which is an IgG1 constant region comprising amino acid substitutions L234A, L235E and G237A, to reduce effector function. The mutants are referred to as 21D9.b.hIgG1.3 (“21D9.VH-G2V/D10G8V.IgG1.3”), 21D9.c.hIgG1.3 (“21D9.VH-G2V/T24A.IgG1.3”), 21D9.d.hIgG1.3 (“21D9.VH-G2V/I48V”) and 21D9.e.hIgG1.3 (“21D9.VH-G2V/D10G/T24A/I48V.IgG1.3”), and comprise the light chain of 21D9. No germline reversions were made to the light chain.

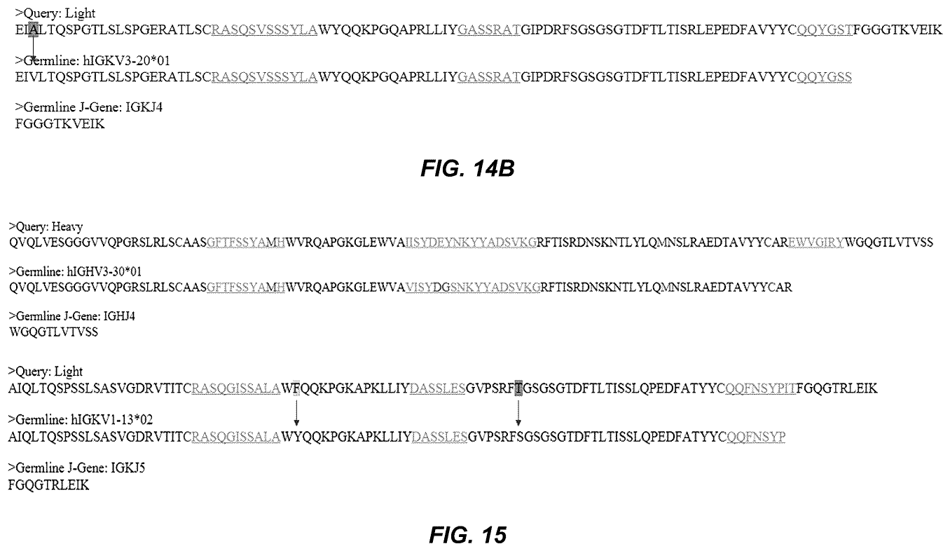

For the antibody 21A5, one framework mutation was identified in the each of the heavy chain (T70I) and light chain (V3A) variable regions, as shown in FIG. 14. As the N-terminal reversion in the kappa light chain, shown at A3V, is unlikely to affect binding, one mutant comprising both heavy (I70T) and light (A3V) chain reversion mutations (21A5.a with a 21A5.1 light chain) was generated and tested in the context of IgG1.3 (21A5.a.IgG1.3, also referred to as “21A5.VH-I70T.VK-A3V.IgG1.3”).

For the antibody 10F10, two framework mutations were identified in the light chain variable region (Y36F and S63T), and are shown in FIG. 15. No framework mutation was found in the heavy chain variable region. As the F to Y reversion at position 36 is a minor difference, a double mutant (10F10.3) comprising both light chain reversion mutations F36Y and T63S was generated and tested in the context of IgG1.3 (10F10.3.IgG1.3, also referred to as “10F10.VK-F36Y/T63S.IgG1.3”). A mutant having only the reversion mutation F36Y was also generated and tested in the context of IgG1.3 (10F10.1.IgG1.3, also referred to as “10F10.VK-F36Y.IgG1.3”). Both reversion mutant Abs comprise the heavy chain of 10F10. Table 1 sets forth the alternative names of recombinant antibodies described herein.

TABLE 1
Alternative names of recombinant
anti-hILT4 antibodies

| | |
|---|---|
| 9G4.IgG1.1 | ILT4.1.IgG1.1 |
| 9C8.IgG1.1 | ILT4.2.IgG1.1 |
| 2H2.IgG1.1 | ILT4.3.IgG1.1 |
| 2E5.IgG1.1 | ILT4.4.IgG1.1 |
| 24E5.IgG1.1 | ILT4.5.IgG1.1 |
| 21D9.IgG1.1 | ILT4.6.IgG1.1 |
| 21D9.IgG1.3 | ILT4.6.IgG1.3 |
| 21D9.e.IgG1.3 | ILT4.8.IgG1.3 |
| 21A5.a.IgG1.3 | ILT4.9.IgG1.3 |
| 10F10.IgG1.3 | ILT4.10.IgG1.3 |
| 10F10.3.IgG1.3 | ILT4.11.IgG1.3 |

### Example 2: Binding of Anti-hILT4 Antibodies to Soluble hILT4

The binding kinetics of the parent and germline reversion mutant anti-hILT4 antibodies (in the context of IgG1.3) were measured via Surface Plasmon Resonance (SPR) using a Biacore® T200 instrument. The assay temperature was 37° C. and the running buffer was PBS pH 7.4 supplemented with 0.05% Tween-20. Allanti-hILT4 antibodies were captured on a CM4 chip with pre-immobilized protein A/G (Pierce Thermo Scientific catalog number 21186). Three different lots of 21D9 were used as controls. Monomeric human ILT4 was injected as analyte. Appropriate concentrations were used for analysis: For 10F10.1 and 10F10.3 a, 3-fold dilution series with a hILT4 top concentration of 1.4 μM was analyzed. For 10F10, two independent hILT4 dilution series were analyzed: 1.4 μM top concentration, 5-fold dilutions and 470 nM, 3-fold dilutions. For all other antibodies, a hILT4 top concentration of 94 nM and a 5-fold dilution series were used. The hILT4 used in this experiment, comprising the extracellular region of hILT4 linked to a His-Avi tag, consists of the following amino acid sequence:

(hILT4-His-Avi tag; SEQ ID NO: 119)
QTGTIPKPTLWAEPDSVITQGSPVTLSCQGSLEAQEYRLYREKKSASWI
TRIRPELVKNGQFHIPSITWEHTGRYGCQYYSRARWSELSDPLVLVMTG
AYPKPTLSAQPSPVVTSGGRVTLQCESQVAFGGFILCKEGEEEHPQCLN
SQPHARGSSRAIFSVGPVSPNRRWSHRCYGYDLNSPYVWSSPSDLLELL
VPGVSKKPSLSVQPGPVVAPGESLTLQCVSDVGYDRFVLYKEGERDLRQ
LPGRQPQAGLSQANFTLGPVSRSYGGQYRCYGAHNLSSESSAPSDPLDI
LITGQIRGTPFISVQPGPTVASGENVTLLCQSWRQFHTFLLTKAGAADA
PLRLRSIHEYPKYQAEFPMSPVTSAHAGTYRCYGSLNSDPYLLSHPSEP
LELVVSGPSMGSSPPPTGPISTPAGPEDQPLTPTGSDPQSGLGRHLGSP
GGGSGGGSEQKLISEEDLGHHHHHHGLNDIFEAQKIEWHE.

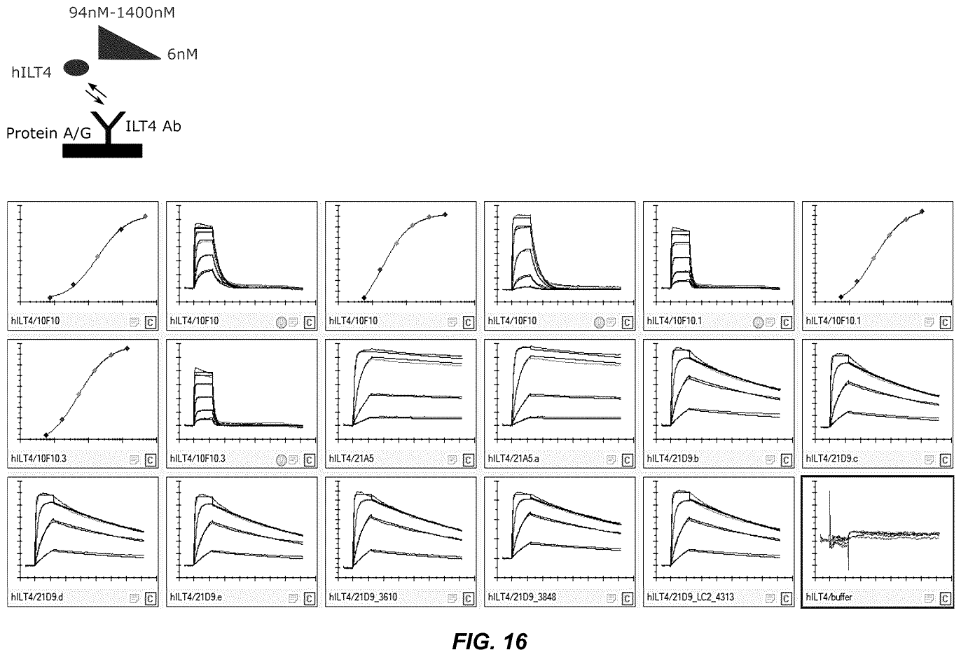

The results are shown in Table 2 and FIG. 16.

TABLE 2
Binding kinetics of anti-hILT4 antibodies to hILT4

| | | | | | | Ligand | |
|---|---|---|---|---|---|---|---|
| | | | | | Rmax | Level | |
| Ligand | Sample | ka (1/Ms) | kd (1/s) | KD (M) | (RU) | (RU) | app. act. |
| 10F10 | hILT4 | steady state | | 1.8E−08 | 31.2 | 39.4 | 114% |
| 10F10 | hILT4 | 1.6E+06 | 2.3E−02 | 1.4E−08 | 22.3 | 24.7 | 130% |
| 10F10 | hILT4 | steady state | | 1.9E−08 | 20.5 | 24.7 | 120% |
| 10F10 | hILT4 | 1.3E+06 | 2.0E−02 | 1.5E−08 | 30.7 | 39.4 | 112% |
| 10F10.1 | hILT4 | 1.5E+06 | ~7E−02 | 4.6E−08 | 21.3 | 32.8 | 94% |
| 10F10.1 | hILT4 | steady state | | 5.6E−08 | 20 | 32.8 | 88% |
| 10F10.3 | hILT4 | steady state | | 5.2E−08 | 18.8 | 23.5 | 115% |
| 10F10.3 | hILT4 | 1.7E+06 | ~8E−02 | 4.7E−08 | 20.1 | 23.5 | 123% |
| 21A5 | hILT4 | 1.2E+06 | 1.7E−04 | 1.4E−10 | 27.1 | 31 | 126% |
| 21A5.a | hILT4 | 1.1E+06 | 1.9E−04 | 1.7E−10 | 27.9 | 41 | 98% |
| 21D9.b | hILT4 | 3.1E+06 | 1.1E−03 | 3.4E−10 | 24.9 | 39.8 | 90% |
| 21D9.c | hILT4 | 3.0E+06 | 1.2E−03 | 4.2E−10 | 27.4 | 40.7 | 97% |
| 21D9.d | hILT4 | 3.1E+06 | 1.1E−03 | 3.5E−10 | 26.3 | 45.3 | 84% |
| 21D9.e | hILT4 | 3.1E+06 | 1.2E−03 | 3.9E−10 | 25.4 | 39.3 | 93% |
| 21D9_3610 | hILT4 | 3.3E+06 | 1.0E−03 | 3.1E−10 | 14.3 | 27.5 | 75% |
| 21D9_3848 | hILT4 | 3.2E+06 | 1.0E−03 | 3.2E−10 | 15.8 | 44.1 | 52% |
| 21D9_LC2_4313 | hILT4 | 3.0E+06 | 1.1E−03 | 3.7E−10 | 27.8 | 40.8 | 98% |

The results show that the 4 reversion mutants of 21D39 have similar kinetics and affinities. 21A5 and its reversion mutant 21A5.a also have similar kinetics and affinities. However, the two 10F10 reversion mutants, 1° F. 10.1 and 1 OF 10.3, have slightly faster dissociation rates than their parent 10F10, driving an about 3 fold loss in overall affinity. Dissociation rates of 10F10.1 and 10F10.3 are too fast to be determined with high confidence.

### Example 3: Binding of Anti-hILT4 Antibodies to hILT4-Expressing Cells

This Example describes the binding characteristics of anti-hILT4 antibodies to CHO cells transfected with hILT4 and to human monocytes expressing hILT4.

CHO cells were transfected with hILT4 and were stained with individual anti-hILT4 antibodies 2H2, 10F10 (“10F10 WT”), 21A5 (“21A5 WT”), 21A5.a, 21D9 (“21D9 WT”) and 21D9e (all in the context of IgG1.3) that were titrated from 20 μg/ml by 3-fold serial dilution and then subjected to a PE-conjugated secondary anti-human IgG. FACS analysis for geometric mean fluorescence intensity (GMFI) was used to determine the EC₅₀.

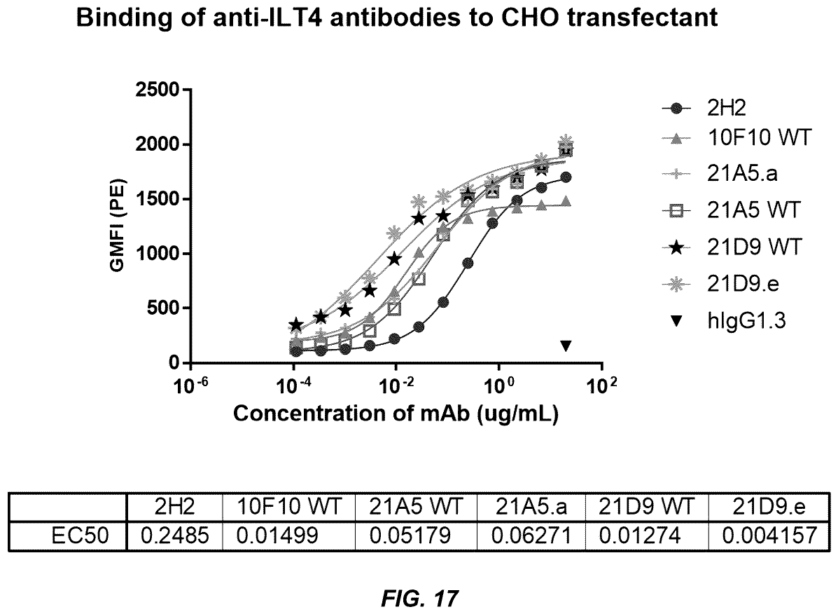

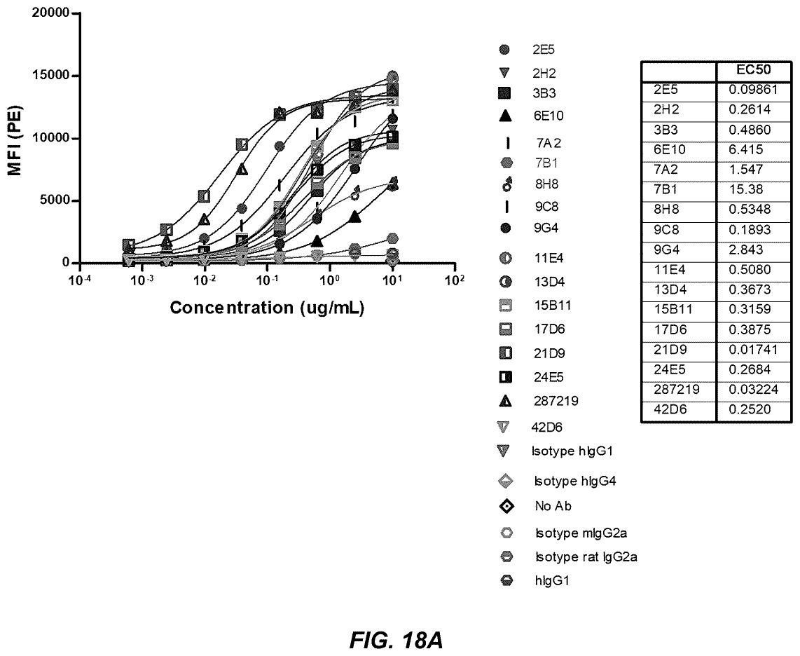

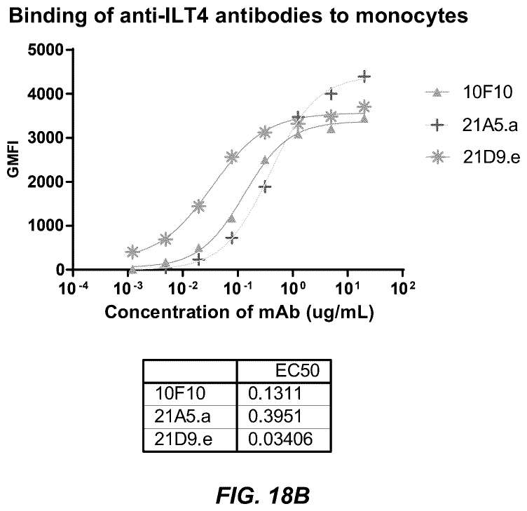

The binding curves and EC values are shown in FIGS. 17 and 18A.

In FIG. 18B, monocytes selected from a normal healthy donor's peripheral blood were stained with individual anti-ILT4 antibodies conjugated to Alexa67. Antibodies were titrated from 20 μg/mL by 3-fold serial dilution, and GMFI was determined by FACS analysis for calculating EC50 (shown in the table below the graph).

### Example 4: Selectivity of Anti-ILT4 Antibodies Against Members of LILRA/B Family

ILT4 is a member of a family of related receptors, also referred to as the LILRA and LILRB family. In this Example, the binding of hILT4 antibodies to various members of the LILRA/LILRB family was measured.

Individual ILT family members were overexpressed in 293T cells. Each transfectant was stained with 20 μg/ml of anti-ILT4 antibodies followed by 1 μg/ml of anti-human IgG secondary mAb. Binding of the antibodies to each transfectant was measured by flow cytometry. In a second experiment, LILRA1, LILRA3 and ILT4 transfectants were incubated with various concentrations of 21A5 and 21A5.a.

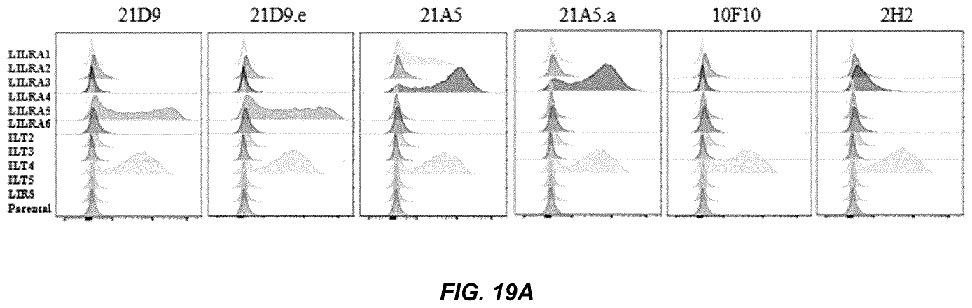

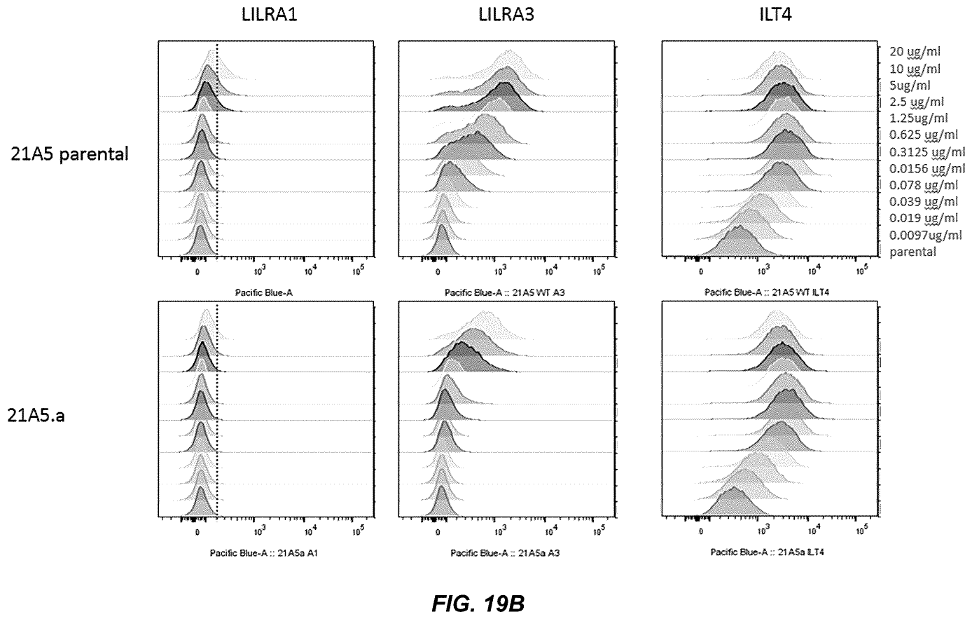

The results, which are shown in FIGS. 19A and B and in Table 3, indicate that anti-hILT4 antibodies bind mostly selectively to hILT4, relative to other LILRA and LILRB family members. LILRA3 is the only secreted protein in the LILRA/B family. More specifically, 21D9 and 21D9.e do not bind significantly to LILRA1, LTLRA2, LILRA3, LILRA4, LILRA6, ILT2, ILT3, ILT5 and LIR8, and only weak binding to LILRA5.

TABLE 3
Selectivity profile of anti-ILT4 antibodies at 20 μg/ml

| | LILRA1 | LILRA2 | LILRA3 | LILRA4 | LILRA5 | LILRA6 | ILT2 | ILT3 | ILT4 | ILT5 | ILT8 | Parental |
|---|---|---|---|---|---|---|---|---|---|---|---|---|
| 21D9 | | | | | + | | | | +++ | | | |
| 21D9.e | | | | | + | | | | +++ | | | |
| 21A5 | | | +++ | | | | | | +++ | | | |
| 21A5.a | | | ++ | | | | | | +++ | | | |
| 2H2 | | | | | | | | | +++ | | | |
| 10F10 | | | | | | | | | +++ | | | |

“Parenteral” in Table 3 refers to the parental cell line in which the LILRA or LILRB molecules were expressed.

Cross-reactivity towards other family members of LILRA was further tested as follows. An antibody titration flow cytometry binding assay was performed to determine the binding potency of 21D9, 21D9.e, 21A5 and 21A5.a to hILT4 and their respective cross-reacting ILT molecules. EC₅₀ was determined using the nonlinear regression formula from the GraphPad Prism® software.

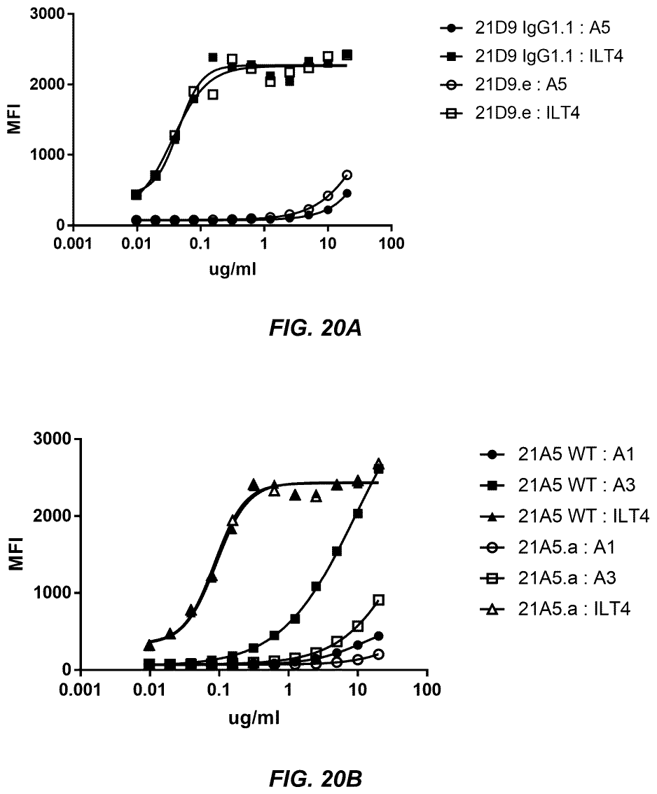

The results, which are shown in Table 4 and FIG. 20, show that anti-ILT4 antibodies 21D9, 21D9.e, 21A5 and 21A5.a show weak cross-reactivity to other family members of LILRA: 21D9 and 21D9.e to LILRA5 and 21A5 and 21A5.a to LILRA1 and LILRA3.

TABLE 4
Summary of EC₅₀ binding potency of anti-ILT4
antibodies to cross-reacting ILTs (μg/ml)

| | 21D9 | 21D9.e | 21A5 | 21A5.a |
|---|---|---|---|---|
| ILT4 | 0.0460 | 0.039 | 0.091 | 0.085 |
| LILRA5 | ~29964 | ~312.6 | No binding | No binding |
| LILRA1 | No binding | No binding | 12.53 | 49.82 |
| LILRA3 | No binding | No binding | 9.18 | 52.94 |

21D9 and 21D9.e bind to hILT4 with high affinity, but do not bind significantly to hLILRA1, hLILRA2, hLILRA3, hLILRA4, hLILRA6, hILT2, hILT3, hILT5 and hLIR8, and only weakly to hLILRA5. Thus, 21D9 and 21D9.e are much more specific in binding to hILT4 relative to other hLILRA and hLIRB family members than other anti-IL T4 antibodies, such as clone 287219 from R&D Systems, which is cross-reactive with ILT5 (LILRB3) and LILRA6.

### Example 5: Anti-hILT4 Antibodies Potentiate T Cell Responses

The effect of the anti-hILT4 antibodies on T cell responses, particularly T cell proliferation and interferon-gamma (IFNγ) T cell secretion, was determined as follows. The cytoplasmic truncated hILT4 was transfected into a CHO cells expressing a low level of single-chain variable fragment of anti-CD3 antibody (CHO-OKT3 cells). The transfected cell line was designated as CHO-OKT3-ILT4. Total T cells isolated from human PBMCs were co-cultured with irradiated (growth arrested) CHO-OKT3-ILT4 cells at a 4:1 ratio. Anti-ILT4 antibodies 21D9 (“21D9 WT”), 21D9.e, 21A5 (“21A5 WT), 21A5.a and 10F10 (“10F10 WT”) were titrated from 20 μg/mL by 5-fold serial dilution, and incubated for 3 days at 37C. Supernatant was harvested to assess IFNγ levels by ELISA, and the plates were pulsed overnight with ³H thymidine to assess proliferation by thymidine incorporation.

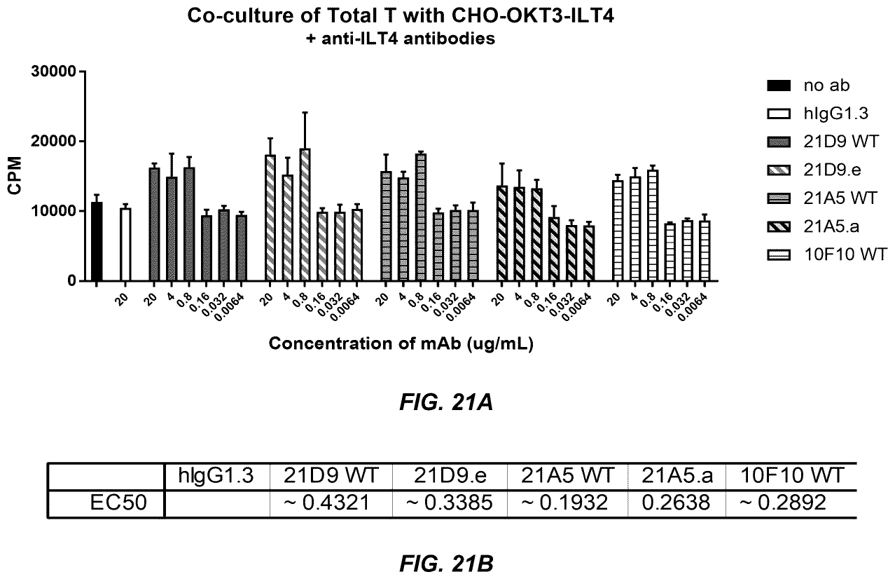

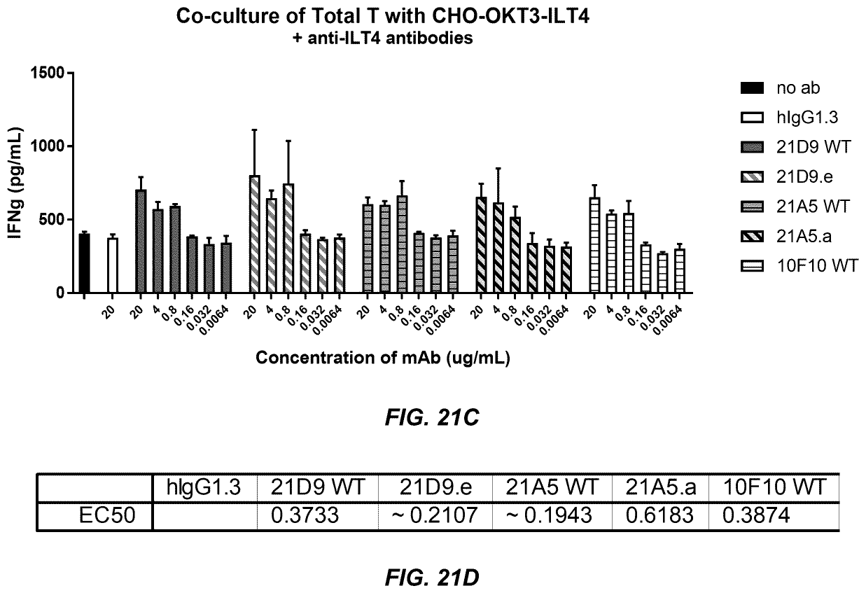

The results, which are shown in FIGS. 21A-D, indicate that anti-ILT4 antibodies 21D9, 21D9.e, 21A5, 21A5.a and 10F10 stimulate T cell proliferation and IFNγ secretion in a dose dependent manner. No significant differences in activity were found between these antibodies in this assay.

### Example 6: Pre-Treatment of Monocytes with Anti-ILT4 Antibodies During Differentiation Results in Monocyte-Derived Dendritic Cells (MoDC) that are More Stimulatory to Allogeneic T Cells

CD14+ monocytes were isolated from human PBMC using StemCell EasySep® Human Monocyte Isolation Kit, plated at 1 million cells per mL, and differentiated into monocyte-derived immature DC (Mo-iDC) in RPMI culture medium supplemented with 50 ng/mL GM-CSF and 100 ng/mL TL-4 for six or seven days. During the differentiation, the cells were incubated with anti-ILT4 or isotype antibody or left untreated. After the cells were differentiated to Mo-iDC, they were washed to remove anti-ILT4 antibodies, GM-CSF and IL-4, and were further activated to matured dendritic cells (DC (Mo-mDC) in the presence of 50 ng/mL of CD40 agonistic antibody. The matured DC (Mo-mDC) were set up for an allogeneic mixed leukocyte reaction assay (allo-MLR) by co-culturing them with T cells at 1:10 ratio (Mo-mDC:T) for 5 days. The cell supernatant was harvested at the end of the allo-MLR for measuring IFNγ by ELISA, and T cell proliferation was assessed by ³H-thymidine incorporation of the final 16 hours.

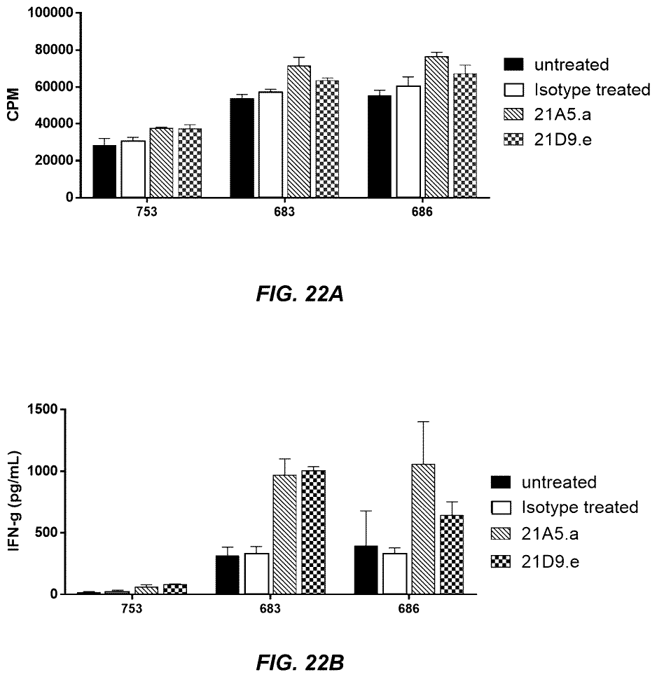

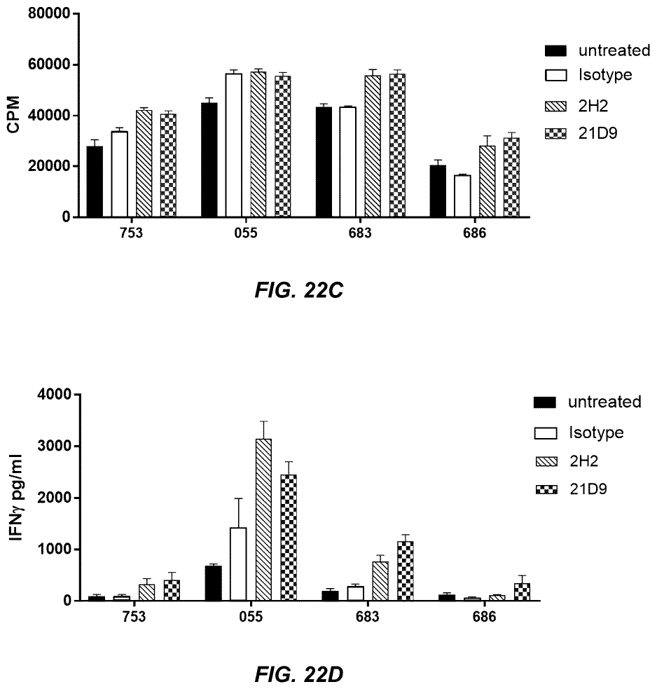

The results of separate assays, which are shown in FIG. 22, indicate that CD4+ T cell proliferation (FIGS. 22A and C) and IFNγ secretion (FIGS. 22B and D) in an allo-MLR with MoDC assay are enhanced when the MoDC are differentiated from monocytes in the presence of anti-ILT4 antibodies 21A5.a or 21D9.e. Monocytes from two different donors were used in the separate assays.

### Example 7: Monocyte-Derived Dendritic Cells (MoDC) Differentiated from Anti-ILT4 Antibody Primed Monocytes have Increased Expression of Costimulatory or Maturation Molecules

CD14+ monocytes were isolated from human PBMC using StemCell EasySep® Human Monocyte Isolation Kit, plated at 1 million cells per mL, and differentiated into monocyte-derived immature dendritic cells (Mo-iDC) in RPMI culture medium supplemented with 50 ng/mL GM-CSF and 100 ng/mL IL-4 for six or seven days in presence of anti-ILT4 antibodies 21D9.e, 21A5.a, 21A5, 10F10, 21D9, 2H2 or isotype antibody or left untreated. The Mo-iDC were then harvested for staining of cell surface markers CD86 and CD83.

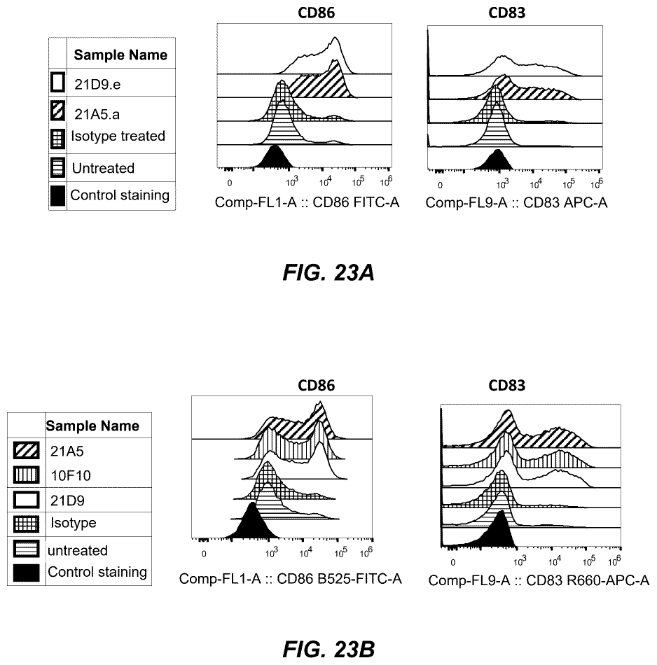

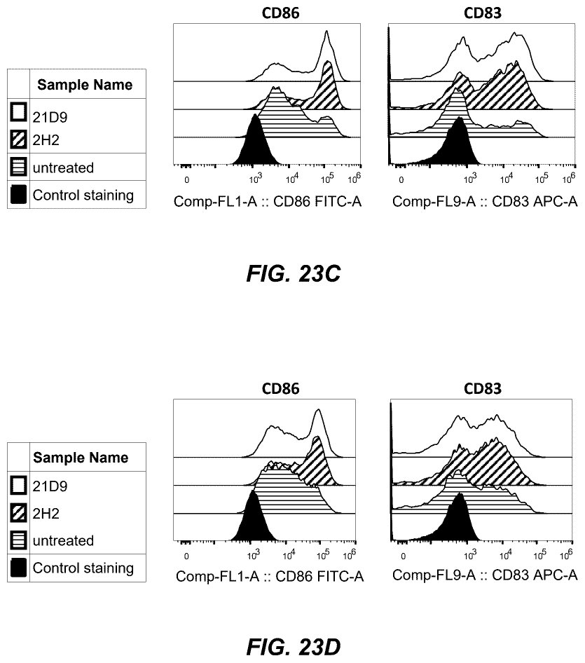

The results, which are shown in FIG. 23, indicate increased expression of CD86 and CD83, a costimulatory and a maturation molecule, respectively, on Mo-iDC when anti-ILT4 antibodies were present during monocyte differentiation to Mo-iDC. Taken together with the results of Example 6, these results show that treating monocytes with anti-hILT4 antibody during monocyte differentiation to MoDC promotes proinflammation of MoDC as shown by upregulation of CD83 and CD86 (FIGS. 23A-D), which consequently triggers enhanced T cell proliferation and IFNγ production from allogeneic T cells in the allo-MLR assay (FIG. 22). By enhancing T cell functions, treatment with anti-hILT4 antibodies may provide for an enhanced anti-cancer immune response.

### Example 8: Anti-ILT4 Antibodies Potentiate TNFα Secretion from In Vitro Differentiated Macrophages

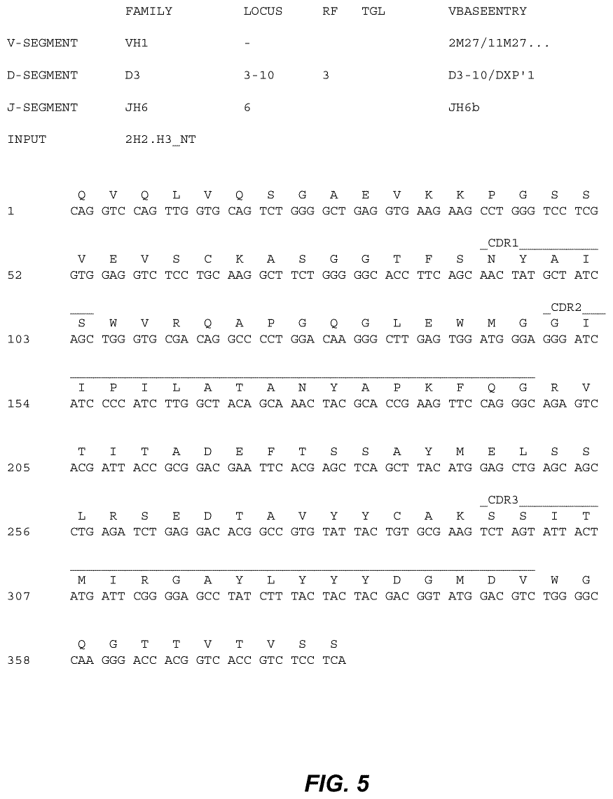

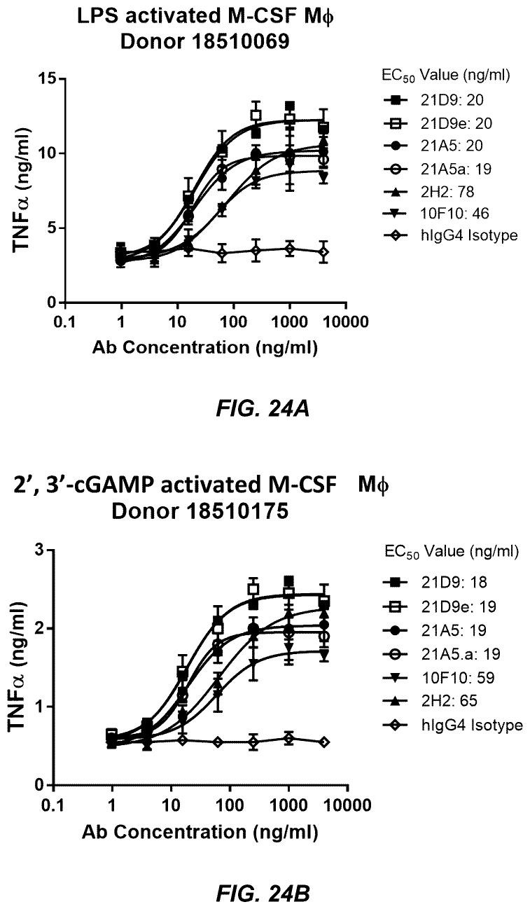

Isolated monocytes from human PBMC were differentiated to macrophages in presence of M-CSF for 5 days. The macrophages were treated with the indicated concentrations (next to their name and as ng/ml) of anti-ILT4 antibodies 21D9, 21D9.e, 21A5, 21A5.a, 2H2 and 10F10 or isotype control in the presence of 10 ng/ml of LPS (FIG. 24A) or 5 μg/ml of 2′ 3′-cGAMP STING agonist (FIG. 24B). TNFα production was measured by ELISA 24 hours after treatment and the EC50 values in the presence of each antibody are noted next to the curves.

The results, which are shown in FIG. 24, indicate that the presence of ILT4 antibodies 21D9, 21D9.e, 21A5, 21A5.a, 2H2 and 10F10 potentiated TNFα secretion of the in vitro differentiated macrophages by either LPS or a STING agonist. Therefore, immunosuppressive macrophages show a shift to a more pro-inflammatory phenotype when ILT4 is blocked, which can potentially provide for an enhanced anti-cancer immune response. These results suggest that anti-ILT4 antibodies promote pro-inflammatory polarization towards M1 macrophages.

### Example 9: Anti-ILT4 Antibodies Promote CD4 T Cell Responses in Macrophage: CD4+ T Cell Allo-MLR

Monocytes isolated from peripheral PBMC were treated with M-CSF for 5 days to differentiate to macrophages. The macrophages were then co-cultured with allogeneic CD4+ T cells that were isolated from a different donor's PBMC, and labeled with CFSE dye. The indicated antibodies at 1 μg/ml (isotype control, anti-ILT4, or anti-PDL1) were included in the allo-MLR co-culture. At day 6, the supernatant was collected for analyzing IFNγ, and CD4+ T cell proliferation and was assessed by FACS based on CFSE dilution.

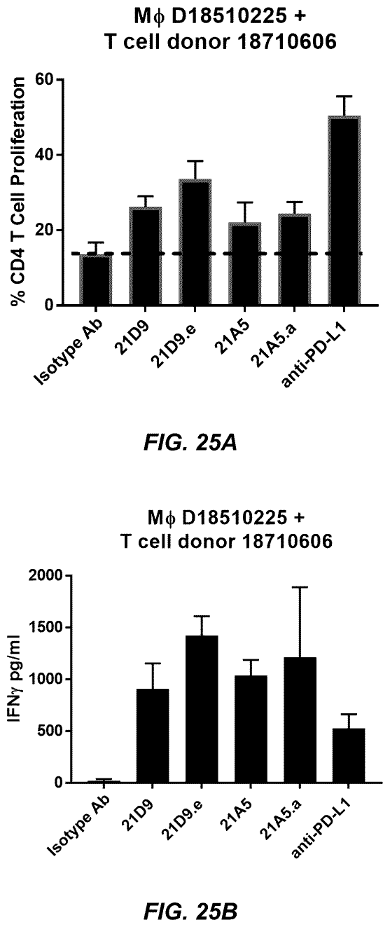

The results, which are shown in FIG. 25A-B, indicate that the presence of the anti-LT4 antibodies 21D9, 21D9.e, 21A5 or 21A5.a in allo-MLR of in vitro differentiated macrophages and CD4+ T cells promotes CD4+ T cell proliferation (FIG. 25A) and IFNγ production (FIG. 25B). These results suggest that the anti-ILT4 antibodies promote T cell activation either through modulating ILT4 suppresive signaling in macrophages or blocking its direct suppressive activity in T cells.

### Example 10: Combined Effect of Anti-ILT4 and Anti-PD-L1 in Macrophages: CD4 T Cell Allo-MLR

Monocytes isolated from peripheral PBMC were treated with M-CSF for 5 days to differentiate to macrophages, which were then co-cultured with allogeneic CD4+ T cells that were isolated from an allogeneic donor's PBMC. Anti-ILT4 antibody 21D9 and anti-PD-L1 antibody added alone or together at 10 μg/ml (isotype control, anti-ILT4, anti-PD-L1 or combined anti-ILT4 and anti-PD-L1) were included in the allo-MLR co-culture. At day 6, the supernatant was collected for analyzing IFNγ.

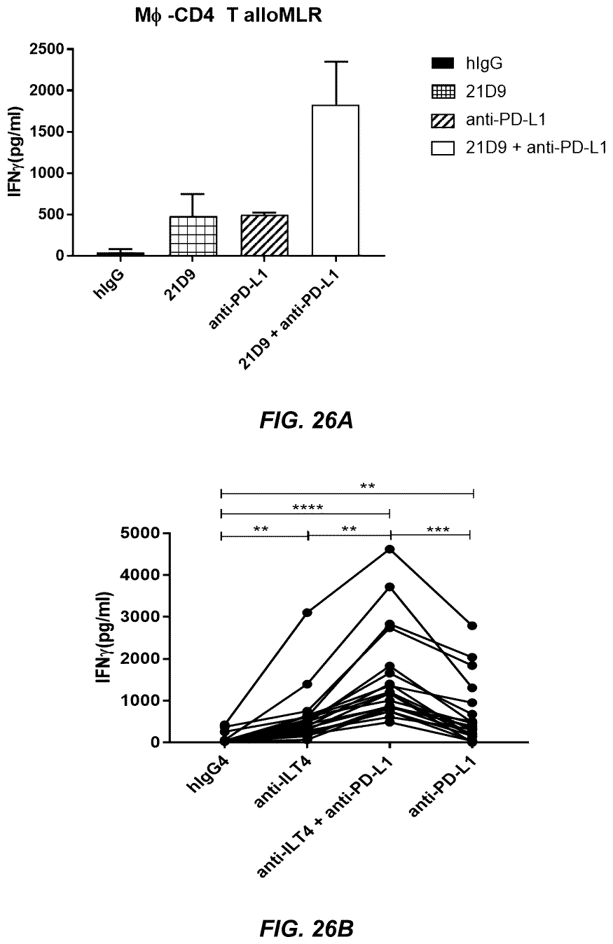

The results, which are shown in FIG. 26, indicate that the combination of the anti-ILT4 antibody 21D9 with the ani-PD-L1 antibody stimulated an enhanced production of IFNγ from the macrophage:CD4+ T cell allo-MLR relative to each antibody alone.

### Example 11: HDX Epitope Mapping of Anti-hILT4 Abs 21D9 and 2H2 Fabs

HDX-MS was used to identify the areas of hILT4 to which anti-hILT4 antibodies 21D9 and 2H2 bind.

HDX-MS probes protein conformation and conformational dynamics in solution by monitoring the rate and extent of deuterium exchange of backbone amide hydrogen atoms (Huang and Chen (2014) Analytical and Bioanalytical Chem. 406:6541 and Wei et al. (2014) Drug Discovery Today 19:95). The level of HDX depends on the solvent accessibility of backbone amide hydrogen atoms and the protein hydrogen bonds. The mass increase of the protein upon HDX can be precisely measured by MS. When this technique is paired with enzymatic digestion, structural features at the peptide level can be resolved, enabling differentiation of surface exposed peptides from those folded inside, or from those sequestered at the interface of a protein-protein complex. Typically, the deuterium labeling and subsequent quenching experiments are performed, followed by enzymatic digestion, peptide separation, and MS analysis.

Prior to epitope mapping experiments, non-deuteriated experiments were carried out to generate a list of common peptides for recombinant human ILT4 (15 μM) and protein complexes of ILT4 with Fabs 21D9 and 2H2 (1:1 molar ratio). In the HDX-MS experiment, 5 μL of each sample (ILT4 or ILT4 with Fabs) was diluted into 55 μL of D20 buffer (10 mM phosphate buffer, D20, pH7.0) to start the labeling reactions. The reactions were carried out for different periods of time: 1 min, 10 min and 240 min. By the end of each labeling reaction period, the reaction was quenched by adding quenching buffer (6M Urea, 1M TCEP, pH 2.5, 1:1, v/v) and 50 μL of quenched sample was injected into Waters HDX-MS system for analysis. The deuterium uptake levels of common peptic peptides were monitored in the absence/presence of Fabs 21D9 and 2H2.

Human ILT4 (hILT4) was histidine tagged and mixed with 21D9 or 2H2 Fabs at a 1:1 ratio and subjected to HDX for 1 minute, 10 minutes or 4 hours. The sequence coverage of hILT4 by pepsin is shown in FIG. 27A. The areas of hILT4 (or hILT4 peptides) bound by each of the Fabs are shown in FIGS. 27B and C. Specifically, HDX-MS experiments provided 85% sequence coverage for human ILT4.

The HDX-MS data analysis on 21D9 and 2H2 in human ILT4 indicates that 21D9's epitope is comprised of one region of human ILT4 (residue numbers correspond to native human ILT4 sequence): Region: ⁷⁰ITRIRPELVKNGQFHIPSITWEHTGRYGCQY¹⁰⁰ (SEQ ID NO: 122) in Ig domain 2. (See FIG. 27B, upper panel.)

The HDX-MS data analysis indicated that 2H2's epitope is comprised of five regions of human ILT4 with regions 2 and 5 being the primary epitope:

Region 1:
(SEQ ID NO: 175)
¹²⁷SAQPSPVVTSGGRVTL¹⁴²
Region 2:
(SEQ ID NO: 123)
¹⁵⁴ILCKEGEEEHPQCLNSQPHARGSSRAIF¹⁸¹
Region 3:
(SEQ ID NO: 176)
¹⁸²SVGPVSPNRRWSHRCYGYDLNSPYVWSSPSDL²¹³
Region 4:
(SEQ ID NO: 177)
³⁷⁸QAEFPMSPVTSAHAG³⁹²
Region 5:
(SEQ ID NO: 124)
⁴²⁵SSPPPTGPIS⁴³⁴
(See FIG. 27B, lower panel.)

### Example 12: Anti-ILT4 Antibodies Block ILT4 Binding to MHC Class I Molecules

A flow cytometry based antibody blocking assay was performed to determine the ability of anti-ILT4 antibodies to block rhILT4-Fc binding to HLA-A or HLA-B overexpressing CHO cells. rhILT4-Fc fusion protein was produced that contained a murine Fc tail. Anti-hILT4 antibodies 21D9, 2H2, 10F10 and 21A5 were titrated in the presence of a fixed 30 μg/ml concentration of rhILT4-Fc. The antibody and rhILT4-Fc mixture was incubated on ice for 30 minutes and then added to CHO cells engineered to overexpress HLA-A or HLA-B. The CHO cells were stained for 30 minutes on ice, washed and then stained with an anti-mouse-Ig secondary antibody to detect bound rhILT4-Fc.

The results, which are shown in FIGS. 28A-D, indicate that antibodies 21D9, 2H2, 10F10 and 21A5, respectively, inhibit binding of hILT4 to HLA-A and HLA-B.

### Example 13: 21D9e.IgG1.3 Enhances Both IFN-γ and TNF-α Secretion by T Cells

This Example shows that 21D9e.IgG1.3 enhances IFN-gamma and TNF-alpha secretion of T cells in an autologous mixed lymphocyte reaction (MLR) of monocytes and T cells.

T cells and monocytes were isolated from peripheral blood mononuclear cells (PBMCs) and co-cultured at 1:1 ratio in the presence of 20 ng/mL anti-CD3 antibody (Clone: OKT3, BioLegend). 21D9e.IgG1.3 and isotype control (DT-1D12-g1.3f) was added at 10 μg/mL. Intracellular staining was performed to determine the frequencies of IFN-γ and TNF-α expressing T cells in each sample after an 88-hour incubation period using fluorochrome-conjugated IFN-γ antibody (Clone #B27, BioLegend) and TNF-α antibody (Clone #MAb11, BioLegend). The samples were acquired on a Cytoflex® cytometer (Beckmen Coulter) and analyzed with FlowJo™ software (Tree Star, Inc, Ashland, OR).

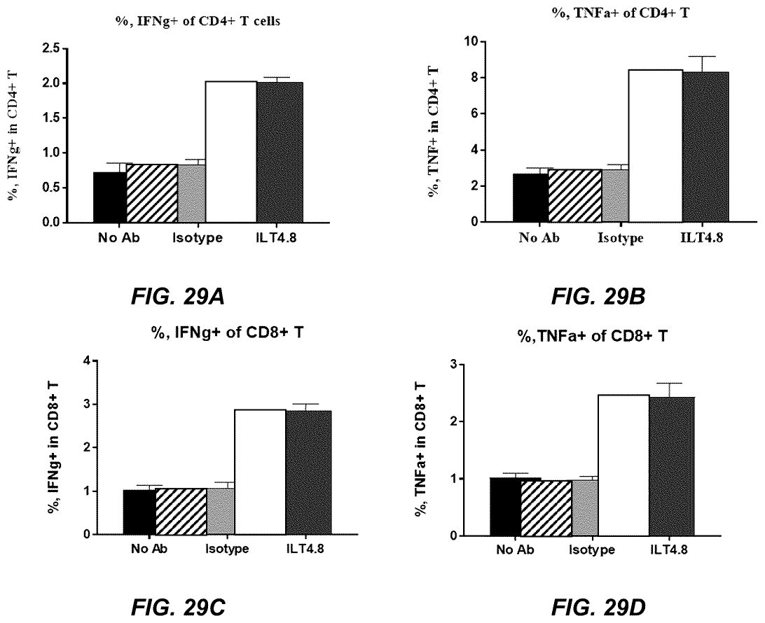

The results, which are shown in FIGS. 29A-D, indicate that 21D9e.IgG1.3 enhances both IFN-γ and TNF-α secretion by CD4+ and CD8+ T cells in the autologous allo-MLR upon anti-CD3 stimulation.

### Example 14: Anti-ILT4 Antibodies Enhance IFN-γ Secretion Upon Antigen Stimulation in a CMV Lysate Assay

Peripheral blood mononuclear cells (PBMCs) from a cytomegalovirus (CMV) reactive donor were plated at 200,000 cells per well in the presence of 0.3 μg/mL of CMV Lysate (CMV Antigen 2, Microbix Cat #EL-02-01-001). Cells were treated with 8-point, 5-fold titration of anti-ILT4 antibodies 21D9.IgG1.3 and 21A5.IgG1.3 starting at 20 μg/mL. Nivolumab was added at 1 μg/mL as a positive control. 21D9-Fab and DT-1D12-g1.3 were added at 20 μg/mL. Supernatant from each sample was harvested and measured for IFN-γ level by ELISA (Human IFN-γ ELISA Kit, BD OptEIA Cat. 555142) after a 6-day incubation.

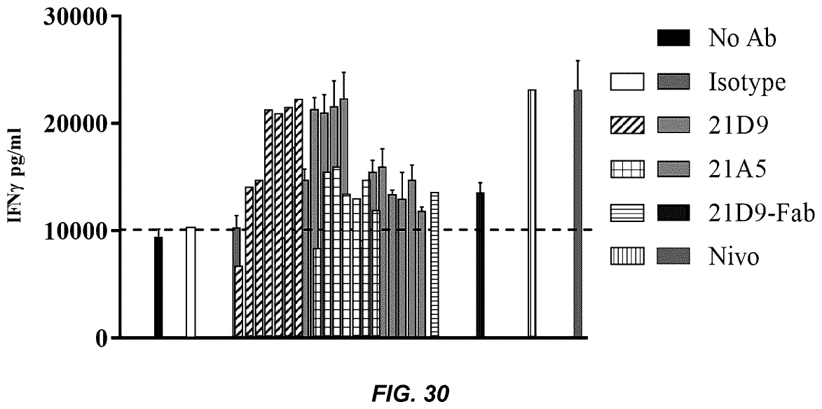

The results, which are shown in FIG. 30, indicate that the anti-ILT4 antibodies 21D9.IgG1.3, 21D9-Fab and 21A5.IgG1.3 enhance IFN-γ secretion upon antigen stimulation in the CMV lysate assay.

### Example 15: ILT4 Antagonism Enhances T Cell Activation in Monocytes:T Allo-MLR

T cells (100,000) from one donor and allogeneic monocytes were co-cultured at a 2:1 ratio per well in a 96-well U-bottom plate. Anti-ILT4 antibodies 2H2.IgG1.1f, 2H2 Fab, 21D9.IgG1.1f and 21D9 Fab were added in a 4-fold, 7-point titration starting at a concentration of 30 μg/mL for full length antibody or 80 μg/mL for Fab antibody. Anti-KLH-g1.1f (Anti-Hemocyanin antibody) was added at 30 μg/mL as an isotype control. The cell culture was incubated for 6 days. Cell proliferation was assessed by ³H-thymidine incorporation during the final 16 hours of culture.

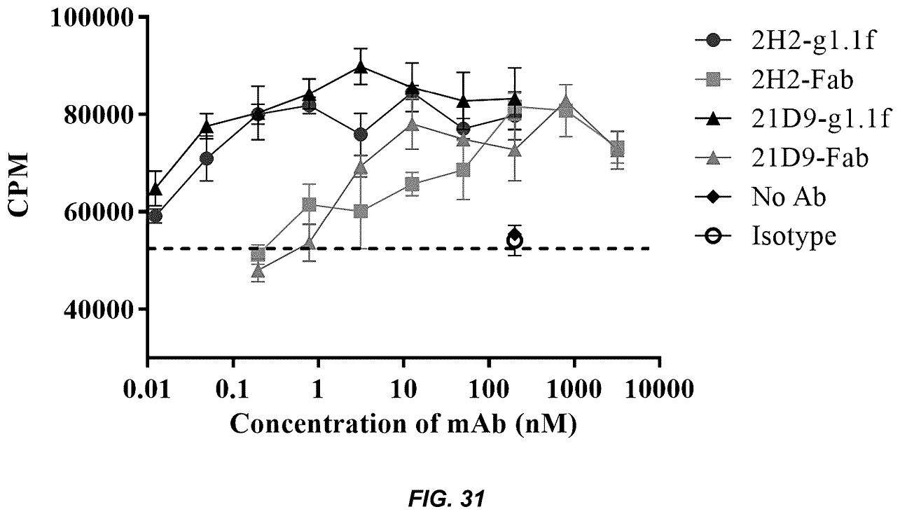

The results, which are shown in FIG. 31, indicate that both full length anti-ILT4 antibodies 21D9.IgG1.1f and 2H2.IgG1.1f, and their respective Fab fragments induce T cell proliferation.

### Example 16: Binding of Anti-hILT4 Antibodies to Cyno ILT4

Several ILT family members were obtained via PCR from cynomolgus monkeys. Since sequence analysis of these proteins did not clearly indicate which was the closest match to hILT4, expression profiling and functional assays were conducted. These assays identified a clone named “9152” as the best match to hILT4 with regards to expression profiling and functional characteristics. The amino acid sequence of the extracellular region of 9152, including a His-Avi Tag, is the following:

(SEQ ID NO: 118)
QAGILPKPMLWAEPDRVITQGSPVTLRCQGNLEARGYHLYRERKSASWIT
LIRPELVKKGQFPIPSITWEDAGRYRCQYYSHSWWSEHSDPLELVVTGAY
RKPTLSALPSPVVASGGNVTLQCDSRVALDGFILCKEGEDEHSQRLNSQP
RTRGSSRAVFSVGPVSPSRRWSYRCYGYELHSRYVWSLPSDLLELLVPGV
SKKPSLSVQPGPVVAGGDKLTLQCGSDAGYDRFALYKEGERDFLQRPGQQ
LQAGLAQANFTLDPVRGSHGGQYRCYGAHNLSSEWSAPSDPLDILISAGP
HSGLRRECDPAVSVTGMDGHFLSDQGGSSSPGGGSGGGSEQKLISEEDLG
HHHHHHGLNDIFEAQKIEWHE.

For binding to hILT4, the extracellular region (ECD) of hILT4-His-Avi tag; SEQ ID NO: 119, described in Example 2, was used.

The kinetics of 21D9.e antibody binding to cyno and human ILT4, using the above ILT4 constructs, was analyzed in a comparative SPR assay. The antibody was amine-coupled to a GLC sensor chip in a Proteon XPR36 instrument (BioRad). Human ILT4 and cyno ILT4 9152 were injected as analytes in a 5-membered, 5-fold dilution series with 1 μM maximum concentration (1.6 nM minimum concentration). All data were double-referenced, exported, and analyzed with BIAevaluation Software 4.1.1 using the kinetic titration model described by Karlsson et al (Karlsson et al. (2005) Analytical Biochem. 349:136). The running buffer consisted of 10 mM HEPES pH 7.4, 150 mM NaCl and 0.05% Tween-20. The assay temperature was 37° C.

The results, which are shown in Table 5, show that 21D9.e bound cyno ILT4 9152 with approximately 11-fold weaker affinity than human ILT4 (K_{D} values of 3.5 nM for cyno and 0.33 nM for human ILT4). Similarly, the parental antibody, 21D9, bound cyno with approximately 12 fold weaker affinity than human ILT4. The difference was driven by a faster dissociation rate constant for cyno ILT4 (K_{D} values of 4.0 nM for cyno and 0.32 nM for human ILT4).

TABLE 5

| | | Ka | kd | KD |
|---|---|---|---|---|
| Ligand | Analyte | (M − 1 s − 1) | (s − 1) | (M) |
| 21D9 | cyLILR 9152 | 8.8E+05 | 3.5E−03 | 4.0E−09 |
| 21D9 | hILT4 | 8.2E+05 | 2.6E−04 | 3.2E−10 |
| 21D9e | cyLILR 9152 | 7.3E+05 | 2.6E−03 | 3.5E−09 |
| 21D9e | hILT4 | 9.9E+05 | 3.3E−04 | 3.3E−10 |

### Example 17: ILT4 Antibodies Promote Expression of Costimulatory Molecules and Activation Markers on Cynomolgus Monocyte-Derived Dendritic Cells In Vitro

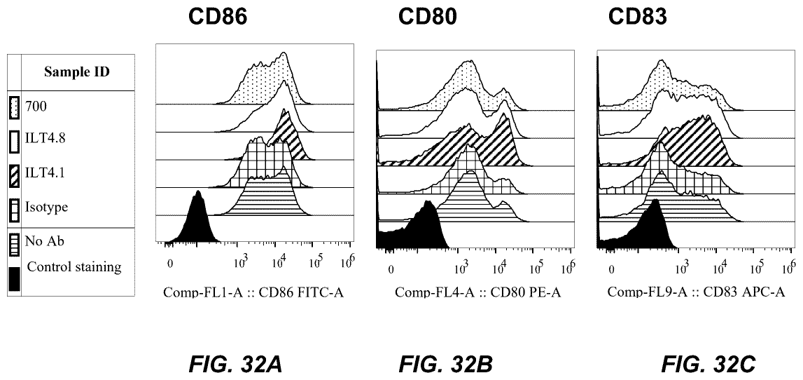

This Example shows that ILT4 antibodies 21D9.e.IgG1.3 (ILT4.8 in FIG. 32; see Table 1) and 9G4. hIgG1.3 (ILT4.1 in FIG. 32; see Table 1) stimulated the expression of CD80, CD83 and CD86 on monocyte derived dendritic cells.

Monocytes isolated from cynomolgus monkey PBMC (Non-human Primate CD14 Microbeads, Miltenyi) were cultured in the presence of 62.5 U/mL recombinant human GM-CSF (Peprotech) and 125 U/mL recombinant human IL-4 (Peprotech) for 5 days. Anti-LT4 antibodies were added to the culture @10 μg/mL at the time the culture was set up. Cells were stained for their surface expression level of CD86, CD80, and CD83 using anti-CD86 (clone #IT2.2, BioLegend), anti-CD80 (clone #L307), and anti-CD83 (clone #HIB15e). The samples were acquired on a Cytoflex® cytometer (Beckmen Coulter) and analyzed with FlowJo™ software (Tree Star, Inc, Ashland, OR).

The results, which are shown in FIGS. 32A-C, indicate that ILT4 antibodies 21D9.e.IgG1.3 (aka. ILT4.8.IgG1.3; “ILT4.8” in the figure) and 9G4.IgG1.3 (aka. ILT4.1.IgG1.3; “ILT4.1” in the figure) promote expression of co-stimulatory molecules CD80 and CD86 as well as the dendritic cell maturation marker CD83 in monocyte-derived DC, as evidenced by the upregulation of CD80, CD83 and CD86 on the dendritic cells.

### Example 18: Modified Anti-ILT4 Antibody 10F10.4

The tyrosine in F36Y in the light chain of 10F10.1 and 10F10.3 (see Example 2) is located near the heavy chain CDR3 in modeling of hILT4 and 10F10 antibodies. Therefore, substituting F36 with Y could cause a clash with an isoleucine in heavy chain CDR3, as evidenced by a faster off-rate of 10F10.1 and 10F10.3 relative to 10F10 (see Example 2). An additional mutant was created in which F36 of the light chain was not mutated and the only light chain framework region mutation made in 10F10 was T63S. The heavy chain was not mutated, and so is that of 10F10. This modified antibody is called 10F10.4, and the amino acid sequence of its light chain is set forth in the Sequence Table as SEQ ID NO: 116.

The binding kinetics of 10F10.4 were determined at 37° C. using a Biacore® T200 instrument. The running buffer was 10 mM HEPES pH 7.4 supplemented with 0.05% Tween-20 and 1 g/L BSA. All anti-ILT4 antibodies were captured on a CM4 chip with pre-immobilized anti-human Fc capture antibody from Southern Biotech (catalog number 2081-01). Human ILT4 (hILT4-His-Avi tag described in Example 2) was injected at 1.2 uM (only over 10F10, 10F10.4 and one replicate of 1E5 ILT4.15), 405 nM, 81 nM (in duplicate), 16 nM, 3 nM and 0.6 nM. All data were double-referenced and fitted to a 1:1 Langmuir model with mass transport using Biacore T200 Evaluation Software version 3.1.

The results, which are shown in Table 6, indicate that binding kinetics of 10F10.4 are very similar to those of 10F10. 21A5, 21A5a, 21D9, and 21D9.e were included as controls. The absolute values differ slightly from values shown in Table 2, which is due to the different capture reagent utilized in this assay.

TABLE 6

| | | | | | | Ligand | |
|---|---|---|---|---|---|---|---|
| | | ka | | | Rmax | Level | app. |
| Ligand | Sample | (1/Ms) | kd (1/s) | KD (M) | (RU) | (RU) | act. |
| 10F10 | hILT4 | 1.2E+06 | 2.6E−02 | 2.2E−08 | 35 | 50 | 100% |
| 10F10.4 | hILT4 | 1.2E+06 | 2.7E−02 | 2.2E−08 | 33 | 41 | 117% |
| 21A5 | hILT4 | 5.2E+05 | 1.2E−05 | 2.3E−11 | 81 | 119 | 98% |
| 21A5.a | hILT4 | 4.9E+05 | 1.7E−05 | 3.6E−11 | 76 | 109 | 100% |
| 21D9 | hILT4 | 1.1E+06 | 5.4E−04 | 5.0E−10 | 31 | 65 | 70% |
| 21D9.e | hILT4 | 1.0E+06 | 5.8E−04 | 5.8E−10 | 101 | 154 | 94% |

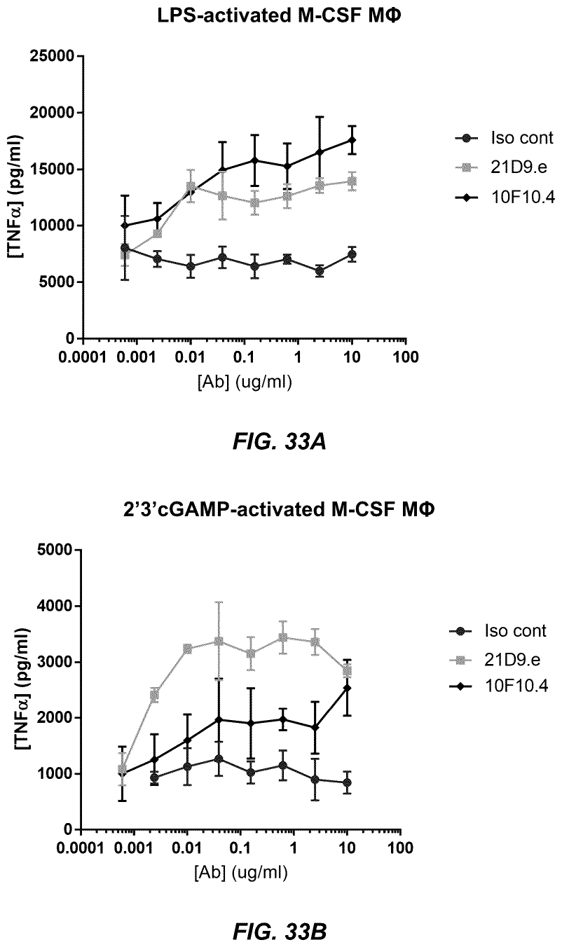

10F10.4 was shown to be functionally active by potentiating TNFα secretion from in vitro differentiated macrophages. This was demonstrated by incubating 10F10.4 with macrophages differentiated from PBMC monocytes as described in Example 8. 21D9.e was also included, as well as an isotype control. The results, which are shown in FIGS. 33A and B, indicate that the presence of ILT4 antibody 10F10.4 potentiated TNFα secretion during the in vitro differentiation of monocytes to macrophages by either LPS or a STING agonist.

### Example 19: 21D9.e has Similar Potency in the Context of IgG1, IgG4 and IgG1.3

This Example shows that the potency of 21D9.e is similar in the context of an IgG1, IgG4 (S228P) and IgG1.3 heavy chain.

21D9.e antibodies were cloned in the context of an IgG1, IgG4 (with S228P) or IgG1.3 (SEQ ID Nos: 176, 178 and 13, respectively) and used in an assay measuring TNFα secretion, conducted as follows.

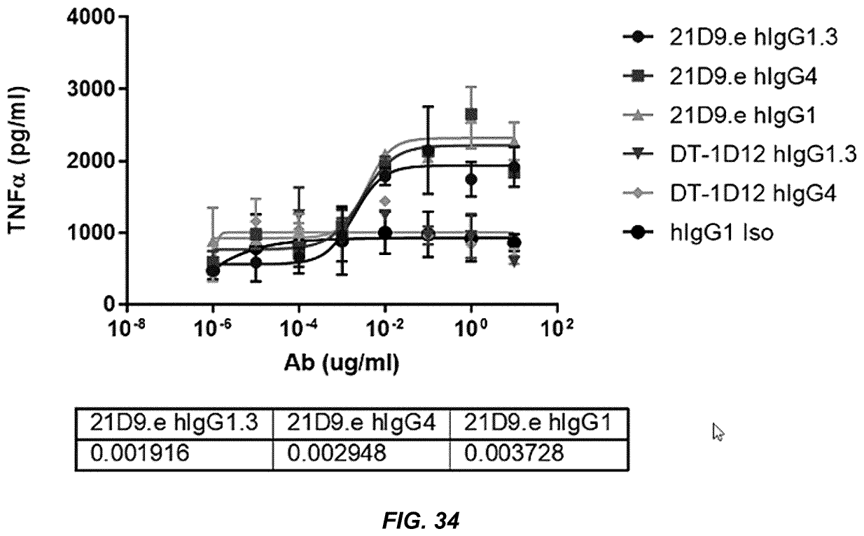

The results, which are shown in FIG. 34, indicate that a similar amount of TNFα is secreted for 21D9.e.IgG1, 21D9.e.IgG4.S228P and 21D9.e.IgG1.3. Thus, 21D9.e variable regions have similar functional potency in the context of these different Fc regions. DT-1D12 is an isotype control.

### Example 20: 21D9e Binds to hILT4 Alleles

This Example describes assays to determine whether 21D9.e binds to alternative alleles of hILT4.

Only those alleles having a frequency over 1% were considered. Two alleles having amino acid changes in regions D1 and D2 were further considered: E161D and R103H (numbering according to SEQ ID NO: 107, i.e., ILT4 with signal sequence). As 3-dimensional modeling showed that both amino acid residues are not near the binding interface with MHC class I, amino acid changes at these residues were not expected to affect antibody binding to hILT4. To confirm this, a hILT4 protein with the substitutions E161D and R103H (hILT4 E161D/R103H) was made and tested for binding to 21D9.e.IgG1.3 and 21A5.a.IgG1.3 (ILT4.9.IgG1.3; see Table 1) via Octet BLI. Both proteins were found to bind similarly to hILT4 E161D/R103H and to hILT4 E161/R103.

### Example 21: 21D9e.IgG1.3 does not Significantly Trigger Basophil Activation

The assay used for detection of basophil activation by anti-ILT4 antibodies involved the following steps. The assay was conducted by using the Buhlmann Laboratories AG methodology used in Flow CAST© Basophil Activation Test, generally as follows. Human basophils, derived from donor blood, were detected using anti-Ccr3. CD63 was used as an indicator of basophil activation. Stimulation buffer and the antibodies were added to whole blood. Following incubation, staining reagents were added to the blood; and following antibody incubation, erythrocytes were lysed and the samples were centrifuged, washed and analyzed on a BD FACS Canto II.

Based on this assay, Ccr3+CD63+ activated basophils from 8 donors were measured by FACS following incubation with either 21D9.e.IgG1.3 (ILT4.8.IgG1.3; see Table 1) 21.5Aa.IgG1.3, (ILT4.9.IgG1.3) or anti-DT at 4.5, 45.45 or 454.55 μg/mL for 180 minutes. Positive controls included anti-FcεRI and fMLP (N-formylmethionine-leucyl-phenyalanine). Negative control was DT1DT12-IgG1.3, which targets an irrelevant antigen, diphtheria toxin.

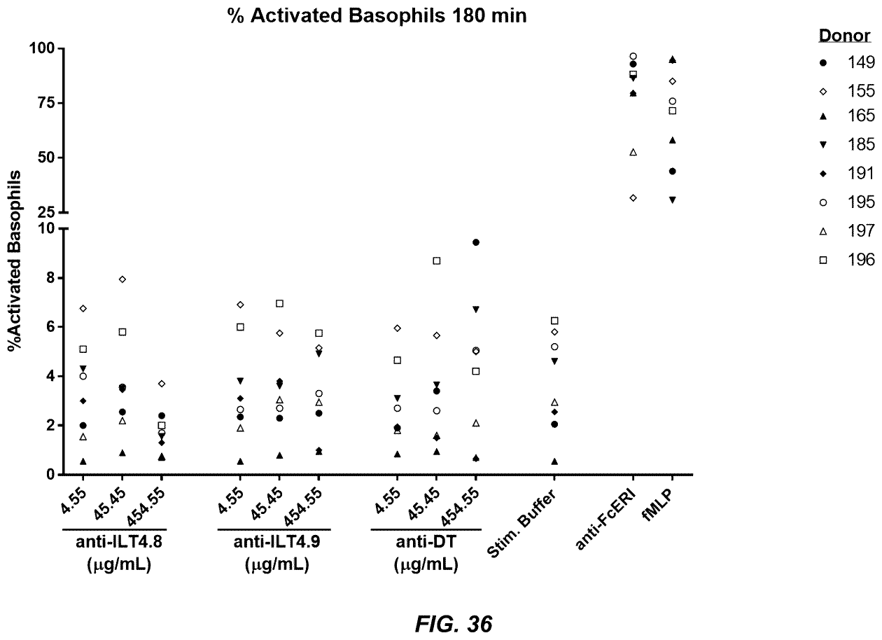

The results, which are shown in FIG. 36, indicate that 21D9.e.IgG1.3 (ILT4.8.IgG1.3) and 21.5Aa.IgG1.3 (ILT4.9.IgG1.3) do not induce basophil activation in the conditions tested. The lack of basophil activation represents an important safety feature.

### Example 22: Anti-ILT4 Antibody 21D9.e.IgG1.3 is Stable

This Example describes 3 month stability studies performed with anti-ILT4 antibody 21D9.e.IgG1.3 (aka. ILT4.8.IgG1.3), which data show that the antibody is stable at least over 3 months.

Samples of 21D9.e.IgG1.3 were incubated at 25 or 40° C. at a concentration of 150 mg/mL. The samples were diluted with mobile phase (40 mM NaH2PO4, 60 mM Na2HPO4, 0.1 M Na2SO4, pH 6.8) to 1 mg/mL and forty microliters were analyzed using an Agilent 1200 Series HPLC System (Agilent Technologies, Santa Clara, CA) using a TSKgel Super3000SW column (4.6 mm ID×30 cm, 4 μm, Tosoh Bioscience, King of Prussia, PA) with an isocratic gradient (0.1 M Sodium Phosphate, 0.1 M Sodium Sulfate, pH 6.8 at 0.2 mL/min). UV detection was set at 280 nM.

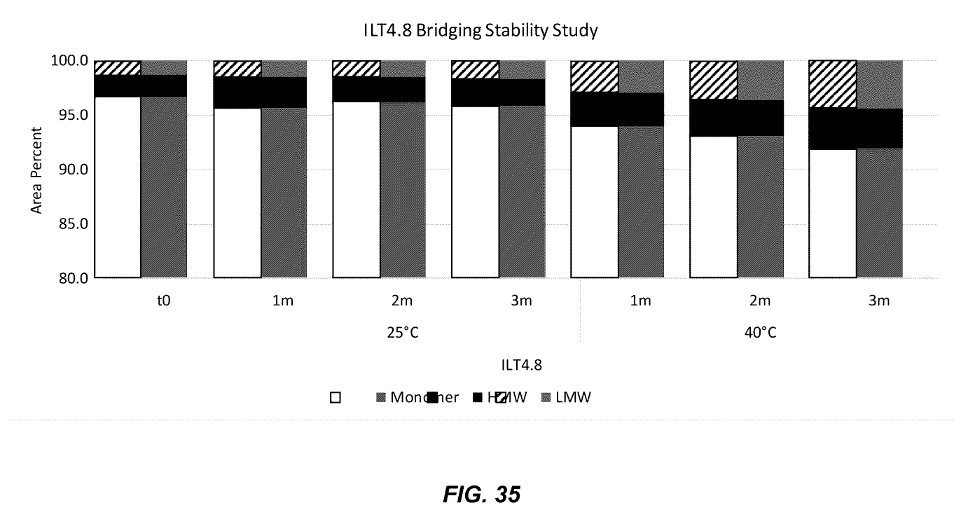

The results, which are shown in FIG. 35, indicate that the 21D9.e.IgG1.3 (ILT4.8) antibody composition comprises less than 5% of high and low molecular weight species after 3 months incubation at 25° C. and less than 10% of high and low molecular weight species after 3 months incubation at 40° C.

### Example 23: Cross-Blocking of 2H2 and 21D9 by Anti-ILT4 Monoclonal Antibodies

ILT4 overexpressing CHO cells were first pre-incubated with serial dilutions of unconjugated anti-ILT4 antibodies 10F10.IgG1.3 and 21A5.IgG1.3 (3-fold, 12-point, starting from 40 μg/mL). Anti-KLH-g1.1f (Anti-Hemocyanin antibody) was added as an isotype control while unconjugated 2H2.IgG1.1f or 21D9.IgG1.1f was added at 40 μg/mL as a positive control to demonstrate blocking activity. Following a 30-minute incubation period, PE conjugated 2H2.IgG1.1f or 21D9.IgG1.1f was spiked in at 1.5 μg/mL and incubated with cells for another 30 minutes. After washing, the samples were acquired on a FACSCanto™ cytometer (BD Bioscience, San Jose) and analyzed with Flowjo™ software (Tree Star, Inc, Ashland, OR).

The results are shown in FIG. 37A (2H2 cross blocking) and FIG. 37B (21D9 cross blocking). The results indicate that 10F10 and 21A5 cross-block (or compete with) 2H2 for binding to hILT4 (FIG. 37A) and that 10F10, but not 21A5, cross-blocks (or competes with) 21D9 for binding to hILT4 (FIG. 37B). Thus, (i) 21A5 and 2H2 are in the same binning group (i.e., they compete with each other for binding to hILT4); (ii) 21D9 is in a separate binning group from that of 21A5/2H2; and (iii) 10F10 competes with both 21D9 and 2H2/21A5 for binding to hILT4.

A diagram showing the hILT4 binding regions of the antibodies on the crystal structure of hILT4 is shown in FIG. 37C. The Ig-like domain 1 corresponds to amino acid residues 27-110 of SEQ ID NO: 107, while Ig-like domain 2 corresponds to amino acid residues 111-229 of SEQ ID NO: 107. These two domains are shown in ribbon form in FIG. 37C. The results of these experiments indicate that antibody 21D9 binds to Ig-like domain 1, antibodies 2H2 and 21A5 bind to Ig-like domain 2, and antibody 10F10 binds to a region that is located in Ig-like domain 2, close to Ig-like domain 1, and can be competed away by antibodies that bind to Ig-like domain 1 or Ig-like domain 2. Antibodies differing from the antibodies used in these experiments only by germline mutations (e.g., 21D9.e, 29A5.a and 10F10.3 and 10F10.4) are expected to bind the same region as their parent antibodies (21D9, 29A5 and 10F10, respectively).

### Example 24: Epitope Mapping Using Carbene Chemical Footprinting

The reagents for human ILT4 and anti-human ILT4 antibody Fab clones 10F10, 21D9, and 21A5 were buffer exchanged into 100 mM Tris-HCl and 50 mM NaCl at pH 7.5 at 1 mg/mL. Individual complexes of human ILT4 and each anti-human ILT4 antibody Fab were made by mixing at a 1:1 ratio. Triplicate experiments of 5 μg of ILT4 and 10 μg of the complexes were mixed 1:1 with 20 mM 4-[3-(Trifluoromethyl)-3H-diazirin-3-yl]benzoic acid in 100 mM Tris-HCl and 50 mM NaCl at pH 7.5. The resulting mixtures were snap frozen in liquid nitrogen. All samples were placed on dry ice and irradiated with light (300-800 nm) from an Atlas Suntest® CPS+ light box for 20 minutes to allow for covalent modification of the protein by the diazirine reagent. The irradiated samples were reduced, alkylated, and digested with chymotrypsin. LC-MS/MS analysis was performed on an Orbitrap Fusion Lumos® mass spectrometer using a Waters BEH C18 column. A 60 minute linear gradient at 10 μL/min of 0-35% buffer B (A: water with 0.1% formic acid; B: acetonitrile with 0.1% formic acid) was used to separate the peptides. The resulting eluent was mixed with 2 μL/min of acetonitrile mixed with 0.6% 3-nitrobenzyl alcohol. Modified peptides were identified and quantified using Byonic® and Byologic® from Protein Metrics. Chromatographic resolution and MS/MS identifications were used to localize and confirm the modified amino acids.

Diazirines are photoactive compounds that can react with any C—H bond in all 20 amino acids. The resulting covalent modification can be chromatographically separated from other amino acid sites in the same peptide. Comparisons of the triplicate peak areas for the unmodified peptides and singly modified peptides were used to determine differences in diazirine labeling for the antibody clones 10F10, 21A5, and 21A9 to identify the antibody epitope. Peptides with >50% decreases in labeling in one or more complexes were considered significant and used to localize the antibody epitopes. Significant peptides, or in cases where the comparison to the unmodified peptide could not be made due to a missed enzyme cleavage site, the modified peptide chromatogram for all samples were compared to each other with decreases of >50% of individual peptide peak elution chromatograms being used to determine differentially modified amino acids that are protected by the antibody epitope. The MS/MS spectra of the differentially modified amino acids were manually confirmed by MS/MS and marked on the human ILT4 x-ray crystallography structure (PDB: 2DYP). (FIG. 37D.) The antibody epitope for clone 21D9 was found to have differential labeling at two individual sites of 1 and 3 amino acids in domain 1 of human ILT4 and is marked in red and purple (due to an overlapping site with 10F10). The antibody epitope for clone 10F10 was found to have differential labeling at six individual sites of 1-2 amino acids in domain 1 and domain 2 of human ILT4 and as marked in FIG. 37D (due to overlapping sites with both 21A5 and 21D9). The antibody epitope for clone 21A5 was found to have differential labeling at 11 individual sites of 1-4 amino acids in domain 2 of human ILT4, as also marked in FIG. 37D (due to an overlapping site with both 10F10).

hILT4 amino acid residues Gly117, Val 119, Try120, Leu134, Lys136, Gln149, Pro150, Ile159, Ser161, Val162, Gly163, Pro164, Pro167, His173, Try178, Pro183, and Tyr184 are depicted as balls in FIG. 37D with respect to antibody 21A5. Residues Glu42, Lys43, Gly76, Cys77, Leu88, Pro91, Pro183, and Tyr184 are depicted in FIG. 37D with respect to antibody 10F10. Residues Lys43, Ile49, Thr50, and Arg51 are depicted with respect to antibody 21A9.

### Sequence Table

The following is a table of certain sequences referred to in this application. A number of the sequences below are also provided in FIGS. 1-15 and the VH and VL CDRs for the antibodies are denoted in FIGS. 1-15. Within the LC and HC sequences provided below, the component VL and VH sequences are underlined and bolded. Within the VL and VH sequences below, the CDRs are underlined and bolded, as depicted also in FIGS. 1-12, and within certain VH and VL sequences, reversion mutations are depicted. (See also FIGS. 13-15 for depiction of reversion mutations.) In some cases reversion mutations are shaded or are bold and underlined and grey-shaded.

| SEQ ID | | |
|---|---|---|
| NO | Name | Sequence |
| 1 | 9G4 kappa LC | EIVLTQSPATLSLSPGERATLSCRASQSVSSYLAWYQQKPGKA |
| | (aka. ILT4.1- | PRLLIYDASNRATGIPARFSGSGSGTDFTLTISSLEPEDFAGY |
| | kappa) | YCQQRSYWPWTFGQGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 2 | 9G4-IgG1.1f | QVQLQESGPGLVKPSETLSLTCTVSGGSISSYYWNWIRQPPGK |
| | HC | GLEWLGYIYYSGSTKYNPSLKSRVTISVDTSKNKFSLKLSSVT |
| | (aka. ILT4.1- | AADTAVYYCASSGWYYFDYWGQGTLVTVSSASTKGPSVFPLAP |
| | IgG1.1f) | SSKSTSGGTAALGCLVKDYFPEPVTVSWNSGALTSGVHTFPAV |
| | | LKSSGLYSLSSVVTVPSSSLGTQTYICNVNHKPSNTKVDKRVE |
| | | PKSCDKTHTCPPCPAPEAEGAPSVFLFPPKPKDTLMISRTPEV |
| | | TCVVVDVSHEDPEVKFNWYVDGVEVHNAKTKPREEQYNSTYRV |
| | | VSVLTVLHQDWLNGKEYKCKVSNKALPSSIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPGK |
| 3 | 9C8 kappa LC | AIQLTQSPSSLSASVGDRVTITCRASQGISSALAWYKKKPGKA |
| | ILT4.2-kappa | PKLLIYDASSLESGVPSRFSGSGSGTDFTLTISSLKPEDFATY |
| | | YCQQFNSYPFTFGPGTKVDIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSENRGEC |
| 4 | 9C8-IgG1.1f | QVKLVKSGAEVKKPGASVKVSCKASGYTFTSSDINWVRQATGQ |
| | HC | GLEWMGWMNPNSGHTGYAQKFQDRVTLTRDTSISTAYMELSSL |
| | ILT4.2- | RSEDSAVYYCARGGNSIDWGFSYYGLDVWGQGTTVTVSSASTK |
| | IgG1.1f | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPEAEGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPSSIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGNV |
| | | FSCSVMHEALHNHYTQKSLSLSPGK |
| 5 | 2H2 kappa LC | EIVLTQSPGTLSLSPGERATLSCRASQSVSSSYLAWYKKKPGQ |
| | ILT4.3-kappa | APRLLIYGASSRATGIPDRFSGSGSGTDFTLTISRLEPEDFAV |
| | | YYCQQYGSSYTFGQGTKLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 6 | 2H2-IgG1.1fHC | QVQLVQSGAEVKKPGSSVEVSCKASGGTFSNYAISWVRKAPGQ |
| | ILT4.3- | GLEWMGGIIPILATANYAPKFQGRVTITADEFTSSAYMELSSL |
| | IgG1.1f | RSEDTAVYYCAKSSITMIRGAYLYYYDGMDVWGKGTTVTVSSA |
| | | STKGPSVFPLAPSSKSTSGGTAALGCLVKDYFPEPVTVSWNSG |
| | | ALTSGVHTFPAVLQSSGLYSLSSVVTVPSSSLGTQTYICNVNH |
| | | KPSNTKVDKRVEPKSCDKTHTCPPCPAPEAEGAPSVFLFPPKP |
| | | KDTLMISRTPEVTCVVVDVSHEDPEVKFNWYVDGVEVHNAKTK |
| | | PREEQYNSTYRVVSVLTVLHQDWLNGKEYKCKVSNKALPSSIE |
| | | KTISKAKGQPREPQVYTLPPSREEMTKNQVSLTCLVKGFYPSD |
| | | IAVEWESNGQPENNYKTTPPVLDSDGSFFLYSKLTVDKSRWQQ |
| | | GNVFSCSVMHEALHNHYTQKSLSLSPGK |
| 7 | 2E5 kappa LC | EIVLTQSPATLSLSPGERATLSCRASQSVSSYLAWYQQKPGQA |
| | ILT4.4-kappa | PRLLIYDASNRATGIPARFSGSGSGTDFTLTISSLEPEDFAVY |
| | | YCQQRSNWPPWTFGQGTKVEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 8 | 2E5-IgG1.1f | QVKLVESGGGVVQPGRSLRLSCTASGFTFSNYGMHWVRQAPGK |
| | HC | GLEWVAVIWYDGSNEYYAESVKGRLTISRDNSKNTLYLQVNSL |
| | ILT4.4- | RAEDTAVYYCARDPFYGSGNYFDYWGQGTLVTVSSASTKGPSV |
| | IgG1.1f | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKRVEPKSCDKTHTCPPCPAPEAEGAPSVFLFPPKPKDTLMIS |
| | | RTPEVTCVVVDVSHEDPEVKFNWYVDGVEVHNAKTKPREEQYN |
| | | STYRVVSVLTVLHQDWLNGKEYKCKVSNKALPSSIEKTISKAK |
| | | GQPREPQVYTLPPSREEMTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| 9 | 24E5 kappa LC | DIQMTKSPSSLSASVGDRVTITCRASQGISSWLAWYQQKPEKA |
| | ILT4.5-kappa | PKSLIYAASSLKSGVPSRFSGSGSGTDFTLTISSLKPEDFATY |
| | | YCQQYNSYPLTFGGGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 10 | 24E5-IgG1.1 | EVKLLESGGGLVQPGGSLRLSCAASGFTFSSYVMSWVRQAPGK |
| | HC | GLEWVSGISGSGGSTYYADSVKGRFTISRDNSKNTLYLKMNSL |
| | ILT4.5- | RAEDTAVYYCAKDQDIIAAYYFVYWGQGTLVTVSSASTKGPSV |
| | IgG1.1f | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKRVEPKSCDKTHTCPPCPAPEAEGAPSVFLFPPKPKDTLMIS |
| | | RTPEVTCVVVDVSHEDPEVKFNWYVDGVEVHNAKTKPREEQYN |
| | | STYRVVSVLTVLHQDWLNGKEYKCKVSNKALPSSIEKTISKAK |
| | | GQPREPQVYTLPPSREEMTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| 11 | 21D9 kappa LC | EIVLTQSPGTLSLSPGERATLSCRASQSVSSSYLAWYQQKPGK |
| | ILT4.6-kappa | APRLLIYGASSRATGIPDRFSGSGSGTDFTLTISRLEPEDFAV |
| | | YYCQQYGSSPLTFGGGTKVEIKRTVAAPSVFIFPPSDEQLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 12 | 21D9-IgG1.1f | EGQLLESGGDLVQPGGSLRLSCATSGFTFSNYAMNWVRQAPGK |
| | HC | GLEWISVISVSGGSTYYADSVKGRFTISRDNSKNTLYLKMNSL |
| | ILT4.6- | RAEDTAVYYCAKDYYYDSGSYYDSFFDYWGQGTLVTVSSASTK |
| | IgG1.1f | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPEAEGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPSSIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGNV |
| | | FSCSVMHEALHNHYTQKSLSLSPGK |
| 13 | 21D9.e.IgG1.3 | EVKLLESGGGLVQPGGSLRLSCAASGFTFSNYAMNWVRQAPGK |
| | HC | GLEWVSVISVSGGSTYYADSVKGRFTISRDNSKNTLYLKMNSL |
| | ILT4.8.IgG1.3 | RAEDTAVYYCAKDYYYDSGSYYDSFFDYWGQGTLVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPEAEGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTP |
| | | PVLDSDGSFFLYSKLTVDKSRWQQGNVFSCSVMHEALHNHYTQ |
| | | KSLSLSPG |
| 14 | 21A5 kappa LC | EI [symbol] LTQSPGTLSLSPGERATLSCRASQSVSSSYLAWYQQKPGQ |
| | | APRLLIYGASSRATGIPDRFSGSGSGTDFTLTISRLEPEDFAV |
| | | YYCQQYGSTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC |
| 15 | 21A5.IgG1.3 | QVQLVQSGAEVKKPGASVKVSCKASGYTFTDYYLHWVRQAPGQ |
| | HC | GLEWMGIFHPSGDITSSAQNFQGRVTM [symbol] RDTSTSTVYMELSSL |
| | | RSEDTAVYYCARGGVLRYLDWSHAFDIWGQGTMVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPEAEGAPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPG |
| 16 | 21A5.1 kappa | EI [symbol] LTKSPGTLSLSPGERATLSCRASQSVSSSYLAWYKKKPGQ |
| | LC | APRLLIYGASSRATGIPDRFSGSGSGTDFTLTISRLEPEDFAV |
| | (ILT4.9.1K) | YYCQQYGSTFGGGTKVEIKRTVAAPSVFIFPPSDEQLKSGTAS |
| | | VVCLLNNFYPREAKVQWKVDNALQSGNSQESVTEQDSKDSTYS |
| | | LSSTLTLSKADYEKHKVYACEVTHQGLSSPVTKSFNRGEC |
| 17 | 21A5.a.IgG1.3 | QVKLVKSGAEVKKPGASVKVSCKASGYTFTDYYLHWVRQAPGQ |
| | HC | GLEWMGIFHPSGDITSSAQNFQGRVTMTRD [symbol] STSTVYMELSSL |
| | (ILT4.9.IgG1.3) | RSEDTAVYYCARGGVLRYLDWSHAFDIWGQGTMVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPEAEGAPSVFLFPPKPKDTL |
| | | MISRTPEVTCVVVDVSHEDPEVKFNWYVDGVEVHNAKTKPREE |
| | | QYNSTYRVVSVLTVLHQDWLNGKEYKCKVSNKALPAPIEKTIS |
| | | KAKGQPREPQVYTLPPSREEMTKNQVSLTCLVKGFYPSDIAVE |
| | | WESNGQPENNYKTTPPVLDSDGSFFLYSKLTVDKSRWQQGNVF |
| | | SCSVMHEALHNHYTQKSLSLSPG |
| 18 | 10F10 kappa | AIQLTQSPSSLSASVGDRVTITCRASQGISSALAWFQQKPGKA |
| | LC | PKLLIYDASSLESGVPSRFTGSGSGTDFTLTISSLKPEDFATY |
| | | YCQQFNSYPITFGQGTRLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 19 | 10F10.IgG1.3 | QVQLVESGGGVVQPGRSLRLSCAASGFTFSSYAMHWVRQAPGK |
| | HC | GLEWVAIISYDEYNKYYADSVKGRFTISRDNSKNTLYLKMNSL |
| | ILT4.10.IgG1.3 | RAEDTAVYYCAREWVGIRYWGQGTLVTVSSASTKGPSVFPLAP |
| | | SSKSTSGGTAALGCLVKDYFPEPVTVSWNSGALTSGVHTFPAV |
| | | LKSSGLYSLSSVVTVPSSSLGTQTYICNVNHKPSNTKVDKRVE |
| | | PKSCDKTHTCPPCPAPEAEGAPSVFLFPPKPKDTLMISRTPEV |
| | | TCVVVDVSHEDPEVKFNWYVDGVEVHNAKTKPREEQYNSTYRV |
| | | VSVLTVLHQDWLNGKEYKCKVSNKALPAPIEKTISKAKGQPRE |
| | | PQVYTLPPSREEMTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 20 | 10F10.1 kappa | AIQLTKSPSSLSASVGDRVTITCRASQGISSALAWYQQKPGKA |
| | LC | PKLLIYDASSLESGVPSRF [symbol] GSGSGTDFTLTISSLKPEDFATY |
| | | YCQQFNSYPITFGQGTRLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 21 | 10F10.3 kappa | AIQLTKSPSSLSASVGDRVTITCRASQGISSALAWYQQKPGKA |
| | LC | PKLLIYDASSLESGVPSRF [symbol] GSGSGTDFTLTISSLKPEDFATY |
| | | YCQQFNSYPITFGQGTRLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 22 | 9G4 LC DNA | GAAATTGTGTTGACACAGTCTCCAGCCACCCTGTCTTTGTCTC |
| | ILT4.1-kappa | CAGGGGAAAGAGCCACCCTCTCCTGCAGGGCCAGTCAGAGTGT |
| | DNA | TAGCAGCTACTTAGCCTGGTACCAACAGAAACCTGGCCAGGCT |
| | | CCCAGGCTCCTCATCTATGATGCGTCCAACAGGGCCACTGGCA |
| | | TCCCAGCCAGGTTCAGTGGCAGTGGGTCTGGGACAGACTTCAC |
| | | TCTCACCATCAGCAGCCTAGAGCCTGAAGATTTTGCAGGTTAT |
| | | TACTGTCAGCAGCGTAGCTACTGGCCGTGGACGTTCGGCCAAG |
| | | GGACCAAGGTGGAAATCAAACGTACGGTGGCTGCACCATCTGT |
| | | CTTCATCTTCCCGCCATCTGATGAGCAGTTGAAATCTGGAACT |
| | | GCCTCTGTTGTGTGCCTGCTGAATAACTTCTATCCCAGAGAGG |
| | | CCAAAGTACAGTGGAAGGTGGATAACGCCCTCCAATCGGGTAA |
| | | CTCCCAGGAGAGTGTCACAGAGCAGGACAGCAAGGACAGCACC |
| | | TACAGCCTCAGCAGCACCCTGACGCTGAGCAAAGCAGACTACG |
| | | AGAAACACAAAGTCTACGCCTGCGAAGTCACCCATCAGGGCCT |
| | | GAGCTCGCCCGTCACAAAGAGCTTCAACAGGGGAGAGTGTTAG |
| 23 | 9G4 IgG1.1f | CAGGTGCAGCTGCAGGAGTCGGGCCCAGGACTGGTGAAGCCTT |
| | HC DNA | CGGAGACCCTGTCCCTCACCTGCACTGTCTCTGGTGGCTCCAT |
| | ILT4.1- | CAGTAGTTACTACTGGAACTGGATTCGGCAGCCCCCAGGGAAG |
| | IgG1.1f DNA | GGACTGGAGTGGCTTGGGTACATCTATTACAGTGGGAGTACCA |
| | | AGTACAACCCCTCCCTCAAGAGTCGAGTCACCATATCAGTAGA |
| | | CACGTCCAAGAACCAGTTCTCCCTGAAGCTGAGCTCTGTGACC |
| | | GCTGCGGACACGGCCGTGTATTATTGTGCCAGCAGTGGCTGGT |
| | | ACTACTTTGACTATTGGGGCCAGGGAACCCTGGTCACCGTCTC |
| | | CTCAGCTAGCACCAAGGGCCCATCGGTCTTCCCCCTGGCACCC |
| | | TCCTCCAAGAGCACCTCTGGGGGCACAGCGGCCCTGGGCTGCC |
| | | TGGTCAAGGACTACTTCCCCGAACCGGTGACGGTGTCGTGGAA |
| | | CTCAGGCGCCCTGACCAGCGGCGTGCACACCTTCCCGGCTGTC |
| | | CTACAGTCCTCAGGACTCTACTCCCTCAGCAGCGTGGTGACCG |
| | | TGCCCTCCAGCAGCTTGGGCACCCAGACCTACATCTGCAACGT |
| | | GAATCACAAGCCCAGCAACACCAAGGTGGACAAGAGAGTTGAG |
| | | CCCAAATCTTGTGACAAAACTCACACATGCCCACCGTGCCCAG |
| | | CACCTGAAGCCGAAGGGGCCCCGTCAGTCTTCCTCTTCCCCCC |
| | | AAAACCCAAGGACACCCTCATGATCTCCCGGACCCCTGAGGTC |
| | | ACATGCGTGGTGGTGGACGTGAGCCACGAAGACCCTGAGGTCA |
| | | AGTTCAACTGGTACGTGGACGGCGTGGAGGTGCATAATGCCAA |
| | | GACAAAGCCGCGGGAGGAGCAGTACAACAGCACGTACCGTGTG |
| | | GTCAGCGTCCTCACCGTCCTGCACCAGGACTGGCTGAATGGCA |
| | | AGGAGTACAAGTGCAAGGTCTCCAACAAAGCCCTCCCAAGCAG |
| | | CATCGAGAAAACCATCTCCAAAGCCAAAGGGCAGCCCCGAGAA |
| | | CCACAGGTGTACACCCTGCCCCCATCCCGGGAGGAGATGACCA |
| | | AGAACCAGGTCAGCCTGACCTGCCTGGTCAAAGGCTTCTATCC |
| | | CAGCGACATCGCCGTGGAGTGGGAGAGCAATGGGCAGCCGGAG |
| | | AACAACTACAAGACCACGCCTCCCGTGCTGGACTCCGACGGCT |
| | | CCTTCTTCCTCTATAGCAAGCTCACCGTGGACAAGAGCAGGTG |
| | | GCAGCAGGGGAACGTCTTCTCATGCTCCGTGATGCATGAGGCT |
| | | CTGCACAACCACTACACGCAGAAGAGCCTCTCCCTGTCCCCGG |
| | | GTAAATGA |
| 24 | 9C8 kappa LC | GCCATCCAGTTGACCCAGTCTCCATCCTCCCTGTCTGCATCTG |
| | DNA | TAGGAGACAGAGTCACCATCACTTGCCGGGCAAGTCAGGGCAT |
| | ILT4.2-kappa | TAGCAGTGCTTTAGCCTGGTATCAGCAGAAACCAGGGAAAGCT |
| | DNA | CCTAAGCTCCTGATCTATGATGCCTCCAGTTTGGAAAGTGGGG |
| | | TCCCATCAAGGTTCAGCGGCAGTGGATCTGGGACAGATTTCAC |
| | | TCTCACCATCAGCAGCCTGCAGCCTGAAGATTTTGCAACTTAT |
| | | TACTGTCAACAGTTTAATAGTTACCCATTCACTTTCGGCCCTG |
| | | GGACCAAAGTGGATATCAAACGTACGGTGGCTGCACCATCTGT |
| | | CTTCATCTTCCCGCCATCTGATGAGCAGTTGAAATCTGGAACT |
| | | GCCTCTGTTGTGTGCCTGCTGAATAACTTCTATCCCAGAGAGG |
| | | CCAAAGTACAGTGGAAGGTGGATAACGCCCTCCAATCGGGTAA |
| | | CTCCCAGGAGAGTGTCACAGAGCAGGACAGCAAGGACAGCACC |
| | | TACAGCCTCAGCAGCACCCTGACGCTGAGCAAAGCAGACTACG |
| | | AGAAACACAAAGTCTACGCCTGCGAAGTCACCCATCAGGGCCT |
| | | GAGCTCGCCCGTCACAAAGAGCTTCAACAGGGGAGAGTGTTAG |
| 25 | 9C8 IgG1.1f | CAGGTGCAACTGGTGCAGTCTGGGGCTGAGGTAAAGAAGCCTG |
| | DNA | GGGCCTCAGTGAAGGTCTCCTGCAAGGCTTCTGGATACACCTT |
| | ILT4.2- | CACCAGCTCTGATATCAACTGGGTGCGACAGGCCACTGGACAA |
| | IgG1.1f DNA | GGGCTTGAGTGGATGGGATGGATGAACCCTAACAGTGGTCACA |
| | | CAGGCTATGCACAGAAGTTCCAGGACAGAGTCACCTTGACCCG |
| | | GGACACCTCCATAAGCACAGCCTACATGGAGCTGAGCAGCCTG |
| | | AGATCTGAGGACTCGGCCGTGTATTACTGTGCGAGAGGTGGGA |
| | | ATAGCATTGACTGGGGGTTCTCCTACTACGGTCTGGACGTCTG |
| | | GGGCCAAGGGACCACGGTCACCGTCTCCTCAGCTAGCACCAAG |
| | | GGCCCATCGGTCTTCCCCCTGGCACCCTCCTCCAAGAGCACCT |
| | | CTGGGGGCACAGCGGCCCTGGGCTGCCTGGTCAAGGACTACTT |
| | | CCCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGAAGG |
| | | GGCCCCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGAG |
| | | GAGCAGTACAACAGCACGTACCGTGTGGTCAGCGTCCTCACCG |
| | | TCCTGCACCAGGACTGGCTGAATGGCAAGGAGTACAAGTGCAA |
| | | GGTCTCCAACAAAGCCCTCCCAAGCAGCATCGAGAAAACCATC |
| | | TCCAAAGCCAAAGGGCAGCCCCGAGAACCACAGGTGTACACCC |
| | | TGCCCCCATCCCGGGAGGAGATGACCAAGAACCAGGTCAGCCT |
| | | GACCTGCCTGGTCAAAGGCTTCTATCCCAGCGACATCGCCGTG |
| | | GAGTGGGAGAGCAATGGGCAGCCGGAGAACAACTACAAGACCA |
| | | CGCCTCCCGTGCTGGACTCCGACGGCTCCTTCTTCCTCTATAG |
| | | CAAGCTCACCGTGGACAAGAGCAGGTGGCAGCAGGGGAACGTC |
| | | TTCTCATGCTCCGTGATGCATGAGGCTCTGCACAACCACTACA |
| | | CGCAGAAGAGCCTCTCCCTGTCCCCGGGTAAATGA |
| 26 | 2H2 kappa LC | GAAATTGTGTTGACGCAGTCTCCAGGCACCCTGTCTTTGTCTC |
| | DNA | CAGGGGAAAGAGCCACCCTCTCCTGCAGGGCCAGTCAGAGTGT |
| | ILT4.3-kappa | TAGCAGCAGCTACTTAGCCTGGTACCAGCAGAAACCTGGCCAG |
| | DNA | GCTCCCAGGCTCCTCATCTATGGTGCATCCAGCAGGGCCACTG |
| | | GCATCCCAGACAGGTTCAGTGGCAGTGGGTCTGGGACAGACTT |
| | | CACTCTCACCATCAGCAGACTGGAGCCTGAAGATTTTGCAGTG |
| | | TATTACTGTCAGCAGTATGGTAGCTCGTACACTTTTGGCCAGG |
| | | GGACCAAGCTGGAGATCAAACGTACGGTGGCTGCACCATCTGT |
| | | CTTCATCTTCCCGCCATCTGATGAGCAGTTGAAATCTGGAACT |
| | | GCCTCTGTTGTGTGCCTGCTGAATAACTTCTATCCCAGAGAGG |
| | | CCAAAGTACAGTGGAAGGTGGATAACGCCCTCCAATCGGGTAA |
| | | CTCCCAGGAGAGTGTCACAGAGCAGGACAGCAAGGACAGCACC |
| | | TACAGCCTCAGCAGCACCCTGACGCTGAGCAAAGCAGACTACG |
| | | AGAAACACAAAGTCTACGCCTGCGAAGTCACCCATCAGGGCCT |
| | | GAGCTCGCCCGTCACAAAGAGCTTCAACAGGGGAGAGTGTTAG |
| 27 | 2H2 IgG1.1f | CAGGTCCAGTTGGTGCAGTCTGGGGCTGAGGTGAAGAAGCCTG |
| | HC DNA | GGTCCTCGGTGGAGGTCTCCTGCAAGGCTTCTGGGGGCACCTT |
| | ILT4.3- | CAGCAACTATGCTATCAGCTGGGTGCGACAGGCCCCTGGACAA |
| | IgG1.1f DNA | GGGCTTGAGTGGATGGGAGGGATCATCCCCATCTTGGCTACAG |
| | | CAAACTACGCACCGAAGTTCCAGGGCAGAGTCACGATTACCGC |
| | | GGACGAATTCACGAGCTCAGCTTACATGGAGCTGAGCAGCCTG |
| | | AGATCTGAGGACACGGCCGTGTATTACTGTGCGAAGTCTAGTA |
| | | TTACTATGATTCGGGGAGCCTATCTTTACTACTACGACGGTAT |
| | | GGACGTCTGGGGCCAAGGGACCACGGTCACCGTCTCCTCAGCT |
| | | AGCACCAAGGGCCCATCGGTCTTCCCCCTGGCACCCTCCTCCA |
| | | AGAGCACCTCTGGGGGCACAGCGGCCCTGGGCTGCCTGGTCAA |
| | | GGACTACTTCCCCGAACCGGTGACGGTGTCGTGGAACTCAGGC |
| | | GCCCTGACCAGCGGCGTGCACACCTTCCCGGCTGTCCTACAGT |
| | | CCTCAGGACTCTACTCCCTCAGCAGCGTGGTGACCGTGCCCTC |
| | | CAGCAGCTTGGGCACCCAGACCTACATCTGCAACGTGAATCAC |
| | | AAGCCCAGCAACACCAAGGTGGACAAGAGAGTTGAGCCCAAAT |
| | | CTTGTGACAAAACTCACACATGCCCACCGTGCCCAGCACCTGA |
| | | AGCCGAAGGGGCCCCGTCAGTCTTCCTCTTCCCCCCAAAACCC |
| | | AAGGACACCCTCATGATCTCCCGGACCCCTGAGGTCACATGCG |
| | | TGGTGGTGGACGTGAGCCACGAAGACCCTGAGGTCAAGTTCAA |
| | | CTGGTACGTGGACGGCGTGGAGGTGCATAATGCCAAGACAAAG |
| | | CCGCGGGAGGAGCAGTACAACAGCACGTACCGTGTGGTCAGCG |
| | | TCCTCACCGTCCTGCACCAGGACTGGCTGAATGGCAAGGAGTA |
| | | CAAGTGCAAGGTCTCCAACAAAGCCCTCCCAAGCAGCATCGAG |
| | | AAAACCATCTCCAAAGCCAAAGGGCAGCCCCGAGAACCACAGG |
| | | TGTACACCCTGCCCCCATCCCGGGAGGAGATGACCAAGAACCA |
| | | GGTCAGCCTGACCTGCCTGGTCAAAGGCTTCTATCCCAGCGAC |
| | | ATCGCCGTGGAGTGGGAGAGCAATGGGCAGCCGGAGAACAACT |
| | | ACAAGACCACGCCTCCCGTGCTGGACTCCGACGGCTCCTTCTT |
| | | CCTCTATAGCAAGCTCACCGTGGACAAGAGCAGGTGGCAGCAG |
| | | GGGAACGTCTTCTCATGCTCCGTGATGCATGAGGCTCTGCACA |
| | | ACCACTACACGCAGAAGAGCCTCTCCCTGTCCCCGGGTAAATG |
| | | A |
| 28 | 2E5 kappa LC | GAAATTGTGTTGACACAGTCTCCAGCCACCCTGTCTTTGTCTC |
| | DNA | CAGGGGAAAGAGCCACCCTCTCCTGCAGGGCCAGTCAGAGTGT |
| | ILT4.4-kappa | TAGCAGCTACTTAGCCTGGTACCAACAGAAACCTGGCCAGGCT |
| | DNA | CCCAGGCTCCTCATCTATGATGCATCCAACAGGGCCACTGGCA |
| | | TCCCAGCCAGGTTCAGTGGCAGTGGGTCTGGGACAGACTTCAC |
| | | TCTCACCATCAGCAGCCTAGAGCCTGAAGATTTTGCAGTTTAT |
| | | TACTGTCAGCAGCGTAGCAACTGGCCTCCGTGGACGTTCGGCC |
| | | AAGGGACCAAGGTGGAAATCAAACGTACGGTGGCTGCACCATC |
| | | TGTCTTCATCTTCCCGCCATCTGATGAGCAGTTGAAATCTGGA |
| | | ACTGCCTCTGTTGTGTGCCTGCTGAATAACTTCTATCCCAGAG |
| | | AGGCCAAAGTACAGTGGAAGGTGGATAACGCCCTCCAATCGGG |
| | | TAACTCCCAGGAGAGTGTCACAGAGCAGGACAGCAAGGACAGC |
| | | ACCTACAGCCTCAGCAGCACCCTGACGCTGAGCAAAGCAGACT |
| | | ACGAGAAACACAAAGTCTACGCCTGCGAAGTCACCCATCAGGG |
| | | CCTGAGCTCGCCCGTCACAAAGAGCTTCAACAGGGGAGAGTGT |
| | | TAG |
| 29 | 2E5 IgG1.1f | CAGGTGCAGCTGGTGGAGTCTGGGGGAGGCGTGGTCCAGCCTG |
| | HC DNA | GGAGGTCCCTGAGACTCTCCTGTACAGCGTCTGGATTCACCTT |
| | ILT4.4- | CAGTAACTATGGCATGCACTGGGTCCGCCAGGCTCCAGGCAAG |
| | IgG1.1f DNA | GGACTGGAGTGGGTGGCAGTTATCTGGTATGATGGAAGTAATG |
| | | AATACTATGCAGAATCCGTGAAGGGCCGACTCACCATCTCCAG |
| | | AGACAATTCCAAGAACACGCTGTATCTGCAAGTGAACAGCCTG |
| | | AGAGCCGAGGACACGGCTGTGTATTATTGTGCGAGAGATCCTT |
| | | TCTATGGTTCGGGGAATTACTTTGACTACTGGGGCCAGGGAAC |
| | | CCTGGTCACCGTCTCCTCAGCTAGCACCAAGGGCCCATCGGTC |
| | | TTCCCCCTGGCACCCTCCTCCAAGAGCACCTCTGGGGGCACAG |
| | | CGGCCCTGGGCTGCCTGGTCAAGGACTACTTCCCCGAACCGGT |
| | | GACGGTGTCGTGGAACTCAGGCGCCCTGACCAGCGGCGTGCAC |
| | | ACCTTCCCGGCTGTCCTACAGTCCTCAGGACTCTACTCCCTCA |
| | | GCAGCGTGGTGACCGTGCCCTCCAGCAGCTTGGGCACCCAGAC |
| | | CTACATCTGCAACGTGAATCACAAGCCCAGCAACACCAAGGTG |
| | | GACAAGAGAGTTGAGCCCAAATCTTGTGACAAAACTCACACAT |
| | | GCCCACCGTGCCCAGCACCTGAAGCCGAAGGGGCCCCGTCAGT |
| | | CTTCCTCTTCCCCCCAAAACCCAAGGACACCCTCATGATCTCC |
| | | CGGACCCCTGAGGTCACATGCGTGGTGGTGGACGTGAGCCACG |
| | | AAGACCCTGAGGTCAAGTTCAACTGGTACGTGGACGGCGTGGA |
| | | GGTGCATAATGCCAAGACAAAGCCGCGGGAGGAGCAGTACAAC |
| | | AGCACGTACCGTGTGGTCAGCGTCCTCACCGTCCTGCACCAGG |
| | | ACTGGCTGAATGGCAAGGAGTACAAGTGCAAGGTCTCCAACAA |
| | | AGCCCTCCCAAGCAGCATCGAGAAAACCATCTCCAAAGCCAAA |
| | | GGGCAGCCCCGAGAACCACAGGTGTACACCCTGCCCCCATCCC |
| | | GGGAGGAGATGACCAAGAACCAGGTCAGCCTGACCTGCCTGGT |
| | | CAAAGGCTTCTATCCCAGCGACATCGCCGTGGAGTGGGAGAGC |
| | | AATGGGCAGCCGGAGAACAACTACAAGACCACGCCTCCCGTGC |
| | | TGGACTCCGACGGCTCCTTCTTCCTCTATAGCAAGCTCACCGT |
| | | GGACAAGAGCAGGTGGCAGCAGGGGAACGTCTTCTCATGCTCC |
| | | GTGATGCATGAGGCTCTGCACAACCACTACACGCAGAAGAGCC |
| | | TCTCCCTGTCCCCGGGTAAATGA |
| 30 | 24E5 kappa LC | GACATCCAGATGACCCAGTCTCCATCCTCACTGTCTGCATCTG |
| | DNA | TAGGAGACAGAGTCACCATCACTTGTCGGGCGAGTCAGGGTAT |
| | ILT4.5-kappa | TAGCAGCTGGTTAGCCTGGTATCAGCAGAAACCAGAGAAAGCC |
| | DNA | CCTAAGTCCCTGATCTATGCTGCATCCAGTTTGCAAAGTGGGG |
| | | TCCCATCAAGGTTCAGCGGCAGTGGATCTGGGACAGATTTCAC |
| | | TCTCACCATCAGCAGCCTGCAGCCTGAAGATTTTGCAACTTAT |
| | | TACTGCCAACAGTATAATAGTTACCCTCTCACTTTCGGCGGAG |
| | | GGACCAAGGTGGAGATCAAACGTACGGTGGCTGCACCATCTGT |
| | | CTTCATCTTCCCGCCATCTGATGAGCAGTTGAAATCTGGAACT |
| | | GCCTCTGTTGTGTGCCTGCTGAATAACTTCTATCCCAGAGAGG |
| | | CCAAAGTACAGTGGAAGGTGGATAACGCCCTCCAATCGGGTAA |
| | | CTCCCAGGAGAGTGTCACAGAGCAGGACAGCAAGGACAGCACC |
| | | TACAGCCTCAGCAGCACCCTGACGCTGAGCAAAGCAGACTACG |
| | | AGAAACACAAAGTCTACGCCTGCGAAGTCACCCATCAGGGCCT |
| | | GAGCTCGCCCGTCACAAAGAGCTTCAACAGGGGAGAGTGTTAG |
| 31 | 24E5 IgG1.1f | GAGGTGCAGCTGTTGGAGTCTGGGGGAGGCTTGGTACAGCCTG |
| | HC DNA | GGGGGTCCCTGAGACTCTCCTGTGCAGCCTCTGGATTCACCTT |
| | ILT4.5- | TAGCAGCTATGTCATGAGCTGGGTCCGCCAGGCTCCAGGGAAG |
| | IgG1.1f DNA | GGGCTGGAGTGGGTCTCAGGTATTAGTGGTAGTGGTGGTAGCA |
| | | CATACTACGCAGACTCCGTGAAGGGCCGGTTCACCATCTCCAG |
| | | AGACAATTCCAAGAACACGCTGTATCTGCAAATGAACAGCCTG |
| | | AGAGCCGAGGACACGGCCGTATATTACTGTGCGAAAGATCAGG |
| | | ATATTATAGCAGCATACTACTTTGTCTACTGGGGCCAGGGAAC |
| | | CCTGGTCACCGTCTCCTCAGCTAGCACCAAGGGCCCATCGGTC |
| | | TTCCCCCTGGCACCCTCCTCCAAGAGCACCTCTGGGGGCACAG |
| | | CGGCCCTGGGCTGCCTGGTCAAGGACTACTTCCCCGAACCGGT |
| | | GACGGTGTCGTGGAACTCAGGCGCCCTGACCAGCGGCGTGCAC |
| | | ACCTTCCCGGCTGTCCTACAGTCCTCAGGACTCTACTCCCTCA |
| | | GCAGCGTGGTGACCGTGCCCTCCAGCAGCTTGGGCACCCAGAC |
| | | CTACATCTGCAACGTGAATCACAAGCCCAGCAACACCAAGGTG |
| | | GACAAGAGAGTTGAGCCCAAATCTTGTGACAAAACTCACACAT |
| | | GCCCACCGTGCCCAGCACCTGAAGCCGAAGGGGCCCCGTCAGT |
| | | CTTCCTCTTCCCCCCAAAACCCAAGGACACCCTCATGATCTCC |
| | | CGGACCCCTGAGGTCACATGCGTGGTGGTGGACGTGAGCCACG |
| | | AAGACCCTGAGGTCAAGTTCAACTGGTACGTGGACGGCGTGGA |
| | | GGTGCATAATGCCAAGACAAAGCCGCGGGAGGAGCAGTACAAC |
| | | AGCACGTACCGTGTGGTCAGCGTCCTCACCGTCCTGCACCAGG |
| | | ACTGGCTGAATGGCAAGGAGTACAAGTGCAAGGTCTCCAACAA |
| | | AGCCCTCCCAAGCAGCATCGAGAAAACCATCTCCAAAGCCAAA |
| | | GGGCAGCCCCGAGAACCACAGGTGTACACCCTGCCCCCATCCC |
| | | GGGAGGAGATGACCAAGAACCAGGTCAGCCTGACCTGCCTGGT |
| | | CAAAGGCTTCTATCCCAGCGACATCGCCGTGGAGTGGGAGAGC |
| | | AATGGGCAGCCGGAGAACAACTACAAGACCACGCCTCCCGTGC |
| | | TGGACTCCGACGGCTCCTTCTTCCTCTATAGCAAGCTCACCGT |
| | | GGACAAGAGCAGGTGGCAGCAGGGGAACGTCTTCTCATGCTCC |
| | | GTGATGCATGAGGCTCTGCACAACCACTACACGCAGAAGAGCC |
| | | TCTCCCTGTCCCCGGGTAAATGA |
| 32 | 21D9 kappa LC | GAAATTGTGTTGACGCAGTCTCCAGGCACCCTGTCTTTGTCTC |
| | DNA | CAGGGGAAAGAGCCACCCTCTCCTGCAGGGCCAGTCAGAGTGT |
| | ILT4.6-kappa | TAGCAGCAGCTACTTAGCCTGGTACCAGCAGAAACCTGGCCAG |
| | DNA | GCTCCCAGGCTCCTCATCTATGGTGCATCCAGCAGGGCCACTG |
| | | GCATCCCAGACAGGTTCAGTGGCAGTGGGTCTGGGACAGACTT |
| | | CACTCTCACCATCAGCAGACTGGAGCCTGAAGATTTTGCAGTG |
| | | TATTACTGTCAGCAGTATGGTAGCTCACCTCTCACTTTCGGCG |
| | | GAGGGACCAAGGTGGAGATCAAACGTACGGTGGCTGCACCATC |
| | | TGTCTTCATCTTCCCGCCATCTGATGAGCAGTTGAAATCTGGA |
| | | ACTGCCTCTGTTGTGTGCCTGCTGAATAACTTCTATCCCAGAG |
| | | AGGCCAAAGTACAGTGGAAGGTGGATAACGCCCTCCAATCGGG |
| | | TAACTCCCAGGAGAGTGTCACAGAGCAGGACAGCAAGGACAGC |
| | | ACCTACAGCCTCAGCAGCACCCTGACGCTGAGCAAAGCAGACT |
| | | ACGAGAAACACAAAGTCTACGCCTGCGAAGTCACCCATCAGGG |
| | | CCTGAGCTCGCCCGTCACAAAGAGCTTCAACAGGGGAGAGTGT |
| | | TAG |
| 33 | 21D9 IgG1.1f | GAGGGACAGCTGTTGGAGTCTGGGGGAGACTTGGTACAGCCTG |
| | HC DNA | GGGGGTCCCTGAGACTCTCCTGTGCAACCTCTGGATTCACCTT |
| | ILT4.6- | TAGCAACTATGCCATGAACTGGGTCCGCCAGGCTCCAGGGAAG |
| | IgG1.1f DNA | GGGCTGGAGTGGATCTCAGTTATTAGTGTTAGTGGTGGTAGCA |
| | | CATACTACGCAGACTCCGTGAAGGGCCGGTTCACCATCTCCAG |
| | | AGACAATTCCAAGAACACGCTGTATCTGCAAATGAATAGCCTG |
| | | AGAGCCGAGGACACGGCCGTATATTACTGTGCGAAAGATTATT |
| | | ACTATGATTCGGGGAGTTATTATGACTCTTTCTTTGACTACTG |
| | | GGGCCAGGGAACCCTGGTCACCGTCTCCTCAGCTAGCACCAAG |
| | | GGCCCATCGGTCTTCCCCCTGGCACCCTCCTCCAAGAGCACCT |
| | | CTGGGGGCACAGCGGCCCTGGGCTGCCTGGTCAAGGACTACTT |
| | | CCCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGAAGG |
| | | GGCCCCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGAG |
| | | GAGCAGTACAACAGCACGTACCGTGTGGTCAGCGTCCTCACCG |
| | | TCCTGCACCAGGACTGGCTGAATGGCAAGGAGTACAAGTGCAA |
| | | GGTCTCCAACAAAGCCCTCCCAAGCAGCATCGAGAAAACCATC |
| | | TCCAAAGCCAAAGGGCAGCCCCGAGAACCACAGGTGTACACCC |
| | | TGCCCCCATCCCGGGAGGAGATGACCAAGAACCAGGTCAGCCT |
| | | GACCTGCCTGGTCAAAGGCTTCTATCCCAGCGACATCGCCGTG |
| | | GAGTGGGAGAGCAATGGGCAGCCGGAGAACAACTACAAGACCA |
| | | CGCCTCCCGTGCTGGACTCCGACGGCTCCTTCTTCCTCTATAG |
| | | CAAGCTCACCGTGGACAAGAGCAGGTGGCAGCAGGGGAACGTC |
| | | TTCTCATGCTCCGTGATGCATGAGGCTCTGCACAACCACTACA |
| | | CGCAGAAGAGCCTCTCCCTGTCCCCGGGTAAATGA |
| 34 | 21D9.IgG1.3 | GAGGGACAGCTGTTGGAGTCTGGGGGAGACTTGGTACAGCCTG |
| | HC DNA | GGGGGTCCCTGAGACTCTCCTGTGCAACCTCTGGATTCACCTT |
| | ILT4.6-IgG1.3 | TAGCAACTATGCCATGAACTGGGTCCGCCAGGCTCCAGGGAAG |
| | HC DNA | GGGCTGGAGTGGATCTCAGTTATTAGTGTTAGTGGTGGTAGCA |
| | (variable | CATACTACGCAGACTCCGTGAAGGGCCGGTTCACCATCTCCAG |
| | region is | AGACAATTCCAAGAACACGCTGTATCTGCAAATGAATAGCCTG |
| | underlined | AGAGCCGAGGACACGGCCGTATATTACTGTGCGAAAGATTATT |
| | and bold) | ACTATGATTCGGGGAGTTATTATGACTCTTTCTTTGACTACTG |
| | | GGGCCAGGGAACCCTGGTCACCGTCTCCTCA |
| | | GCTAGCACCAAGGGCCCATCGGTCTTCCCCCTGGCACCCTCCT |
| | | CCAAGAGCACCTCTGGGGGCACAGCGGCCCTGGGCTGCCTGGT |
| | | CAAGGACTACTTCCCCGAACCGGTGACGGTGTCGTGGAACTCA |
| | | GGCGCCCTGACCAGCGGCGTGCACACCTTCCCGGCTGTCCTAC |
| | | AGTCCTCAGGACTCTACTCCCTCAGCAGCGTGGTGACCGTGCC |
| | | CTCCAGCAGCTTGGGCACCCAGACCTACATCTGCAACGTGAAT |
| | | CACAAGCCCAGCAACACCAAGGTGGACAAGAGAGTTGAGCCCA |
| | | AATCTTGTGACAAAACTCACACATGCCCACCGTGCCCAGCACC |
| | | TGAAGCCGAAGGGGCCCCGTCAGTCTTCCTCTTCCCCCCAAAA |
| | | CCCAAGGACACCCTCATGATCTCCCGGACCCCTGAGGTCACAT |
| | | GCGTGGTGGTGGACGTGAGCCACGAAGACCCTGAGGTCAAGTT |
| | | CAACTGGTACGTGGACGGCGTGGAGGTGCATAATGCCAAGACA |
| | | AAGCCGCGGGAGGAGCAGTACAACAGCACGTACCGTGTGGTCA |
| | | GCGTCCTCACCGTCCTGCACCAGGACTGGCTGAATGGCAAGGA |
| | | GTACAAGTGCAAGGTCTCCAACAAAGCCCTCCCAGCCCCCATC |
| | | GAGAAAACCATCTCCAAAGCCAAAGGGCAGCCCCGAGAACCAC |
| | | AGGTGTACACCCTGCCCCCATCCCGGGAGGAGATGACCAAGAA |
| | | CCAGGTCAGCCTGACCTGCCTGGTCAAAGGCTTCTATCCCAGC |
| | | GACATCGCCGTGGAGTGGGAGAGCAATGGGCAGCCGGAGAACA |
| | | ACTACAAGACCACGCCTCCCGTGCTGGACTCCGACGGCTCCTT |
| | | CTTCCTCTATAGCAAGCTCACCGTGGACAAGAGCAGGTGGCAG |
| | | CAGGGGAACGTCTTCTCATGCTCCGTGATGCATGAGGCTCTGC |
| | | ACAACCACTACACGCAGAAGAGCCTCTCCCTGTCCCCGGGTTG |
| | | A |
| 35 | 21D9.b.IgG1.3 | GAGGTGCAGCTGCTGGAGTCTGGAGGAGGACTGGTGCAGCCAG |
| | HC | GAGGCAGCCTGAGGCTGTCCTGCGCCACCTCTGGCTTCACATT |
| | DNA | TTCCAACTATGCCATGAATTGGGTGCGCCAGGCACCTGGCAAG |
| | | GGACTGGAGTGGATCTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCCGCTAGCACCAAG |
| | | GGCCCATCGGTCTTCCCCCTGGCACCCTCCTCCAAGAGCACCT |
| | | CTGGGGGCACAGCGGCCCTGGGCTGCCTGGTCAAGGACTACTT |
| | | CCCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGAAGG |
| | | GGCCCCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGAG |
| | | GAGCAGTACAACAGCACGTACCGTGTGGTCAGCGTCCTCACCG |
| | | TCCTGCACCAGGACTGGCTGAATGGCAAGGAGTACAAGTGCAA |
| | | GGTCTCCAACAAAGCCCTCCCAGCCCCCATCGAGAAAACCATC |
| | | TCCAAAGCCAAAGGGCAGCCCCGAGAACCACAGGTGTACACCC |
| | | TGCCCCCATCCCGGGAGGAGATGACCAAGAACCAGGTCAGCCT |
| | | GACCTGCCTGGTCAAAGGCTTCTATCCCAGCGACATCGCCGTG |
| | | GAGTGGGAGAGCAATGGGCAGCCGGAGAACAACTACAAGACCA |
| | | CGCCTCCCGTGCTGGACTCCGACGGCTCCTTCTTCCTCTATAG |
| | | CAAGCTCACCGTGGACAAGAGCAGGTGGCAGCAGGGGAACGTC |
| | | TTCTCATGCTCCGTGATGCATGAGGCTCTGCACAACCACTACA |
| | | CGCAGAAGAGCCTCTCCCTGTCCCCGGGTTGA |
| 36 | 21D9.b.IgG1.3 | EVQLLESGGGLVQPGGSLRLSCATSGFTFSNYAMNWVRQAPGK |
| | HC | GLEWISVISVSGGSTYY |
| | | ADSVKGRFTISRDNSKNTLYLQMNSLRAEDTAVYYCAKDYYYD |
| | | SGSYYDSFFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKRVEPKSCDKTHTCPPCPAPEAEGAPSVE |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTP |
| | | PVLDSDGSFFLYSKLTVDKSRWQQGNVFSCSVMHEALHNHYTQ |
| | | KSLSLSPG |
| 37 | 21D9.c.IgG1.3 | GAGGTGCAGCTGCTGGAGTCTGGAGGAGACCTGGTGCAGCCAG |
| | HC | GAGGCAGCCTGAGGCTGTCCTGCGCCGCCTCTGGCTTCACATT |
| | DNA | TTCCAACTATGCCATGAATTGGGTGCGCCAGGCACCTGGCAAG |
| | | GGACTGGAGTGGATCTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCCGCTAGCACCAAG |
| | | GGCCCATCGGTCTTCCCCCTGGCACCCTCCTCCAAGAGCACCT |
| | | CTGGGGGCACAGCGGCCCTGGGCTGCCTGGTCAAGGACTACTT |
| | | CCCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGAAGG |
| | | GGCCCCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGAG |
| | | GAGCAGTACAACAGCACGTACCGTGTGGTCAGCGTCCTCACCG |
| | | TCCTGCACCAGGACTGGCTGAATGGCAAGGAGTACAAGTGCAA |
| | | GGTCTCCAACAAAGCCCTCCCAGCCCCCATCGAGAAAACCATC |
| | | TCCAAAGCCAAAGGGCAGCCCCGAGAACCACAGGTGTACACCC |
| | | TGCCCCCATCCCGGGAGGAGATGACCAAGAACCAGGTCAGCCT |
| | | GACCTGCCTGGTCAAAGGCTTCTATCCCAGCGACATCGCCGTG |
| | | GAGTGGGAGAGCAATGGGCAGCCGGAGAACAACTACAAGACCA |
| | | CGCCTCCCGTGCTGGACTCCGACGGCTCCTTCTTCCTCTATAG |
| | | CAAGCTCACCGTGGACAAGAGCAGGTGGCAGCAGGGGAACGTC |
| | | TTCTCATGCTCCGTGATGCATGAGGCTCTGCACAACCACTACA |
| | | CGCAGAAGAGCCTCTCCCTGTCCCCGGGTTGA |
| 38 | 21D9.c.IgG1.3 | EVQLLESGGDLVQPGGSLRLSCAASGFTFSNYAMNWVRQAPGK |
| | HC | GLEWISVISVSGGSTYY |
| | | ADSVKGRFTISRDNSKNTLYLQMNSLRAEDTAVYYCAKDYYYD |
| | | SGSYYDSFFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKRVEPKSCDKTHTCPPCPAPEAEGAPSVF |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTP |
| | | PVLDSDGSFFLYSKLTVDKSRWQQGNVFSCSVMHEALHNHYTQ |
| | | KSLSLSPG |
| 39 | 21D9.d.IgG1.3 | GAGGTGCAGCTGCTGGAGTCTGGAGGAGACCTGGTGCAGCCAG |
| | HC | GAGGCAGCCTGAGGCTGTCCTGCGCCACCTCTGGCTTCACATT |
| | DNA | TTCCAACTATGCCATGAATTGGGTGCGCCAGGCACCTGGCAAG |
| | | GGACTGGAGTGGGTGTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCCGCTAGCACCAAG |
| | | GGCCCATCGGTCTTCCCCCTGGCACCCTCCTCCAAGAGCACCT |
| | | CTGGGGGCACAGCGGCCCTGGGCTGCCTGGTCAAGGACTACTT |
| | | CCCCGAACCGGTGACGGTGTCGTGGAACTCAGGCGCCCTGACC |
| | | AGCGGCGTGCACACCTTCCCGGCTGTCCTACAGTCCTCAGGAC |
| | | TCTACTCCCTCAGCAGCGTGGTGACCGTGCCCTCCAGCAGCTT |
| | | GGGCACCCAGACCTACATCTGCAACGTGAATCACAAGCCCAGC |
| | | AACACCAAGGTGGACAAGAGAGTTGAGCCCAAATCTTGTGACA |
| | | AAACTCACACATGCCCACCGTGCCCAGCACCTGAAGCCGAAGG |
| | | GGCCCCGTCAGTCTTCCTCTTCCCCCCAAAACCCAAGGACACC |
| | | CTCATGATCTCCCGGACCCCTGAGGTCACATGCGTGGTGGTGG |
| | | ACGTGAGCCACGAAGACCCTGAGGTCAAGTTCAACTGGTACGT |
| | | GGACGGCGTGGAGGTGCATAATGCCAAGACAAAGCCGCGGGAG |
| | | GAGCAGTACAACAGCACGTACCGTGTGGTCAGCGTCCTCACCG |
| | | TCCTGCACCAGGACTGGCTGAATGGCAAGGAGTACAAGTGCAA |
| | | GGTCTCCAACAAAGCCCTCCCAGCCCCCATCGAGAAAACCATC |
| | | TCCAAAGCCAAAGGGCAGCCCCGAGAACCACAGGTGTACACCC |
| | | TGCCCCCATCCCGGGAGGAGATGACCAAGAACCAGGTCAGCCT |
| | | GACCTGCCTGGTCAAAGGCTTCTATCCCAGCGACATCGCCGTG |
| | | GAGTGGGAGAGCAATGGGCAGCCGGAGAACAACTACAAGACCA |
| | | CGCCTCCCGTGCTGGACTCCGACGGCTCCTTCTTCCTCTATAG |
| | | CAAGCTCACCGTGGACAAGAGCAGGTGGCAGCAGGGGAACGTC |
| | | TTCTCATGCTCCGTGATGCATGAGGCTCTGCACAACCACTACA |
| | | CGCAGAAGAGCCTCTCCCTGTCCCCGGGTTGA |
| 40 | 21D9.d.IgG1.3 | EVQLLESGGDLVQPGGSLRLSCATSGFTFSNYAMNWVRQAPGK |
| | HC | GLEWVSVISVSGGSTYY |
| | | ADSVKGRFTISRDNSKNTLYLQMNSLRAEDTAVYYCAKDYYYD |
| | | SGSYYDSFFDYWGQGTL |
| | | VTVSSASTKGPSVFPLAPSSKSTSGGTAALGCLVKDYFPEPVT |
| | | VSWNSGALTSGVHTFPAVLQSSGLYSLSSVVTVPSSSLGTQTY |
| | | ICNVNHKPSNTKVDKRVEPKSCDKTHTCPPCPAPEAEGAPSVF |
| | | LFPPKPKDTLMISRTPEVTCVVVDVSHEDPEVKFNWYVDGVEV |
| | | HNAKTKPREEQYNSTYRVVSVLTVLHQDWLNGKEYKCKVSNKA |
| | | LPAPIEKTISKAKGQPREPQVYTLPPSREEMTKNQVSLTCLVK |
| | | GFYPSDIAVEWESNGQPENNYKTTP |
| | | PVLDSDGSFFLYSKLTVDKSRWQQGNVFSCSVMHEALHNHYTQ |
| | | KSLSLSPG |
| 41 | 21D9.e.IgG1.3 | ATGAGGGCTTGGATCTTCTTTCTGCTCTGCCTGGCCGGGAGAG |
| | HC | CGCTCGCAGAGGTGCAGCTGTTGGAGTCTGGGGGAGGCTTGGT |
| | DNA | ACAGCCTGGGGGGTCCCTGAGACTCTCCTGTGCAGCCTCTGGA |
| | ILT4.8.IgG1.3 | TTCACCTTTAGCAACTATGCCATGAACTGGGTCCGCCAGGCTC |
| | HC DNA | CAGGGAAGGGGCTGGAGTGGGTCTCAGTTATTAGTGTTAGTGG |
| | With signal | TGGTAGCACATACTACGCAGACTCCGTGAAGGGCCGGTTCACC |
| | peptide | ATCTCCAGAGACAATTCCAAGAACACGCTGTATCTGCAAATGA |
| | (signal | ATAGCCTGAGAGCCGAGGACACGGCCGTATATTACTGTGCGAA |
| | peptide | AGATTATTACTATGATTCGGGGAGTTATTATGACTCTTTCTTT |
| | encoding | GACTACTGGGGCCAGGGAACCCTGGTCACCGTCTCCTCAGCTA |
| | sequence is | GCACCAAGGGCCCATCGGTCTTCCCCCTGGCACCCTCCTCCAA |
| | underlined) | GAGCACCTCTGGGGGCACAGCGGCCCTGGGCTGCCTGGTCAAG |
| | | GACTACTTCCCCGAACCGGTGACGGTGTCGTGGAACTCAGGCG |
| | | CCCTGACCAGCGGCGTGCACACCTTCCCGGCTGTCCTACAGTC |
| | | CTCAGGACTCTACTCCCTCAGCAGCGTGGTGACCGTGCCCTCC |
| | | AGCAGCTTGGGCACCCAGACCTACATCTGCAACGTGAATCACA |
| | | AGCCCAGCAACACCAAGGTGGACAAGAGAGTTGAGCCCAAATC |
| | | TTGTGACAAAACTCACACATGCCCACCGTGCCCAGCACCTGAA |
| | | GCCGAAGGGGCCCCGTCAGTCTTCCTCTTCCCCCCAAAACCCA |
| | | AGGACACCCTCATGATCTCCCGGACCCCTGAGGTCACATGCGT |
| | | GGTGGTGGACGTGAGCCACGAAGACCCTGAGGTCAAGTTCAAC |
| | | TGGTACGTGGACGGCGTGGAGGTGCATAATGCCAAGACAAAGC |
| | | CGCGGGAGGAGCAGTACAACAGCACGTACCGTGTGGTCAGCGT |
| | | CCTCACCGTCCTGCACCAGGACTGGCTGAATGGCAAGGAGTAC |
| | | AAGTGCAAGGTCTCCAACAAAGCCCTCCCAGCCCCCATCGAGA |
| | | AAACCATCTCCAAAGCCAAAGGGCAGCCCCGAGAACCACAGGT |
| | | GTACACCCTGCCCCCATCCCGGGAGGAGATGACCAAGAACCAG |
| | | GTCAGCCTGACCTGCCTGGTCAAAGGCTTCTATCCCAGCGACA |
| | | TCGCCGTGGAGTGGGAGAGCAATGGGCAGCCGGAGAACAACTA |
| | | CAAGACCACGCCTCCCGTGCTGGACTCCGACGGCTCCTTCTTC |
| | | CTCTATAGCAAGCTCACCGTGGACAAGAGCAGGTGGCAGCAGG |
| | | GGAACGTCTTCTCATGCTCCGTGATGCATGAGGCTCTGCACAA |
| | | CCACTACACGCAGAAGAGCCTCTCCCTGTCCCCGGGT |
| 42 | 21A5 LC | GAGATCGTGCTGACCCAGAGCCCAGGCACACTGTCTCTGAGCC |
| | DNA | CAGGAGAGAGGGCCACCCTGTCCTGCAGAGCCTCCCAGTCTGT |
| | (variable | GAGCTCCTCTTACCTGGCCTGGTATCAGCAGAAGCCAGGACAG |
| | region coding | GCACCTAGGCTGCTGATCTACGGAGCCAGCTCCAGGGCAACCG |
| | sequence | GCATCCCTGACCGCTTCAGCGGCTCCGGCTCTGGCACAGACTT |
| | underlined | CACCCTGACAATCTCTAGGCTGGAGCCCGAGGACTTCGCCGTG |
| | and bold) | TACTATTGTCAGCAGTATGGCTCCACCTTTGGCGGCGGCACAA |
| | | AGGTGGAGATCAAG |
| | | GAATTCGCCACCATGAGGGCTTGGATCTTCTTTCTGCTCTGCC |
| | | TGGCCGGGCGCGCCCTCGCAGAGATCGTGCTGACCCAGAGCCC |
| | | AGGCACACTGTCTCTGAGCCCAGGAGAGAGGGCCACCCTGTCC |
| | | TGCAGAGCCTCCCAGTCTGTGAGCTCCTCTTACCTGGCCTGGT |
| | | ATCAGCAGAAGCCAGGACAGGCACCTAGGCTGCTGATCTACGG |
| | | AGCCAGCTCCAGGGCAACCGGCATCCCTGACCGCTTCAGCGGC |
| | | TCCGGCTCTGGCACAGACTTCACCCTGACAATCTCTAGGCTGG |
| | | AGCCCGAGGACTTCGCCGTGTACTATTGTCAGCAGTATGGCTC |
| | | CACCTTTGGCGGCGGCACAAAGGTGGAGATCAAGCGTACGGTG |
| | | GCCGCCCCGTCCGTGTTTATCTTCCCTCCATCGGACGAGCAGC |
| | | TCAAGTCCGGTACCGCGAGCGTGGTCTGCCTGCTGAACAATTT |
| | | CTACCCGCGCGAAGCTAAAGTGCAATGGAAGGTCGATAACGCA |
| | | CTTCAGTCCGGGAACAGCCAGGAATCTGTGACCGAGCAGGACT |
| | | CCAAGGATTCGACCTATTCCCTGTCCTCGACTCTCACCCTGTC |
| | | AAAGGCCGACTACGAGAAGCACAAGGTCTACGCCTGCGAAGTG |
| | | ACCCATCAGGGCTTGTCCTCACCCGTGACTAAGAGCTTCAACC |
| | | GGGGAGAGTGTTAGTGA |
| 43 | 21A5.IgG1.3 | CAGGTGCAGCTGGTGCAGTCCGGAGCAGAGGTGAAGAAGCCAG |
| | HC | GAGCCTCTGTGAAGGTGAGCTGCAAGGCCTCCGGCTACACCTT |
| | DNA | CACAGATTACTATCTGCACTGGGTGCGGCAGGCACCAGGACAG |
| | (variable | GGACTGGAGTGGATGGGCATCTTCCACCCTTCTGGCGACATCA |
| | region bold | CAAGCTCCGCCCAGAACTTTCAGGGCCGGGTGACCATGACAAG |
| | and | AGATACCAGCACATCCACCGTGTACATGGAGCTGTCTAGCCTG |
| | underlined) | AGGTCTGAGGACACCGCCGTGTACTATTGTGCAAGGGGAGGCG |
| | | TGCTGAGGTATCTGGACTGGAGCCACGCCTTTGATATCTGGGG |
| | | CCAGGGCACAATGGTGACCGTGTCCTCT |
| | | GCTAGCACCAAAGGACCTTCAGTGTTCCCGCTCGCGCCGTCAT |
| | | CCAAGTCCACCTCCGGCGGAACCGCTGCCCTGGGATGCCTTGT |
| | | GAAGGACTACTTCCCTGAGCCTGTGACTGTGTCCTGGAACTCC |
| | | GGAGCCCTGACCTCCGGCGTGCATACCTTCCCTGCTGTGCTGC |
| | | AATCAAGCGGCCTCTACTCATTGAGCTCCGTCGTGACCGTGCC |
| | | GAGCTCCAGCCTCGGTACTCAAACCTACATCTGCAATGTCAAC |
| | | CACAAGCCCAGCAACACCAAGGTCGATAAGAGAGTGGAGCCCA |
| | | AGTCGTGCGACAAGACTCACACTTGTCCCCCATGCCCGGCCCC |
| | | CGAGGCCGAGGGGGCCCCGAGCGTCTTTCTGTTCCCGCCTAAG |
| | | CCCAAGGATACCCTGATGATTTCGCGGACTCCCGAAGTGACCT |
| | | GTGTCGTGGTGGACGTGTCCCACGAAGATCCCGAAGTCAAGTT |
| | | CAACTGGTACGTGGACGGAGTCGAGGTGCACAACGCAAAGACC |
| | | AAGCCTCGCGAGGAACAGTACAACTCGACCTATCGGGTGGTGT |
| | | CCGTGCTGACAGTGCTGCATCAGGACTGGCTCAACGGAAAGGA |
| | | GTACAAGTGCAAAGTGTCCAACAAGGCCCTGCCAGCGCCCATT |
| | | GAAAAGACTATCAGCAAGGCCAAGGGGCAGCCAAGGGAACCCC |
| | | AAGTGTACACCCTGCCTCCGTCCCGCGAAGAAATGACCAAGAA |
| | | CCAGGTGTCCCTGACGTGCTTGGTCAAGGGCTTTTACCCTTCC |
| | | GACATCGCCGTGGAGTGGGAGTCGAACGGGCAGCCGGAAAACA |
| | | ACTACAAGACCACTCCACCGGTGCTCGACTCGGATGGCTCGTT |
| | | CTTCCTGTATTCGAAGCTGACTGTCGACAAAAGCCGGTGGCAG |
| | | CAGGGCAATGTGTTCTCCTGCTCCGTGATGCATGAGGCCCTCC |
| | | ACAACCACTACACCCAGAAGTCTCTGAGCCTTTCCCCGGGATG |
| | | A |
| 44 | 21A5.1 kappa | ATGAGGGCTTGGATCTTCTTTCTGCTCTGCCTGGCCGGGCGCG |
| | LC DNA | CCTTGGCCGAAATTGTGTTGACGCAGTCTCCAGGCACCCTGTC |
| | (ILT4.9.1K) | TTTGTCTCCAGGGGAAAGAGCCACCCTCTCCTGCAGGGCCAGT |
| | DNA | CAGAGTGTTAGCAGCAGCTACTTAGCCTGGTACCAGCAGAAAC |
| | With signal | CTGGCCAGGCTCCCAGGCTCCTCATCTATGGTGCATCCAGCAG |
| | peptide | GGCCACTGGCATCCCAGACAGGTTCAGTGGCAGTGGGTCTGGG |
| | | ACAGACTTCACTCTCACCATCAGCAGACTGGAGCCTGAAGATT |
| | | TTGCAGTGTATTACTGTCAGCAGTATGGTAGCACTTTCGGCGG |
| | | AGGGACCAAGGTGGAGATCAAACGTACGGTGGCTGCACCATCT |
| | | GTCTTCATCTTCCCGCCATCTGATGAGCAGTTGAAATCTGGAA |
| | | CTGCCTCTGTTGTGTGCCTGCTGAATAACTTCTATCCCAGAGA |
| | | GGCCAAAGTACAGTGGAAGGTGGATAACGCCCTCCAATCGGGT |
| | | AACTCCCAGGAGAGTGTCACAGAGCAGGACAGCAAGGACAGCA |
| | | CCTACAGCCTCAGCAGCACCCTGACGCTGAGCAAAGCAGACTA |
| | | CGAGAAACACAAAGTCTACGCCTGCGAAGTCACCCATCAGGGC |
| | | CTGAGCTCGCCCGTCACAAAGAGCTTCAACAGGGGAGAGTGT |
| 45 | 21A5.a.IgG1.3 | ATGAGGGCTTGGATCTTCTTTCTGCTCTGCCTGGCCGGGAGAG |
| | HC DNA | CGCTCGCACAGGTGCAGCTGGTGCAGTCTGGGGCTGAGGTGAA |
| | (ILT4.9.IgG1.3) | GAAGCCTGGGGCCTCAGTGAAGGTTTCCTGCAAGGCATCTGGA |
| | DNA | TACACCTTCACCGACTACTATTTGCACTGGGTGCGACAGGCCC |
| | With signal | CTGGACAAGGGCTTGAGTGGATGGGAATATTCCACCCTAGTGG |
| | peptide | TGATATCACAAGCTCCGCACAGAACTTCCAGGGCAGAGTCACC |
| | | ATGACCAGGGACACGTCCACGAGCACAGTCTACATGGAGCTGA |
| | | GCAGCCTGAGATCTGAGGACACGGCCGTGTATTACTGTGCGAG |
| | | AGGAGGCGTGCTGAGGTATCTGGACTGGAGCCATGCTTTTGAT |
| | | ATCTGGGGCCAAGGGACAATGGTCACCGTCTCTTCAGCTAGCA |
| | | CCAAGGGCCCATCGGTCTTCCCCCTGGCACCCTCCTCCAAGAG |
| | | CACCTCTGGGGGCACAGCGGCCCTGGGCTGCCTGGTCAAGGAC |
| | | TACTTCCCCGAACCGGTGACGGTGTCGTGGAACTCAGGCGCCC |
| | | TGACCAGCGGCGTGCACACCTTCCCGGCTGTCCTACAGTCCTC |
| | | AGGACTCTACTCCCTCAGCAGCGTGGTGACCGTGCCCTCCAGC |
| | | AGCTTGGGCACCCAGACCTACATCTGCAACGTGAATCACAAGC |
| | | CCAGCAACACCAAGGTGGACAAGAGAGTTGAGCCCAAATCTTG |
| | | TGACAAAACTCACACATGCCCACCGTGCCCAGCACCTGAAGCC |
| | | GAAGGGGCCCCGTCAGTCTTCCTCTTCCCCCCAAAACCCAAGG |
| | | ACACCCTCATGATCTCCCGGACCCCTGAGGTCACATGCGTGGT |
| | | GGTGGACGTGAGCCACGAAGACCCTGAGGTCAAGTTCAACTGG |
| | | TACGTGGACGGCGTGGAGGTGCATAATGCCAAGACAAAGCCGC |
| | | GGGAGGAGCAGTACAACAGCACGTACCGTGTGGTCAGCGTCCT |
| | | CACCGTCCTGCACCAGGACTGGCTGAATGGCAAGGAGTACAAG |
| | | TGCAAGGTCTCCAACAAAGCCCTCCCAGCCCCCATCGAGAAAA |
| | | CCATCTCCAAAGCCAAAGGGCAGCCCCGAGAACCACAGGTGTA |
| | | CACCCTGCCCCCATCCCGGGAGGAGATGACCAAGAACCAGGTC |
| | | AGCCTGACCTGCCTGGTCAAAGGCTTCTATCCCAGCGACATCG |
| | | CCGTGGAGTGGGAGAGCAATGGGCAGCCGGAGAACAACTACAA |
| | | GACCACGCCTCCCGTGCTGGACTCCGACGGCTCCTTCTTCCTC |
| | | TATAGCAAGCTCACCGTGGACAAGAGCAGGTGGCAGCAGGGGA |
| | | ACGTCTTCTCATGCTCCGTGATGCATGAGGCTCTGCACAACCA |
| | | CTACACGCAGAAGAGCCTCTCCCTGTCCCCGGGT |
| 46 | 10F10 kappa | ATGAGGGCTTGGATCTTCTTTCTGCTCTGCCTGGCCGGGCGCG |
| | LC DNA | CCCTCGCAGCCATCCAGCTGACCCAGTCTCCTAGCTCCCTGTC |
| | ILT4.10K | TGCCAGCGTGGGCGACAGGGTGACCATCACATGCAGAGCCTCC |
| | DNA | CAGGGAATCTCTAGCGCCCTGGCCTGGTTCCAGCAGAAGCCAG |
| | With signal | GCAAGGCCCCTAAGCTGCTGATCTACGATGCCTCCTCTCTGGA |
| | peptide | GAGCGGAGTGCCATCCAGGTTCACCGGCTCCGGCTCTGGCACA |
| | | GACTTTACCCTGACAATCAGCTCCCTGCAGCCAGAGGATTTTG |
| | | CCACATACTATTGTCAGCAGTTCAACAGCTATCCCATCACCTT |
| | | TGGCCAGGGCACACGGCTGGAGATCAAGCGTACGGTGGCCGCC |
| | | CCGTCCGTGTTTATCTTCCCTCCATCGGACGAGCAGCTCAAGT |
| | | CCGGTACCGCGAGCGTGGTCTGCCTGCTGAACAATTTCTACCC |
| | | GCGCGAAGCTAAAGTGCAATGGAAGGTCGATAACGCACTTCAG |
| | | TCCGGGAACAGCCAGGAATCTGTGACCGAGCAGGACTCCAAGG |
| | | ATTCGACCTATTCCCTGTCCTCGACTCTCACCCTGTCAAAGGC |
| | | CGACTACGAGAAGCACAAGGTCTACGCCTGCGAAGTGACCCAT |
| | | CAGGGCTTGTCCTCACCCGTGACTAAGAGCTTCAACCGGGGAG |
| | | AGTGT |
| 47 | 10F10.IgG1.3 | ATGAGGGCTTGGATCTTCTTTCTGCTCTGCCTGGCCGGGCGCG |
| | HC DNA | CCCTCGCACAGGTGCAGCTGGTGGAGTCTGGAGGAGGAGTGGT |
| | ILT4.10. IgG1.3 | GCAGCCAGGCCGGTCCCTGAGACTGTCTTGCGCCGCCAGCGGC |
| | DNA | TTCACCTTTAGCTCCTACGCAATGCACTGGGTGAGGCAGGCAC |
| | With signal | CTGGCAAGGGACTGGAGTGGGTGGCCATCATCAGCTACGACGA |
| | peptide | GTATAACAAGTACTATGCCGATTCCGTGAAGGGCAGGTTCACC |
| | | ATCTCCCGCGACAACTCTAAGAATACACTGTACCTGCAGATGA |
| | | ATAGCCTGAGAGCCGAGGATACAGCCGTGTACTATTGTGCCCG |
| | | GGAGTGGGTGGGCATCAGATATTGGGGCCAGGGCACCCTGGTG |
| | | ACAGTGTCTAGCGCTAGCACCAAAGGACCTTCAGTGTTCCCGC |
| | | TCGCGCCGTCATCCAAGTCCACCTCCGGCGGAACCGCTGCCCT |
| | | GGGATGCCTTGTGAAGGACTACTTCCCTGAGCCTGTGACTGTG |
| | | TCCTGGAACTCCGGAGCCCTGACCTCCGGCGTGCATACCTTCC |
| | | CTGCTGTGCTGCAATCAAGCGGCCTCTACTCATTGAGCTCCGT |
| | | CGTGACCGTGCCGAGCTCCAGCCTCGGTACTCAAACCTACATC |
| | | TGCAATGTCAACCACAAGCCCAGCAACACCAAGGTCGATAAGA |
| | | GAGTGGAGCCCAAGTCGTGCGACAAGACTCACACTTGTCCCCC |
| | | ATGCCCGGCCCCCGAGGCCGAGGGGGCCCCGAGCGTCTTTCTG |
| | | TTCCCGCCTAAGCCCAAGGATACCCTGATGATTTCGCGGACTC |
| | | CCGAAGTGACCTGTGTCGTGGTGGACGTGTCCCACGAAGATCC |
| | | CGAAGTCAAGTTCAACTGGTACGTGGACGGAGTCGAGGTGCAC |
| | | AACGCAAAGACCAAGCCTCGCGAGGAACAGTACAACTCGACCT |
| | | ATCGGGTGGTGTCCGTGCTGACAGTGCTGCATCAGGACTGGCT |
| | | CAACGGAAAGGAGTACAAGTGCAAAGTGTCCAACAAGGCCCTG |
| | | CCAGCGCCCATTGAAAAGACTATCAGCAAGGCCAAGGGGCAGC |
| | | CAAGGGAACCCCAAGTGTACACCCTGCCTCCGTCCCGCGAAGA |
| | | AATGACCAAGAACCAGGTGTCCCTGACGTGCTTGGTCAAGGGC |
| | | TTTTACCCTTCCGACATCGCCGTGGAGTGGGAGTCGAACGGGC |
| | | AGCCGGAAAACAACTACAAGACCACTCCACCGGTGCTCGACTC |
| | | GGATGGCTCGTTCTTCCTGTATTCGAAGCTGACTGTCGACAAA |
| | | AGCCGGTGGCAGCAGGGCAATGTGTTCTCCTGCTCCGTGATGC |
| | | ATGAGGCCCTCCACAACCACTACACCCAGAAGTCTCTGAGCCT |
| | | TTCCCCGGGA |
| 48 | 10F10.1 kappa | GCCATCCAGCTGACCCAGTCTCCTAGCTCCCTGTCTGCCAGCG |
| | LC | TGGGCGACAGGGTGACCATCACATGCAGAGCCTCCCAGGGAAT |
| | DNA | CTCTAGCGCCCTGGCCTGGTACCAGCAGAAGCCAGGCAAGGCC |
| | | CCTAAGCTGCTGATCTATGATGCCTCCTCTCTGGAGAGCGGAG |
| | | TGCCATCCAGGTTCACCGGCTCCGGCTCTGGCACAGACTTTAC |
| | | CCTGACAATCAGCTCCCTGCAGCCAGAGGATTTCGCCACATAC |
| | | TATTGTCAGCAGTTCAACAGCTACCCCATCACCTTTGGCCAGG |
| | | GCACACGGCTGGAGATCAAGCGTACGGTGGCCGCCCCGTCCGT |
| | | GTTTATCTTCCCTCCATCGGACGAGCAGCTCAAGTCCGGTACC |
| | | GCGAGCGTGGTCTGCCTGCTGAACAATTTCTACCCGCGCGAAG |
| | | CTAAAGTGCAATGGAAGGTCGATAACGCACTTCAGTCCGGGAA |
| | | CAGCCAGGAATCTGTGACCGAGCAGGACTCCAAGGATTCGACC |
| | | TATTCCCTGTCCTCGACTCTCACCCTGTCAAAGGCCGACTACG |
| | | AGAAGCACAAGGTCTACGCCTGCGAAGTGACCCATCAGGGCTT |
| | | GTCCTCACCCGTGACTAAGAGCTTCAACCGGGGAGAGTGT |
| 49 | 10F10.3 kappa | ATGAGGGCTTGGATCTTCTTTCTGCTCTGCCTGGCCGGGCGCG |
| | LC DNA | CCCTCGCAGCCATCCAGCTGACCCAGTCTCCTAGCTCCCTGTC |
| | ILT4.11K | TGCCAGCGTGGGCGACAGGGTGACCATCACATGCAGAGCCTCC |
| | DNA | CAGGGAATCTCTAGCGCCCTGGCCTGGTACCAGCAGAAGCCAG |
| | With signal | GCAAGGCCCCTAAGCTGCTGATCTATGATGCCTCCTCTCTGGA |
| | peptide | GAGCGGAGTGCCATCCAGGTTCAGCGGCTCCGGCTCTGGCACA |
| | | GACTTTACCCTGACAATCAGCTCCCTGCAGCCAGAGGATTTCG |
| | | CCACATACTATTGTCAGCAGTTCAACAGCTACCCCATCACCTT |
| | | TGGCCAGGGCACACGGCTGGAGATCAAGCGTACGGTGGCCGCC |
| | | CCGTCCGTGTTTATCTTCCCTCCATCGGACGAGCAGCTCAAGT |
| | | CCGGTACCGCGAGCGTGGTCTGCCTGCTGAACAATTTCTACCC |
| | | GCGCGAAGCTAAAGTGCAATGGAAGGTCGATAACGCACTTCAG |
| | | TCCGGGAACAGCCAGGAATCTGTGACCGAGCAGGACTCCAAGG |
| | | ATTCGACCTATTCCCTGTCCTCGACTCTCACCCTGTCAAAGGC |
| | | CGACTACGAGAAGCACAAGGTCTACGCCTGCGAAGTGACCCAT |
| | | CAGGGCTTGTCCTCACCCGTGACTAAGAGCTTCAACCGGGGAG |
| | | AGTGT |
| 50 | 9G4 VL | APAQLLFLLLLWLPDTTGEIVLTQSPATLSLSPGERATLSCRA |
| | ILT4.1K | SQSVSSYLAWYQQKPGQAPRLLIYDASNRATGIPARFSGSGSG |
| | VL (including | TDFTLTISSLEPEDFAGYYCQQRSYWPWTFGQGTKVEIK |
| | signal | |
| | sequence, | |
| | residues 1-18) | |
| 51 | 9G4.IgG1.1 VH | MKHLWFFLLLVAAPRWVLSQVQLQESGPGLVKPSETLSLTCTV |
| | ILT4.1.IgG1.1 | SGGSISSYYWNWIRQPPGKGLEWLGYIYYSGSTKYNPSLKSRV |
| | VH (including | TISVDTSKNQFSLKLSSVTAADTAVYYCASSGWYYFDYWGQGT |
| | signal | LVTVSS |
| | sequence, | |
| | residues 1-19) | |
| 52 | 9G4 VL DNA | GCCCCAGCTCAGCTTCTCTTCCTCCTGCTACTCTGGCTCCCAG |
| | ILT4.1K | ATACCACCGGAGAAATTGTGTTGACACAGTCTCCAGCCACCCT |
| | VL DNA | GTCTTTGTCTCCAGGGGAAAGAGCCACCCTCTCCTGCAGGGCC |
| | | AGTCAGAGTGTTAGCAGCTACTTAGCCTGGTACCAACAGAAAC |
| | | CTGGCCAGGCTCCCAGGCTCCTCATCTATGATGCGTCCAACAG |
| | | GGCCACTGGCATCCCAGCCAGGTTCAGTGGCAGTGGGTCTGGG |
| | | ACAGACTTCACTCTCACCATCAGCAGCCTAGAGCCTGAAGATT |
| | | TTGCAGGTTATTACTGTCAGCAGCGTAGCTACTGGCCGTGGAC |
| | | GTTCGGCCAAGGGACCAAGGTGGAAATCAAA |
| 53 | 9G4.IgG1.1 VH | ATGAAACATCTGTGGTTCTTCCTTCTCCTGGTGGCAGCTCCCA |
| | DNA | GATGGGTCCTGTCCCAGGTGCAGCTGCAGGAGTCGGGCCCAGG |
| | ILT4.1.IgG1.1 | ACTGGTGAAGCCTTCGGAGACCCTGTCCCTCACCTGCACTGTC |
| | VH DNA | TCTGGTGGCTCCATCAGTAGTTACTACTGGAACTGGATCCGGC |
| | | AGCCCCCAGGGAAGGGACTGGAGTGGCTTGGGTACATCTATTA |
| | | CAGTGGGAGTACCAAGTACAACCCCTCCCTCAAGAGTCGAGTC |
| | | ACCATATCAGTAGACACGTCCAAGAACCAGTTCTCCCTGAAGC |
| | | TGAGCTCTGTGACCGCTGCGGACACGGCCGTGTATTATTGTGC |
| | | CAGCAGTGGCTGGTACTACTTTGACTATTGGGGCCAGGGAACC |
| | | CTGGTCACCGTCTCCTCA |
| 54 | 9C8 kappa VL | AIQLTQSPSSLSASVGDRVTITCRASQGISSALAWYQQKPGKA |
| | ILT4.2K | PKLLIYDASSLESGVPSRFSGSGSGTDFTLTISSLQPEDFATY |
| | VL | YCQQFNSYPFTFGPGTKVDIK |
| 55 | 9C8.IgG1.1 VH | QVQLVQSGAEVKKPGASVKVSCKASGYTFTSSDINWVRQATGQ |
| | ILT4.2.IgG1.1 | GLEWMGWMNPNSGHTGYAQKFQDRVTLTRDTSISTAYMELSSL |
| | VH | RSEDSAVYYCARGGNSIDWGFSYYGLDVWGQGTTVTVSS |
| 56 | 9C8 kappa VL | GCCATCCAGTTGACCCAGTCTCCATCCTCCCTGTCTGCATCTG |
| | DNA | TAGGAGACAGAGTCACCATCACTTGCCGGGCAAGTCAGGGCAT |
| | ILT4.2K | TAGCAGTGCTTTAGCCTGGTATCAGCAGAAACCAGGGAAAGCT |
| | VL DNA | CCTAAGCTCCTGATCTATGATGCCTCCAGTTTGGAAAGTGGGG |
| | | TCCCATCAAGGTTCAGCGGCAGTGGATCTGGGACAGATTTCAC |
| | | TCTCACCATCAGCAGCCTGCAGCCTGAAGATTTTGCAACTTAT |
| | | TACTGTCAACAGTTTAATAGTTACCCATTCACTTTCGGCCCTG |
| | | GGACCAAAGTGGATATCAAA |
| 57 | 9C8.IgG1.1 VH | CAGGTGCAACTGGTGCAGTCTGGGGCTGAGGTAAAGAAGCCTG |
| | DNA | GGGCCTCAGTGAAGGTCTCCTGCAAGGCTTCTGGATACACCTT |
| | ILT4.2.IgG1.1 | CACCAGCTCTGATATCAACTGGGTGCGACAGGCCACTGGACAA |
| | VH DNA | GGGCTTGAGTGGATGGGATGGATGAACCCTAACAGTGGTCACA |
| | | CAGGCTATGCACAGAAGTTCCAGGACAGAGTCACCTTGACCCG |
| | | GGACACCTCCATAAGCACAGCCTACATGGAGCTGAGCAGCCTG |
| | | AGATCTGAGGACTCGGCCGTGTATTACTGTGCGAGAGGTGGGA |
| | | ATAGCATTGACTGGGGGTTCTCCTACTACGGTCTGGACGTCTG |
| | | GGGCCAAGGGACCACGGTCACCGTCTCCTCA |
| 58 | 2H2.IgG1.1 VH | QVQLVQSGAEVKKPGSSVEVSCKASGGTFSNYAISWVRQAPGQ |
| | ILT4.3.IgG1.1 | GLEWMGGIIPILATANYAPKFQGRVTITADEFTSSAYMELSSL |
| | VH | RSEDTAVYYCAKSSITMIRGAYLYYYDGMDVWGQGTTVTVSS |
| 59 | 2H2 kappa VL | EIVLTQSPGTLSLSPGERATLSCRASQSVSSSYLAWYQQKPGQ |
| | ILT4.3K | APRLLIYGASSRATGIPDRFSGSGSGTDFTLTISRLEPEDFAV |
| | VL | YYCQQYGSSYTFGQGTKLEIK |
| 60 | 2H2.IgG1.1 VH | CAGGTCCAGTTGGTGCAGTCTGGGGCTGAGGTGAAGAAGCCTG |
| | DNA | GGTCCTCGGTGGAGGTCTCCTGCAAGGCTTCTGGGGGCACCTT |
| | ILT4.3.IgG1.1 | CAGCAACTATGCTATCAGCTGGGTGCGACAGGCCCCTGGACAA |
| | VH DNA | GGGCTTGAGTGGATGGGAGGGATCATCCCCATCTTGGCTACAG |
| | | CAAACTACGCACCGAAGTTCCAGGGCAGAGTCACGATTACCGC |
| | | GGACGAATTCACGAGCTCAGCTTACATGGAGCTGAGCAGCCTG |
| | | AGATCTGAGGACACGGCCGTGTATTACTGTGCGAAGTCTAGTA |
| | | TTACTATGATTCGGGGAGCCTATCTTTACTACTACGACGGTAT |
| | | GGACGTCTGGGGCCAAGGGACCACGGTCACCGTCTCCTCA |
| 61 | 2H2 kappa VL | GAAATTGTGTTGACGCAGTCTCCAGGCACCCTGTCTTTGTCTC |
| | DNA | CAGGGGAAAGAGCCACCCTCTCCTGCAGGGCCAGTCAGAGTGT |
| | ILT4.3K | TAGCAGCAGCTACTTAGCCTGGTACCAGCAGAAACCTGGCCAG |
| | VL DNA | GCTCCCAGGCTCCTCATCTATGGTGCATCCAGCAGGGCCACTG |
| | | GCATCCCAGACAGGTTCAGTGGCAGTGGGTCTGGGACAGACTT |
| | | CACTCTCACCATCAGCAGACTGGAGCCTGAAGATTTTGCAGTG |
| | | TATTACTGTCAGCAGTATGGTAGCTCGTACACTTTTGGCCAGG |
| | | GGACCAAGCTGGAGATCAAA |
| 62 | 2E5 kappa VL | EIVLTQSPATLSLSPGERATLSCRASQSVSSYLAWYQQKPGQA |
| | ILT4.4K | PRLLIYDASNRATGIPARFSGSGSGTDFTLTISSLEPEDFAVY |
| | VL | YCQQRSNWPPWTFGQGTKVEIK |
| 63 | 2E5.IgG1.1 VH | QVQLVESGGGVVQPGRSLRLSCTASGFTFSNYGMHWVRQAPGK |
| | ILT4.4.IgG1.1 | GLEWVAVIWYDGSNEYYAESVKGRLTISRDNSKNTLYLQVNSL |
| | VH | RAEDTAVYYCARDPFYGSGNYFDYWGQGTLVTVSS |
| 64 | 2E5 kappa VL | GAAATTGTGTTGACACAGTCTCCAGCCACCCTGTCTTTGTCTC |
| | DNA | CAGGGGAAAGAGCCACCCTCTCCTGCAGGGCCAGTCAGAGTGT |
| | ILT4.4K | TAGCAGCTACTTAGCCTGGTACCAACAGAAACCTGGCCAGGCT |
| | VL | CCCAGGCTCCTCATCTATGATGCATCCAACAGGGCCACTGGCA |
| | | TCCCAGCCAGGTTCAGTGGCAGTGGGTCTGGGACAGACTTCAC |
| | | TCTCACCATCAGCAGCCTAGAGCCTGAAGATTTTGCAGTTTAT |
| | | TACTGTCAGCAGCGTAGCAACTGGCCTCCGTGGACGTTCGGCC |
| | | AAGGGACCAAGGTGGAAATCAAA |
| 65 | 2E5.IgG1.1 VH | CAGGTGCAGCTGGTGGAGTCTGGGGGAGGCGTGGTCCAGCCTG |
| | DNA | GGAGGTCCCTGAGACTCTCCTGTACAGCGTCTGGATTCACCTT |
| | ILT4.4.IgG1.1 | CAGTAACTATGGCATGCACTGGGTCCGCCAGGCTCCAGGCAAG |
| | VH | GGACTGGAGTGGGTGGCAGTTATCTGGTATGATGGAAGTAATG |
| | | AATACTATGCAGAATCCGTGAAGGGCCGACTCACCATCTCCAG |
| | | AGACAATTCCAAGAACACGCTGTATCTGCAAGTGAACAGCCTG |
| | | AGAGCCGAGGACACGGCTGTGTATTATTGTGCGAGAGATCCTT |
| | | TCTATGGTTCGGGGAATTACTTTGACTACTGGGGCCAGGGAAC |
| | | CCTGGTCACCGTCTCCTCA |
| 66 | 24E5 kappa VL | DIQMTQSPSSLSASVGDRVTITCRASQGISSWLAWYQQKPEKA |
| | ILT4.5K | PKSLIYAASSLQSGVPSRFSGSGSGTDFTLTISSLQPEDFATY |
| | VL | YCQQYNSYPLTFGGGTKVEIK |
| 67 | 24E5.IgG1.1 | EVQLLESGGGLVQPGGSLRLSCAASGFTFSSYVMSWVRQAPGK |
| | VH | GLEWVSGISGSGGSTYYADSVKGRFTISRDNSKNTLYLQMNSL |
| | ILT4.5.IgG1.1 | RAEDTAVYYCAKDQDIIAAYYFVYWGQGTLVTVSS |
| | VH | |
| 68 | 24E5 kappa VL | GACATCCAGATGACCCAGTCTCCATCCTCACTGTCTGCATCTG |
| | DNA | TAGGAGACAGAGTCACCATCACTTGTCGGGCGAGTCAGGGTAT |
| | ILT4.5K | TAGCAGCTGGTTAGCCTGGTATCAGCAGAAACCAGAGAAAGCC |
| | VL | CCTAAGTCCCTGATCTATGCTGCATCCAGTTTGCAAAGTGGGG |
| | | TCCCATCAAGGTTCAGCGGCAGTGGATCTGGGACAGATTTCAC |
| | | TCTCACCATCAGCAGCCTGCAGCCTGAAGATTTTGCAACTTAT |
| | | TACTGCCAACAGTATAATAGTTACCCTCTCACTTTCGGCGGAG |
| | | GGACCAAGGTGGAGATCAAA |
| 69 | 24E5.IgG1.1 | GAGGTGCAGCTGTTGGAGTCTGGGGGAGGCTTGGTACAGCCTG |
| | VH DNA | GGGGGTCCCTGAGACTCTCCTGTGCAGCCTCTGGATTCACCTT |
| | ILT4.5.IgG1.1 | TAGCAGCTATGTCATGAGCTGGGTCCGCCAGGCTCCAGGGAAG |
| | VH | GGGCTGGAGTGGGTCTCAGGTATTAGTGGTAGTGGTGGTAGCA |
| | | CATACTACGCAGACTCCGTGAAGGGCCGGTTCACCATCTCCAG |
| | | AGACAATTCCAAGAACACGCTGTATCTGCAAATGAACAGCCTG |
| | | AGAGCCGAGGACACGGCCGTATATTACTGTGCGAAAGATCAGG |
| | | ATATTATAGCAGCATACTACTTTGTCTACTGGGGCCAGGGAAC |
| | | CCTGGTCACCGTCTCCTCA |
| 70 | 21D9K kappa | EIVLTQSPGTLSLSPGERATLSCRASQSVSSSYLAWYQQKPGQ |
| | VL | APRLLIYGASSRATGIPDRFSGSGSGTDFTLTISRLEPEDFAV |
| | ILT4.6K | YYCQQYGSSPLTFGGGTKVEIK |
| | VL | |
| 71 | 21D9.IgG1.1 | EGQLLESGGDLVQPGGSLRLSCATSGFTFSNYAMNWVRQAPGK |
| | VH | GLEWISVISVSGGSTYYADSVKGRFTISRDNSKNTLYLQMNSL |
| | ILT4.6.IgG1.1 | RAEDTAVYYCAKDYYYDSGSYYDSFFDYWGQGTLVTVSS |
| | VH | |
| 72 | 21D9K kappa | GAAATTGTGTTGACGCAGTCTCCAGGCACCCTGTCTTTGTCTC |
| | VL DNA | CAGGGGAAAGAGCCACCCTCTCCTGCAGGGCCAGTCAGAGTGT |
| | ILT4.6K | TAGCAGCAGCTACTTAGCCTGGTACCAGCAGAAACCTGGCCAG |
| | VL DNA | GCTCCCAGGCTCCTCATCTATGGTGCATCCAGCAGGGCCACTG |
| | | GCATCCCAGACAGGTTCAGTGGCAGTGGGTCTGGGACAGACTT |
| | | CACTCTCACCATCAGCAGACTGGAGCCTGAAGATTTTGCAGTG |
| | | TATTACTGTCAGCAGTATGGTAGCTCACCTCTCACTTTCGGCG |
| | | GAGGGACCAAGGTGGAGATCAAA |
| 73 | 21D9.IgG1.1 | GAGGGACAGCTGTTGGAGTCTGGGGGAGACTTGGTACAGCCTG |
| | VH DNA | GGGGGTCCCTGAGACTCTCCTGTGCAACCTCTGGATTCACCTT |
| | ILT4.6.IgG1.1 | TAGCAACTATGCCATGAACTGGGTCCGCCAGGCTCCAGGGAAG |
| | VH DNA | GGGCTGGAGTGGATCTCAGTTATTAGTGTTAGTGGTGGTAGCA |
| | | CATACTACGCAGACTCCGTGAAGGGCCGGTTCACCATCTCCAG |
| | | AGACAATTCCAAGAACACGCTGTATCTGCAAATGAATAGCCTG |
| | | AGAGCCGAGGACACGGCCGTATATTACTGTGCGAAAGATTATT |
| | | ACTATGATTCGGGGAGTTATTATGACTCTTTCTTTGACTACTG |
| | | GGGCCAGGGAACCCTGGTCACCGTCTCCTCA |
| 74 | 21D9.b.IgG1.3 | EVQLLESGGGLVQPGGSLRLSCATSGFTFSNYAMNWVRQAPGK |
| | VH | GLEWISVISVSGGSTYYADSVKGRFTISRDNSKNTLYLQMNSL |
| | | RAEDTAVYYCAKDYYYDSGSYYDSFFDYWGQGTLVTVSS |
| 75 | 21D9.c.IgG1.3 | EVQLLESGGDLVQPGGSLRLSCAASGFTFSNYAMNWVRQAPGK |
| | VH | GLEWISVISVSGGSTYYADSVKGRFTISRDNSKNTLYLQMNSL |
| | | RAEDTAVYYCAKDYYYDSGSYYDSFFDYWGQGTLVTVSS |
| 76 | 21D9.b.IgG1.3 | GAGGTGCAGCTGCTGGAGTCTGGAGGAGGACTGGTGCAGCCAG |
| | VH DNA | GAGGCAGCCTGAGGCTGTCCTGCGCCACCTCTGGCTTCACATT |
| | | TTCCAACTATGCCATGAATTGGGTGCGCCAGGCACCTGGCAAG |
| | | GGACTGGAGTGGATCTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCC |
| 77 | 21D9.c.IgG1.3 | GAGGTGCAGCTGCTGGAGTCTGGAGGAGACCTGGTGCAGCCAG |
| | VH DNA | GAGGCAGCCTGAGGCTGTCCTGCGCCGCCTCTGGCTTCACATT |
| | | TTCCAACTATGCCATGAATTGGGTGCGCCAGGCACCTGGCAAG |
| | | GGACTGGAGTGGATCTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCC |
| 78 | 21D9.d.IgG1.3 | EVQLLESGGDLVQPGGSLRLSCATSGFTFSNYAMNWVRQAPGK |
| | VH | GLEWVSVISVSGGSTYYADSVKGRFTISRDNSKNTLYLQMNSL |
| | | RAEDTAVYYCAKDYYYDSGSYYDSFFDYWGQGTLVTVSS |
| 79 | 21D9.d.IgG1.3 | GAGGTGCAGCTGCTGGAGTCTGGAGGAGACCTGGTGCAGCCAG |
| | VH DNA | GAGGCAGCCTGAGGCTGTCCTGCGCCACCTCTGGCTTCACATT |
| | | TTCCAACTATGCCATGAATTGGGTGCGCCAGGCACCTGGCAAG |
| | | GGACTGGAGTGGGTGTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCC |
| 80 | 21D9.e.IgG1.3 | EVQLLESGGGLVQPGGSLRLSCAASGFTFSNYAMNWVRQAPGK |
| | VH | GLEWVSVISVSGGSTYYADSVKGRFTISRDNSKNTLYLQMNSL |
| | ILT4.8.IgG1.3 | RAEDTAVYYCAKDYYYDSGSYYDSFFDYWGQGTLVTVSS |
| | VH | |
| 81 | 21D9.e.IgG1.3 | GAGGTGCAGCTGCTGGAGTCTGGAGGAGGACTGGTGCAGCCAG |
| | VH DNA | GAGGCAGCCTGAGGCTGTCCTGCGCCGCCTCTGGCTTCACATT |
| | ILT4.8.IgG1.3 | TTCCAACTATGCCATGAATTGGGTGCGCCAGGCACCTGGCAAG |
| | VH DNA | GGACTGGAGTGGGTGTCTGTGATCTCTGTGAGCGGCGGCTCTA |
| | | CCTACTATGCCGACAGCGTGAAGGGCCGGTTCACCATCAGCAG |
| | | AGATAACTCCAAGAATACACTGTATCTGCAGATGAACAGCCTG |
| | | CGGGCCGAGGACACAGCCGTGTACTATTGTGCCAAGGACTACT |
| | | ATTACGATTCCGGCTCTTATTACGACTCCTTCTTTGATTACTG |
| | | GGGCCAGGGCACCCTGGTGACAGTGAGCTCC |
| 82 | 21A5 kappa VL | EI [symbol] LTQSPGTLSLSPGERATLSCRASQSVSSSYLAWYQQKPGQ |
| | | APRLLIYGASSRATGIPDRFSGSGSGTDFTLTISRLEPEDFAV |
| | | YYCQQYGSTFGGGTKVEIK |
| 83 | 21A5.IgG1.3 | QVQLVQSGAEVKKPGASVKVSCKASGYTFTDYYLHWVRQAPGQ |
| | VH | GLEWMGIFHPSGDITSSAQNFQGRVTM [symbol] RDTSTSTVYMELSSL |
| | | RSEDTAVYYCARGGVLRYLDWSHAFDIWGQGTMVTVSS |
| 84 | 21A5 kappa VL | GAAATTGCGTTGACGCAGTCTCCAGGCACCCTGTCTTTGTCTC |
| | DNA | CAGGGGAAAGAGCCACCCTCTCCTGCAGGGCCAGTCAGAGTGT |
| | | TAGCAGCAGCTACTTAGCCTGGTACCAGCAGAAACCTGGCCAG |
| | | GCTCCCAGGCTCCTCATCTATGGTGCATCCAGCAGGGCCACTG |
| | | GCATCCCAGACAGGTTCAGTGGCAGTGGGTCTGGGACAGACTT |
| | | CACTCTCACCATCAGCAGACTGGAGCCTGAAGATTTTGCAGTG |
| | | TATTACTGTCAGCAGTATGGTAGCACTTTCGGCGGAGGGACCA |
| | | AGGTGGAGATCAAA |
| 85 | 21A5.IgG1.3 | CAGGTGCAGCTGGTGCAGTCTGGGGCTGAGGTGAAGAAGCCTG |
| | VH DNA | GGGCCTCAGTGAAGGTTTCCTGCAAGGCATCTGGATACACCTT |
| | | CACCGACTACTATTTACACTGGGTGCGACAGGCCCCTGGACAA |
| | | GGGCTTGAGTGGATGGGAATATTCCACCCTAGTGGTGATATCA |
| | | CAAGCAGCGCACAGAACTTCCAGGGCAGAGTCACCATGATCAG |
| | | GGACACGTCCACGAGCACCGTCTACATGGAACTGAGCAGCCTG |
| | | AGATCTGAAGACACGGCCGTGTATTACTGTGCGAGAGGGGGTG |
| | | TATTACGATATCTTGACTGGTCCCATGCTTTTGATATCTGGGG |
| | | CCAAGGGACAATGGTCACCGTCTCTTCA |
| 86 | 21A5.a kappa | EI [symbol] LTQSPGTLSLSPGERATLSCRASQSVSSSYLAWYQQKPGQ |
| | VL | APRLLIYGASSRATGIPDRFSGSGSGTDFTLTISRLEPEDFAV |
| | 21A5.1K | YYCQQYGSTFGGGTKVEIK |
| | ILT4.9K | |
| | VL | |
| 87 | 21A5.a.IgG1.3 | QVQLVQSGAEVKKPGASVKVSCKASGYTFTDYYLHWVRQAPGQ |
| | VH | GLEWMGIFHPSGDITSSAQNFQGRVTM [symbol] RDTSTSTVYMELSSL |
| | ILT4.9.IgG1.3 | RSEDTAVYYCARGGVLRYLDWSHAFDIWGQGTMVTVSS |
| | VH | |
| 88 | 21A5.a kappa | GAGATCGTGCTGACCCAGAGCCCAGGCACACTGTCTCTGAGCC |
| | VL DNA | CAGGAGAGAGGGCCACCCTGTCCTGCAGAGCCTCCCAGTCTGT |
| | 21A5.1K | GAGCTCCTCTTACCTGGCCTGGTATCAGCAGAAGCCAGGACAG |
| | ILT4.9K | GCACCTAGGCTGCTGATCTACGGAGCCAGCTCCAGGGCAACCG |
| | VL | GCATCCCTGACCGCTTCAGCGGCTCCGGCTCTGGCACAGACTT |
| | | CACCCTGACAATCTCTAGGCTGGAGCCCGAGGACTTCGCCGTG |
| | | TACTATTGTCAGCAGTATGGCTCCACCTTTGGCGGCGGCACAA |
| | | AGGTGGAGATCAAG |
| 89 | 21A5.a.IgG1.3 | CAGGTGCAGCTGGTGCAGTCCGGAGCAGAGGTGAAGAAGCCAG |
| | VH DNA | GAGCCTCTGTGAAGGTGAGCTGCAAGGCCTCCGGCTACACCTT |
| | ILT4.9.IgG1.3 | CACAGATTACTATCTGCACTGGGTGCGGCAGGCACCAGGACAG |
| | VH | GGACTGGAGTGGATGGGCATCTTCCACCCTTCTGGCGACATCA |
| | | CAAGCTCCGCCCAGAACTTTCAGGGCCGGGTGACCATGACAAG |
| | | AGATACCAGCACATCCACCGTGTACATGGAGCTGTCTAGCCTG |
| | | AGGTCTGAGGACACCGCCGTGTACTATTGTGCAAGGGGAGGCG |
| | | TGCTGAGGTATCTGGACTGGAGCCACGCCTTTGATATCTGGGG |
| | | CCAGGGCACAATGGTGACCGTGTCCTCT |
| 90 | 10F10 kappa | AIQLTQSPSSLSASVGDRVTITCRASQGISSALAW [symbol] QQKPGKA |
| | VL | PKLLIYDASSLESGVPSRF [symbol] GSGSGTDFTLTISSLKPEDFATY |
| | ILT4.10K | YCQQFNSYPITFGQGTRLEIK |
| | VL | |
| 91 | 10F10.IgG1.3 | QVQLVESGGGVVQPGRSLRLSCAASGFTFSSYAMHWVRQAPGK |
| | VH | GLEWVAIISYDEYNKYYADSVKGRFTISRDNSKNTLYLQMNSL |
| | ILT4.10. IgG1. | RAEDTAVYYCAREWVGIRYWGQGTLVTVSS |
| | 3 | |
| | VH | |
| 92 | 10F10 kappa | GCCATCCAGTTGACCCAGTCTCCATCCTCCCTGTCTGCATCTG |
| | VL DNA | TAGGAGACAGAGTCACCATCACTTGCCGGGCAAGTCAGGGCAT |
| | ILT4.10K | TAGCAGTGCTTTAGCCTGGTTTCAGCAGAAACCAGGGAAAGCT |
| | VL | CCTAAGCTCCTGATCTATGATGCCTCCAGTTTGGAAAGTGGGG |
| | | TCCCATCAAGGTTCACCGGCAGTGGATCTGGGACAGATTTCAC |
| | | TCTCACCATCAGCAGCCTGCAGCCTGAAGATTTTGCAACTTAT |
| | | TACTGTCAACAGTTTAATAGTTACCCGATCACCTTCGGCCAAG |
| | | GGACACGACTGGAGATTAAA |
| 93 | 10F10.IgG1.3 | CAGGTGCAGCTGGTGGAGTCTGGGGGAGGCGTGGTCCAGCCTG |
| | VH DNA | GGAGGTCCCTGAGACTCTCCTGTGCAGCCTCTGGATTCACCTT |
| | ILT4.10.IgG1.3 | CAGTAGCTATGCTATGCACTGGGTCCGCCAGGCTCCAGGCAAG |
| | VH | GGGCTGGAGTGGGTGGCAATTATATCATATGATGAATACAATA |
| | | AATACTACGCAGACTCCGTGAAGGGCCGATTCACCATCTCCAG |
| | | AGACAATTCCAAGAACACGCTGTATCTGCAAATGAACAGCCTG |
| | | AGAGCTGAGGACACGGCTGTGTATTACTGTGCGAGAGAGTGGG |
| | | TGGGGATACGTTACTGGGGCCAGGGAACCCTGGTCACCGTCTC |
| | | CTCA |
| 94 | 10F10.1 kappa | AIQLTQSPSSLSASVGDRVTITCRASQGISSALAWYQQKPGKA |
| | VL | PKLLIYDASSLESGVPSRFTGSGSGTDFTLTISSLQPEDFATY |
| | | YCQQFNSYPITFGQGTRLEIK |
| 95 | 10F10.1 kappa | GCCATCCAGCTGACCCAGTCTCCTAGCTCCCTGTCTGCCAGCG |
| | VL DNA | TGGGCGACAGGGTGACCATCACATGCAGAGCCTCCCAGGGAAT |
| | | CTCTAGCGCCCTGGCCTGGTACCAGCAGAAGCCAGGCAAGGCC |
| | | CCTAAGCTGCTGATCTATGATGCCTCCTCTCTGGAGAGCGGAG |
| | | TGCCATCCAGGTTCACCGGCTCCGGCTCTGGCACAGACTTTAC |
| | | CCTGACAATCAGCTCCCTGCAGCCAGAGGATTTCGCCACATAC |
| | | TATTGTCAGCAGTTCAACAGCTACCCCATCACCTTTGGCCAGG |
| | | GCACACGGCTGGAGATCAAG |
| 96 | 10F10.3 kappa | AIQLTQSPSSLSASVGDRVTITCRASQGISSALAWYQQKPGKA |
| | VL | PKLLIYDASSLESGVPSRFSGSGSGTDFTLTISSLKPEDFATY |
| | ILT4.11K | YCQQFNSYPITFGQGTRLEIK |
| | VL | |
| 97 | 10F10.3 kappa | GCCATCCAGCTGACCCAGTCTCCTAGCTCCCTGTCTGCCAGCG |
| | VL DNA | TGGGCGACAGGGTGACCATCACATGCAGAGCCTCCCAGGGAAT |
| | ILT4.11K | CTCTAGCGCCCTGGCCTGGTACCAGCAGAAGCCAGGCAAGGCC |
| | VL DNA | CCTAAGCTGCTGATCTATGATGCCTCCTCTCTGGAGAGCGGAG |
| | | TGCCATCCAGGTTCAGCGGCTCCGGCTCTGGCACAGACTTTAC |
| | | CCTGACAATCAGCTCCCTGCAGCCAGAGGATTTCGCCACATAC |
| | | TATTGTCAGCAGTTCAACAGCTACCCCATCACCTTTGGCCAGG |
| | | GCACACGGCTGGAGATCAAG |
| 98 | Human IgG1f | ASTKGPSVFPLAPSSKSTSGGTAALGCLVKDYFPEPVTVSWNS |
| | Constant | GALTSGVHTFPAVLQSSGLYSLSSVVTVPSSSLGTQTYICNVN |
| | Region | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 99 | IgG1f | GCTAGCACCAAGGGCCCATCGGTCTTCCCCCTGGCACCCTCCT |
| | Constant | CCAAGAGCACCTCTGGGGGCACAGCGGCCCTGGGCTGCCTGGT |
| | Region DNA | CAAGGACTACTTCCCCGAACCGGTGACGGTGTCGTGGAACTCA |
| | | GGCGCCCTGACCAGCGGCGTGCACACCTTCCCGGCTGTCCTAC |
| | | AGTCCTCAGGACTCTACTCCCTCAGCAGCGTGGTGACCGTGCC |
| | | CTCCAGCAGCTTGGGCACCCAGACCTACATCTGCAACGTGAAT |
| | | CACAAGCCCAGCAACACCAAGGTGGACAAGAGAGTTGAGCCCA |
| | | AATCTTGTGACAAAACTCACACATGCCCACCGTGCCCAGCACC |
| | | TGAACTCCTGGGGGGACCGTCAGTCTTCCTCTTCCCCCCAAAA |
| | | CCCAAGGACACCCTCATGATCTCCCGGACCCCTGAGGTCACAT |
| | | GCGTGGTGGTGGACGTGAGCCACGAAGACCCTGAGGTCAAGTT |
| | | CAACTGGTACGTGGACGGCGTGGAGGTGCATAATGCCAAGACA |
| | | AAGCCGCGGGAGGAGCAGTACAACAGCACGTACCGTGTGGTCA |
| | | GCGTCCTCACCGTCCTGCACCAGGACTGGCTGAATGGCAAGGA |
| | | GTACAAGTGCAAGGTCTCCAACAAAGCCCTCCCAGCCCCCATC |
| | | GAGAAAACCATCTCCAAAGCCAAAGGGCAGCCCCGAGAACCAC |
| | | AGGTGTACACCCTGCCCCCATCCCGGGAGGAGATGACCAAGAA |
| | | CCAGGTCAGCCTGACCTGCCTGGTCAAAGGCTTCTATCCCAGC |
| | | GACATCGCCGTGGAGTGGGAGAGCAATGGGCAGCCGGAGAACA |
| | | ACTACAAGACCACGCCTCCCGTGCTGGACTCCGACGGCTCCTT |
| | | CTTCCTCTATAGCAAGCTCACCGTGGACAAGAGCAGGTGGCAG |
| | | CAGGGGAACGTCTTCTCATGCTCCGTGATGCATGAGGCTCTGC |
| | | ACAACCACTACACGCAGAAGAGCCTCTCCCTGTCCCCGGGTAA |
| | | ATGA |
| 100 | IgG1.3 (or | ASTKGPSVFPLAPSSKSTSGGTAALGCLVKDYFPEPVTVSWNS |
| | IgG1.3f) | GALTSGVHTFPAVLQSSGLYSLSSVVTVPSSSLGTQTYICNVN |
| | heavy chain | HKPSNTKVDKRVEPKSCDKTHTCPPCPAPEAEGAPSVFLFPPK |
| | constant | PKDTLMISRTPEVTCVVVDVSHEDPEVKFNWYVDGVEVHNAKT |
| | region | KPREEQYNSTYRVVSVLTVLHQDWLNGKEYKCKVSNKALPAPI |
| | (L234A, | EKTISKAKGQPREPQVYTLPPSREEMTKNQVSLTCLVKGFYPS |
| | L235E, G237A) | DIAVEWESNGQPENNYKTTPPVLDSDGSFFLYSKLTVDKSRWQ |
| | | QGNVFSCSVMHEALHNHYTQKSLSLSPG |
| 101 | Exemplary | RTVAAPSVFIFPPSDEQLKSGTASVVCLLNNFYPREAKVQWKV |
| | light chain | DNALQSGNSQESVTEQDSKDSTYSLSSTLTLSKADYEKHKVYA |
| | constant | CEVTHQGLSSPVTKSFNRGEC |
| | region | |
| 102 | IgG1f (human | ASTKGPSVFPLAPSSKSTSGGTAALGCLVKDYFPEPVTVSWNS |
| | wild-type | GALTSGVHTFPAVLQSSGLYSLSSVVTVPSSSLGTQTYICNVN |
| | allotype f) | HKPSNTKVDKRVEPKSCDKTHTCPPCPAPELLGGPSVFLFPPK |
| | heavy chain | PKDTLMISRTPEVTCVVVDVSHEDPEVKFNWYVDGVEVHNAKT |
| | constant | KPREEQYNSTYRVVSVLTVLHQDWLNGKEYKCKVSNKALPAPI |
| | region | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPG |
| 103 | IgG1.1f | ASTKGPSVFPLAPSSKSTSGGTAALGCLVKDYFPEPVTVSWNS |
| | Heavy chain | GALTSGVHTFPAVLQSSGLYSLSSVVTVPSSSLGTQTYICNVN |
| | constant | HKPSNTKVDKRVEPKSCDKTHTCPPCPAPEAEGAPSVFLFPPK |
| | region | PKDTLMISRTPEVTCVVVDVSHEDPEVKFNWYVDGVEVHNAKT |
| | (L234A, | KPREEQYNSTYRVVSVLTVLHQDWLNGKEYKCKVSNKALPSSI |
| | L235E, G237A, | EKTISKAKGQPREPQVYTLPPSREEMTKNQVSLTCLVKGFYPS |
| | A330S, P331S) | DIAVEWESNGQPENNYKTTPPVLDSDGSFFLYSKLTVDKSRWQ |
| | | QGNVFSCSVMHEALHNHYTQKSLSLSPG |
| 104 | IgG1fa.P238K | ASTKGPSVFPLAPSSKSTSGGTAALGCLVKDYFPEPVTVSWNS |
| | (or | GALTSGVHTFPAVLQSSGLYSLSSVVTVPSSSLGTQTYICNVN |
| | IgG1.P238K) | HKPSNTKVDKRVEPKSCDKTHTCPPCPAPELLGGKSVFLFPPK |
| | heavy chain | PKDTLMISRTPEVTCVVVDVSHEDPEVKFNWYVDGVEVHNAKT |
| | constant | KPREEQYNSTYRVVSVLTVLHQDWLNGKEYKCKVSNKALPAPI |
| | region | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPG |
| 105 | IgG1.3 heavy | GCGTCGACCAAGGGCCCATCGGTCTTCCCCCTGGCACCCTCCT |
| | chain | CCAAGAGCACCTCTGGGGGCACAGCGGCCCTGGGCTGCCTGGT |
| | constant | CAAGGACTACTTCCCCGAACCGGTGACGGTGTCGTGGAACTCA |
| | region DNA | GGCGCCCTGACCAGCGGCGTGCACACCTTCCCGGCTGTCCTAC |
| | | AGTCCTCAGGACTCTACTCCCTCAGCAGCGTGGTGACCGTGCC |
| | | CTCCAGCAGCTTGGGCACCCAGACCTACATCTGCAACGTGAAT |
| | | CACAAGCCCAGCAACACCAAGGTGGACAAGAGAGTTGAGCCCA |
| | | AATCTTGTGACAAAACTCACACATGCCCACCGTGCCCAGCACC |
| | | TGAAGCCGAAGGGGCCCCGTCAGTCTTCCTCTTCCCCCCAAAA |
| | | CCCAAGGACACCCTCATGATCTCCCGGACCCCTGAGGTCACAT |
| | | GCGTGGTGGTGGACGTGAGCCACGAAGACCCTGAGGTCAAGTT |
| | | CAACTGGTACGTGGACGGCGTGGAGGTGCATAATGCCAAGACA |
| | | AAGCCGCGGGAGGAGCAGTACAACAGCACGTACCGTGTGGTCA |
| | | GCGTCCTCACCGTCCTGCACCAGGACTGGCTGAATGGCAAGGA |
| | | GTACAAGTGCAAGGTCTCCAACAAAGCCCTCCCAGCCCCCATC |
| | | GAGAAAACCATCTCCAAAGCCAAAGGGCAGCCCCGAGAACCAC |
| | | AGGTGTACACCCTGCCCCCATCCCGGGAGGAGATGACCAAGAA |
| | | CCAGGTCAGCCTGACCTGCCTGGTCAAAGGCTTCTATCCCAGC |
| | | GACATCGCCGTGGAGTGGGAGAGCAATGGGCAGCCGGAGAACA |
| | | ACTACAAGACCACGCCTCCCGTGCTGGACTCCGACGGCTCCTT |
| | | CTTCCTCTATAGCAAGCTCACCGTGGACAAGAGCAGGTGGCAG |
| | | CAGGGGAACGTCTTCTCATGCTCCGTGATGCATGAGGCTCTGC |
| | | ACAACCACTACACGCAGAAGAGCCTCTCCCTGTCCCCGGGTTG |
| | | A |
| 106 | Exemplary | CGTACGGTGGCTGCACCATCTGTCTTCATCTTCCCGCCATCTG |
| | light chain | ATGAGCAGTTGAAATCTGGAACTGCCTCTGTTGTGTGCCTGCT |
| | constant | GAATAACTTCTATCCCAGAGAGGCCAAAGTACAGTGGAAGGTG |
| | region DNA | GATAACGCCCTCCAATCGGGTAACTCCCAGGAGAGTGTCACAG |
| | | AGCAGGACAGCAAGGACAGCACCTACAGCCTCAGCAGCACCCT |
| | | GACGCTGAGCAAAGCAGACTACGAGAAACACAAAGTCTACGCC |
| | | TGCGAAGTCACCCATCAGGGCCTGAGCTCGCCCGTCACAAAGA |
| | | GCTTCAACAGGGGAGAGTGTTAG |
| 107 | Human ILT4 | MTPIVTVLIC LGLSLGPRTH VQTGTIPKPT LWAEPDSVIT |
| | precursor, | QGSPVTLSCQ GSLEAQEYRL YREKKSASWI TRIRPELVKN |
| | with signal | GQFHIPSITW EHTGRYGCQY YSRARWSELS DPLVLVMTGA |
| | peptide | YPKPTLSAQP SPVVTSGGRV TLQCESQVAF GGFILCKEGE |
| | (Uni Prot KB | EEHPQCLNSQ PHARGSSRAI FSVGPVSPNR RWSHRCYGYD |
| | Ref. | LNSPYVWSSP SDLLELLVPG VSKKPSLSVQ PGPVVAPGES |
| | Q8N423.4, 24- | LTLQCVSDVG YDRFVLYKEG ERDLRQLPGR QPQAGLSQAN |
| | JUL-2013) | FTLGPVSRSY GGQYRCYGAH NLSSECSAPS DPLDILITGQ |
| | | IRGTPFISVQ PGPTVASGEN VTLLCQSWRQ FHTFLLTKAG |
| | | AADAPLRLRS THEYPKYQAE FPMSPVTSAH AGTYRCYGSL |
| | | NSDPYLLSHP SEPLELVVSG PSMGSSPPPT GPISTPAGPE |
| | | DQPLTPTGSD PQSGLGRHLG VVIGILVAVV LLLLLLLLLF |
| | | LILRHRRQGK HWTSTKRKAD FQHPAGAVGP EPTDRGLQWR |
| | | SSPAADAQEE NLYAAVKDTQ PEDGVEMDTR AAASEAPQDV |
| | | TYAQLHSLTL RRKATEPPPS QEREPPAEPS IYATLAIH |
| 108 | Human mature | QTGTIPKPTL WAEPDSVITQ GSPVTLSCQG SLEAQEYRLY |
| | ILT4, without | REKKSASWIT RIRPELVKNG QFHIPSITWE HTGRYGCQYY |
| | signal | SRARWSELSD PLVLVMTGAY PKPTLSAQPS PVVTSGGRVT |
| | peptide | LQCESQVAFG GFILCKEGEE EHPQCLNSQP HARGSSRAIF |
| | | SVGPVSPNRR WSHRCYGYDL NSPYVWSSPS DLLELLVPGV |
| | | SKKPSLSVQP GPVVAPGESL TLQCVSDVGY DRFVLYKEGE |
| | | RDLRKLPGRQ PQAGLSQANF TLGPVSRSYG GQYRCYGAHN |
| | | LSSECSAPSD PLDILITGQI RGTPFISVQP GPTVASGENV |
| | | TLLCQSWRQF HTFLLTKAGA ADAPLRLRSI HEYPKYQAEF |
| | | PMSPVTSAHA GTYRCYGSLN SDPYLLSHPS EPLELVVSGP |
| | | SMGSSPPPTG PISTPAGPED QPLTPTGSDP QSGLGRHLGV |
| | | VIGILVAVVL LLLLLLLLFL ILRHRRQGKH WTSTKRKADE |
| | | QHPAGAVGPE PTDRGLKWRS SPAADAQEEN LYAAVKDTQP |
| | | EDGVEMDTRA AASEAPQDVT YAQLHSLTLR RKATEPPPSQ |
| | | EREPPAEPSI YATLAIH |
| 109 | Human ILT4 | GTIPKPTL WAEPDSVITQ GSPVTLSCQG SLEAQEYRLY |
| | ECD region | REKKSASWIT RIRPELVKNG QFHIPSITWE HTGRYGCQYY |
| | | SRARWSELSD PLVLVMTGAY PKPTLSAQPS PVVTSGGRVT |
| | | LQCESQVAFG GFILCKEGEE EHPQCLNSQP HARGSSRAIF |
| | | SVGPVSPNRR WSHRCYGYDL NSPYVWSSPS DLLELLVPGV |
| | | SKKPSLSVQP GPVVAPGESL TLQCVSDVGY DRFVLYKEGE |
| | | RDLRQLPGRQ PQAGLSQANF TLGPVSRSYG GQYRCYGAHN |
| | | LSSECSAPSD PLDILITGQI RGTPFISVQP GPTVASGENV |
| | | TLLCQSWRQF HTFLLTKAGA ADAPLRLRSI HEYPKYQAEF |
| | | PMSPVTSAHA GTYRCYGSLN SDPYLLSHPS EPLELVVSGP |
| | | SMGSSPPPTG PISTPAGPED QPLTPTGSDP QSGLGRHLGV |
| 110 | Human ILT4 | MTPIVTVLIC LGLSLGPRTH VQTGTIPKPT LWAEPDSVIT |
| | precursor, | QGSPVTLSCQ GSLEAQEYRL YREKKSASWI TRIRPELVKN |
| | with signal | GQFHIPSITW EHTGRYGCQY YSRARWSELS DPLVLVMTGA |
| | peptide, | YPKPTLSAQP SPVVTSGGRV TLQCESQVAF GGFILCKEGE |
| | isoform 2 | EEHPQCLNSQ PHARGSSRAI FSVGPVSPNR RWSHRCYGYD |
| | (NCBI Ref. | LNSPYVWSSP SDLLELLVPG VSKKPSLSVQ PGPVVAPGES |
| | NP_001074447.1, | LTLQCVSDVG YDRFVLYKEG ERDLRQLPGR QPQAGLSQAN |
| | 12-SEP- | FTLGPVSRSY GGQYRCYGAH NLSSECSAPS DPLDILITGQ |
| | 2013) | IRGTPFISVQ PGPTVASGEN VTLLCQSWRQ FHTFLLTKAG |
| | | AADAPLRLRS IHEYPKYQAE FPMSPVTSAH AGTYRCYGSL |
| | | NSDPYLLSHP SEPLELVVSG PSMGSSPPPT GPISTPGPED |
| | | QPLTPTGSDP QSGLGRHLGV VIGILVAVVL LLLLLLLLFL |
| | | ILRHRRQGKH WTSTQRKADF QHPAGAVGPE PTDRGLQWRS |
| | | SPAADAQEEN LYAAVKDTQP EDGVEMDTRA AASEAPQDVT |
| | | YAQLHSLTLR RKATEPPPSQ EREPPAEPSI YATLAIH |
| 111 | Human mature | QTGTIPKPTL WAEPDSVITQ GSPVTLSCQG SLEAQEYRLY |
| | ILT4, isoform | REKKSASWIT RIRPELVKNG QFHIPSITWE HTGRYGCQYY |
| | 2, without | SRARWSELSD PLVLVMTGAY PKPTLSAQPS PVVTSGGRVT |
| | signal | LQCESQVAFG GFILCKEGEE EHPQCLNSQP HARGSSRAIF |
| | peptide | SVGPVSPNRR WSHRCYGYDL NSPYVWSSPS DLLELLVPGV |
| | | SKKPSLSVQP GPVVAPGESL TLQCVSDVGY DRFVLYKEGE |
| | | RDLRQLPGRQ PQAGLSQANF TLGPVSRSYG GQYRCYGAHN |
| | | LSSECSAPSD PLDILITGQI RGTPFISVQP GPTVASGENV |
| | | TLLCQSWRQF HTFLLTKAGA ADAPLRLRSI HEYPKYQAEF |
| | | PMSPVTSAHA GTYRCYGSLN SDPYLLSHPS EPLELVVSGP |
| | | SMGSSPPPTG PISTPGPEDQ PLTPTGSDPQ SGLGRHLGVV |
| | | IGILVAVVLL LLLLLLLFLI LRHRRQGKHW TSTQRKADFQ |
| | | HPAGAVGPEP TDRGLQWRSS PAADAQEENL YAAVKDTQPE |
| | | DGVEMDTRAA ASEAPQDVTY AQLHSLTLRR KATEPPPSQE |
| | | REPPAEPSIY ATLAIH |
| 112 | Human ILT4 | GTIPKPTL WAEPDSVITQ GSPVTLSCQG SLEAQEYRLY |
| | isoform 2, | REKKSASWIT RIRPELVKNG QFHIPSITWE HTGRYGCQYY |
| | ECD | SRARWSELSD PLVLVMTGAY PKPTLSAQPS PVVTSGGRVT |
| | | LQCESQVAFG GFILCKEGEE EHPQCLNSQP HARGSSRAIF |
| | | SVGPVSPNRR WSHRCYGYDL NSPYVWSSPS DLLELLVPGV |
| | | SKKPSLSVQP GPVVAPGESL TLQCVSDVGY DRFVLYKEGE |
| | | RDLRQLPGRQ PQAGLSQANF TLGPVSRSYG GQYRCYGAHN |
| | | LSSECSAPSD PLDILITGQI RGTPFISVQP GPTVASGENV |
| | | TLLCQSWRQF HTFLLTKAGA ADAPLRLRSI HEYPKYQAEF |
| | | PMSPVTSAHA GTYRCYGSLN SDPYLLSHPS EPLELVVSGP |
| | | SMGSSPPPTG PISTPGPEDQ PLTPTGSDPQ SGLGRHLGV |
| 113 | 21D9.IgG1.3 | EGQLLESGGD LVQPGGSLRL SCATSGFTFS NYAMNWVRQA |
| | HC | PGKGLEWISV ISVSGGSTYY ADSVKGRFTI SRDNSKNTLY |
| | | LQMNSLRAED TAVYYCAKDY YYDSGSYYDS FFDYWGQGTL |
| | | VTVSSASTKG PSVFPLAPSS KSTSGGTAAL GCLVKDYFPE |
| | | PVTVSWNSGA LTSGVHTFPA |
| | | VLQSSGLYSL SSVVTVPSSS LGTQTYICNV NHKPSNTKVD |
| | | KRVEPKSCDK THTCPPCPAPEAEGAPSVFL FPPKPKDTLM |
| | | ISRTPEVTCV VVDVSHEDPE VKFNWYVDGV |
| | | EVHNAKTKPREEQYNSTYRV VSVLTVLHQD WLNGKEYKCK |
| | | VSNKALPAPI EKTISKAKGQ PREPQVYTLPPSREEMTKNQ |
| | | VSLTCLVKGF YPSDIAVEWE SNGQPENNYK TTPPVLDSDG |
| | | SFFLYSKLTVDKSRWQQGNV FSCSVMHEAL HNHYTQKSLS |
| | | LSPG |
| 114 | 10F10.4 VL | AIQLTQSPSSLSASVGDRVTITCRASQGISSALAW [symbol] QQKPGKA |
| | | PKLLIYDASSLESGVPSRF [symbol] GSGSGTDFTLTISSLQPEDFATY |
| | | YCQQFNSYPITFGQGTRLEIK |
| 115 | 10F10.4 VL | GCCATCCAGCTGACCCAGAGCCCTAGCTCCCTGTCTGCCAGCG |
| | DNA | TGGGCGACAGGGTGACCATCACATGCAGAGCCTCCCAGGGAAT |
| | | CTCTAGCGCCCTGGCCTGGTTCCAGCAGAAGCCAGGCAAGGCC |
| | | CCTAAGCTGCTGATCTACGATGCCTCCTCTCTGGAGTCCGGCG |
| | | TGCCCTCTAGGTTCTCCGGCTCTGGCAGCGGCACCGACTTTAC |
| | | CCTGACAATCAGCTCCCTGCAGCCAGAGGATTTTGCCACATAC |
| | | TATTGTCAGCAGTTCAACTCTTATCCCATCACCTTTGGCCAGG |
| | | GCACACGGCTGGAGATCAAG |
| 116 | 10F10.4 LC | AIQLTQSPSSLSASVGDRVTITCRASQGISSALAW [symbol] QQKPGKA |
| | | PKLLIYDASSLESGVPSRF [symbol] GSGSGTDFTLTISSLQPEDFATY |
| | | YCQQFNSYPITFGQGTRLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 117 | 10F10.4 LC | GCCATCCAGCTGACCCAGAGCCCTAGCTCCCTGTCTGCCAGCG |
| | DNA | TGGGCGACAGGGTGACCATCACATGCAGAGCCTCCCAGGGAAT |
| | | CTCTAGCGCCCTGGCCTGGTTCCAGCAGAAGCCAGGCAAGGCC |
| | | CCTAAGCTGCTGATCTACGATGCCTCCTCTCTGGAGTCCGGCG |
| | | TGCCCTCTAGGTTCTCCGGCTCTGGCAGCGGCACCGACTTTAC |
| | | CCTGACAATCAGCTCCCTGCAGCCAGAGGATTTTGCCACATAC |
| | | TATTGTCAGCAGTTCAACTCTTATCCCATCACCTTTGGCCAGG |
| | | GCACACGGCTGGAGATCAAGCGTACGGTGGCCGCCCCGTCCGT |
| | | GTTTATCTTCCCTCCATCGGACGAGCAGCTCAAGTCCGGTACC |
| | | GCGAGCGTGGTCTGCCTGCTGAACAATTTCTACCCGCGCGAAG |
| | | CTAAAGTGCAATGGAAGGTCGATAACGCACTTCAGTCCGGGAA |
| | | CAGCCAGGAATCTGTGACCGAGCAGGACTCCAAGGATTCGACC |
| | | TATTCCCTGTCCTCGACTCTCACCCTGTCAAAGGCCGACTACG |
| | | AGAAGCACAAGGTCTACGCCTGCGAAGTGACCCATCAGGGCTT |
| | | GTCCTCACCCGTGACTAAGAGCTTCAACCGGGGAGAGTGT |
| 118 | Cyno ILT4 | QAGILPKPMLWAEPDRVITQGSPVTLRCQGNLEARGYHLYRER |
| | 9152 | KSASWITLIRPELVKKGQFPIPSITWEDAGRYRCQYYSHSWWS |
| | extracellular | EHSDPLELVVTGAYRKPTLSALPSPVVASGGNVTLQCDSRVAL |
| | domain, | DGFILCKEGEDEHSQRLNSQPRTRGSSRAVFSVGPVSPSRRWS |
| | including | YRCYGYELHSRYVWSLPSDLLELLVPGVSKKPSLSVQPGPVVA |
| | His-Avi Tag | GGDKLTLQCGSDAGYDRFALYKEGERDFLQRPGQQLQAGLAQA |
| | | NFTLDPVRGSHGGQYRCYGAHNLSSEWSAPSDPLDILISAGPH |
| | | SGLRRECDPAVSVTGMDGHFLSDQGGSSSPGGGSGGGSEQKLI |
| | | SEEDLGHHHHHHGLNDIFEAQKIEWHE |
| 119 | hILT4H | QTGTIPKPTLWAEPDSVITQGSPVTLSCQGSLEAQEYRLYREK |
| | extracellular | KSASWITRIRPELVKNGQFHIPSITWEHTGRYGCQYYSRARWS |
| | domain plus | ELSDPLVLVMTGAYPKPTLSAQPSPVVTSGGRVTLQCESQVAF |
| | HIS-Avi tag | GGFILCKEGEEEHPQCLNSQPHARGSSRAIFSVGPVSPNRRWS |
| | (used in | HRCYGYDLNSPYVWSSPSDLLELLVPGVSKKPSLSVQPGPVVA |
| | Example 2) | PGESLTLQCVSDVGYDRFVLYKEGERDLRQLPGRQPQAGLSQA |
| | | NFTLGPVSRSYGGQYRCYGAHNLSSESSAPSDPLDILITGQIR |
| | | GTPFISVQPGPTVASGENVTLLCQSWRQFHTFLLTKAGAADAP |
| | | LRLRSIHEYPKYQAEFPMSPVTSAHAGTYRCYGSLNSDPYLLS |
| | | HPSEPLELVVSGPSMGSSPPPTGPISTPAGPEDQPLTPTGSDP |
| | | QSGLGRHLGSPGGGSGGGSEQKLISEEDLGHHHHHHGLNDIFE |
| | | AQKIEWHE |
| 120 | Residues 70- | ITRIRPEL |
| | 77 of hILT4 | |
| 121 | Residues 78- | VKNGQFHIPSITWEHTGRYGCQY |
| | 100 of hILT4 | |
| 122 | Residues 70- | ITRIRPELVKNGQFHIPSITWEHTGRYGCQY |
| | 100 of hILT4 | |
| 123 | Residues 154 | ILCKEGEEEHPQCLNSQPHARGSSRAIF |
| | to 181 of | |
| | hILT4 | |
| 124 | Residues 425 | SSPPPTGPIS |
| | to 434 of | |
| | hILT4 | |
| 125 | 9G4 (ILT4.1) | SYYWN |
| | VH CDR1 | |
| 126 | 9G4 VH CDR2 | YIYYSGSTKYNPSLKS |
| 127 | 9G4 VH CDR3 | SGWYYFDY |
| 128 | 9G4 VL CDR1 | RASQSVSSYLA |
| 129 | 9G4 VL CDR2 | DASNRAT |
| 130 | 9G4 VL CDR3 | QQRSYWPWT |
| 131 | 9C8 VH CDR1 | SSDIN |
| 132 | 9C8 VH CDR2 | WMNPNSGHTGYAQKFQD |
| 133 | 9C8 VH CDR3 | GGNSIDWGFSYYGLDV |
| 134 | 9C8 VL CDR1 | RASQGISSALA |
| 135 | 9C8 VL CDR2 | DASSLES |
| 136 | 9C8 VL CDR3 | QQFNSYPFT |
| 137 | 2H2 VH CDR1 | NYAIS |
| 138 | 2H2 VH CDR2 | GIIPILATANYAPKFQG |
| 139 | 2H2 VH CDR3 | SSITMIRGAYLYYYDGMDV |
| 140 | 2H2 VL CDR1 | RASQSVSSSYLA |
| 141 | 2H2 VL CDR2 | GASSRAT |
| 142 | 2H2 VL CDR3 | QQYGSSYT |
| 143 | 2E5 VH CDR1 | NYGMH |
| 144 | 2E5 VH CDR2 | VIWYDGSNEYYAESVKG |
| 145 | 2E5 VH CDR3 | DPFYGSGNYFDY |
| 146 | 2E5 VL CDR1 | RASQSVSSYLA |
| 147 | 2E5 VL CDR2 | DASNRAT |
| 148 | 2E5 VL CDR3 | QQRSNWPPWT |
| 149 | 24E5 VH CDR1 | SYVMS |
| 150 | 24E5 VH CDR2 | GISGSGGSTYYADSVKG |
| 151 | 24E5 VH CDR3 | DQDIIAAYYFVY |
| 152 | 24E5 VL CDR1 | RASQGISSWLA |
| 153 | 24E5 VL CDR2 | AASSLQS |
| 154 | 24E5 VL CDR3 | QQYNSYPLT |
| 155 | 21D9 VH CDR1 | NYAMN |
| 156 | 21D9 VH CDR2 | VISVSGGSTYYADSVKG |
| 157 | 21D9 VH CDR3 | DYYYDSGSYYDSFFDY |
| 158 | 21D9 VL CDR1 | RASQSVSSSYLA |
| 159 | 21D9 VL CDR2 | GASSRAT |
| 160 | 21D9 VL CDR3 | QQYGSSPLT |
| 161 | 21A5 VH CDR1 | DYYLH |
| 162 | 21A5 VH CDR2 | IFHPSGDITSSAQNFQG |
| 163 | 21A5 VH CDR3 | GGVLRYLDWSHAFDI |
| 164 | 21A5 VL CDR1 | RASQSVSSSYLA |
| 165 | 21A5 VL CDR2 | GASSRAT |
| 166 | 21A5 VL CDR3 | QQYGST |
| 167 | 10F10 VH CDR1 | SYAMH |
| 168 | 10F10 VH CDR2 | IISYDEYNKYYADSVKG |
| 169 | 10F10 VH CDR3 | EWVGIRY |
| 170 | 10F10 VL CDR1 | RASQGISSALA |
| 171 | 10F10 VL CDR2 | DASSLES |
| 172 | 10F10 VL CDR3 | QQFNSYPIT |
| 173 | Residues 127- | SAQPSPVVTSGGRVTL |
| | 142 of hILT4 | |
| 174 | Residues 182- | SVGPVSPNRRWSHRCYGYDLNSPYVWSSPSDL |
| | 213 of hILT4 | |
| 175 | Residues 378- | QAEFPMSPVTSAHAG |
| | 392 of hILT4 | |
| 176 | 21D9.e, IgG1f | EVQLLESGGGLVQPGGSLRLSCAASGFTFSNYAMNWVRQAPGK |
| | (Same Fc as | GLEWVSVISVSGGSTYYADSVKGRFTISRDNSKNTLYLQMNSL |
| | SEQ ID NO: 98 | RAEDTAVYYCAKDYYYDSGSYYDSFFDYWGQGTLVTVSSASTK |
| | w/o C-term. | GPSVFPLAPSSKSTSGGTAALGCLVKDYFPEPVTVSWNSGALT |
| | K) | SGVHTFPAVLQSSGLYSLSSVVTVPSSSLGTQTYICNVNHKPS |
| | | NTKVDKRVEPKSCDKTHTCPPCPAPELLGGPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPAPIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGNV |
| | | FSCSVMHEALHNHYTQKSLSLSPG |
| 177 | 21D9.e.IgG1.1f | EVQLLESGGGLVQPGGSLRLSCAASGFTFSNYAMNWVRQAPGK |
| | (Fc is the | GLEWVSVISVSGGSTYYADSVKGRFTISRDNSKNTLYLQMNSL |
| | same as SEQ | RAEDTAVYYCAKDYYYDSGSYYDSFFDYWGQGTLVTVSSASTK |
| | ID NO: 103) | GPSVFPLAPSSKSTSGGTAALGCLVKDYFPEPVTVSWNSGALT |
| | | SGVHTFPAVLQSSGLYSLSSVVTVPSSSLGTQTYICNVNHKPS |
| | | NTKVDKRVEPKSCDKTHTCPPCPAPEAEGAPSVFLFPPKPKDT |
| | | LMISRTPEVTCVVVDVSHEDPEVKFNWYVDGVEVHNAKTKPRE |
| | | EQYNSTYRVVSVLTVLHQDWLNGKEYKCKVSNKALPSSIEKTI |
| | | SKAKGQPREPQVYTLPPSREEMTKNQVSLTCLVKGFYPSDIAV |
| | | EWESNGQPENNYKTTPPVLDSDGSFFLYSKLTVDKSRWQQGNV |
| | | FSCSVMHEALHNHYTQKSLSLSPG |
| 178 | 21D9.e.IgG4_ | EVQLLESGGGLVQPGGSLRLSCAASGFTFSNYAMNWVRQAPGK |
| | S228P | GLEWVSVISVSGGSTYYADSVKGRFTISRDNSKNTLYLQMNSL |
| | | RAEDTAVYYCAKDYYYDSGSYYDSFFDYWGQGTLVTVSSASTK |
| | | GPSVFPLAPCSRSTSESTAALGCLVKDYFPEPVTVSWNSGALT |
| | | SGVHTFPAVLQSSGLYSLSSVVTVPSSSLGTKTYTCNVDHKPS |
| | | NTKVDKRVESKYGPPCPPCPAPEFLGGPSVFLFPPKPKDTLMI |
| | | SRTPEVTCVVVDVSQEDPEVQFNWYVDGVEVHNAKTKPREEQF |
| | | NSTYRVVSVLTVLHQDWLNGKEYKCKVSNKGLPSSIEKTISKA |
| | | KGQPREPQVYTLPPSQEEMTKNQVSLTCLVKGFYPSDIAVEWE |
| | | SNGQPENNYKTTPPVLDSDGSFFLYSRLTVDKSRWQQGNVFSC |
| | | SVMHEALHNHYTQKSLSLS |
| 179 | hIgG4 S228P | ASTKGPSVFPLAPCSRSTSESTAALGCLVKDYFPEPVTVSWNS |
| | (Fc of SEQ ID | GALTSGVHTFPAVLQSSGLYSLSSVVTVPSSSLGTKTYTCNVD |
| | NO: 178) | HKPSNTKVDKRVESKYGPPCPPCPAPEFLGGPSVFLFPPKPKD |
| | | TLMISRTPEVTCVVVDVSQEDPEVQFNWYVDGVEVHNAKTKPR |
| | | EEQFNSTYRVVSVLTVLHQDWLNGKEYKCKVSNKGLPSSIEKT |
| | | ISKAKGQPREPQVYTLPPSQEEMTKNQVSLTCLVKGFYPSDIA |
| | | VEWESNGQPENNYKTTPPVLDSDGSFFLYSRLTVDKSRWQQGN |
| | | VFSCSVMHEALHNHYTQKSLSLS |

1-63. (canceled)

64. A method of treating a subject having cancer, comprising administering to the subject an isolated antibody that specifically binds to human ILT4 (hILT4), wherein the antibody comprises a heavy chain and a light chain, wherein the heavy chain comprises a heavy chain variable region (VH) comprising VH complementary determining region 1 (CDR1), CDR2 and CDR3 comprising the amino acid sequences of SEQ ID Nos: 155, 156, and 157, respectively, and wherein the light chain comprises a light chain variable region (VL) comprising VL CDR1, CDR2 and CDR3 comprising the amino acid sequences of SEQ ID Nos: 158, 159, and 160, respectively.

65. The method of claim 64, wherein:
a. the VH comprises an amino acid sequence that is at least 90% identical to the amino acid sequence of SEQ ID NO: 71 and the VL comprises an amino acid sequence that is at least 90% identical to the amino acid sequence of SEQ ID NO: 70;
b. the VH comprises an amino acid sequence that is at least 90% identical to the amino acid sequence of SEQ ID NO: 74 and the VL comprises an amino acid sequence that is at least 90% identical to the amino acid sequence of SEQ ID NO: 70;
c. the VH comprises an amino acid sequence that is at least 90% identical to the amino acid sequence of SEQ ID NO: 75 and the VL comprises an amino acid sequence that is at least 90% identical to the amino acid sequence of SEQ ID NO: 70;
d. the VH comprises an amino acid sequence that is at least 90% identical to the amino acid sequence of SEQ ID NO: 78 and the VL comprises an amino acid sequence that is at least 90% identical to the amino acid sequence of SEQ ID NO: 70; or
e. the VH comprises an amino acid sequence that is at least 90% identical to the amino acid sequence of SEQ ID NO: 80 and the VL comprises an amino acid sequence that is at least 90% identical to the amino acid sequence of SEQ ID NO: 70.

66. The method of claim 64, wherein:
a. the VH comprises an amino acid sequence that is at least 95% identical to the amino acid sequence of SEQ ID NO: 71 and the VL comprises an amino acid sequence that is at least 95% identical to the amino acid sequence of SEQ ID NO: 70;
b. the VH comprises an amino acid sequence that is at least 95% identical to the amino acid sequence of SEQ ID NO: 74 and the VL comprises an amino acid sequence that is at least 95% identical to the amino acid sequence of SEQ ID NO: 70;
c. the VH comprises an amino acid sequence that is at least 95% identical to the amino acid sequence of SEQ ID NO: 75 and the VL comprises an amino acid sequence that is at least 95% identical to the amino acid sequence of SEQ ID NO: 70;
d. the VH comprises an amino acid sequence that is at least 95% identical to the amino acid sequence of SEQ ID NO: 78 and the VL comprises an amino acid sequence that is at least 95% identical to the amino acid sequence of SEQ ID NO: 70; or
e. the VH comprises an amino acid sequence that is at least 95% identical to the amino acid sequence of SEQ ID NO: 80 and the VL comprises an amino acid sequence that is at least 95% identical to the amino acid sequence of SEQ ID NO: 70.

67. The method of claim 64, wherein:
a. the VH comprises the amino acid sequence of SEQ ID NO: 71;
b. the VH comprises the amino acid sequence of SEQ ID NO: 74;
c. the VH comprises the amino acid sequence of SEQ ID NO: 75;
d. the VH comprises the amino acid sequence of SEQ ID NO: 78; or
e. the VH comprises the amino acid sequence of SEQ ID NO: 80.

68. The method of claim 67, wherein the antibody comprises a heavy chain constant region comprising the amino acid sequence of any one of SEQ ID Nos: 98, 100, 102, 103, 104 or 179.

69. The method of claim 68, wherein the heavy chain constant region comprises the amino acid sequence of any one of SEQ ID Nos: 100, 102, 103, 104 or 179 and further comprises a C-terminal lysine residue.

70. The method of claim 68, wherein the heavy chain constant region comprises the amino acid sequence of SEQ ID NO: 100.

71. The method of claim 70, wherein the heavy chain constant region further comprises a C-terminal lysine residue.

72. The method of claim 64, wherein the VL comprises the amino acid sequence of SEQ ID NO: 70.

73. The method of claim 64, wherein:
a. the VH comprises the amino acid sequence of SEQ ID NO: 71 and the VL comprises the amino acid sequence of SEQ ID NO: 70;
b. the VH comprises the amino acid sequence of SEQ ID NO: 74 and the VL comprises the amino acid sequence of SEQ ID NO: 70;
c. the VH comprises the amino acid sequence of SEQ ID NO: 75 and the VL comprises the amino acid sequence of SEQ ID NO: 70;
d. the VH comprises the amino acid sequence of SEQ ID NO: 78 and the VL comprises the amino acid sequence of SEQ ID NO: 70; or
e. the VH comprises the amino acid sequence of SEQ ID NO: 80 and the VL comprises the amino acid sequence of SEQ ID NO: 70.

74. The method of claim 73, wherein the antibody comprises a heavy chain constant region comprising the amino acid sequence of any one of SEQ ID Nos: 98, 100, 102, 103, 104 or 179.

75. The method of claim 74, wherein the heavy chain constant region comprises the amino acid sequence of any one of SEQ ID Nos: 100, 102, 103, 104 or 179 and further comprises a C-terminal lysine residue.

76. The method of claim 74, wherein the heavy chain constant region comprises the amino acid sequence of SEQ ID NO: 100.

77. The method of claim 76, wherein the heavy chain constant region further comprises a C-terminal lysine residue.

78. The method of claim 64, wherein the antibody is an IgG antibody.

79. The method of claim 78, wherein the antibody is an IgG1, IgG2 or IgG4 antibody.

80. The method of claim 79, wherein the antibody is a human IgG4 antibody comprising an S228P substitution (EU numbering).

81. The method of claim 64, wherein the heavy chain lacks a C-terminal lysine residue.

82. The method of claim 64, wherein the antibody is a full-length antibody, and wherein the heavy chain comprises or does not comprise a C-terminal lysine residue.

83. The method of claim 64, wherein the heavy chain comprises the amino acid sequence of SEQ ID NO: 13.

84. The method of claim 83, wherein the heavy chain further comprises a C-terminal lysine residue.

85. The method of claim 64, wherein the light chain comprises the amino acid sequence of SEQ ID NO: 11.

86. The method of claim 64, wherein the heavy chain comprises the amino acid sequence of SEQ ID NO: 12, SEQ ID NO: 13, SEQ ID NO: 36, SEQ ID NO: 38, SEQ ID NO: 40, SEQ ID NO: 176, SEQ ID NO: 177, or SEQ ID NO: 178.

87. The method of claim 64, wherein: (a) the heavy chain comprises the amino acid sequence of SEQ ID NO: 12 and the light chain comprises the amino acid sequence of SEQ ID NO: 11, (b) the heavy chain comprises the amino acid sequence of SEQ ID NO: 13 and the light chain comprises the amino acid sequence of SEQ ID NO: 11, (c) the heavy chain comprises the amino acid sequence of SEQ ID NO: 36 and the light chain comprises the amino acid sequence of SEQ ID NO: 11, (d) the heavy chain comprises the amino acid sequence of SEQ ID NO: 38 and the light chain comprises the amino acid sequence of SEQ ID NO: 11, (e) the heavy chain comprises the amino acid sequence of SEQ ID NO: 40 and the light chain comprises the amino acid sequence of SEQ ID NO: 11, (f) the heavy chain comprises the amino acid sequence of SEQ ID NO: 176 and the light chain comprises the amino acid sequence of SEQ ID NO: 11, (g) the heavy chain comprises the amino acid sequence of SEQ ID NO: 177 and the light chain comprises the amino acid sequence of SEQ ID NO: 11, or (h) the heavy chain comprises the amino acid sequence of SEQ ID NO: 178 and the light chain comprises the amino acid sequence of SEQ ID NO: 11.

88. The method of claim 64, wherein the VH comprises the amino acid sequence of SEQ ID NO: 80.

89. The method of claim 88, wherein the antibody comprises a heavy chain constant region comprising the amino acid sequence of any one of SEQ ID Nos: 98, 100, 102, 103, 104 or 179.

90. The method of claim 89, wherein the heavy chain constant region comprises the amino acid sequence of any one of SEQ ID Nos: 100, 102, 103, 104 or 179 and further comprises a C-terminal lysine residue.

91. A method of treating a subject having cancer, comprising administering an isolated antibody that specifically binds to human ILT4 (hILT4), the antibody comprising a VH comprising the amino acid sequence of SEQ ID NO: 80 and a VL comprising the amino acid sequence of SEQ ID NO: 70.

92. The method of claim 91, wherein the antibody is an IgG antibody.

93. The method of claim 92, wherein the antibody is an IgG1, IgG2 or IgG4 antibody.

94. The method of claim 93, wherein the antibody is a human IgG4 antibody comprising an S228P substitution (EU numbering).

95. The method of claim 91, wherein the antibody comprises a heavy chain that lacks a C-terminal lysine residue.

96. The method of claim 91, wherein the antibody a full-length antibody comprising a heavy chain that comprises or does not comprise a C-terminal lysine residue.

97. The method of claim 91, wherein the antibody comprises a heavy chain constant region comprising the amino acid sequence of any one of SEQ ID Nos: 98, 100, 102, 103, 104 or 179.

98. The method of claim 97, wherein the heavy chain constant region comprises the amino acid sequence of any one of SEQ ID Nos: 100, 102, 103, 104 or 179 and further comprises a C-terminal lysine residue.

99. A method of treating a subject having cancer, comprising administering to the subject an isolated antibody that specifically binds to human ILT4 (hILT4), wherein the antibody comprises a VH comprising the amino acid sequence of SEQ ID NO: 80, a VL comprises the amino acid sequence of SEQ ID NO: 70, and a heavy chain constant region comprising the amino acid sequence of SEQ ID NO: 100.

100. The method of claim 99, wherein the heavy chain constant region further comprises a C-terminal lysine residue.

101. A method of treating a subject having cancer, comprising administering to the subject an isolated antibody that specifically binds to human ILT4 (hILT4), wherein the antibody comprises a heavy chain comprising the amino acid sequence of SEQ ID NO: 13 and a light chain comprising the amino acid sequence of SEQ ID NO: 11.

102. The method of claim 101, wherein the heavy chain further comprises a C-terminal lysine residue.

103. The method of claim 101, wherein the antibody comprises a heavy chain consisting of the amino acid sequence of SEQ ID NO: 13 and a light chain consisting of the amino acid sequence of SEQ ID NO: 11.

104. The method of claim 101, wherein the antibody comprises a heavy chain consisting of the amino acid sequence of SEQ ID NO: 13 and a C-terminal lysine, and a light chain consisting of the amino acid sequence of SEQ ID NO: 11.

105. The method of claim 64, wherein the method further comprises administering a second therapy.

106. The method of claim 105, wherein the second therapy is radiotherapy, surgery or administration of a second agent.

107. The method of claim 105, wherein the second therapy is administration of a second agent and the second agent is an immunostimulatory agent.

108. The method of claim 107, wherein the immunostimulatory agent is an antagonist of an immunosuppressive molecule, e.g., the PD-1/PD-L1, an CTLA-4 and LAG-3, or an agonist of an immunostimulating molecule, e.g., GITR and OX40.
